SEQUENCE LISTING (22 sequences)

SEQ ID NO: 1 - DNA, 3200 bp, Homo sapiens
  source 1..3200: mol_type = genomic DNA
aagcgagcggcgtctccaccagcatctgccgcggccgcctttgcccgaagcccggggacgaaccgacggaccgaccgcctggcgcacggacgcgggcgctcgctttgtgttcggggctagcgtcggcgaggcttgagcttgcagcgcgcggcttccctgctttctcgcggccaccccggctccggcggcctcggcgcgcgaggggctggaggtgcgggagccgctctccgccggtcggtccccgcgcggctgagcccaggccgccagcgccgcggccccgtgcggtgtccctgagctcctgctccccgccgggctgctccgagcaacggtgcttcggagctccaaactcgggctgccggggcaagtgtcttcatgaacccagaggatgtccgggaagcactacaagggtcctgaagtcagttgttgcatcaaatacttcatatttggcttcaatgtcatattttggtttttgggaataacatttcttggaattggactgtgggcatggaatgaaaaattttctgtgttcctgggaattattttcttcctggagctcactgccggagttctagcatttgttttcaaagactggatcaaagaccagctgtatttctttataaacaacaacatcagagcatatcgggatgacattgatttgcaaaacctcatagacttcacccaggaatattggcagtgctgtggggcttttggagctgatgattggaacctaaatatttacttcaattgcacagattccaatgcaagtcgagagcgatgtggcgttccattctcctgctgcactaaagatcccgcagaagatgtcatcaacactcagtgtggctatgatgccaggcaaaaaccagaagttgaccagcagattgtaatctacacgaaaggctgtgtgccccagtttgagaagtggttgcaggacaatttaaccatcgttgctggtattttcataggcattgcattgctgcagatatttgggatatgcctggcccagaatttggttagcgatatcgaagctgtcagggcgagctggtagaccccctgcaaccgctgctgcaagacactggacagacccagctttcgggaccctcccgcgtgccgaactgatcttcgagctgcatggacctaatcacagatgcagcctgcagtctcgcctaatggagctgccattaggggagtgtaaaactgggaaatgctgctcactgacagaattaaaaaaaaaaataaccagtatgaaagtcgttgcgccgtgaatctctactgtagccatgaatttatggacagttagatgcttaccaaaaaagaaaaaaagggagggtaggggacccagatgtacttgaatgtgcagaaaatacattcttgtcctcatcttccgtaattggagggctgggagaggcagctttgctcttcaccacaccttggacggaccaccttctttctgttccatggcctgaaggagtgcatctcctcaaagactcagcccctcacctgggagggcagtggtttgtgggcatccctccatgtacattttaggaaacacttgcaactctcatctgaagaagaaaacaactcatctttgggttcagattttgtgatggtattcagcaagtcacttgggcgagcacacttggtctatcctggaaagtctccttataagagaagttgtgtatttcatgtgcaccgagcaagggcattggaagacgtcatgaggctgtattttagcaggactgatcgtttttctaagtagacctgagctttgtttatcagtgaaattcaaggagaaaatgaggttaatgaagaggtatcagttaaatatccccttcttctcaccctgccaaaattagcagttggatttttggaaactctggaatattctgggtcattttgttttgtatgtttgttgtttttcgtcttccaaaggtgaaagctatgatacagttccacttaaattttagtgttttcttactcagctcaagcattaatttttgattaagtcttaatctgcatgacctgtgaatctgaatccatcatctccctttcctgccagcttttctacaaacattgaaatatgttatttggtcagcacttatttcctaggttcacagccttgggaggttgtggcatgtcctcccagtctggctgggaagagaccagctgtaccatccaaatgcttccctggtcttgatgatctcttccagagtcgatctgagtggccttttctgcaccctccccttctttctctttgaatggaattaaacccaatttggaaacaacattgacccagtcaaaagcttctaatggtttctttttcttcctccagttttagtttgcttttattaaaaaaagaaaatagtgcatggccatagctccttcagttctcttattgcagactaaccatcaggatggtatcaaagcacaaatactttggaggggaatgcgttgaactggggcaagtactctgtaacacaaagtgggaaaccacttcctggtgctgccgctcctgcccccactttaggtgggagggacgagttttgccctctagattttaatccagctggtgtccaccggatgttgccctcctggggagcagatatcagtctgtggaactctgggaaaaccacaggcacatttttcggtgcggacagatttgccagcacataactgggcagccagctagaatactttgtggaaattaagcgaggttttccatttcagccccatggtgcatggtggtggccgatgaatgtgtcagtctgctcagagaaaggacaaaaaggaaattattttcaaaactgtgttcactgtttgggtgtgtgtatggctctgcatgtgtgtgtttttgtctctgtataggtagaggtattcacatcttactccgactgtaaggttgtcttacttcatctctgcccccaccacagttgccattttgtaatgtccttccaacatggagaagacacgagctctctccagttggcatcatttgtcttttttgttgattgcctcattctccagtgaactccatctggccaattgattcagaatcaggcaagatccctgccctttggcacatccactgaaaggccaaacagcaagtccgagtgagttttaaatattaattaatcaccctttattttttacacttgagagtgattgtaataaaggctgtcattaataaacttggttctacctta

SEQ ID NO: 2 - DNA, 393 bp, Homo sapiens
  source 1..393: mol_type = genomic DNA
atgtctggacgtggaaagcaaggcggcaaagctcgggcaaaagctaaaacgcgttcttccagggccggtcttcagtttccagttggccgtgtgcaccgcctcctccgcaaaggcaactactccgaacgagtcggggccggcgctccagtgtacctggcagcggtgctggaatatctgacggccgagatcttagagctagctggcaacgcggctcgcgacaataagaagacccgcatcatcccgcgccacctgcagctagccatccgcaacgacgaggagctaaataagcttctaggtcgcgtgaccatcgcgcagggcggtgtcctgcccaacatccaggccgtattgctgcctaagaagacggagagccaccataaggccaagggcaagtga

SEQ ID NO: 3 - DNA, 1887 bp, synthetic construct
  source 1..1887: mol_type = other DNA
gttcgttgcaacaaattgatgagcaatgcttttttataatgccaactttgtacaaaaaagttggcatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattaaagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtacccaactttcttgtacaaagttggcattataagaaagcattgcttatcaatttgttgcaacgaac

SEQ ID NO: 4 - DNA, 788 bp, Homo sapiens
  source 1..788: mol_type = genomic DNA
gcctttgccgcctactgaagccggcgctgctctgtggggctctggccgcgcctggcctggccgggaccatgtgcgcgtcccgggacgactggcgctgtgcgcgctccatgcacgagttttccgccaaggacatcgacgggcacatggttaacctggacaagtaccggggcttcgtgtgcatcgtcaccaacgtggcctcccagtgaggcaagaccgaagtaaactacactcagctcgtcgacctgcacgcccgatacgctgagtgtggtttgcggatcctggccttcccgtgtaaccagttcgggaagcaggagccagggagtaacgaagagatcaaagagttcgccgcgggctacaacgtcaaattcgatatgttcagcaagatctgcgtgaacggggacgacgcccacccgctgtggaagtggatgaagatccaacccaagggcaagggcatcctgggaaatgccatcaagtggaacttcaccaagttcctcatcgacaagaacggctgcgtggtgaagcgctacggacccatggaggagcccctggtgatagagaaggacctgccccactatttctagctccacaagtgtgtggccccgcccgagcccctgcccacgcccttggagccttccaccggcactcatgacggcctgcctgcaaacctgctggtggggcagacccgaaaatccagcgtgcaccccgccggaggaaggtcccatggcctgctgggcttggctcggcgcccccacccctggctaccttgtgggaataaacagacaaattag

SEQ ID NO: 5 - DNA, 2208 bp, synthetic construct
  source 1..2208: mol_type = other DNA
gttcgttgcaacaaattgatgagcaatgcttttttataatgccaactttgtacaaaaaagttggcaccatgggacaggccctgggtatcaagagctgtgactttcaggcagcaagaaacaatgaggagcaccacaccaaggccctcagctcccggcgcctctttgtgaggagggggcagcccttcaccatcatcctgtacttccgcgctccagtccgtgcatttctgcctgccctgaagaaggtggccctcactgcacaaactggagagcagccttccaagatcaacaggacccaagccacattcccaatttccagtctgggggaccgaaagtggtggagtgcagtggtggaggagagagatgcccagtcctggaccatctctgtgaccacacctgcggacgctgtcattggccactactcgcttctgctgcaggtctcaggcaggaagcaactcctcttgggtcagttcacactgctttttaacccctggaatagagaggatgctgtgttcctgaagaatgaggctcagcgcatggagtacttgttgaaccagaatggtctcatctacctgggtacagctgactgcatccaggcagagtcctgggactttggccagttcgagggggatgtcattgacctcagcctgcgcttgctgagcaaggacaagcaggtagagaagtggagccagccggtgcacgtggcccgtgtgttgggtgccttgctgcattttctcaaggagcagagggtcctgcccaccccgcagacccaggccacccaggaaggggccttgctgaacaagcgccggggcagcgtgcccatcctgcggcagtggctcaccggccgaggccgacctgtgtatgatggccaggcctgggtgttggctgctgttgcttgcacagtgctgcgatgcctgggaatccctgcctgcgtggtgaccacgtttgcctcagcacagggcaccggtgggcgtcttctcatagatgaatactataatgaggagggacttcagaacggagaaggccagagaggcagaatctggatcttccagacttccacagagtgctggatgacgcggcctgccttgccccagggttatgatggatggcagattctgcacccaagtgctcctaatggaggtggagtcctggggtcctgtgatctggtgccggtcagagcagtcaaggaggggacgctggggctgaccccagcagtgtcagacctttttgctgccataaatgcctcatgtgtggtctggaagtgctgtgaggatgggacactggagttgactgactccaacacaaagtatgttggcaacaacatcagcaccaagggtgtgggcagtgaccgctgcgaggacatcactcagaactacaagtatcctgaagggtctcttcaggaaaaagaggtgctggagagagtcgagaaagagaaaatggaacgtgagaaagacaacggcatccgtcctcccagtctcgagactgccagtcctctgtacctgctcttgaaagcacccagctccctacccctgagaggggatgcccagatctcagtgacgctggttaatcacagtgagcaggagaaggcagtgcagctggcaattggggtccaggctgtacactacaacggtgtccttgctgccaagctctggaggaagaagctgcacctcacgctcagtgccaacctggaaaagataataaccatcggcctgttcttctccaattttgagcgaaacccacccgagaacaccttccttagactcaccgccatggcaacacactctgaatccaaccttagctgctttgctcaggaagacattgccatttgtagaccacaccttgccatcaagatgccagagaaagcagagcagtatcaacccctcacagcctcagtcagcctccagaactccctagatgcccccatggaggactgtgtgatctccatcctgggaagggggctcattcacagagagaggagctacagattccgttcagtgtggcctgaaaacaccatgtgtgccaagttccagttcacgccaacacatgtggggctccagagactcactgtggaagtggactgcaacatgttccagaacctaaccaactataaaagcgtcaccgtggtagcccctgaactatcagctttgccaactttcttgtacaaagttggcattataagaaagcattgcttatcaatttgttgcaacgaac

SEQ ID NO: 6 - DNA, 2868 bp, synthetic construct
  source 1..2868: mol_type = other DNA
gttcgttgcaacaaattgatgagcaatgcttttttataatgccaactttgtacaaaaaagttggcaccatggaggagctgcaggatgattatgaagacatgatggaggagaatctggagcaggaggaatatgaagacccagacatccacgagtcccagatggaggagccggcagctcacgacaccgaggcaacagccacagactaccacaccacatcacacccgggtacccacaaggtctatgtggagctgcaggagctggtgatggacgaaaagaaccaggagctgagatggatggaggcggcgcgctgggtgcaactggaggagaacctgggggagaatggggcctggggccgcccgcacctctctcacctcaccttctggagcctcctagagctgcgtagagtcttcaccaagggtactgtcctcctagacctgcaagagacctccctggctggagtggccaaccaactgctagacaggtttatctttgaagaccagatccggcctcaggaccgagaggagctgctccgggccctgctgcttaaacacagccacgctggagagctggaggccctggggggtgtgaagcctgcagtcctgacacgctctggggatccttcacagcctctgctcccccaacactcctcactggagacacagctcttctgtgagcagggagatgggggcacagaagggcactcaccatctggaattctggaaaagattcccccggattcagaggccacgttggtgctagtgggccgcgccgacttcctggagcagccggtgctgggcttcgtgaggctgcaggaggcagcggagctggaggcggtggagctgccggtgcctatacgcttcctctttgtgttgctgggacctgaggccccccacatcgattacacccagcttggccgggctgctgccaccctcatgtcagagagggtgttccgcatagatgcctacatggctcagagccgaggggagctgctgcactccctagagggcttcctggactgcagcctagtgctgcctcccaccgatgccccctccgagcaggcactgctcagtctggtgcctgtgcagagggagctacttcgaaggcgctatcagtccagccctgccaagccagactccagcttctacaagggcctagacttaaatgggggcccagatgaccctctgcagcagacaggccagctcttcgggggcctggtgcgtgatatccggcgccgctacccctattacctgagtgacatcacagatgcattcagcccccaggtcctggctgccgtcatcttcatctactttgctgcactgtcacccgccatcaccttcggcggcctcctgggagaaaagacccggaaccagatgggagtgtcggagctgctgatctccactgcagtgcagggcattctcttcgccctgctgggggctcagcccctgcttgtggtcggcttctcaggacccctgctggtgtttgaggaagccttcttctcgttctgcgagaccaacggtctagagtacatcgtgggccgcgtgtggatcggcttctggctcatcctgctggtggtgttggtggtggccttcgagggtagcttcctggtccgcttcatctcccgctatacccaggagatcttctccttcctcatttccctcatcttcatctatgagactttctccaagctgatcaagatcttccaggaccacccactacagaagacttataactacaacgtgttgatggtgcccaaacctcagggccccctgcccaacacagccctcctctcccttgtgctcatggccggtaccttcttctttgccatgatgctgcgcaagttcaagaacagctcctatttccctggcaagctgcgtcgggtcatcggggacttcggggtccccatctccatcctgatcatggtcctggtggatttcttcattcaggatacctacacccagaaactctcggtgcctgatggcttcaaggtgtccaactcctcagcccggggctgggtcatccacccactgggcttgcgttccgagtttcccatctggatgatgtttgcctccgccctgcctgctctgctggtcttcatcctcatattcctggagtctcagatcaccacgctgattgtcagcaaacctgagcgcaagatggtcaagggctccggcttccacctggacctgctgctggtagtaggcatgggtggggtggccgccctctttgggatgccctggctcagtgccaccaccgtgcgtcccgtcacccatgccaacgccctcactgtcatgggcaaagccagcaccccaggggctgcagcccagatccaggaggtcaaagagcagcggatcagtggactcctggtcgctgtgcttgtgggcctgtccatcctcatggagcccatcctgtcccgcatccccctggctgtactgtttggcatcttcctctacatgggggtcacgtcgctcagcggcatccagctctttgaccgcatcttgcttctgttcaagccacccaagtatcacccagatgtgccctacgtcaagcgggtgaagacctggcgcatgcacttattcacgggcatccagatcatctgcctggcagtgctgtgggtggtgaagtccacgccggcctccctggccctgcccttcgtcctcatcctcactgtgccgctgcggcgcgtcctgctgccgctcatcttcaggaacgtggagcttcagtgtctggatgctgatgatgccaaggcaacctttgatgaggaggaaggtcgggatgaatacgacgaagtggccatgcctgtgttgccaactttcttgtacaaagttggcattataagaaagcattgcttatcaatttgttgcaacgaac

SEQ ID NO: 7 - DNA, 421 bp, Homo sapiens
  source 1..421: mol_type = genomic DNA
gcttgctgggaagagaggcgaagccaggtcacctttcaaggacccagaagtagggttttggcctaggtaacggggcagagatgtggttcgagattctccccggactctccgtcatgggcgtgtgcttgttgattccaggactggctactgcgtacatccacaggttcactaacgggggcaaggaaaaaagggttgctcattttgggtatcactggagtctgatggaaagagataggcgcatctctggagttgatcgttactatgtgtcaaagggtttggagaacattgattaaggaagcattttcctgattgatgaaaaaaataactcagttatggccatctacccctgctagaaggttacagtgtattatgtagcatgcaatgtgttatgtagtgcttaataaaaataaaatgaaaaaaa

SEQ ID NO: 8 - DNA, 1560 bp, Homo sapiens
  source 1..1560: mol_type = genomic DNA
tcacttaagtgcagatttgtaataaaaatcttaatgccagtggcatgctttttggatatataagaagctaaccacttggagtatcatatttgagaggtcagaaaagtccacagttaaagatcggtttataatttacgaagaaaatagaaagttttgtttctctgagttgaaatttgccaagcacggcgggaaatattgcaagtttttggcacaaggctttgtgcttcccttataatttgagatctgcgtgaagcctgagggttcggggatcattatctgagaaaaaccgggcagttcggtgtagacaatttttatatttttggctttttttgaggtgtaacaaacacaactcgggatccgagaggacactctgcggctgccagcgaggcgggctggacagcgcaccaatcacggcgcagctccgccctatataaacgggcgggcgcagcgccgcggctcgagtcccggccagtgcctctgcttccggctcgaattgctctcgctcacgcttgccttcaacatgtccgagactgcgcctgccgcgcccgctgctccggcccctgccgagaagactcccgtgaagaagaaggcccgcaagtctgcaggtgcggccaagcgcaaagcgtctgggcccccggtgtccgagctcattactaaagctgttgccgcctccaaggagcgcagcggcgtatctttggccgctctcaagaaagcgctggcagccgctggctatgacgtggagaagaacaacagccgcatcaagctgggtctcaagagcctggtgagcaagggcaccctggtgcagaccaagggcaccggcgcgtcgggttccttcaaactcaacaagaaggcggcctctggggaagccaagcctaaggctaaaaaggcaggcgcggccaaggccaagaagccagcaggagcggcgaagaagcccaagaaggcgacgggggcggccacccccaagaagagcgccaagaagaccccaaagaaggcgaagaagccggctgcagctgctggagccaaaaaagcgaaaagcccgaaaaaggcgaaagcagccaagccaaaaaaggcgcccaagagcccagcgaaggccaaagcagttaaacccaaggcggctaaaccaaagaccgccaagcccaaggcagccaagccaaagaaggcggcagccaagaaaaagtagaaagttcctttggccaactgcttagaagcccaacacaacccaaaggctcttttcagagccacccaccgctctcagtaaaagagctgttgcactattagggggcgtggctcgggaaaacgctgctaagcaggggcgggtctcccgggaacaaagtcggggagaggagtgggattttgtgtgtctccggagctatttttgactaaggcgtcgcgtcgcccaagccggagtgcagtggcgtcatctcgattttgcgttctcgagtgtcggagttgaacccatttgggcctcccttgtgctttgcacttttagcaggccctggcctccagatagcatgggaaaaaaaatgttgggattttcccgggtttctaagctgggttttt

SEQ ID NO: 9 - DNA, 3815 bp, Homo sapiens
  source 1..3815: mol_type = genomic DNA
cagtcgccgccctctctcccgcgcgcgcccggcgcttcggctccgctccctgtgcggcatttaaacttccctcttagaactgcttttgtggcatcccataagttttgaacccattaaaagagccagtaaatcctgtggaaaatggccttcagtgatcttacatcgaggactgtgcatctttatgataattggatcaaagatgctgatccaagagttgaagattggctcctcatgtcctcgcctctgccacaaaccatcctcctaggattctatgtctattttgtcacttccttgggaccaaagctcatggaaaatcgcaagccctttgaactcaagaaagcaatgataacgtacaattttttcatagtactcttttctgtgtatatgtgttatgagtttgtgatgtctggctggggtataggttattcatttcgatgtgacattgttgactattcacggtcacccacagctttgaggatggcacgtacctgctggctttattacttctccaaatttattgagctattagatacgatcttttttgttctgcgcaagaaaaatagccaagtgactttccttcatgtattccatcataccatcatgccgtggacctggtggtttggagtcaaatttgctgcaggtggtttgggaacattccatgcccttctaaatacagctgtacatgtagtcatgtattcctactatggactttctgcattggggccagcctaccagaagtatttgtggtggaaaaaatatttgacatcattacagcttgtccagtttgttattgtcgccatccacataagccagttctttttcatggaggattgcaagtatcagtttccagtctttgcgtgcatcattatgagttacagtttcatgtttccgctgctctttctccatttttggtaccgtgcttacaccaaaggtcagaggttgcccaaaactgtgaaaaatggaacttgcaaaaacaaagataattgaagcccaacataagtctatgatcgaaactgatacattgtcttccttgacaatcaagagatatttacctatgcagtgcattttgtatatttttcaaaactaagagcttgtatttttatggtaagttattgggatgtctgatatttgagagcccgaagcttccagataacagtcttctgataattagagcctacagcagccagaatttgtttttgtttttgtttttgtttttttagctgcttttaaacctaatccaaaggttttgtttaaaacattttgttgcagtgaaaaaaagatatgaagcaatacagtactcttcaaagaagtccaataaagatgaaaaatatcattaggtattttgagcacagacggagatccatgtgataaaatagattccttctgctgggtctggaaagtctggaaaccacctgtaggccctggctttgtcatttattgactgtattcagaagatactatatttgctctagagttaactcccattttagcaagctagcacaggtgaaaattgagtgagtttttgataacttgtcatttaaaatcattaatgataattttcaatggatcttttcagtagccatcaccagttttgctgataagacttcctcacaacccatttgttgtacaaactgtttcaaagtagcaatcctttgggttagtacacttgataccaagtttcattcagttgattattttcaaaacaaggtgatttgttttaatgggttaatgaatactttgctattactgttttacaattaactttgtatatctctggaaagaggtgaatttgtcaatgaaaaaagtattgtgtagttcagtgggaaaaacctgttgtctgttatagtatcacatcactttcttacattgtcatggtttaaattattatcttggtgaaatatataggttagatacttaaatgttcatattattagcacactacaggtactacccttaaatcataaataatattacccattgtacacagtttccagatatgagttagagtggctgtgaagcaactagaggcaaattgtggcacagagaatatccaggggaaaattgattatgtaagcaagggctgttctactttgagagagagacagacagactacataatagtaatatattatataattaatgttatagatacactgaacaataaaacattttctggaattatgaattaattaacaacctggatttctgtttccagacctataaaccatgtaatgaaggactgaaattcgtttggcatacactttgtttctttaaaattgctagtttctttctgttatttttacattttcttgtcagagaatcaaaactattagtcagtagagttttgtccatgaaataatatttggacatttgtgaatttttccctattttttttcttcttttatttatactcaattttgaagcactgtttatgtttgtaggactttaaacaattagtacttaaagccccagttaattttgaacacacagagatacttattgtactctgtgtacagtaagtatttttatgtttacacttaccagattcataaggttatttgcctttaagtgatcttttgtgattttacttgattacagcatgagaaggtaaaggttgataaatgggagtaattacatatatatatataatttattatttttttttctgaagcagagttttactcttgttgcccaggctagagtgcaatggcacaatctcggttcactgcaacctctgcctcccaggttcaagtgattctcctgtctcagcctcccaagtagctgggattacaggtgcccgccaccatgctcagctaatttttgtatttttagtagagacagggttggccaggctggtctcgagctcctgacctcaggtgatcctcccacctcggcctcccaaagttctgggattacaggggtgagccactgtgcccggccataattttatatttttagaaaataaagaatgaaggacaaaagagcacagctaaatgaaactgctgctctaatttattccactggaaaggtctcaggactccttaactgttttccaggtttggctcttcatatctaacctgtgctaaaatgagaaggtatgctaggtgctgttagaaatcacaccatttccaaagaacccaagtagtatagagaccaaaatgaggcaaaaataaaagagatgaaacaggagagtttatttcttgcatactttccgaccttcatcatacaacctctcctaacctccctgtagtctttaaaatgtttaacttgcctataagctaacatgtaataaaacacatactcaattatatgaatagaagtggagagccagaatgctacaaaagaaatcacagctgctagaagtatcctccatagaaacatcaacatgattgtggatcaaaatgattttcactggatatggaatttgtatgggccatatttattaaaagagttctgtgtggtcacatagagttcctttgggatttcatcccacttttcaggacttaatttgtttgggtttgcttacctaacaagcaacaacaacaacaaaataaatagttccaaaatctagtttattacataaatctctatgaatccttgagggcctatatctatattataaataaatacaaatatagatttttaaatatctatgggactttgccatttacagccttaagtataaaattacgagattatattctttccattaccctttatttctgctaactttttaaagactggaacattctgtggatgttgtcaaagtttgagtttgtttttccctgtgtattataataaatttgtggtattgc

SEQ ID NO: 10 - DNA, 1782 bp, synthetic construct
  source 1..1782: mol_type = other DNA
gttcgttgcaacaaattgatgagcaatgcttttttataatgccaactttgtacaaaaaagttggcatggtgactgcagccatgctgctacagtgctgcccagtgcttgcccggggccccacaagcctcctaggcaaggtggttaagactcaccagttcctgtttggtattggacgctgtcccatcctggctacccaaggaccaaactgttctcaaatccaccttaaggcaacaaaggctggaggagattctccatcttgggcgaagggccactgtcccttcatgctgtcggaactccaggatgggaagagcaagattgtgcagaaggcagccccagaagtccaggaagatgtgaaggctttcaagacaggaaactatgtcttcagttatgaccagtttttcagggacaagatcatggagaagaaacaggatcacacctaccgtgtgttcaagactgtgaaccgctgggctgatgcatatccctttgcccaacatttctctgaggcatctgtggcctcaaaggatgtgtccgtctggtgtagtaatgattacctgggcatgagccgacaccctcaggtcttgcaagccacacaggagaccctgcagcgtcatggtgttggagctggtggcacccgcaacatctcaggcaccagtaagtttcatgtggagcttgagcaggagctggctgagctgcaccagaaggactcagccctgctcttctcctcctgctttgttgccaatgactctactctcttcaccttggccaagatcctgccagggtgcgagatttactcagacgcaggcaaccatgcttccatgatccaaggtatccgtaacagtggagcagccaagtttgtcttcaggcacaatgaccctgaccacctaaagaaacttctagagaagtctaaccctaagatacccaaaattgtggcctttgagactgtccactccatggatggtgccatctgtcccctcgaggagttgtgtgatgtgtcccaccagtatggggccctgaccttcgtggatgaggtccatgctgtaggactgtatgggtcccggggcgctgggattggggagcgtgatggaattatgcataagattgacatcatctctggaactcttggcaaggcctttggctgtgtgggcggctacattgccagcacccgtgacttggtggacatggtgcgctcctatgctgcaggcttcatctttaccacttctctgccccccatggtgctctctggagctctagaatctgtgcggctgctcaagggagaggagggccaagccctgaggcgagcccaccagcgcaatgtcaagcacatgcgccagctactcatggacaggggccttcctgtcatcccctgccccagccacatcaaccccatccgggtgggcaatgcagcactcaacagcaagctctgtgatctcctgctctccaagcatggcatctatgtgcaggccatcaactacccaactgtcccccggggtgaagagctcctgcgcttggcaccctccccccaccacagccctcagatgatggaagattttgtggagaagctgctgctggcttggactgcggtggggctgcccctccaggatgtgtctgtggctgcctgcaatttctgtcgccgtcctgtacactttgagctcatgagtgagtgggaacgttcctacttcgggaacatggggccccagtatgtcaccacctatgcctgcccaactttcttgtacaaagttggcattataagaaagcattgcttatcaatttgttgcaacgaac

SEQ ID NO: 11 - DNA, 873 bp, Homo sapiens
  source 1..873: mol_type = genomic DNA
agaggtgctttgaagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccatgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcatgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctg

SEQ ID NO: 12 - DNA, 474 bp, synthetic construct
  source 1..474: mol_type = other DNA
gttcgttgcaacaaattgatgagcaatgcttttttataatgccaactttgtacaaaaaagttggcatgtcggccctcagcctcctcattctgggcctgctcatggcagtgccacctgccagctgtcagcaaggcctggggaaccttcagccctggatgcagggccttatcgcggtggccgtgttcctggtcctcgttgcaatcgcctttgcagtcaaccacttctggtgccaggaggagccggagcctgcacacatgatcctgaccgtcggaaacaaggcagatggagtcctggtgggaacagatggaaggtactcttcgatggcggccagtttcaggtccagtgagcatgagaatgcctatgagaatgtgcccgaggaggaaggcaaggtccgcagcaccccgatgtacccaactttcttgtacaaagttggcattataagaaagcattgcttatcaatttgttgcaacgaac

SEQ ID NO: 13 - DNA, 3479 bp, Homo sapiens
  source 1..3479: mol_type = genomic DNA
acttcctggcaattctagccaccatgagtccaggggctatagccctttgctctgcccgttgctcagcaagttacttggggttccagtttgataagaaaagacttcctgtggaggaatctgaagggaaggaggaggagctggcccattcctgcctgggaggttgtggaagaaggaagatggccagagctttgtgtccactgcaagccctctggcttctggagtgggtgctgctgctcttgggaccttgtgctgcccctccagcctgggccttgaacctggacccagtgcagctcaccttctatgcaggccccaatggcagccagtttggattttcactggacttccacaaggacagccatgggagagtggccatcgtggtgggcgccccgcggaccctgggccccagccaggaggagacgggcggcgtgttcctgtgcccctggagggccgagggcggccagtgcccctcgctgctctttgacctccgtgatgagacccgaaatgtaggctcccaaactttacaaaccttcaaggcccgccaaggactgggggcgtcggtcgtcagctggagcgacgtcattgtggcctgcgccccctggcagcactggaacgtcctagaaaagactgaggaggctgagaagacgcccgtaggtagctgctttttggctcagccagagagcggccgccgcgccgagtactccccctgtcgcgggaacaccctgagccgcatttacgtggaaaatgattttagctgggacaagcgttactgtgaagcgggcttcagctccgtggtcactcaggccggagagctggtgcttggggctcctggcggctattatttcttaggtctcctggcccaggctccagttgcggatattttctcgagttaccgcccaggcatccttttgtggcacgtgtcctcccagagcctctcctttgactccagcaacccagagtacttcgacggctactgggggtactcggtggccgtgggcgagttcgacggggatctcaacactacagaatatgtcgtcggtgcccccacttggagctggaccctgggagcggtggaaattttggattcctactaccagaggctgcatcggctgcgcggagagcagatggcgtcgtattttgggcattcagtggctgtcactgacgtcaacggggatgggaggcatgatctgctggtgggcgctccactgtatatggagagccgggcagaccgaaaactggccgaagtggggcgtgtgtatttgttcctgcagccgcgaggcccccacgcgctgggtgcccccagcctcctgctgactggcacacagctctatgggcgattcggctctgccatcgcacccctgggcgacctcgaccgggatggctacaatgacattgcagtggctgccccctacgggggtcccagtggccggggccaagtgctggtgttcctgggtcagagtgaggggctgaggtcacgtccctcccaggtcctggacagccccttccccacaggctctgcctttggcttctcccttcgaggtgccgtagacatcgatgacaacggatacccagacctgatcgtgggagcttacggggccaaccaggtggctgtgtacagagctcagccagtggtgaaggcctctgtccagctactggtgcaagattcactgaatcctgctgtgaagagctgtgtcctacctcagaccaagacacccgtgagctgcttcaacatccagatgtgtgttggagccactgggcacaacattcctcagaagctatccctaaatgccgagctgcagctggaccggcagaagccccgccagggccggcgggtgctgctgctgggctctcaacaggcaggcaccaccctgaacctggatctgggcggaaagcacagccccatctgccacaccaccatggccttccttcgagatgaggcagacttccgggacaagctgagccccattgtgctcagcctcaatgtgtccctaccgcccacggaggctggaatggcccctgctgtcgtgctgcatggagacacccatgtgcaggagcagacacgaatcgtcctggactgtggggaagatgacgtatgtgtgccccagcttcagctcactgccagcgtgacgggctccccgctcctagttggggcagataatgtcctggagctgcagatggacgcagccaacgagggcgagggggcctatgaagcagagctggccgtgcacctgccccagggcgcccactacatgcgggccctaagcaatgtcgagggctttgagagactcatctgtaatcagaagaaggagaatgagaccagggtggtgctgtgtgagctgggcaaccccatgaagaagaacgcccagataggaatcgcgatgttggtgagcgtggggaatctggaagaggctggggagtctgtgtccttccagctgcagatacggagcaagaacagccagaatccaaacagcaagattgtgctgctggacgtgccggtccgggcagaggcccaagtggagctgcgagggaactcctttccagcctccctggtggtggcagcagaagaaggtgagagggagcagaacagcttggacagctggggacccaaagtggagcacacctatgagctccacaacaatggccctgggactgtgaatggtcttcacctcagcatccaccttccgggacagtcccagccctccgacctgctctacatcctggatatacagccccaggggggccttcagtgcttcccacagcctcctgtcaaccctctcaaggtggactgggggctgcccatccccagcccctcccccattcacccggcccatcacaagcgggatcgcagacagatcttcctgccagagcccgagcagccctcgaggcttcaggatccagttctcgtaagctgcgactcggcgccctgtactgtggtgcagtgtgacctgcaggagatggcgcgcgggcagcgggccatggtcacggtgctggccttcctgtggctgcccagcctctaccagaggcctctggatcagtttgtgctgcagtcgcacgcatggttcaacgtgtcctccctcccctatgcggtgcccccgctcagcctgccccgaggggaagctcaggtgtggacacagctgctccgggccttggaggagagggccattccaatctggtgggtgctggtgggtgtgctgggtggcctgctgctgctcaccatcctggtcctggccatgtggaaggtcggcttcttcaagcggaaccggccacccctggaagaagatgatgaagagggggagtgatggtgcagcctacactattctagcaggagggttgggcgtgctacctgcaccgccccttctccaacaagttgcctccaagctttgggttggagctgttccattgggtcctcttggtgtcgtttccctcccaacagagctgggctaccccccctcctgctgcctaataaagagactgagccctga

SEQ ID NO: 14 - DNA, 5053 bp, Homo sapiens
  source 1..5053: mol_type = genomic DNA
agaagaaggggagaggaggttgtgtgggacaaggtgctcctgacagaaggatgccacagctgagcctgtcctcgctgggcctttggccaatggcagcatccccgtggctgctcctgctgctggttggggcctcctggctcctggcccgcatcctggcctggacctataccttctatgacaactgctgccgcctccggtgtttcccgcaacccccgaaacggaattggttcttgggtcacctgggcctgattcacagctcggaggaaggtctcctatacacacaaagcctggcatgcaccttcggtgatatgtgctgctggtgggtggggccctggcacgcaatcgtccgcatcttccaccccacctacatcaagcctgtgctctttgctccagctgccattgtaccaaaggacaaggtcttctacagcttcctgaagccctggctgggggatgggctcctgctgagtgctggtgaaaagtggagccgccaccgtcggatgctgacgcctgccttccatttcaacatcctgaagccctatatgaagattttcaatgagagtgtgaacatcatgcatgccaagtggcagctcctggcctcagagggtagtgcccgtctggacatgtttgagcacatcagcctcatgaccttggacagtctgcagaaatgtgtcttcagctttgacagccattgccaggagaagcccagtgaatatattgccgccatcttggagctcagtgcccttgtgacaaaaagacaccagcagatcctcctgtacatagacttcctgtattatctcacccctgatgggcagcgtttccgcagggcctgccgcctggtgcacgacttcacagatgccgtcatccaggagcggcgccgcaccctccctagccagggtgttgatgacttcctccaagccaaggccaaatccaagactttggacttcattgatgtactcctgctgagcaaggatgaagatgggaagaagttgtccgatgaggacataagagcagaagctgacacctttatgtttgagggccatgacaccacagccagtggtctctcctgggtcctgtaccaccttgcaaagcacccggaataccaggagcgctgtcggcaggaggtgcaagagcttctgaaggaccgtgagcctaaagagattgaatgggacgacctggcccagctgcccttcctgaccatgtgcattaaggagagcctgaggctgcatcccccagtccctgccgtctctcgctgctgcacccaagacattgtgctcccagacggccgggtcatccccaaaggcattatctgcctcatcagtgtttttggaacccatcacaacccagccgtgtggccggaccctgaggtctatgacccctttcgctttgacccaaagaacatcaaggagaggtcacctctggcttttattcccttctcagcagggcccaggaactgcatcgggcaggcgttcgcgatggcggagatgaaggtggtcctggggctcacgctgctgcgcttccgcgtcctgcctgaccacaccgagccccgcaggaagccggagctggtcctgcgcgcagagggcggactttggctgcgggtggagcccctgagctgagttctgcagagacccactctgaccccactaaaatgacccctgattcatcaaaagtgaggcctagaattaccctaagaccctgttccacagtcctgtattccatcctagatatctactcaaaataattgagacaagtgttcaaacagaaagacgcttgtgcgtgaatgttcatggcagccctattcacagtagccaaacgatgaaaacaaccccaagctatatattaccagatgaaaggataaacaaaatatggtccatccatacaatggagtattacacagccataaaaaggaatgaagcagtgatccccactacactgtggatgaaccttgaatgcatgatactgaatgaaagacatcagatgcaaaaggtcacatagtgtactgtccttttatatgaaatttccagaacaggccaatctgaagagatgtatagtggattggtggctttcagcagctgtggggaggtgggactgaggagcgactgctaatcaggatggggtttcctcctgggatggtgaaaatgttccggacctagatagtgatgaaggtagcacgacactgtgagtgcactaaatgctattgaattggacactttagaatggttgaaatagtgatttttatgtgaattctacctaaacatgctattacagctcatatatactttttccatctggattcttcacaaaagaatatgttgtgagcatctttccatgatattaaatcatcttaggaaacattattttgtgttcttcaaaatgtgcatgttaagtattcaaatcagtcttaaatttttaaaaatatgtaattttagaaaataatttaaaaggttttgtttcagtttgtaagatttcttttctggcactttaatggcttgaggtatcattatcagttacaaattgagttattcttcatcaaatgacttttggagtagagattttatttttatagcaatagatgcacagatattcctgtaagatacaggtgtggttagacacttttctagaacaggcatgccctgcaaactccacagacactgactgtttttgtcctattaagaagtagaccactgagaagggagaaggtgacattttagctttcccaggtaaaagtggttttcatcctcacaccaattttatggactggacgttaactctcttgctcaaggtcactctgagtggaagagtggggataaatctggttcgtttggcatcagaggccatgacttttcctaccacagaagtaattttcaaagtaagtctctgccctaggcacatcagatcacctggggaccactccagagtgagtagacaagactttgacaggggtgcctaatttttttttttttttgagatggagtctcgctctgttgcccaggctggagtgcagtggcatgatctcagttcactgcaacctccgcctcctgcgttcaagtgattgtcctgtctcagcctcccaagtagctgggattacaggcacccaccaccacatccagctaattgttgtatttttaatagaggcggggtttcgccatgttgcactggctggtgttgaactcctaacctcaggtgatccacccgcctcggcctcccaaagtgctgggattacaggcccggtccagtttgatagtttgttatcatgttatacgtacacttagaataatgatccagccatctcattctaacagcaatgagaacttaggctcccctctctactaaaatacaaaagttagctggtcatggtggtgcgggcctgtaatcccagctactcgggaggctgatgcgggagaatcacttgaaacccacaggcggaggttgcagtgagctgaggtggtgccactacgctccagcctgggggagggggacatagcgagactcggtctcaaaaacaaaacaaaacaaaacaaaacaaaacaaaaacaaaacaaaacaaaacaaaacactgccaaactgtttccaaagcatctgcatcgtgtttaatttccataagtaacgtatgagaattctaggtcctccaaataattagcagtccttcgtagtgtcagtttttgtttcagccttccttatgggtatgctgtggcatatccctgagattttaattcgcattttccagtgactaagggtgttgaaccactcgtgccttcctgtgcctatttgacactcctatattttctttggtgaagtgtctgttcaatcatctgctcattaaattttccttcggttgtttgccttcttattgttggattttgagagttctatgtacaagtcctttgtcagtcgtgtgatttgcaagtcttcttcctagtctctgaagtttctttctttcatagagcaaacgttttaaatttttatgaagtaagacttctcagttttatttttggtgaattatgtttatactgccatatataggaactctttgcttaactggaggtcatggagattttctcctacattttcttttaaaagttttatagtttcagagtttacatgtaattccataatctccatttagttaaattttgtatggggtgtgaactgtggtttgactgttttcttgtatatggatatccaattgtttcacacaatttttttgaaaagaatatccttggcagggcacggtagctcacacctgtaatcccagcactttaggaggtcaaggtgggcagatcatgaggtcaggagtttgagaccagcctggccaatatggtgaaaccacgtctctattaaaaatagaaaaattagccaggcgtggtggtgtgtgcctgtattcacagctgcttggatggctgaggcaggagaatcgcttgaacccaggagtcagaggtttcagtgagccgagattgtgccactgcactccagcctgggtgaaagagctagattctctctctcaaaaaaaaaaaaaaaaaaaaggaaagaaagaaagaaaagaaaagaaaatcctttttgctttaacttgccttgcaggtttgtagaaaactcaattgttgaaatttgggtggatatatttctggattttctattctattccatgttggaccaataccacactgccctagtcactgttgcattatagtatatctttaaaggagtaatgggaatccttcaactacattttttccccaataatttttggctattctgcttcctttgtgtttctatgtaaattttatcatcagtgtgtctatttctacaaatagtcctgatagggtttgaattgggatttctgtgaatctatagatcaatctgaggagacttaataatgatattgattctcccaattcatgaatatagtatacccctgtatttatttgttttcttgaatttcttttatcattgttttgtagttttcaccatgacagtcttgcacatattttgttaaatgtacagctgagaatttaattttttctggtgtacaatgctaataaaatggtgctttaaaaga

SEQ ID NO: 15 - DNA, 2727 bp, synthetic construct
  source 1..2727: mol_type = other DNA
gttcgttgcaacaaattgatgagcaatgcttttttataatgccaactttgtacaaaaaagttggcatgacgaccgaatctggatcagactcggaatccaagccggaccaggaggccgagccccaggaggcggcgggggcgcaggggcgcgcgggggcgcccgtgccggagccgcccaaggaggagcagcagcaggccctggagcagttcgccgccgctgcagcgcacagcaccccggtgcggagggaggtcactgacaaggaacaggagtttgctgccagggctgcaaaacagctcgaatatcagcaattagaagacgataaactttctcagaaatcatctagcagtaaactctctcggtctccattaaagattgtcaaaaagcctaaaagcatgcagtgcaaagtgatacttctcgatggatcagaatatacctgtgatgtagagaaacgctccagaggacaagtgctgtttgataaagtgtgtgaacacttgaacttgctagagaaagactactttgggcttacgtatcgagatgctgaaaaccagaagaattggttggaccctgctaaggaaataaaaaaacaggttcgaagtggtgcttggcacttttcatttaatgtgaaattttatccaccagaccctgcccaactatctgaagatatcaccaggtactacctctgcttgcagttgcgagatgacatcgtgtccggaaggctgccctgctcctttgttaccctggccttgctgggctcctacactgtccagtcagagctcggagactatgacccagatgaatgtgggagcgattacattagtgagttccgctttgcaccaaaccacactaaagaactggaagacaaagtgatcgagctgcacaagagccacagaggaatgacgccagcagaagcagagatgcatttcttggaaaatgccaaaaaattatcaatgtatggggtagatttacatcatgctaaggactcagaaggggtagaaattatgttaggagtttgtgcaagtggtctgttgatatatcgcgaccggctgcgaataaacagatttgcctggcccaaggttctaaagatttcatacaaacggaacaacttttacattaagatccggccgggagagtttgaacaatttgaaagcaccattgggtttaagctgccaaaccatcgagctgccaagcgtttatggaaagtatgtgttgagcatcatacatttttcagactactgttaccagaagcacctcccaagaaattcctaaccttgggttccaagtttcgttatagtggcaggacacaagcgcaaacgagaagagccagtgcgttgatagatcgcccagcaccttactttgaacgctcatccagcaaacgttataccatgtctcgcagcttggatggagcatcagtgaatgaaaaccatgaaatatacatgaaggattctatgtctgctgcagaggttggtactggccagtacgccacaacaaaaggcatctctcagaccaacttgatcaccactgtgactccggagaagaaggctgaggaggagcgggacgaggaagaggacaaacggaggaagggggaagaagtcacgcccatctcggccatccggcacgagggaaagactgacagtgagcgcacggacaccgcagccgacggggagaccaccgccactgaggagctagaaaaaactcaagatgacctgatgaaacatcaaaccaacattagcgagctgaaaagaaccttcttagaaacctcaacagacactgccgtaacgaatgaatgggagaagaggctttccacctcccccgtgcgactggccgccaggcaggaggatgcccccatgatcgaaccacttgtccctgaagagaaaatggaaaccaagacggagtccagtggaatagagacggaacccaccgtgcaccacctgccgcttagcactgagaaggtggtgcaggagaccgtgttggtggaggagcggcgtgtggtgcacgcgagtggggatgcttcttactcggcgggagacagcggggatgctgcagcacagcccgcattcacaggcattaaagggaaagagggctctgccttgacggagggggctaaagaggaaggaggggaggaggtcgctaaagctgtcctggaacaggaagagacagccgctgcttcccgtgagcgacaagaggagcagagtgcagccatccacatttcagaaactttggaacaaaaacctcattttgagtcctcaacggtgaagacggaaaccatcagttttggcagtgtttcaccgggaggagtaaagctagaaatttccacgaaggaagtgccagtagttcacaccgaaaccaaaaccatcacatatgaatcatcacaggtcgatccaggcacagatctggagccaggcgtgctgatgagtgcacagacgatcacatctgaaaccaccagtaccaccaccactacgcacatcaccaaaactgtgaaagggggcatttcagagacaagaattgagaagcgaatagtcatcacgggggatgcagacattgaccatgaccaggcgctggctcaggcaattaaagaggccaaagagcagcaccctgacatgtcagtgaccaaagtagtggtccataaagagacagagatcacaccagaagatggagaggattgcccaactttcttgtacaaagttggcattataagaaagcattgcttatcaatttgttgcaacgaac

SEQ ID NO: 16 - DNA, 5148 bp, Homo sapiens
  source 1..5148: mol_type = genomic DNA
aacacagacaaagtgctgccgtgacactcggccctccagtgttgcggagaggcaagagcagcgaccgcggcacctgtccgcccggagctgggacgcgggcgcccgggcggccggacgaagcgaggagggaccgccgaggtgcgcgtctgtgcggctcagcctggcgggggacgcggggagaatgtggactgggtagagatgaacgagacttttctcagatgttggatatttgcttggaaaaacgtgtgggtacgaccttggctgcccccaagtgtaactccagcactgtgaggtttcagggattggcagaggggaccaaggggaccatgaaaatggacatggaggatgcggatatgactctgtggacagaggctgagtttgaagagaagtgtacatacattgtgaacgaccacccctgggattctggtgctgatggcggtacttcggttcaggcggaggcatccttaccaaggaatctgcttttcaagtatgccaccaacagtgaagaggttattggagtgatgagtaaagaatacataccaaagggcacacgttttggacccctaataggtgaaatctacaccaatgacacagttcctaagaacgccaacaggaaatatttttggaggatctattccagaggggagcttcaccacttcattgacggctttaatgaagagaaaagcaactggatgcgctatgtgaatccagcacactctccccgggagcaaaacctggctgcgtgtcagaacgggatgaacatctacttctacaccattaagcccatccctgccaaccaggaacttcttgtgtggtattgtcgggactttgcagaaaggcttcactacccttatcccggagagctgacaatgatgaatctcacacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagacccacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcagcctgccccaccccatgctcaaccccacttctctcccgagctcgctgccctcagatggagcccggaggttgctccagccggagcatcccagggaggtgcttgtcccggcgccccacagtgccttctcctttaccggggccgccgccagcatgaaggacaaggcctgtagccccacaagcgggtctcccacggcgggaacagccgccacggcagaacatgtggtgcagcccaaagctacctcagcagcgatggcagcccccagcagcgacgaagccatgaatctcattaaaaacaaaagaaacatgaccggctacaagacccttccctacccgctgaagaagcagaacggcaagatcaagtacgaatgcaacgtttgcgccaagactttcggccagctctccaatctgaaggtccacctgagagtgcacagtggagaacggcctttcaaatgtcagacttgcaacaagggctttactcagctcgcccacctgcagaaacactacctggtacacacgggagaaaagccacatgaatgccaggtctgccacaagagatttagcagcaccagcaatctcaagacccacctgcgactccattctggagagaaaccataccaatgcaaggtgtgccctgccaagttcacccagtttgtgcacctgaaactgcacaagcgtctgcacacccgggagcggccccacaagtgctcccagtgccacaagaactacatccatctctgtagcctcaaggttcacctgaaagggaactgcgctgcggccccggcgcctgggctgcccttggaagatctgacccgaatcaatgaagaaatcgagaagtttgacatcagtgacaatgctgaccggctcgaggacgtggaggatgacatcagtgtgatctctgtagtggagaaggaaattctggccgtggtcagaaaagagaaagaagaaactggcctgaaagtgtctttgcaaagaaacatggggaatggactcctctcctcagggtgcagcctttatgagtcatcagatctacccctcatgaagttgcctcccagcaacccactacctctggtacctgtaaaggtcaaacaagaaacagttgaaccaatggatccttaagattttcagaaaacacttattttgtttcttaagttatgacttggtgagtcagggtgcctgtaggaagtggcttgtacataatcccagctctgcaaagctctctcgacagcaaatggtttcccctcacctctggaattaaagaaggaactccaaagttactgaaatctcagggcatgaacaaggcaaaggccatatatatatatatatatatatctgtatacatattatatatacttatttacacctgtgtctatatatttgcccctgtgtattttgaatatttgtgtggacatgtttgcatagccttcccattactaagactattacctagtcataattattttttcaatgataatccttcataatttattatacaatttatcattcagaaagcaataattaaaaaagtttacaatgactggaaagattccttgtaatttgagtataaatgtatttttgtcttgtggccattctttgtagataatttctgcacatctgtataagtacctaagatttagttaaacaaatatatgacttcagtcaacctctctctctaataatggtttgaaaatgaggtttgggtaattgccaatgttggacagttgatgtgttcattcctgggatcctatcatttgaacagcattgtacataacttgggggtatgtgtgcaggattacccaagaataacttaagtagaagaaacaagaaagggaatcttgtatatttttgttgatagttcatgtttttcccccagccacaattttaccggaagggtgacaggaaggctttaccaacctgtctctccctccaaaagagcagaatcctcccaccgccctgccctccccaccgagtcctgtggccattcagagcggccacatgacttttgcatccattgtattatcagaaaatgtgaagaagaaaaaaatgccatgttttaaaaccactgcgaaaatttccccaaagcataggtggctttgtgtgtgtgcgatttgggggcttgagtctgggtggtgttttgttgttggtttttgttgctttttttttttttttttttttaatgtcaaaattgcacaaacatggtgctctaccaggaaggattcgaggtagataggctcaggccacactttaaaaacaaacacacaaacaacaaaaaacgggtattctagtcatcttggggtaaaagcgggtaatgaacattcctatccccaacacatcaattgtattttttctgtaaaactcagattttcctcagtatttgtgtttttacattttatggttaatttaatggaagatgaaagggcattgcaaagttgttcaacaacagttacctcattgagtgtgtccagtagtgcaggaaatgatgtcttatctaatgatttgcttctctagaggagaaaccgagtaaatgtgctccagcaagatagactttgtgttattctatcttttattctgctaagcccaaagattacatgttggtgttcaaagtgtagcaaaaaatgatgtatatttataaatctatttataccactatatcatatgtatatatatttataaccacttaaattgtgagccaagccatgtaaaagatctactttttctaagggcaaaaaaaaaaaaaaaaaaaaaagaacactcctttctgagactttgcttaatacttggtgacctcacaatcacgtcggtatgattgggcacccttgcctactgtaagagaccctaaaaccttggtgcagtggtggggaccacaaaacaaccagggaggaagagatacatcattttttagtattaaggaccatctaagacagctctattttttttttgccactttatgattatgtggtcacacccaagtcacagaaataaaaaactgactttaccgctgcaatttttctgttttcctccttactaaatactgatacattactccaatctattttataattatatttgacattttgttcacatcaactaatgttcacctgtagaagagaacaaatttcgaataatccagggaaacccaagagccttactggtcttctgtaacttccaagactgacagctttttatgtatcagtgtttgataaacacagtccttaactgaaggtaaaccaaagcatcacgttgacattagaccaaatacttttgattcccaactactcgtttgttctttttctccttttgtgctttcccatagtgagaatttttataaagacttcttgcttctctcaccatccatccttctcttttctgcctcttacatgtgaatgttgagcccacaatcaacagtggttttattttttcctctactcaaagttaaaactgaccaaagttactggctttttactttgctagaacaacaaactatcttatgtttacatactggtttacaatgttatttatgtgcaaattgtcaaaatgtaaattaaatataaatgttcatgctttaccaaaa

SEQ ID NO: 17 - DNA, 196204 bp, Homo sapiens
  source 1..196204: mol_type = genomic DNA
gaattccccctttggacagcctcctgccccacagacacccccccccgagtagagtccctcagggacccatcactcacatcggggtgcatgcctggtgtgtggggggatgaggaaccccatgagagcatcacgcagtgcagctccactccctgaagaccctctgctgggcctagattttaaatattttctttccatagcaagtggaattgttacctggaaaactctggaggtgccactttggttgtagtttgctagttcatgtttgaattaaacaccgtctgtcacgcccaacctgtgcctgcgccccgctggctggaatgtagcctgggtaagcgagtggccggcacgccgcggtgccacgtgctggtgagcccaagcgaaggtggactcagcagccccagccacggagtggatgccatttcgttgcttaaaacacttgtttgtgccaggaaatgagaaaagaggagtgtttgtgtcggtagttgtcaatcttagctgacctgaacgggctgggatgaggtcctggcatctgtgtttagtgttcacattgtttgtacggacctactgagcagcccgggctcaggacagtgcatgtgggcccctgatcgtggcttatgaggagacatcttacatgacctgaagggcagccttgtttccagaattttctattaacctcaacatagtataaatacacaccagccaggctacatcgcattttactgttgtgttgtagatgcttttcttctttgaaaactgagctgtttaattggtttagaaagagattcgcgttcctccttagatggaactactaattttatctctggaagaaacaagtctttgatgcatgacgtttatagcaatacgtttttaatgccagttgaatttgtcactgaacatttagcagctcccaaatggcacgggttgtgatagcgccttcaacccgattgttcttcagggatcaaggcaaaggtgtcaggaggcactaaaccgtacaacgcgagccttcccgtgggtagaggggccagccatagagctgtgtgatggggaacttttatgttgttcagcattcaccctgatgcctgccgtgcctaggaatttgtatataaatatttgcccaatttgccgggcgcagtggctcatgcctgtaatcccaacactctgggaggccgaggcgggcagatcacctgaggttgggagttcaagaccagcctggccaacatggagaaaccctgtctctactaaaaatacagaattagctgggtgtggtggcgcatgcctataatcccagctgctccagaggctgaggcaggagaatcgcttgaacccaggaggcggaggttgtggtgagccgaggtcacgccactgcgctccagcctgggcgatgagagaaactctgtctcgataaataaataaatatttgcccagtgattaaccagcaaaaatggatctcccccgaaataaacacagtgttccttgttttgagccacttctacggtgacgagggatttgtcagttttggttttcagtgatgagtccctggtcaagggtgaattttcacagtgaaacggggggtgatgagcattctgctcccttaagccctctagacagcaggccctgctggccgcatgtgacatcgtgaggtcacctggcaggggcacggtgtggcttagctcggaaagcctgggctttggggtcacacgccacctcttacggggcccgggacacgttactgaacctctcaaaccttggcttggagcctttcaagccaggaatgaagactgagtcagcagcggcagctgtcacctcgctgtcacccactgagacactgtcgttgtctttccagggtagaattccgtgtggattcccagctctgcttcagagtattccatgggtgtatcgccgccagagaagtcacattccccaagttacgctttttgttttttgagacagagtcttgctctgtcgctcagactggagagcagtggcgcgatctcagctcactgcagcctctgcctcttgggttcaggcaattctcgtgcctcagcctcatgagtagctgggactacaggtgcccgccaccacgcctggccaatttttgtattttttgtagagacggagtttcaccttgttggccaggctggtctcgaactcctgacctcaggtgatacgcccgccccagcctcccaaagtgcggggattataggtgggagccatagcacccagccattttttaaaatgcttttatgtcaataaatatatgtaattgcacatttacatttaaatatttatgtaaaatgtataatttggtgaggtctgacatgaatgaactatgaccagtcaagacacagaagataccacggcactgtggggctctgcaaccaggcctcctcgcccccactcccaccccaccccaccccatccccggcacccgtgcatctgcgggctgcacttaggtgaggtggtgtttcctggagttttacgtatgtggaatcacagtgcgcactttctgtctaaccgcttgaattcattcaccacggaggctgggagactcctcctggagcgtccgcagttccttttatcgctgagtggttttccctgtgtgtctccagcacaattgtttatcccttcagctgtcgggtaaatttaccaaaagatactgtttgttgacttatttttaaatattcttatgtttattttagcctcgggcaggtgtgcaggctttgttgatttatttttaaatatgctaatgtttattttagcctcaggtctgtgtgcaggcttggtatataggtaaactagtgactagggtttgtcctacagatcatttcagcaccgggtaccaagcatgggtacctgacagttttgttaaaatggttttctgagcctctccctcttccctcaggccggccccagtgtgtgttgttggcccctttctgtccctgtgttctcatcattcagcttccacttgtaagtggggaagtgtagtgcccgggattctgtccccgcattagtttgcgaaggataatggcctccagctccatccaagttcctgcaaagggcatgatcttgttcctttttatggctgcatagtattccgcggtgtatatggaccacatcctctttatccgtctgccgctgacaggccccagcttgattccatgtcggtgctgttgtgagtggtgctgcagtgggcatttgagtgcgtgtgtcttcatggtagaaccatttagagtcctctggacataacccagtagtggggttgctgggtcgaacggtagttctgtttttagttcttttaagaattgccagactgctttccaggatggttcaactaatttacactcccaccagcaggtacaagcgttcccaagagatgccattttaactcacagccacagcagttgaaggtgaaggagagggaaggaattgggaacccagcaggttggtgtccttggatatgtgccccgcagaagtcgggcagcagttcctggttcgtgtggttacgctagagagaaactgacctggttcatctggttacgctagagagcagctgaccttgccccatggatggcgcgagttatgggcggccgaccttgccccggggatggcgcgggttgtgggcggccgaccttgctccagggatggcgcgggttgtgggcggccgaccttgccccggggatggcgcgggttgtgggcggccgaccttgccccggggatggcgcgggttgtgggcggccgaccttgccccggggatggcgcgggttgtggtcatcacctgctttctccagacttctccctgctgggacggggagtcatctcccaggccaactggattattggggcacttcggagccaccatccaggtcttgaggagcctcagccttggctgtagtatttgtacattggcatttagaaacttggtgtggctgagaccacagcccaggaggctgtgacctgcagcagctgtccccctgtctgcccacacggtcgtgggtctggctcttcctgccacttggctcctcctgcagtgtgtttgcgtgattttttttattgcggcctctgaccttcttttgtctcttcagtcatctaaagagatttttaaaaggaaaacggtctagtgtgtgcggtgcacccctagcccagggcctaagaggagcgtcttgtgaaggagctgtacctgccggctcaggtcccttctcggagtgactcctggcagagcagagcaggcctcagccctccttacaggcgcgtccagccagccctgcgtccgggcgtgagtgcgctttagctcgcggcagtcggcggaggggtccttgtgaaatctcctctggccccctgtcccgaggcacctgtgtgtgtcttgagtatttcctcttcatccttcacgtgtccctgacagctgaacgcgcaagccagcttccgtctgccaagagagaagggcgggattcgccccatgagctcggtctttcttcatttgacaccgacagtctcggccactagttctaatgcttgcgtttctgtgttctgctttttaatgtttttgtaagagaagagcaaaatcagctagattgtttgcccagacactttgctctgtggatacagatgttttggccggcagagagcaccagcgctcactggctctcagcgcctgtcagcaggcagaagccatttccctatctggaaggcacgtctgggtgtccacatggcacggccaatagtgcgcagcatgcagagccgggccgggagaaggcccggccatgcccagctgccccccactctccccggcctcgggcttgagagggtacctgtcctggcttagtcacctggaaaccaaaatccttcgcagcttccagaattctccagtacaggaggagaagccgtccacgttcagagccgccttagacggtttgcctgtcaccggcattcctggacctggaaacgggtgcccccagccaggccggggaccactgtgtgcccagaattctcctcccgtcctttttccccttgcccggctcccagctgcccagggaagaagggagccggctgcaaggcgcagtccaaaccaggccgggggccgtgaccatcggcagtgccccccagagcaggctcctcgtgcaggaatatgggtcactgccttccagggagtcctttttttcttctggtttctaagtcgccacctcttgctttacctcagatagaagcatccagaacgctgtagtatcggcaaagcagaagctggtgtggtgcttgctcagggtcggtgcatgcggttctgcccgtggccccactggcggcatcgtgaggccaggcgtgtctgggagcttgtttttccagagtgccctgtgccagacggctcccggcctcctctgagtcagtcatgtccctgcaggactggaactaggacggccggtcacagagtcagtggtcctgtcgaggctcctgctgtggtgttggggtgggtctccctccagaaccttcactgtgcggggagcacagcaaaaccggaggcctgccaacggcctgcaggctgacgggggtgcgggggcactttctctcttgggtgcgggcttttccctcctggtgccctgcctctgtgcagcacgaagcggtctcctgtggggggagggcctgtgtgccaggctaatgagatgcccggatgtggcggggctgtctgtgtttggggtccctggctgtggtgccttctgaagaggagcccttttctgtggttaactgagcatccaaccctttgcgttctcggctggccttcccgtcctggcaacaccaaggtcattctggtcctcagtggcgttgctgtggctctttatcacctccactgcaattggtttttgtttgtttgttttggggggatggcgcctcgctctgtcatccgtctaggcagtggtgcaatcatggcttactgcggccttgactacccggactcaagcagtcctcccacctcagcctcccgagtagctgggaccacaggtgcacaccaccatgcccagctaatttctgtgttttttgtagagacagggtctcactatgttgctcaggccagtctcaaactcatgagctcaagcgatcctcccacctcagcctcccaaagggctgagattacaggtgtgagcccccgaatccggtgtgcactgctgtttacttagtatttttctttaactagatttatttttaaacaaggctttgtccaaggacatttggctcgcaggcacagagctgattaactcgttatgtatcttttgataataaggcagcgatcattaagaaaaacgtgtagccaatgaaataacatgttctgggccccaccactggactgggaggtgcagcgcatccaagcagaggctgcctcctgccctccacgcctgctgctctcgcaggcaggggctctgctgcttacagcagtgcggccatctcggcttctctccacatcgtctgtcacgcgctggtccccaccatacctctcgccaccccgtgcctctgtccccgtgcggcctgaggagctccagctttccctgccagcggtgctctgggagtggggacgtgatgcagggcgagcatgatgcaacggggcaccccagacccttccctcccgtggggggaggggtgtggcacgcagaggggcagagggcggggacactggccccgtgggggaagaaggtgctgtcacagccgttactgtcccccgtgggaccccagcctggagccccccatcctttggctcctgcctgtggccactcagctctcaggtggccacatgcacatcccctgctccttccctgcgcacctgccctgcccagtggcctttctggtcccagctactgaaaccggtgagctgctccagggtgaggctgctttctggctcctggtgtatttggacacagataggcccttagtgtccagaggcgccccatgcagccctcatggtcagcaggacacccaggatagaccccctccacgcagcacctgggccctgggagcggctgcttttaggatgccacctgttcctgggcgccttgtttttagcttctgacctgaagatgagcgggggagcgcggtggcgagggcacgtgggcgtggctcacggtctcctctctgtggcaggtacatgtcccagagcaagcacacggaggcccgggagctcatgtactcgggagccctgctcttcttcagccatggccaggtaagccgtcttgcttcctcctgcatccctttctgctccgggcaggcaagtggaggtgggaatgacctcccacctccactgatgggcaaccttgcgctgccctgggcctcggccactggggctgtgggtggcagaggggcccagagtcccccctggcttggctgctccactctcaagactggggctctgcgctgcgtttgtgcacgatggggctgagagggcgttgtgttggtataaacgtccccgtccattttcatgtgattctcggccaggagctctctttttcggcggcattgccaagggacgtggtgctcagaagagactcctcagcaccggccgcactctggcttctgctggaaacccagccttgtctttgctgtcctgaacaggtcagggctttgttcttgattcttgtgtgttgctttctcttgtagcaaaacagtgcagcagacttgtccatgctggtcctggagtccctggagaaggcggaagtggaggtggctgacgagctgctgggtgagcatccggcccttcaccccgggaccctgtgacagcggccccagtacgcccctccccgggtctctgcgctgtgtttcgtgtctgcgtttgggggtcttgtgtgctgtggggtgaactctgctgcagaggcagttcccacccggtctcagtgagccaggccccctggctgaccctggcgcgagcacacggcaggctgagcaggacctgctgtgattcgtaggcagggcatgactggggccctgtcctctccactgctccgatggacagtggcctccccgccagctggcaaggtggggcaccactttggcttgtcggcctgtcctgtcgggtgacgtgcggttggcagagcccttctgtgagctgcctggctgcaagtcccactcctagtccctgctgctctgggagatgccccgggccagagccaggtacccagagcggccacagctgttcctggactccagctccccacggcaccttccccagcccccgcctcggccagggcgtccgtccttccttcctcgctgtgctctggggcatatggcgctggaactgcggtgggcagctcagtgtggtggtccacggctcactcagggacgcccagcaggttcctctccctgttaagtagttgcgaatggaaatccacttctgtggggaatgtgggacagagagccggagtggccgtgcgttcctctgatgagggtctgtgctgtttgcagaaaatctggctaaagtgttcagcctgatggaccccaactctcctgagcgcgtgacctttgtgtccagagccctgaagtggtccagtgggggctccgggaagctgggccacccccggctgcaccagctgctggccctcaccctgtggaaaggtaggcctggggccagggcaggcgtgggcacctctctgctctcggcgttgatttgcactgtgcgttccaatacatcagggtcacccaccagggcagagatggggtttcaccctgtggtcacgcgggccggtccatggagacccatgcgccatgctaagcccgggatcagagccactgcacccgagaagctgcgagaaggggggcaggcggccgcctctccaggccttcccgtgggacgttctgaccctcagggaggcagccgtgccttcacggggcctatgcgtgctgctgctcctttctggaacattctttccagcgctgggatggtgctgtgccctgtttttctcctttacgctgacctcatcttctgatgaagcctttgcggggattaatcaccctgtggtggggtctggctgtctgcgtggtgtggttggtgtttttagttccaggtctgcagctctgaggcgggagtggtgctgggtagatattcattgagtgacttgaacgaggtgctaatgggggtttagaagcgtcctccccagggcattccctgggatggcaagtggcaccagcgctgtgagatggtttccttcctgccgtcaggcgtcaggaggcctgctcgggtctgcatgtgcctgggcttggcaggtggggaggcggcacccagggcagcccccgcattggaagctctaggaacttggatctaggctgaaggcctttgctctagagttccggattgctgagcttggaacagctcctcctagacacagtgtggcgcgggagcagctgcatgagggcccacgtactctgcacacatcagcagcccatccgaccttgtccctggtcatgcgaaacgcatgcccatccgacctccgaccttgttcctggtcatgcaggttccgcctgggctgtgacaggaggctcggcgccctcaggcacctggcaggctttcccgaccttctccctgagcgacaggaatgggaagtgagcaaatccaagtagaattaggaggctctcctcagaggagcctggcggaacccggtcctggctgtgtgccgagtggcggggcacccacagggagatgagccagtgcctatctcttgcctcagtctccccgtggtcgcctccttcctcagaaggggctggcccaggctggtgcctccgtggcccctggagcatacctgctgccgccttccctggcctctggcgacagcaggccttccgtactccacctgtcccccagccagcatggctccacacagaacacggccgcagacaaccctcctgtgtcctgcatagcgaactgccactctgcgtgggccccactgccaggttagggaggcaggagctgcccccaccccctgcccgggagctgtcgtctgtctttcggatgagtcaggaacaaatgcgtcaacactgggtggccgccctcgccactcctgcgcacgggccctgtggggagagccaggcagtgggtggcgtgggacaggtgatgcccatgcccagtactcaggagacctgcatggcagctcctaagatgggtacaagcgatcataaggagggatgtacgtgtctcatctccccacccaccacgggatcaggttgaggccgtccagggagtgcatgggagggctgcgctcggcgcatctgcagagatcccctgaagaggggccctggctgcctcccactggggtaggtgcgggcccaggtccccgacaccgtgacggtgctgtgggactttgatggacctcacccggggagcccaggcctgctccacggcaaggtggccacgcactgcctctgcagtcctgagcacaggctccatacgggacctgaggcctgggctccatgacacttgggctatgtgcttccctggtgcttccctcctggggatgaaatcacacccataggtaaaggtttgggagtggagggagacagcaggagacaaagcaaggggacgaggactctggcagccacaaaggcagcgggagggccctggaggctcctgttgggcagcgcagtcaggaaagggcccactaggtgggagaggcaggcgcctggtgagtgctgagatctgcaccacgagaagggctgggctcccaaggcatgcatggggcctgggagtggagggagtgtgagcgggtgttaggacggggtctggggcgagcgggtgttaggacgggcctgggagcggagggagtgcgagcgggtgttaggacggggtctgggagtggagggagcgcgagcgggtgttaggacggggcctgggagtggagggagcgagagcgggtgttaggacggggtctgggagtggagggagcgcgagcgggtgttaggacggggtctgggagtggagggagcgcgagcgggtgttaggacggggtctgggagtggagggagcgagagcgggtgtcaggacggggtctgggagtggagggagcgcgagcgggtgtcaggacggggtctgggagtggagggagcgcgagcgggtgttaggacggggtctgggagtggagggagcgcgagcgggtgttaggacggggtctgggagtggagggagcgcgagcgggtgttaggacgggtctgggagtggagggagcgcgagcgggtgttaggacgggtctgggagtggagggagcgcgagcgggtgttaggacgggtctgggagtggagggagcgcgagcgggtgttaggacggggtctgggagtggagggagcgcgagcgggtgttaggacggggtctgggagtggagggagcgtgagcgggtgtcaggacggggtctgggagtggagggagcgcgagcgggtgtcaggacggggtctgggagtggagggagtgcgagcgggtgtcaggacggggtctgggagtggagggagtgtgagcgggtgttaggacgggcctgggagtggagggagtgtgagcgggtgttaggacgggcttgggagtggtgtgtgttcatgagatgttgagggttggttgttgggtcagtgtggtgagaacatcaggctggtttctgctgttgatctacatccgaggtgcactggcctgggccctgattatattgcgccctgtaagttgcttctggtaggtgagaagggtcacatggtaacactaattggttatcacaacgaacattagtgcagttggattaagcagctccaagagcttggagaatcaagatgttgcatagctgagtgacttttccccgtggatctgttgatttggtgctgcgtgtgaggcctgggctggctctgagtccctatcaggacctctgcctgtgggctctggctgggctctcctccagggcgggcaggtgggctagaattaacatggagtgtctttccccctttccttttctgtgttatattcgtgaaatgtatttacatttttctgtctttcctttgcagaacaaaactattgtgagtcgaggtatcattttctgcactcagcggacggggagggctgtgccaacatgctggtggagtattccacgtcccgcggcttccgcagcgaggtggacatgttcgtggcccaggccgtgctacagtaggtgtctgtggctcttcgggtctcggcttccattgctgccttggctgggcagccttagaacaggtccccttgtcccctgtccgagccgagctgcaggataaactgagttcactctgggcagagcccacagtgcacttgtcagcctgacccatgatttttcataagtttaaccaatgttaagaagtattttagaaactccccctttcccgacgggcactggagtgccctacacacgcccctcgcctctcgcccactgccgggaggccctgtggtctctgctgtactcaggcctgcctcggccagttctttcccgcactatctggaaatgcgtggaattgtgagcatctaccccgcggcccctcccgccagctcgctggggcgtcctgcaggccaggctccgggcgctgtctgctcctgcgtggtcccttccgccagctcgggcgctgtctgctcctgcgtggtccctcccgccagctcgctggggcgtcctgcaggccaggctccgggcgctgcctgctccaggggctggccttcgcttcctttctcacgaaagccttacttgtgcccgtcagtttcttcccacagaacaaatatggatttcaaggcgggcgttggggatttgatgtaggatttggggacagacatcctctgacctcagcgttgcccgctgcggagctttgccaggagctggcgtccgtgacttaagtgaaaagctgggtcaaacccagagctccctggctctgcgctacgccgtgtacatgttttctctgggctgacaggggccctgcccctggggcactgagccctccctgtgggtcctcgaacagaagccagggtctgtgcggcacccaccagctgctgggccatggcggagtgttctggtgcgggccagcgcctgaccggtgcgggcggcctcaggagaggagagcttgctcagtgcgtcacgtagtcagggctcaggctggggcccggctccagagcctggtcacattcccaagcttcattctcttcacctgtgaattgcaggcttccctggtgtgccctgcacatgagggaagacacgcgtgaagcactgggtccctccatggccttgggccgcaggaaccgtgggcgcacgagcttgggaaggacatgtcggaggccggcgcctgtgcgggcagaagctgtgtcctccagcccttccaccaccagcatgttctcatttccaggtttctctgtttaaaaaacaaaagtagcgcatcggtggtcttcacgacgtacacccagaagcacccgtccatcgaggacgggcctccgtttgtggagccgctgcttaacttcatctggttcctgctgctggctgtggacgggtgcgtcttgggatcctgcagggggagggggctgtgaatgtgcgggttgtgtgtagacgtggtgtggatagctgtgtgggtgtgtgtgcaagtgtagccatggtgtgggtagccgtgtgggtatatgcatagggtatgagtgctgggtgtagacgtggcataggtgtgtgtgcaggtctgttgggtgtagacatggtagtgcgggtagctgtgtgggtgtatgtgcaagtgtagacatggcgtgggggagtgtaggtgttgggcctctggtagtgtgggtgtgtgcaggtgtggggtggtgtgggtgcagacgtctggggggttgtgtgcgggtgttgggtatccatgtggtgtgggggtgtgtagacgtgtatacaggtgtgagtgcaggtgtagacggcgtatgtgcaggtgttgcgtgtctggtgtgggtagttggggtgcgtgcaggtatgtgtgttgtgtgtagacgtgtgggtagctgtgggggtgtgcaggtgtgtgtactgggtatagacgtggcatgggttgctgggtgtgtgcaggtgttgggtgtttgcaggtaagtgttgggggcgggcgtggtggtgtttgcaggtgaggggtgtaggcgtgtgtgcaggtgagtgttgggtgtgggcgtggtggtgtgtgcaggcgagtgttgggtgcgggcgtggtgatgtgtgcaggcaagtgttgggtgtaggcgtggtgtgtgcaggtgagtgttgggtgtaggcgtggtggtgtttgcaggtgagtgttgggcgcgggcgcggtggtgtgtgcaggtgagtgttgggcgtgggcgtggtggtgtgtgcaggtgagtgttgggcgcgggcgcggtggtgtgtgcaggtgagtgttgggcgcgggcgcggtggtgtgtgcaggtgagtgttgggcgcgggcgtggtggtggttgcaggtgagtgttgggtgtgggcgtggtgtgtgcaggtgagtgttgggcgtgggcgcggtggtgtgtgcaggtgagtgttgggtgcaggcatggttgcaggtgagtgttgggcgcgggcgcggtggtttgtgcaggtgagtgttgggtgcgggcatggtggttgcaggtgagtgttgggtgtaggcgtggtggtgtgtgcaggtgagtgttgggcgtaggcgtggtggttgcaggtgagtgttgggcatgggcgcggtgtgtgcaggtgagtgttgggcgcgggcgtggtggtgtgtgcaggtgagtgttgggcgcaggcgcggtggtttgtgcaggtgagtgtggtcctgttctgctcacccagcacatccctgtgtgtctctgtcccagtgggaagctgacggtgttcactgtgctgtgtgagcagtaccagccatccctccggcgggaccccatgtacaacgaggtgagagcttggggctggggagggaggaggggaccccacggtctgggtccaccccctctgagcccgctgcctgtgccttgcagtacctcgaccgcataggacagctgttcttcggcgtcccgcccaagcagacgtcttcctacgggggcctgctcggtaagccggggcgcccttgtcacacccactccagccctgggtcggtgtggggtcatcatctctgcccaggcaggtccagtgcggtccttcacgtggaagtggttttctgatcttttaaatgtttctcagtttacttttgttagtaggaaattattagatttcagagttgagaattctggcacggtggctcgcagccccgtctccactccagagctccccatccctgaggtgtcttccatttcctggcagggatcacatcacgtggctggctgcgtgctcccccgtctctctgtgcgccatgctaggtggcctccgtgagcctggtgtgcccgggacgctgccagccgtcgtgctggggcaggcagtgctgccgtggctttggggtcgcggcggggcgggtgtctcactgtgggtggtggttcgtggcaccgcgggagaggcccctcactgagccgcctgccttcccagcgtcacctgtctgggtttattttagtgggaggtgtgaggtggtgacctctccccacagatgtctgccctggtgcagctgcgtcctgtggcaggccctgtcctccccggatgccccccccagacaccctggccctcaccgtgccccgtgacacaggcacctccaccccaacacccgtcaggagcgccctgactcttctgactctccagtcgaaaccgcagcctgagctcccgttccacagaagcgccgtcagggcgtgtcctgagatggggtgacgtcgtggataagagagggtgacactgccacggccgtgtctttgtattcaaagaacacggtgtaacgtctcgtttgggttcaggagacttttggagctttggctttgtacgtacagagtagttgttgattggataaaaatcagatgacagtgaccgcagttggcccctgtgggagcagccgttgaagctttggcacatccagttgattcatgtgtcactgtagggcgcagtgtgctctacagccccacttctctgctcaaaaatctctttccgtgaggtggtgttggttctgtaggtccctccgtggctcctgttggtgggtgtggagggtggttctgtaggcccccgtggctgctccatggccccgttggtggacgtgggagggccgttctgtggagtcccccgtggctgctccatgtttgagtatctctgtggtcgtcggctccctggcctccagagtgtcctgtcccggggttcctgggtcgtagacagtagcgatgtgaggcttttgcttgttccatgggacactgggacaccttgtgagacgtttgtgggaaggaggggcttgggggcctgtgggaggctgcccaggcgtgactgccacggtgttcttctttccagggaaccttctgaccagcctcatgggctcctcagagcaggaggatggggaggagagccccagcgacggcagccccatcgagctggactgaactggccaggccacgtggagacaccacggtcgacgacggctggagggacgtttcagaggcgagtcctgggtggctcctcgccttgggggctcctggccctgaggctggcggtggccgcatgccggcgcgtgtctgtttctgtgcggcggctcagggtggcgcggctgctgctcactgtgctgctgggacccaagagtggggcgtcgcccctgctggccgccgcgtcccccgagattgacccacaataaagcacaggccttaccgcggcgtcaccctctcccactcctttgttctgggtcctttcgggagggctgatgggcagcacaggaggcccgtcctcggggggctgcgcacatcacgctccttgccgggcgtccggcacagctgcggtcaccaaagcaggtgctggccctcggacctgagagcccagccagggcccatgtggtctgcaaatgggagcggctgtttttgaacacggggtcattctgcagtcaggacgaaccggtccccgtcgcagacggagtgcacgtgccctgcgccacatcctcacgctcggtggagggacgcgtgcggcgggacggtgcctacgggtacttgcagctgtgtcccatgtggcatcccagagctgcgccctgctggtctctgtgagcgccacgctgctgtgctggaaatgccgctttaaaaagggataccgtgggactctgcccgtctctttcataacgcaatatttatttgtattgggtgatgattgattctttcgacctaacattttgggttttaaccaaataaccggtccaggagtgagcagctccgttctgtcagatgctactccaaatgttaccagaacgatgacaaaaggggagacgctctattttttcacagttaaatgacagttgtagattgatacgcagttgtgcatgggaaggggaaacgcacagctttatttactgtaaagtggaatttcaggaaggcttgtgtgaaccgttgcgcataaataaaccctttctaccgggctgtgcaacgctggtcctttcttgggcgctggtgtgggggatctctctctgctctcagggctgtgcctgggagcccccagaggaggcgtaccctgtggccagactgcccgtgcgtggacggagcagagggctatcaggaagtcacgttcacttggctgtgattttagaaataagttttgtgtaatttcatcaaaattcataaaaatcaaatccaccaggtctgggtgtcacagcctcaaagagcaggcccttgggctgcaggggagacagtggcgcagagccctgaccctgtcctaggtgctgacaggacacccgtccctcccgtgtggcacttgctttgagcagcgagttcgatccggagtgaccaaggattttgtgtgcggcgtcactcgggcttaccgtgagacggcaggaacagccggccggagccccgcaggcggcccctcgtggcccgtgctgagcctgcgccgcggcgtctgcctctgcctgcctcaggacttcgcggcggagtcacggtccgtccctgtgcctgctgccagcaaggggatgcccttccccacagctgtgtccacgcggggaggtgtggacagcgagtgagcgaccaccttctgtgcctgcgccacctcccctggttcaaaaaattctttccccccattagaaagttgttctgtggtttttaaaatgtgacagtcgtcagtgtcctcagcacacacgtggacacgtgcacattcggggacacgcacacactgagcacgtgcccacacttccacgccatggggagggctgtgggcactgctggtttcccccgggggtgtgtccagcacagcccagggctctccctgacccaggcccactcctctcacctcgggttaggaccgtggcacctggtgaggttgcggggcacttgcctggggtttcagctccaagtgggggtacctcgcccacatcctcgtcaccagcacagctgccttggggacccctaggctgggattgggagacctctactccgccctagagtcccagctcctcattggagaagtgggattcagcatgttctgtgtgagggctgaggggcccagcattgggggggtgggctggcaggcacaggggacccccgacccgggatgcatcccccacactgtccttcatgctgagcttggcttggctttcaggctgtggactctgcccaggaactgcctggcgggcctcagatggagggggcacctggggaaggggggcacctggggaaggggtgctggggggcacctcgggtagggaccgtctcccgtgtacaaggagcatggcgctgtgactagctcagacaccgtgtcctccctccaaacctcagggagcgagggccctgtcaggtccctgggagcagacgaagattctcctgaagccagggaggcagggccacgtgttcccagcccaacttcctccccactccatcctcggacacacagctggaaccatgccagagccacattttattccaggatgcgacacggggcggcggatgcctggcctcggaaaccccctccctgcctatgagggcttgtccagggctcccctggaacacagctcagccaggcaaggctgggagagggctgggccccccaggtgacctgcagacgcctcccccacctacatgccccacgcaaggggagccctggagcccggcctgccttgacaggaaggagataggtgaaaaataaatacacggctgggccgcctgcagcaagcgcgacagtgcccgggacccccagggccactcctgcctctggtggcctcaccgcggcccaggccctcgtccccaagggttcagacaccactgtcggcgagcaccgtgctgggcgcagaggccacgtgtggtgggacggcagcctgctggcccctccaggtgaggaccaggagcacaggctgtggcaactccagcccgggcaggggccgggacctgtgtggataatccacccaaacaccccacggccctcatagccccgtgacacacaacaggcgctcaataaatagtcgtggaggtggggggagggcggggcggcatgcgagcagggcccagtccccagcggccggcagctgcccaccgtgctggccccaagcagggctctgcgctgaggcctggaagttcccacagccgtggtgggcggctcctgggggctgtgtctgagctcgggagccagactgggccctggaggaaagggggccccgggtcagggaggggcaggttggctgggtgtggtcagcagcagcatgacggggttagagatcaggcccagggcctgggctgcctgccttgaggttccagagaagcatcctggaagctgaagctcgggccctacctggccgcgccgggctgccctgaggagcaggtggggccaggtggcctcaggacgccagagcccccccatccacgggtcccctccgtccagccacagtgagtccaatgtccgtggtcctccagccgcactcgctaaggtttatgcttgaagtgcgcctccactgcaacggaacagggtccagcgttgtcacagcggcggggagctgggagcccttccaggccgccggccgcccgcccccctcacctgcgtactcctggggcagcatgggcctgtccaccgccttctggaaggccgccacccactccctctggtcggactccgtctcgcaggcaaacagaaacttgcggtcgggcgtgacgatggtgatgccatgtggccagtggtggccctgggtggacggcgggaacccatgcagcaccgtgtagccactctccttgctgccaatgaagacttcccctcgggcgaaggcgtcctgtgggtggggaccgcactggaggcggggccatgtcccttccagccccgcttcactcaggccaggactcgggaggccacccgccctcctggtcacgcatctggcaaccccagccagtgagctgccctcccgtgctggggccacagctcctccttaccagggggtctttgaagtacatgagcctgcggtcatccatggtgaaccagcgcttccggaagccttccgtttgctgtgggtcagagagggcccgtgaccggcaggtcgccgaggcaggccctacatccagctgaccacagcacaccccggagggcagggcccagactggcccgggtcagtcacctccattcactcacgcctgccgctggccacgccccacgaggcctccgcacacgcctgtcctccagcctccacacggcacctccctcctgctcctccaggtcccccagccctccctgggccccagatgggggcccaggcctcacttacgagccctcacacctgcacttctcctccgggaccaccaagactcccaggatttgtgtctccctcccacattatgcctcagggaatggcaagatgtgggcccctggccgcgcaggtgcacggtcccgtgggtgaggaagctggtcagacgtgagcgggcagggagggtctaaccaaagcctaggacagtggcccgagtcctcggggaccccggccaggcacagacaaggggctgtgaggacccaccagacaccagtttaaaacacaggcggcagctggcaaggctgctgcacttcagtccgagacccaccatggcgtaccccaggccaccccaggccgcacgtgcggcacacccaccttgggccccgtcttctccatgtagccttccttcaggtagttcctggagagctttggcaccagctagggcaggacacaccaggcaggggacctcagaagtgcagctcaggccgagcccctccctggctgtgcccctctgtgctcccctcaccccgggccaccaggtcagggcccaggcctggcttagcctccgcaggatccacatttctgcctctgcttgccaccccttccctccccgctttcccctttggctgagctgaagttctagagtgaggcaggagggctgcaggcacgtcagggtgagcccttggccagtcccaggcccaccctagggctcaacatcatcccagactcagggcacgagggctccgtccaccccccaccccaccacccctgtctgccctggagctgaccgcagggccgccactcacatctgcgtcgccggcccctgggaatgccacctgcaggtagtggaagcgagcagctcggagtgcattgaaccagtccacaatctcctaggggcaaaggtgggcacaggcttgggctgaggaggctgcagcgctggggtccccacagaggggctggggtccccacagaggggccgcttcccccagagcccagccccttcctcctccccggcacctcagctcccagcagtcctgccgcaggcctggcccctgtgcccacgctgaggccggggctgctacacaggggctgcccctctgcgggagggcaggtgcagcctcccccggcgaagtcctgagaagggccgaagggccgaagggccgggccgggccgggctggacagggtcgggggctgccatccaagctcactctgtggccctggtgctgctggggcctgggccaacttgccttcctttcagatggtgctatttttatgaagatccttattttgtagctcaagcaagtcttggggtggtgggagaagggaggctcatctgtttataaacgcccttggagctgtggcccctaccgagagtgggcagagagcaaaactgctctccagggagccgccctggtcctctggagagcgtggggtgggccagcaccctggaacagagggcccatagcccaggcctggcacccagccccacgtcgggtgccctctgggtctagacttcagcctggctgtcccctgcccctagacccagcgcccctcctggggtctgcccagacccttggccccatgacattataacctgcgccctgggcctcctgttcagcctacagcctggagctggtgacctctggaaaggtgcccatctttactcttggctgcaccctctcctgcccaggaagagccagggagccccatccctcctcaggcttctccccagcccctttggccttgacttttgtcccacaggcaaagcccccagcactggaccaatccagtcacggcttctccagctggcacccggcccagcaccagctggaggatggtgtagccccaggctcctcccatagcagtgggattgccctgtcctcttccagtggccccacccagcaaagccccacccaccccgactgcccctcatcggcctccagccctgctttctcctgtggcccttcctcctaggccacgcccacaagccacacccacgccacccaaccagcccgaggccccgcctcccctccttcccccgcccaccctttcctccggcccgccccgtcccatttggccccgccctccctcctcctagaggaagctgaacccgtccacaccacactgccccaaaggccttgggaattcacagctccaggtcccccggaaatgcagggggctctaagcagaactggttggaaaatcgccccttttcccccagcaggagatggagccgactctggggctccgggcagacggggcatgcctggtgcactccacgctggagctctgtgaccctgcaggctgcagcggaaactccctcagctcccacagctgggggccaagcgtctgccctgggcggggggaattatctttggaggatcaggagttggacaccagcctgaccaacatggtgaaacccggtctctactaaaaatacaaaaattgcggctgggcgcagtggctcacacctgtaatcccagcactttgggaggctgaggcaggtggatcacctgaggtcgggagctcgagaccagcctgaccaacatgaagaaaccccgtctctactaaaaatacaaaattagctgggtgtggtggcgcatgcctgtaatcccagctactcgggaggctgaggcaggagaatgacttgaaccagggaggcaaaggttgcagtgagccgagattgcaccactgcactccagcctgggtgacaagggcgaaactctgcctcaaaaaaaaaaaaagaaagaaaagaaagaaagaaaaataaatactccaagaaccaaaggctctctgaaaaactaaatgcatgacagcagaaacaaaaccctcaacacagtgttggagacaaagttgaagaaatttcccagaaagtagaagaaaaatgaaccagtttaagagcaccaataacaggagtttcagaaaatgagaacagggaaagtggagggaggaaatcattagataagaaagcaatccctgacctcatggaaaccagtaactggactggaagggccaccctgggcctgtacccagcacatgaaaagacgagggacccttgccaacacacgcacagcattggagaatttcaaaaccgcgggacaaacgtactaggatggaggaatccgtggtgcaaagccttcagaattagcaggaaaattaattccaacctagaattctatatctagccacagtctaagccaggcaggaggtagaacagtccaaggtgccaggtgacccctttccctccaggcacatcacttctaccaagaggaataagtaaacgagaaagacgtaggacccgggcagcagggctccacccaggaaccagatgaggctgcaggagcgagatctccaggccgtgggagtcgcaggctcctggtgcccgactgcctccagagacaacagtctgcggagagtctgcggcaacacatggtcaactgcggcaaggaacacagccgcttactgtgggaagagcgatcacagcgtgctctgaggccaggggctgcacccgactcggccccctaacaaacccagggtcagatgccagcggagcacggcaggcaggggacgggcacgtgggaggggatggaaagagctggaacctctacttccacgtgggaagttgtcagatggtgcctaaagtgggtaaatctgcagcgacaccggcagcttatttttggatatggggaccaattcccaacgggggttaggagggacctcaaacttctgtttctcaagatttaggaacgatatagcgctttagaccgtgtgcacacagacgtgtgataaacatagaaacaagaacctaagtaacaaagaggctgggcccaccgctgcgccccctgcagcctggcccaaagacaaaaggatagcttttgtgggggttttacctgggttccccccaagccctccagagatgcggggtacacccaccctccccgccgccacaccaagggcagccaaaggaaccctacccaaaccaccccagagccggctaccgtccacatctcctccccgtaggaccccagttccccccgaggtcccctctgggcattgtgggggcggtggatcctctagcccgagctggcagcccccactgacagggccaagctgggtggccccagagacagcgtggtgatccggctcctgggcagcccggccctgggaagctgccttcctgcacctgtcttcccatgctgcccggcgccagtgcccacactcccacacgtccaagcacccgccttcccacgctgcccagcccgacttcccgccttctcatgccgcctagctcaagtgcctaccctcccgtaccgtccaagcacccaccttcctgcgccacccgggcctgagggcccaccctcctgtgccacccgggcccgagtgctcgccagcacccaccttcccgtcctcatggtagatgaagatgttacgggtgctgttgtccttcaggtaggtgacctgcaggccgtgggggtggccgatcttggccggctggaaggtggcgttcaggtgctcgatcttcatcacggccttgggctccttggcctgagaaggggtggggtctaagcacctcacaggggccgtcgtccaagctcaagggagcaggaagggcagaccctgtgggtgctggcggcgcacctgctgtgctgtgtggctttgggcaagttacttaagcgctctgagccttggcctcccggtctgtaagcagaaggtaacagcacctccctcccaggagtgatggggacctcaacgacactgtgcaggtggcacttgacatcagacactgcacctccctccccgggggcagccccgggccccacacggcacggcagctcccacaggggcaggccccagccaccagcccacctccagccagtgcccgccacggctcagggcgggtgcttctgaccatcagcccatgggtctggggccaccttgtgaagaccccactgttcttgccaatgtgtttgtcctgtcccagtccagtctccccagggtacctccaggaaaactaggggctttctggggtcctggctggctccagaggggcccagtgactaggttctccctctcccacggctgggcctgagttcagggggccccgcccggccgcccaggctcctggaaagcctcagctgccccaccctgggggacgctgggtccggcacacagaggctcagcccaacacaggccggttctcctggagcgtccccatcggggggggcagcagggagggcagctgcggatggacgcggccaccacaggccccagtgtgggaggccgggatgccgccctgggacaggcccccaccccacccccgtctgtgactcacatcatttctgttgaaatacttcagagcaccctctcgttctgtcagcacaaacttccggctcaaaaactgcccgttgtcccggccacgcttccagagaaaaccctcacggtaccctgtgggggaaaggggacacgagtcggtgacagtggatgggggggaggaaacaaaaggagtgacgatggacaggacagaggggacatggggagaagacacgggggacacggacgggggacacggacagggggagacggacggggagaggggacatggaggagacagggagataggaagatgggcagggaaagggaaagggaaaggagaaaggagaaaggagaaaggagaaaggagaaagggagaaagagaaaggagaaaggagaaagggagaaagggagaaagggaaaggagaaaggagaaagggaaaggagaaaggagaagggagaagggagaagggagaagggagaagggagaagggagaagggagaagggagaagggagaaaggagaaaggagaaaggagaaaggagaaaggagaaaggagaaaggagaaaggagaaaggagaaagggaaaggagaaagggaaaggagaaagggaaaggagaaaggagaaaggagaaagggaaaggagaaaggagaaaggagaaaggagaaaggagaaaggagaaagggaaaggagaaagggaaaggagaagggagaagggagaagggagaaaggagaaaggagaaaggagaaaggagaaaggagaaaggagaaaggagaagggagaagggagaagggagaagggagaagggagaagggagaagggagaagggagaagggagaaaggagaaaggagaaaggagaaaggagaaagggaaaggagaaagggagaaaggagaaagggagaaaggagaaagggagaaaggagaaagggagaaaggagaaagggaaaggagaaaggagaaaggagaaaggagaaagggaaaggagaaaggagaaagggaaaggagaaaggagaaaggagaaagggagaaaggagaaagggaaaggagaaagggagaaaggagaaagggagaaaggagaaagggagaaaggagaaagggagaaaggagaaagggaaaggagaaaggagaaaggagaaagggaaaggagaaaggagaaaggagaaaggagaaaggagaaagggaaaggagaaagggagaaaggagaaagggagaaaggagaaagggagaaaggagaaagggagaaaggagaaagggaaaggagaaaggagaaaggagaaaggagaaagggaaaggagaaaggagaaagggaaaggagaaagggagaaagagaaaggagaaaggagaaaggagaaaggagaaagggaaaggagaaagggagaaagggaaaggagaaaggagaaaggagaaaggagaaagggaaaggagaaaggagaaaggagaaaggagaaagggaaaggagaaagggagaaaggagaaaggagaaagggaaaggagaaaggagaagggagaaagggagaaagggaaaggagaaagggaaaggagaaaggagaaaggagaagggagaaagggagaaagggaaaggagaaaggagaaaggagaaagggaaaggagaaagggggcgggaaaggggacatggacaggggacacgggggacatggacatgggggacgggacatcggggacgggacatgggggacagagtacataggggacatggacaggggacatgggggacaggggacacgggggacgggacaggggacatggggggacatgggtcagatggtgatggtggatggttggtgaggggatatgacagtggaccagcgctgttggctgcaaccctggcccaagccagcctgaggacggcctggatgagcgggactccagctgtcctttactggcccccagggaccctccccatcctgagtggacatggccagagccaccatgggcagaggcagggctgggtccaacctcgtgctgcccgggaggaccaggctggcctcctccctcctccctcagccgtccgcagctgctgggtccaggtggtgccatcccctcttttttcaggaaagctcatctgaggggagtccagctcaagccctcatctccgcctgtggcctcacgcacaccacacacagccacagaaaagtagcccgggcatccccctgggagcctaagcacctggacacgccaccttctatggcaaaacacaccgtgcacgggtggttaggacgctgctgtggcagacgatcacaggggagggaggcagagatatgaggacaggagggaaggggacgtggccgtggaaactgaggccagagggggccccagagcccaggagcaccagggagaggccagaggacccgtcctgagcccagaaccagctctgcccactccgagtctcgggcccgtgggatcgtcctggagctctgccagagctgtgggagaacaaacacgtgtggcttcacgtcactgagctgcggtcagtgacccacgtgtggccactcagaggggcgttcccatcccccccaggccccgctcccacttctgctccctgtccgtggttcctatcgttgcccgtcctttctcctgctctggaactctgtgtcctgcggactcagatgcagcttcttctatggccctggctgtcccgccccaacagcatgtggcttgcagcatccgtgtcctgggctccgtctgccaggccttgggtcgcgtgcaggtcccgccgttggcagcagctgcgtggccagggtcgagcaggtcctcagtgggtgcagactctcctgggatcacggggccgtctgccaagcatccccagtgggcgccgcctctccggaggtcgcggggccgtctgccgaggagccgtctgccgagcatccgtggcccctccaggggtcgcggggccgtctgcccagtgtccgtgggcccttcccggggtcgtggggccgtctgctgagcatccgtgggcctgtccgtgacccctcagtgacttcagagagcacttgcttggggacagcatcccatcctgctggccatgtggcctgggagccccggtcatctgagggcaggggctgctgacctacgccacagggacgcgagggggctgtgggctccaggcggggctaaacagccccctgcagcagccaacaggccagacagaggctcagattgcagacaccgcccatcagccctgaccaccaaagccccctgctccccagcatcccctccccagctcagacagccccatcccgacaccagagaccaggggctattttctctgctgagatgcaagccataaaacgcctggcgttggggaggggacagctggtgccctcggggagggggccctgggctgtcttggatggtgccagacggggcctgctcccctcactgcccggagctccccgctccccaagcctctacaaggctgctgccccagccccgtgcgcacagcagcagccaggagcccccttcccatggaagaacccccttctcctgcttctcactgtgagagcgaggccaccagtgtccctcagacccaagtaaggcctgctcagaacgcctgttgactcagtgagcccctgcccaggcacagctgggtgtccccacggctctgcctccgacatcaggacctctttggacgcggcgcttcgtggctgagggtggacgccgagaagtgcccggggaggcatcgcagcagcgggcccagcggcgcttcccggcctctgccgctcacggccttgtgtgcaccccggggagcctccaaggcgtgcgtgtccgtgtcaccaatgcagacgacaagtttgggccacctcgcccacgggggtggagctggctgggtccagtgtgtcggttccacagacccctgcccaggacacagcgggggccccttccagcgaccgggctccctaggccacagggtggggacgcgcccctgccatgcccgccgcgcccagcaggtgacagcagtgctccggaagcgcgcaggccgcggggtctgggaggccactgcccctctgcacgcgcgcagctgctccggtgttctcggggtccagacgccagcgggaaccccggtcctcccgacagcaggcgccagcgggaaccccggtcctcccgacagcaggcgccagcgggaaccccggacctcccgacagcaggcgccagcgggaaccccggtcctcccgacagcaggcgccagcgggaaccccggacctcccgacagcaggcgccagcgggaaccccggtcctcccgacagcaggcgccagcgggaaccccggtcctcccgacagcaggcgccagcgggaaccccggtcctcccgacagcaggcgccagcgggaaccccggtcctcccgacagcaggcgccagcgggaaccccggtcctcccgacagcaggcgccaggacgccgcattccaggggcaatgacgacgcaggagtgtatttcaggacggcgctggccacggtcccggacgcgccccacgcccagtccagacctcaggctgagcctctgctctccccatgcctggccagccccttggcctcgctggggagggccaggacctcaggggcctgcagcccccagtcggcaccagcaacagccagtgagacggggcttctccccacagagcagcccctcacacggcagcgtcctggacagccgggtgggctgtggccagaaatggccattttctcttaaaggactcgtgagtcctggggcaggactcgaccagtatttatcaaacagctaccgcgtgccgcgccctgctgtgagctggtcccgctgtgtgtcgggtcctatggtgtggggtggggggggttctactgcacaccaggccctactgtgtgccggtcccactgtgtgccaggtcttgtcccacctgtggggagctgggtccagtccacaccccatgccctgctcagacgcctctgaggagcccttctgcccaccgcacggctaagagcaggaaagacgttcagcatcgccatgacccacgagaacggcaaaaccaaaccagtgtgcacacacaggcgcccgactgtgggcggggacgccggcccttccacgctgcccgtggggatgtaaggtgccccagtccctggaaagcagccaggccgtttcttccaccacagtgaagcgcgcacatcacgtgcctcagagagaagtgaaatccgaggaaacaggaacagccttcacataaagcccacacccgtttacgaggcttttttttttttttccccacccaggctggagtgcaggggcactatcacagctcactgcagccttgaccttccgggctcaagtgatcctccagccttggcctcccgagtagctgggaccacagatgcacgccaccacacccggctaagttctgtatttttttgtagagatgggagctcactgcgttgcccaggctggtctcgaacttctgagctcaggtgaccctcctgcctctcctcccaccatactggggttgcagcgtgagccccacgcctggcccgagactgctgggtcttggatttgtcagagctgggactgcactctaaataggatatgcaaagtagatgtaaatcacacctcacgtatgtgatttatatgctaattgtcttgtaaactggggcagggaagcacgtggccctgctctgagtgacagctcacagggtggtggaccctctggggtgggaactgcatccccatatctcatggggcctctggccttccacagggaggggtgcaccatggatgggtggcctctgagaacggggccgaggccctgatcactgcaccccggccgtccctgtgaagggcctggacctggcttccggcctttgggtcctagtggctcctgccctccctcaggaggcaaacaccggagcccacggcagagcagtgtctccttgctgggtaaggccttagagttgaaggcctcctctgtgagcccgcacagggctccgtctctccctgcctggcccgtccacccacgctctccgacccattaatgcaccttctccaggttggtgcgtctggggccggcagaggctgagaaccagcgggccgtatcctgcggagggctccaggtcccagcttcgccctgcgtggagctctctctccaaacagccaggacagggaggcttccctgttcccaggggtgaggccagagggtggacgagggctcctggggtcagctctcaccctgttcctattctcatatagcccccagcatggggcccggcccccgacaggcctggagagacccccccactcacacacgggttcacagtttccacagcgccttcctgcccagagtgacgtgtggatgcccccatccccgccaggctgcccaggctgcaggaggggccaccctcagggccaagtcagccccagggtacccccctccccaagccccaaaccctgaggcccccacacaggggccccagcgcccaagaacagcgaaccctgactcctgtaagacaaacatgccaggaaaggggcggggatcacacggagggccaggaagggggcgggggtcccacggagggccaggaagggggcgggggtcccacggagggccaggaagggggcgggggtcccacggagggccaggaagggggcgggggtcccacggagggccaggaagggggcgggggtcccacggagggccaggaagggggcgggggtcccacggagggccaggaagggggcgggggtcccacggagggccaggaagggggcgggggtcccacggagggccaggaagggggcgggggacccacggagggccaggagccgacacccacccagagaggggcaatggggcagccctaccacctctggccaggccccaaacacccgtccccccgaggggcagccccacagctctggcctggccccaaacacccgtcccctcatgggccacgtaaagcctcgcgctgcctgcgaggaacggctggtgatgttcccagtagcgccctcgtgccgcacaccatcgtccccactatatggacggggaaaccgaggccccagacctcgtgcacagccccagccccctcctgggctccgtctgccctgtgacatttacggcaacggtcggcccatccctgcctggtaccaccaggaggggccgtcctgaaccacaggggaaccacagaagctccgacaggagcacggggtccggagcctagagcaggaagccccgcgtctcagcgagaccagggcctggccctcaggacaacatgaggcattgtccaggcccctgtgaaagccgccattgaggctgcggcccgaggcggggccagcccgcggtgggagcagctgtcaccagggtcacccccaggggcaggcggggggcaagtctggctcagacacaaggataaacccccacatctgggggaggggtgcggcgcgtccccacgcagtgaccttggcccagggaggcggcgcagagggtgggggagccctgctggctcagcgggctgaggttcacctggggccacagcgacgcaccttccgctacgcaccgacaccaagggcttctcccccggtcctttgtcgtcaaagagcatcttggggagacacagcggccacagaggggcttccctgccacaggttactgcaacgtgcacctcgagaggtgtttccgcttccctcactcctcttgcatttattttattttgatacggaatctcgctgttgcccaggctggagtgcaatggcgcgatctcggctccctgcaacctccgcttcccaggttcaagctattctcctgtctcagcctcctgagtagctggagctatcacgccaggcctatttatttatgttttaagacaaggtctctctgtggcccacactggagtgcagtggtgcaatcacagctcactgcagtcttgacctcctgggctcagaccatcctcccacctcagtgtcctgagtagctgggaccataggcgcctccatgcccagctaattattaaactttgtcttgagacggagtctccccacgttgcccaggctggtgttgaactcctggctcaagcaaccctcccacctcggcctcccagagtgctgcaatgacaggcatgagccccggtgcccggccttcttttgtattaactggaattcgtgtgcaaaggctgctggcctcctccccttccttttttttttttttttttttgagacggagtctcgctctgtcgcccaggctggagtttagtggcgtgatctccactcactgcaagctccacctcctgggttcacaccattctcctgcctcagcctcccaagtagctgggactacaggcgcccgccaccacacccagctaatttttttgtatttttagtagagacggggtttcaccgtgttagccaggatggtcttgatctcctgacctcgtgatccgcctgcctcagcctcccaaagtgctgggattacaggcgtgaggcaccgcgcccggtcctcccctttctctttacttgttccattctttatgtatatgtctatggacttgggggtgtgtaccttgctcccctgttataatccccttataccatcttccttttgttgctcaaattactcccccagctttagccattaaaagctccttcagaggccaggtgtggtggctcacgcctataatctcagcactttgggaggccgaggcgggcgggtcacctgaggtcaggagttccagaccagcctggccaacatgatgaaatcctgtctctactaaaaatataaaaattagccgggcgtggtggggggcgcctgtagtcccagctactcaggatgctgaggcaggagaatggcgggaacccgggaggtggacattgcggtgagctgagaccgcaccattgcaccgcagcctgggcaacagagcaggaaaaaacaaacaaacaaaaaactccttcagaaaagcccggtggtgatccttccttttgggtggcctaacagagggatggagtggggctccgccttcctcagccatggtcctgggtggcgtggcccacacacaggacatctggggcccccgccctgtgagaacaacccggccatttccacccacagccaaggtgcaggctgtgggcactgggcgtggcacagcctctaggagttcttggttccggctcattccagggcagacctgcagccactcctctgtgcagagcgggagccacctggcccaggcctcacctggccaacgtggggttgtgggtgtggcaggcccttcccaagccccgagcttccccttcctcccgggccctgccgcagggacttctatatttccctgtggtcgtgaggtgtcgccactgccgtgataaagggctctggtggcaaagccagggagcagggcaggaacgccgccgggaatccccagtgcactctgggaggggttgggaggcccgggaactgccaactggcgctggagccctgtggaggaggctctgcccacggggccgccccagacaggaggacacgtcctctcctgctgctgcacccacccagacatgggctcagggtgggggccatggggaggctcggggggtccctgaggtctaaccctgagcaccagatggacacctgctccctcatccatccagcaaagggcagaacctagggttccaaatagcagcgggcagctaggtctgtgggagaggtgccctggagcccctgaacacacaaggagacaggcagggcatctcacagggccctggaaccccgccctgcacagcacccagagacagcagcctggagcacagacagtcaccgcctgctggtccccaggcacttggccgtctgaagcagccaggccttcgtgcctactgaccaggctgcaggggcagggtagaggggaggcttcaaggaggggtgagccctgccctgagcaggtgggtgtcagtgggccctgagcacagggccatacgactcagcacctctgggtccctgggatcccagcagcctccctgcagccggggctctggggcccgaggccctctggctggcgtgcctggtgcctcctgcgcgacctggaaggtgccgtggcccaagggagacagcagccacacactctggagcagccgcagtctacgaccccatgccctctgcaagaggctgccaccctgctgccaggtaacacctggccacggcactcacggcaccttcacaaagggccagccctgcactcacacctgcacacctcgcctgtgcatctcacctgtgccgcacacacccgtacatctcgtctgtgtacctcacccgtgcctcatgcacactgtgcacctccccaccatcctcacacccatccaggggagagaacagagggcacagcccatgcggcctggtctccacaggcagatcatgagcctggtgctgcggacgtggccgccccgccgggcctggtatgagaagcggccattcactcagcgtgggcttcagaggccggcaggcaaggccaaggccttgggctccgaccctgcgagcaagcgtgtgcctgaaaacctctgccccgactctaccctgctgggaggcctctccctccctccaacccctgcccgagggtcacacgggaggctttagctcatctcaaatgctctcaagggaatatctcagacaacagcaaaggcaggaatgggattccagtggggcaggaacaggattccagttgggcagcctcgggcctggggcaggaggcgtcttgggtcaccaggcctgtcaccacctcctggccctgagcccaatgacacccatatacccctgcagaacctacgcccactgccgacgagacgctgtctgccacccgcctgcagaacccagaacccgcgcccactttccacgagacgctgtctgccacccgcctgcagaacccagaacccgcgcccactttccacgagatgccgtctgccacctgcctgcagaacccacgcccactgcccacgagacactgtctgccacctgcacgcacccccacttctggagcccagacctgatcttccccagaagggccactccaggggccagaggcgggcacgccacctagggcgccacgtttcccaagtcacatcccggcccgaccagtcagaggtgacgagacagggactttttccagcacttctgtgagccacacctgacctttcctggcagcccgggagagcagggatgtaaaacccaaacttgccaaaggaggtggccaccaggaggattggtgccaacctagagacgcagaactaggagatagaggtgctgaaaccaggtcccaacgacaccagctaaacccctagatcaagctgtgccctgttattccagtcaatccatttccagttttgctgacacccagtgaagttggattttccatcctttacaaccagagggttctgatacaagaacagacacaaaactcagtcctgtggtcacgccagcccagcccatgcctgcctctgaagtgaggccacgtgccaccttgctacaaattgctaagatgtttctgccgagggaaacggaggggctctggggtgattctttgggggtttaaggcaggagtggagcgaggggtgtgctgggtgctgagcaggtccaggtgcccgcgtgttcatcccctgaccgtttcttgctctctccctcacacacacagggattcttgagaggaagaaattgctgtctgggtagacagtaattgaatcagcgcctgcacaggctggctgtgtgccaagtgggggttgggggggcaggagctgccctggaggagggcagagccgacaggaccgctgcatgagaccccagcctgacctggggaggtgccttgctcaggcctggggcagcctcccaccggtcacccctgctcaccccacgccgctcctgggagaaaatccacggtcctgacagagcccagggaggtgcacgggaggggggcgcagtgccagctctaccggggaggtgcacaggaggggtgcgcagtgccagctctaccggggaggtgcacgggaggggggcgcagtgccagctctaccggggaggtgcacgggaggggggcgcagtgccagctctaccggggaggtgcacgggaggggggcgcagtgccagctctaccggggagttgcacgggaggggggcgcagtgccagctctaccggggaggtgcacgggagggggggcgcagtgccagctctaccggggaggtgcacgggaggggggcgcagtgccagctctaccggggagttgcacgggaggggggcgcagtgccagctctaccggggaggtgcacgggagggggggcgcagtgccagctctaccggggaggtgcacgggaggggggcgcagtgccagctctaccagggagggctgggaggggttggagcactgggggtcaggggcttctccactgaacccacctacaacaccaggactgtgttcagccatttcagggccgcctctctttcacatattttgagacagacttttgctctgccgcccaggctggagtgcagaggcgcaatctcggctcattgcaacctccgccttccaggttcaagcgattctcctgcatcagcctgccgaggagctgggattacaggcacctgccaccacacctggctaatttttgtattttttactagaaacagttttgccacgttgaccagattggtctcgaactcgtggtctcaagtgatccatccgcccgcctaggcctcccaaggtgcttcttttattcttggggtgagattcacaaatacgattcatcttttttcttgttacagacacactctcgctctgacgtccaggctggagtgtagtggtgcaaccttggctcactgcagcctccgcctcccaggctcaagagatcctcccgcctcagtctcccaagtagctggaaccacagatgtgcgccaaccacgcccagctaattttgcattttttggtagagtccaaaaaatgagcaacatggggtttcgccacgttgctcaggctggtcttgaactcctgagatcaggagatctgcccgccttgacctccatataaagtgctgggattacaggtgtgcgccaaccgtgcacagctaatttttgtatttttagtagagacggggtttcaccatgttggccaggctggcctcaaactgctgactgcaacggatccacctgcctcccaaactgctggaaccaccggcgtgagccaccgtgcccgacccttttttgtttctaagacagagtcttgctctgtcacccggcctggagtgcagtggtgcaaacacagctcaccgtagcctcaaactcctgcacgtaagggctgctcctgcctcagcctcccgagcagctgggactacaggtggtgccaccatatccagctaattttttcttttttttaaagggagggcatcttagatgttgcccaggcttgtttgaccaccagtgatcctcctgccttggcctcccaaagtgctaggattacaggcctgagacacggctcccgacctcgtgttccgccattccagggcagcctctctcttacacaagggcagtgggggcagtgctggggttgcaggtccgggccctgggggctgcttccttcctcacgtggccatgcccagcagcaccaaacatccccatccagcctttggaccccgccctgcccgtgctgacgttcagaaccaggccctagggctaggcttgcctccgtttccctgtcgacacggtggagaaaggggcactcaggaagctccagtgtgcagacgcccagctgccctggggaaaaaaccactcaaggcccacagtacccgagcccacctcctacagagcagacaggaggaggaagccagcccacccggctgggggctccaactcaaagccccagggtttaaatgagatgccaggagaagcaggagttggggactgctggggggcagagttggggactgctggggggcacagccagaggcttggccagctaagcagcactggggagaccgaggctggggtggggagaccgaggctggggtgggctggcctcccaggcccccatggcactgcctcccagcgcccacctccacagccccgccagcccacgaagctccaagacgctgcctggatgtttctgccacccgagggcccccaagggcacccaggcaggcctaggggaggtggaggccgcaggccggggccgcaccctccccacctgcagcacagtgttgccacacagaggacgccctcacgcccctcacccgggccttcaggtcctgcccacactgctggagacaggggtccctgcaggccacccccgaatggaagtgcgtggccaccaccagcagaggaggggccagccacggccccacgaggtccccatgcaccccactccttcagggctggactgtagacagctgtggcgaccccaagccctcagtcacgtcactcagagggcaaaacaggtccggggacgcgggttcaaactcacgagtgtgttacccaggttaataatgactctgtccacacgagagggagggagggaaggagagagagagggagagacagagatagagagaggatgagatagacgtgaagagagaggaagagagacagagaatgagagacagacatgaagagagagtaagagaaacagagacacagagagataaagagagacagaaggagggagagagagagagataaagacaggggagaaagagacaggtgggggtgggtgggagagacggggagagggagggagatgggggggaaggggggagagggagggagatgggggagggagggagatggggggagggagggagatgggggagggagggagatggggggagggagggaaagagatgggggagggagggaaagagatgggggagggagggaaagagatgggggagggagggagagagccagggagaggtagccgggaggccccaccccacccgggtggcctcacctgccgagtagggctcctgcttctccgggtagatgaactcctgtcgctcgtacttggcccggatccactgctctcgaaggagcctgtggggagaggagagactgagccactgggccaaggcggcctccgacccagcacacgccgctctctggcccggaccctggacatctcaagaggctcatagggacccccggcagactcgagccgcccctctgggcacaagatcccggaagaaatgcagggtcagcctcctgcccgggacgcttctcactctgatattagtttattggtttcttgtgcgttcggcccccggagcatcaggcctcacgttctgcacaagcgttcccggtggacgggctggtgcctcccctctccttcccaccttggcctcagctgatgccccacagggctggggtacaggggttgagccaggcccccccaccccgagtcacacgcccctgggccctccccgaccctccccacctcgttggaccggatctgggaagtggccgcggcgccgccctccacccaccgtgctgccaccttgcaccccccgtgccgccctccacccgccgtgccgccctccacgtggttctgagacacaggacggagctatcagggcacccgtcgaggtcaggaggcgtccggggcatcaggagaaactaccggcaaatacccaagaatccaggaagacccgggatgaggagaagcccccgagagtaaagcccgggacgagggccccccacggcacccactgagcccagacatccctgcccagagccacccaccacggcctccccatccaccgtgactcactcgagtgaagccaataccattgcagaaaccacgacccacacacaggcccggcacggcccaggtgcacaggcagccgccccagccttttccactcccagggaattacgcggcaaagaacaaataggaaccctgtggcttcctgctgggcccacgtgaacagccttggagactgggcgggaatcctcgcccacaggatctgatgggtgatgggtcccagctgggtctctggcccctggccccaggtgtgtgtgtgtcccccacctgctacaacgatcaccccaaaattcggtccccgaaagcaacagaaatgtatcatctcggtcacggaggccaaacgtctgagatcaaggcgtcggccgagctggtccctccaggtgttctggggagaatctcctccagggtccctcccacccctggaagccgccagtgcccccacctcggcgccgtcctcacctggccttccctgtggggtttgggcccaccctactccaggacaacctcacaaccttatcttaacctgaagacatctgcaaagatcctacttttctttcttttagagacagggtctcactgtgtctcccaggctggggcacagtggtgcgatcacagctcaacgcagcctccaacacctgcactcaaggatcctcctgcctcggcttcccgagtagctgggctagaggtgtgcaccaccacgacttggtaattttttattttttgtagaggtgggagctcactctgttgcccaggctggtctggaactcctgaggccaatcctcccacctgggcctcctacagggctgcgattacaggtgtgagcctcgcgtccggcccctatttccagatgagttcaccttcacaggggcggcgggttaggactttaacacgtcttttcgggggacaccgtgcagctgcctgagccgctccggctgtgtgaccacggaggagttgccccctagcacccagcgtcccatgaccccagggtcacacagcggcctctcagagtcacgggaggaccgaaggccgcatgtgtggaagccgggggcctgtggatgagaggcaggtgccagggacggctttgacaaccacgcctgccaacggccctcagggcggggcacccacagggtcggcagccctgccctggtgcagaatgggcccccaaggctcacacccgcacacaagggcatcccagggcctggcacagaagcttccagaagcagcagcagcaggaggaagcctgggagtgagcaccaggaagtcgggggctggaccccatcctgcccagcggagcctccggaagcccctcatctcctgaaaaccagtactgcccggagccctcccgactcccacagccctggaggggccgcactgacaccccacgtggtcacgaggaggtgggcttcgggccacctggcagccgggacggagagccaccaggaaagctccagccacacctcagcccacgccccaggaagtgccccaagttccagggctcaggggagccaggcttcaaggacccctaggccaccccttcaaggccagggcctcagggtcaggtgggtcccctcaacgcagagcaaaatgtgagctgggggcagggggccgggcaggtgctcagggcgactgcctcccctaggtgtggactcacaggggaagacgctctgagccccagaagccacttccccacagggctcccccagcccaagatgccatgggagacccctccccgctttccccaggacgggtccagtggccactgtgacccccatagcgggtgctgttcacacagctggcacgtccatccctgagggtccactcctgagtgtgccttctgcccgagttctgccagtttccaccgcatggagttcaggacaccatcaacacccttcctggaggctttgcctcttgcgaaagtgaccccccagggcccccggctttgcttctacgtggtggaccagtggctcacccagatgggttttttccctttttattacaggaaaatagcccgggtggtgttcacgcccccgcccacacccccgcccacgcacctgcctctcagcaacacccccacccccgccacccccgcccccattgtctatcctgggtgtttccgaggctgctaccacctgaaccgcgaccccatcctactgggagccctgacccccgggcctgagtgtgcctgtatttggaaataccgtggttgcagattagttaagatgaggccacaggagcagggtggcccccaatccaacgtgcccggtgtccttaggaaggggaattaggacacagacaggtacgcagggcccacgatgtgaggaagcaccccggagaggcctcgggagaagcagccttgagcctccagcctccagagctggaagaaagtagatttctgttgctctgggccgccgggcatgtggtgctgtgttccaggagccccagacatgcatgcagagccctgtccagcccggacacctgctccacggggggcttgctggggctgagggctccagggcttcgctgggatgggcagagaggagccctccagccggtgctgccagcagccaagggctcctcccacagcttccctcactcgccctgatcacccctcaccacacaggaagcagggaggggccggtttccacccagaagtaaaaccaagcagagcggccgcacgactttctttccccgacgtcaagatagaaactggagagctctgagggtgacacaggaagagacgggtcccggcctctgagtcacaggctggtgctgaagataaagagggcacacgagaccctctgccctcccctgcgggtgcagcctcccctcttatagctgggcaaactgaggccctgggcctctgcttagctggaggtgggcacggtgctcgacctgcctggagggaccccagtgtgtgccgggaggccgcagggcatggcgtggggggccttccaggaccctgctccggacgctgcccgccactcttagctcctgcgaagacgccacccggacatctgtgcagcagagctttcccctgaagaccttcaactcagacccgggcttgtccaggctgcacggaggccgtgggcaccagcgctgctggggctgggagaatcttggtgggggcagacgtgggaggaaggtggacgagtcccctgcccacctcgccaggctggacgtttgggccccacgagccacagagccggaccacaaaatggcaggatgtaggcaccccccgccctccaggttacactgcaggtccacgctgcaccccccgccctccgggttacactgcaggtccacgctgcaccccccgccctccgggttacactgcaggtccacgctgcaccccccgccctccgggttacactgcaggtccacgctgcaccccccgccctccaggttacacagcagttcacgctgcaccccccgccctccaggttacacagcaggtccatgctgcaccccctccccgccctccaggtccacactgcaccccctgccctccaggttacacagcgggtccacgctgcacccctcccccaccctcgaggtccacgctgcaccccccaccctccgggttacactgcaggtccacgctgcaccccccgccctccgggttacactgcaggtccacgctgcaccccccgccctccgggttacactgcaggtccacgctgcaccccccaccctccaggttacacagcaggtccacgctgcaccccccgccctccagattacacagcaggtccacactgcaccccctgccctccaggtccacgctgcaccccccgccctccaagttatactgcaggtccacactgcacccctgccatgccccagcatccacggctcatacagggattcacaccccaccccagatagtgggtgtcagccctgggagtctcctcacccaggacccaacagcagcagcccctgagtccacgagcacccccagccccccgcacccatgtcagcccccggtcccgactcagaacagccagggagtgggaggaacttggagacagtgacccaggccacagagcagctgggggcggggtgataaaataatgtcttaaaaatagctgtgctggggctccatgattcataccgagagcccggggcacggccagggcagggttcccctgtgagggctcagggggagacagcagctggcacctggggcctcagaatgagagtacggggggccagggcgccaggcctgcccgggtgggaagggagaggtgcatgcacggggctgagtcgccagcctcggccttctcagtggcgatgtggggattgcagctggacctgtctccaggctcacgggaggctcatcaagaaaacgcctgccaagcacctggacgggggcctcgtgaccccaataacaaatcaacatgcagccaaagaaactgagtcgctcgtggcctgaacacacctccagcccctgctccccgaagaccccgggagggccggttcatgctgcaatcccagcactttgggaggctgaggcaggaggatcgcttgagcccaggaatttgcaaccagcctgggcaatgtagggagaccccgtctctatatttaaaaaaaaaaaaaaaaaaaaaaggcccaggatggtgtgtagggttgcagccgggaagtccgtccttcctcccctctccctcctccctcttccctccggcctcccctccaccctatcccccaacccccacccgccttccaggggccttcggatccagtgaggacccctgatcccactgccccttcatggagctcactgcagggggcacagaccccaaacagggccccggatgctggggaagaccccctggtcactgcagtccccaccagcagctcctgggcaggcagagcccaggctgtctcagcccccgagtcagggcagagccagcaacggcacgtgggcccctcatgggctgggaggccaggcgcccgccgctttcctgcggcactgttggctcacacccaaaccctgcacccagctcgtgctcggggtatctctgcggggtgggccctctgctgagtgggttcacggagtgagtcaacggagggcggtgcccagtgaggctgggattgaaccagggcaggaagcatggggggccgggagggcacccagtgggccaacacgtctttcccctgtgacaggtacgagggggtcggtgcgaacagtcaggctgggccctgtgtggttgtcacctctcattcccaccgtccgggcagatggggagactgaggcttggaggggcgggtgttggtgaaggcggctgatgtgataccgagcaggcctcattccctctctccacaacctcccgatcccaggatgctcagaggactgggtctcaggcttccccaaccggccaccggtacccacgtccgctcccgccctgaggagccagggcaggccccgaaacaacagccgcccctcccgttcccctcccagaccgccaggagcacctgggagggcccctcagatccacccgagacccccaaggcctcagcgaacctgggtgggctgtagctactgatgcacaatgaccttcccagccccaccccagcgccccccgccccagaaccaaagcccggtggtggccagcagacacaggcggcacctcggggtctgggggacgcagctgcggctggcttctccccgaccctgtgggcggcacccaactccccaccctgccgggtcctcccggggccatctcggagccacccagcacttgaccatggccctgacaaggcccctttgtactcactggcagtcggagggcgtgggccggtagtagaaggagggtactttggactcaaacctggctctcgcggcgtcgttcccgtgggaggccatgaactgcaagagaggaggggccgggtcagaggcctggggtcccaggggcagcctaggaggtgccagctgcccttggggccgtcaccacagtgacccgcagacccaaggctctgagggctccaccagcaccaggacgggaacgccacctcctcctgccccagggacaccatttctgagtgatcgaccctcccctgggacagggaaggagccagcgtccctaacgacggggtcccggcaaagggggcccaggggccaggccccttttgaggatgggtctgaggtttgccccaccgtttcaacccccaagtccacccacaccgggtgtcccgtgggagagagctggctgcatcctgtgacccccatctctgccccagagagcgagggcttcctcgttcacccagctccccagctcacatttagcaaagagccaacaggcgccagacacgggggcctggagatgggctggtgagcgagagacccagatgtggctaggacagagtctctgaggggactctgcagggacacactgcagggacccagaacatgtttacgaaacaggctgagggagaaggaaacggcgccgtgaaggaggggaggtgctgcaggaggctgtcgttccaggaggctcgtgctccaggaggctgtcacgcacactgtgctcctggcaggcgcaaaggccctgagggtggacgctggcaatgaccctgccgccagccaggtatggtgagccttgagcagcaggaggggccgccacaccccacgccagccccgagggcatgggagaggttctgcttgctatgctgtaagcccccaaggcagctgctgtttgcaccttcaagctcctgaacaccactcgcctgaggccgcagccaggcccagccagactgaagtcccctgacatcaccctgctccgaggatcacatggcagtaaacagggagggcccagaagagcagctggagaaatcacaagcaacaagaggctggcaccaatttaaaattcgaaaaccaatggcttttccatagaaaactcaaatacaaggagagatcctgtcaccaacagcaacaggacaaatcacctgagtggctggatgagaaatgtcccaggtctccaagggaaaactggcgtgtgctcccgaggacgagagcctcgacccgagacacagggaggtagaaaaatgtctccttatgggagtctacatgttttttataataattgccaaaaaacagcaacagaatttattatatttttttacctagaaatggttctaaagttgaaaaaattaacaagggccagccggggacaggggctcacatctgtagtcccagcactttgggaagctgaggtgggaggaccccttgagcccaggagttcaagaccagcctgggcaacatagtgagaccctgtctctcaaaaaaaaaaaaaaaaaaattagccaggtgtgatggcgcacacctgtagtcccagctactagggaggccgaggtgggaagatggcctcaacctgggagtttgaggtcgcagtgggctatggtcgcaccactgaactccagcctgggcggcagaacaagaccatgtctccactggacacagcggctcacacctgtaatcccagcactttgggaggccgaggcgggaggatcccttgagcccaagaggtcaaggctatgagctatggttacaccactgcactccagcttgggaaaaagagcaagaccctatctctgcaaaaaaaggaaggtaaaaataaaaaataaataaaaatgggtaagggtttttaaaattacttttacatgcatatgtatacttttaatttgttttaatggaggtggggttccactctgttgcccgggctggtctcaaactcctgggctcaagccatcgtcccacctcggcctaaaaatggggaaggatttgatcggacatttctccaaagaagatgcacaaatggccactcggccacgcaaaggcactcaacactgttagctgtcaggggaacgcagatgaaaacctcaatgagctaccacctctccccactaggatggcaaggtcaaagtggcacaggggagtgagctggagagaatgtggagtgatcagagcccccgcacgctgctggtgggaacctccggctctggtgactaacagtctggcagttcctcaaaacggtaaacacagagttaccatagagtccagcgattcctctgcaccgtaagagacgcgaagacgcagagacatgcacatgagtgttcacagcacattcttcgtgacagctgagaacgggaacaagccccagctccgtgaacggaaccacagataaactaaatgtgatcgagccacaccatggagggttcctcggccataaagaggaatgccggccgggcgcgggggctcacgcctgtcatcccagcacttcaggaggcagaggtgggcagatcacgaggtcaggagttcaagaccagcctgggcaacacggtgagaccccgtctctactgaaaatacaaaaattagccgggcgtggtggtgcacgcccgtaatcccagctactcgggaggctgaggcaggagaatcgcttgaacccgggaggcagaggttgcagtgagcagagatcgtgccacagcactccagcgtggctgacagagcgagactccatctcaaaaaaaaaaaaaaaagagcaacacaattctgacacacgctacaacccaggcgatccttggacacacggtgcccatggaggaaaccagacacaaaacaccacgaatcggccgcacgtggtggctcacacctgtgatcccagcactttgggaggctgaggtgggaggatcgcttgagcctgggagttcgggaccagcttgagcaacatagtgacaccctgtctcaaaaaaaaaaaaaaaaaaaaacaagaccacgtattgcatgattccatctgtttgaaatgttcagaacaagcaaatccacagagacacaaagcagctctgtctacattcagtaacggctggggcggtggtgatgggaggggtggtgagtaccggctgtgttgggacgttaaaatgtcctgaagttagacagtggtgatggtcgtgcagccctgtggcttcactaaaactactgaactgtacgctttacgggagtgaattgtatgtggattatacctcaacaaagctgttattaaaaaataaccttcagggttgggtgtggtggctgaagcctgtaatcccagcgcttgggagactgaggcgggaggatcacttgaggccaggagttcaagaccagcctgggcaacatagtgagacccccgtctctacaaaaaatacaaaaattagccaggcgtggtggtgcacgtctgtagttccatctactcaggaggctgaggtgagaggatcgtttgagcccaggagcttgaggctgcactgagctgtgattgagccactgccctccagcctgggtgacagagcaagactgtctcaaaaaaaaaaaaaaaaaaaaaaaaaaccctcaggcagagaaggcctatttatgtatgctatagaccctaaaaccacaaaataaaacgctgaccactttgactatgtcgaagtaaaaatttccacggcaaaaaccaccatgagcaaattcgaaagacaacctagaaaaaaatgtttttggtatctcagattaatttctctaacataccagcaacccctagaagatgaaaacagctaacattgcagcagaaaggcaggcaaaggtgtgaagagagttcacaggcaagaaaacacagggccgggcgcggtggctcacacctgtaatcccagcactttgggaggccaaggcgggcggatcacgaggtcaggagtttgagaccagcctggccaacatagcaagaccccatcactacaaaatttttttttttaattagccagggctggcgcggtggctcacgtctgtaatcccagaactttgggaggccgagggggatggatcacctgaggccaagagttcaaggccagcctggccaacatggcaaaaccccgtctctattaaaaatacaaaaattagctgggcatggtggcacacacctgtgatcccagctactcgggaggctgaagcaggagaatcgcttgaatgtgggaggtggaggttgcagtgaggtgagatcacaccactgtactccagcctgggagacagtgagactgtctctaaaaaaaaaaaagtagccaggcacggtggtgtatgcctgtggtcctagctactccttgggaggctgaggcaagaggatcacttgagcctgaaagttcaaggctttagtgagctatgatagtgccactgcattccagcctgggcgacagagtcagactgtgtctcaaaaaaaaaaaaaaagagaaaggaaggaaggaaggagggtagggagggacggagggactgagggagggagggagggaagaaaggaaggcaggagggaaggaaggacggaaggaaggacggagggacggagagacagagggagggacggaagaaaggaagacaggagggaaggaaggagggagggagggagcgaggcccacacagacccctggctgcctggccagggtggggacggtgctcaaaggtgggagacagggaaggagacagctcccttctgtgactgtccttggaaactgctgaatgtgtcactaaattgtcgaaatacattaaataaggtagagacgacccacgtgtccatcagagggtgaatgaataaagcaaacgtgttccatccacacaaggcatgttctccggccttgaaaggaacgaagctgtcacacaggctgcagcgtgatgggcctcggggacctggcaccgcgggaaaggagtcggacgccgaagctcacacgccgtgcgatcccgtttttaaaagtccagaacccacaaatctacacagagagaaagtggatttgcagctgctttgggctgggcgggggtaagagccaaaaggcacggacttcctttctggggtgatgaaaatgtcctaatattggcttcagcgaaggttgcacaactctgaatttactaaaaaaccttcaactgtgccctttacacggtgaaatgtatggctgtgaacggcatctctaggaagcagttgttttaaaaggtattatcaaggaatagaaagtagggagaaaaaaaaaaaaaaacataaaatgtgaagaaaaggaaagaagagaagaatctttggttcctggcccttcaacctcggtccggtcctcagcaacggccacctcaggcccgtttctcccttaggggtgggagcaggcttagcccccggatgtccccgctgaccaccagcccaggaatggccaaggggcctgagtgcagggagccagggggggcccagtgcagctcccgccccacagagtccccagcaagaagccgggctgagacccagccctgcctgggggccggggtgcaggtgctctcaggagacaagcccccaggcctgcctgagattcatctttggtttttatcgcaccagtgaacgtgcagacaacagaatggggatggaatctccttgtggcccccagctcagtgctctcccaaaggccccatcgcaccagacctctgcggcaggaatcaggggtttcttggtaatacccacggtgggccctccccagccttcctctccagccttcctctccggccatcaccccaggcagatcaggacagcaccaagttggcccctgtcctctcccgctgattacatttagggtttttccaacaacagtgaacgccccacccctgggcccttgccccgtttgccctggaattatggggacaatagccacgtcttctccccgagccaaggagtctgtgcccctggaggccctccagggactcagggctgtttcctccaggagtgatggcagagcaacttggagacgggacacccgtcccatcatctttctgacattggtcccctgttgtccggggagggggatggtccaggggccaccctgcccattttttagggaaggaaacggaggctcagacaagcccacagcctggcgaagggatggatgtcacatcccagtgtccccccctgggtcagtgaaggtgggtcgaagggcgaagggacggatatcacatcccagtgtcccccacccaggtcagtgaaggtgggtcgaaggcggtgagcccaaggtgggccggggtcctgaggggattcgcagggacgccctcaactcagggtgtgggggatcacggcattaagtgcagaagctgatttggggtcgccctgggtgggtcagggactctgctgtccttgggtctttttctgtctcccaggctggagcacaaaggtgcaatcacaactcactgcagcctcgaattccttggctcagccaggcatggtggctcacgcctgtcatcccagcacttcgggaggctgaggtgggtggatcacctgcggtcaggagttcaagaccagccttgccaacatggtgaaacccttctctactaaaaatacgaaaattagccgggcgtggtggcgtgcacctgcaatcccagccactcgggaggctgaggcaggagaattgcttgagcccgggaggtggagcttgcagtgagccgagatcatgctactgcactccagcctgggcgacgaagccagactccgtctcaaaaaaaaaaaagaattcctgggctcaaacaatcctcctgcctcagcctcctgagtggctgggattacaggcaccacccccacacctggtcgaggcactgggatcccagctgtgatttctaacaagcctccaggtgtgacccatgcaccaaggctgcctgttagaagcaaatgagctactcccaaccaggggcaggggtcagtggtgctggagagccgggggccggggtcagtggtgctggagagccgggggccgggggccggggtcagtggtgctggagagccgggggccggggtcagtggtggtgcagagccggggaccggggtcagtggtgctggagagccggggaccggggtcagtggtgctggagagccgggggccggggtcagtggtgctggagagccgggggccggggtcagtggtgctggagagccgggggccggggtcagtggtggtgcagagccggggaccggggtcagtggtgctggagagccggggaccggggtcagtggtgctggagagccgggggccggggtcagtggtggtgcagagccggggaccggggtcagtggtggtgcagagccggggaccggggtcagtggtgctggagagccggggaccggggtcagtggtgctggagagccgggggccggggtcagtggtggtgcagagccggggaccggggtcagtggtgctggagagccgggggccggggtcagtggtgctggagagccgggggccggggtcagtggtggtgcagagccggggaccggggtcagtggtgctggagagccggggactggggtcagtggtgctggagagccgggagccggggtcagtggtgctggagagccgggggccggggtcagtggtggtgcagagccggggaccggggtcagtggtgctggagagccgggggccggggtcagtggtgctggagagccgggggccggggtcagtggtggtgcagagccggggaccggggtcagtggtgctggagagccggggactggggtcagtggtgctggagagccgggagccggggtcagtggtgctggagaaccaggggctgggatcagtggtcctggagagggggtgccagagtcagtggtgttggagagggggggcccagggtcaatggtgctggagaggggagagacatgtacaggaggccaccagcctctacgaccctccccatccccacagcagcccccagaccaccactctcacctccatgcggcccttcggtcccactcacagcagtcaggaagatcaatggcttccagaccccaagtcccacgagtggggctggggtgaccctcccaccgggtcttcaggccccacgctccatccgacctgcagcccccagccggggtcttgttccttctccacctccctcggggtctccctacggggcccttcctgccgcctccagcatgcgtggcccctccctgcacgccagggcttcctggttggctcacacccgtctccctccccataggggcgtctctagggccccacgaagcatctgtggctggtgtccctgcctggccatgccaagtgctccgtgaagcctcatcagatgcatgggtgagaggtgggcagtggctgtgtcacctcagaggccgcccaggggacaggtaatggtgccctacacggcagcgggccggggtgcacgtggactgccgtgtaaatgcgaccctgactctgggtcgtgccgcgcggcctgggacgctgcattctgtttccttagagacagggtctcttttaccctgagcccagtgctccaggtgtggggatttgaggggagcactccggagctcccagggcacgggaatcggcgacccgccttcgctccctggcccaccacacacacctggagctgctcacagagaggttggacccggcacggggtgggtggagcagccacacaggcggagccgctggagagggggcggggtcctcgggtccagccagccaggcccccagagtagcccaaggctccaggggccacagccaggccgcccggcatctctggctccagaggcccgggctgaggccacccggggactgcgcgggtccccccgcccctccccctccgtacctccacttgggcctcctcccaggcgtccaggcggacggacttcaccttgctgacctgggggatattccggtggattcccgagcagctcaggcagatgaagacgcccagagtgtaggaggcccagtcgggatctgcaagggaaagccggacgttcacggaggctcagcccagggaccccggagggagggtggacggagcctcgagtcaggagcccccggccatgcagtggggggggcttcagcggagacccccgggtacaccaggctccgtgcgccccacaggcacctaaccccagctccccccccgccctctgcccggtgcagacggggctctccccagacaccccacggcatctgaggaagccgatctgcatgcacctgctctgccccgcgggtgccgggaacctccagcagaggccggccccacaggagcccatgccccgggctggagggcacccggcactcgtgaggatgggtccacctgctttcctcgtgtccgctgccctgggagaagtctgtgggcccgaatcctcctgggtgatggggccaagattgggaaattcagataggatctgagtgcagggggtgcctcacacccaacgcgctgcacacccccgaaggacggatggtagccgcggatgacgacgcaccctgcgtggggtgacagcgcccggcggccactaactttgcaggtgtgacagcggcgctgtggcttcaccctgcatgtggcctgtctagggattccacgtctcgatgtctggtttgccttaaaaggtccaacaaagctggacacagtggctcaggcccgtaatcccagcgctttgggaggctgagggagcagaattttttttgagacatagtctgttgcccaggctggagtatgatggcacgatctcggctcgctgcaacctctgcctcccgggttcaagcgattctcctgccttagcctcccgagtagcgaggcacgcaccaccatgcccggctaacttttgtatttttagtggagatggggtttcatcatgttggccaggctggcctcgaactcttgacctcaagtaatccacctgctcggcctcccaaagtgctgggatgacaggcgtgagccactgcccagggtgtgggtgccacagcgcagtctttctgctgctgcacgtgtgaaatgtctcattctgaaacggcaaaatgtggggagcgtccagccccgccctggggcgatggctaagagggctgctgtgtgactctcctgttttcttctggctcccaacgacccttcacaataagtcaagggagtggaatggctgaaaggcaccctagagagaacagtctctctgggggagatgagtcatctctgaactgcttttatttttaagtggaggctctcctgggtgaggctggtggaaatcgaggcgcttccccacaagcaggtgcggctgtggacccaagcctgtttggggaatcatgagtggagaggaccccacccttcaggcctggcggaggatagcccccctcccaacgctgatcggggcccacccctggctggtgggggactcccatgccaggcggcggaggcccagtggggctggggctgggcctgggctctgagtcactgccaggaggagaggcacccaagtgagcccccggccggggatggtgcgtgagaaccagcaggctgaacctcggatttgggggtcatgcccggcctctgggatttgggggtcacgccgggcctcggatttgggggtcacgcccgacctctgggatttgggggtcacgcccggcctctgggatttgggggtcacgcccgacctctgggatttgggggtcacgcccgacctctgggatttgggggtcacgcccggcctctgggatttgggggtcacgcccgacctctgggatttgggggtcatgcctggcctctgggatttgggggtcatgcctggcctctgggatttgggggtcacgctgcacatctgggatttgggggtcacgccgggcctctgggatttgggggtcacgcccgacctctgggatttgggggtcacgcccggcctctgggatttgggggtcacgcccgacctttgggatttgggggtcatgcccggcctctgggatttggaggtcatgcccggcctctgggatttggaggtcatgcccgacctctgggatttgggggtcacgcccggcctctgggatttgggggtcacgcccgacctctgggatttgggggtcacgcccggcctctgggatttgggggtcacgcccgacctctgggatttgggggtcatgcccggcctctgggatttgggggtcatgcccggcctctgggatttgggggtcatgcccgacctctgggatttgggggtcatgcccggcctctgggatttgggggtcacgctgcacatctgggatttgggggtcatgcccggcctctgggatttgggggttttctgtcacagctgttaggctccctgattaatgaagcctgtctccccattaacgtgggcttcttgaggaccccagacccagcacccccggatctggcacagagctcagtccggagcaggtctccacgggagtcatgaatgaaggggccactaacagccctacttcagcagagcccctgtcttcgagagagaccacaggctttctcctcacccctcagtcagtcaaacatttgcccaagtgaccctgtctcgggtaccaggagttttgcctgtgctgagtagggcggcttttgggagaaagaatgcggttacaggatcagccgttgtttggttttgcggggttttttttgtcttgtattgtattgttttgttttgagacagggtcttgccctgttgcccaaggtggagtgcagtggtgcaatcacagctcacagctcactgcagcctccaacgcctgggctcaagcgatcctcctgcctcagccttccaagtagctgggaccgcaggcatgcaccagcatacccagctaatttttaaaatttttggtagagatgaagtctctctatgttgcccagcttgggcttgaactcctgggctcaagcgatcctcctgtcttggcctcctaaagtgttgggattacagatgtgtgacgccgtgtctggcctgcaaagtatttagttacacacacccctggagaacaggtggggaagaaccacactgaagtcccagaagagagagacctgactccccccgggctccgtcttctcccgaagccttggtgaaagatgccgcaaatgctcggtacgtggctgtgcgaggggcgcccagaaggcacgccagtctccgggcctcggtctcctcatctgtcggatgggacagcacgagccacttacaggggtagcaggcaacatgagcccagctgggaacatcaccgctcggggtgcagtggggggccctccttctcaccgcatggcaatgctgttggttctgctccgacagtgtggaccacggacccaggcttcctgtctcatggcctttatctgcccatctagaaattctgagggaggctgagcaacggccaccaacagctcccgcccagcccagggtccagaatgcctgtgcccacctccaacctcactcgggtgctccgggacaggctgtccctctcctggcctggaaggaggcctgttcacagaacccagggagctgggcctggaaggaggcctgttcatggaacccgtagcaggagctggactctacggcgtggaccagctgcctgttgttgtaaataaggtgttactggaactcagccatgcccatttatgaactgtctatagctgcttttgtgagcggtcgttgactagctgtgatggagactctacggccctaaaatatttactgtcatttttgttgttttttgagacagagtttcgctcttgttgtccaggctggagtgcaatggtgtgatctcagctcactgcaacttctgcctcccaggttcaagtgattctcctgcttcagcctcccgagtagctgggattacaggcgtgcaccaccatgcccggctaattttctatttttagtagagatgtggtttcaccatgttggccaggctggtctcaaactcccgacttaggtgatccgcctgcctcggcctcacaaagtgctgggattacaggcgtgagccaccacgcccagcctactatctgtctttttaagaaaaagtttgctggctgcgtgtggtggctcacacctgtaatcccagcacttcgggaggccaaggcgggtggatcacctgaggtcaggagtgtgagaacagtctggccaacatagtgaaaccccgtctctactaaaaatacaaaaattagccgggcgtggtggcgggtgcctgtaatcccagctacttgggaggctgaggcaagagaatcactggaacccgggagatggagattgcagtgagccgagatcgtgccactgcactccagcctgggcaacagagtgagactttgtctcaaaaaaaaaaaaaaaagtttgccaatgtctgcttagagggaaaaatgggggtggcacagatgagtgagcccaagaagggcttgtaagtaggactgatagcttacagccattactaatatagcctatctttgacaaaccagctacaagtgaggctaaggagtggttacaacctgtcccgacctcttgcaactggcacaaggtcccccggtgtaaagttagaggttgtctgatgcaaacctgtcacatcctccttggtatttgtgtgtgagcatatacatacgtgcgtgcatacccacagacacaacatgaacagcctactggggagggctcagggagttaaatgtgaagaaatagagacaccagccaggtgtgggtgctcacacctgtcatcccagcattttgggagattgaggtaggaggatcacttgagcccaggggtggaggttgcagtgagccgagatggcacgactacactccagcctgggtgtcacagaccctgtcacacacacacacacacacacacacacacacacacacacacacaatatggagacatgaccttttacataagaatcaaaagacgagagccagttaaatcaagtgtgttggagacaggagcaacacaaggttgtcactggtagggcaggaagtttgtatcatttttaaaaagcttttgtggagttttactaggatgactaaaatttaaaagatggatactaccaagtgttgatgaggatacagagcaagtggaactctcatccactgctggtagaaatacaaattagtccaacccactctgaaaacagcttggcagtttcttacataaagatatatctcccatatgacacagccaccctactcctaggaatttacccaagggaaatcaaaatgtaggtccataaaaagatgtacatccaacaatatataaaaagagtaatacaccatgatcaacaggggtttttaccagtaatgcaaggttggtttaacatttgaccatcaatcaatgtcattcaccatatttacaggttaaaaaagaaaaaacatacaattgtttcaatagatgcaaaaaatgctggcaaaatgcaacatcctgataaaattctcagaaaactaagaatagaggggaatttccccaatttgatatagaggattcaacagatgcaaaaaatgttggcaaaacgcaacatcctgataaaactctcagaaaactaagaatagaggggaatttccccaatttgacatagatgattcaacagatgcaaaaaatgttggcaaaatgcaacatcctgataaaactctcagaaaactaagaatagagggtaatttccccaatttgatatagaggatctatgaaacatgtacagcaaatgtcatattcaagggagaatgactgactgctttcccccaagatcaagaacaggacaagaatgtctgttctaaccactcctagtcaacgttgagctagagattctagtcagtgcaataaggcaatataaagaaataaaaagcatctagattggaaaggaagagctaaaactatcttcattaacagacaacatgattgtctatgtagaaaatagaaaagattctattaaaaacaactatgagaactaacaagtttagcaaggttgaaggatacaagataaatatacaaaaatctattgtatttctatacactagcaacaagcattcagaaattgaacatttaaaaataccatttataatagcataaaatgtatgaaagagggataaatgtgatgagaaatgaaacataggacactgaaaactacaaaatattgctcagagaaattaaagaagacctacagattttggaaagagataacgttttcgtgggatgggagactcaacattaataacacatatattctctccaaattatagattcaatgccatcccaatcagaatttcaacaggattttaaaaatagaaatgaacaagctgattctaaaattcatatgtaaatgcaaaggacctagaatagctaaaacaacttagaaaaaaaagacaaggacggggaactaacactacttgttttcagaactcattaaaaaattacagttatcaagacagtgtggtggtgttggcacaaaaacggataaacggatcaatggaacagaatcgggaatctagaaataaacccacacatatgtggacaactaattatcggcaaagatataaagccagttctggagagagcgaacaatctttcctaccaacggtgctggaacaatccggtatcatccacaacacagtaaacaaccatccctctcaccatatccaaaaattatccccaaatgcatcatggtgctaaaggtaaaacctaaagccatacaattttaaaagaaaacaggagaaaagtctccgtaactttgggttaggcaaagatttttttcagatacaacaccaaaggcagaatccactgagagagagaggaggaggaagagagaggaggaggaggaagagaggaggaggaagggagagaggaggaggaagggagagaggaagaggaagagagaggaggaggaggaagggagagaggaggaggaagggagagaggaggaggaagggagagaggaggaggaagggagagaggaggaggaagggagagaggaggaggaagggaggaggaggaagaggaggaagggagagaggatgaggaagggagagaggaggaggaagggagtgaggaggaggaagagagagtaggaggaagagaggaggaggaagggagagaggaggaggaagggagagaggaggaggaagggagagaggaggaggaagggaggaggaggaagaggaggaagggagagaggatgaggaagggagagaggaggaggaagggaggaggaggaagggagagaggaggacgaagggagagaggaggaggaagggagagaggaggaggaagggaggaggaggaagaggaggaagggagagaggatgaggaagggagagaggaggaggaagggaggaggaggaagggagagaggaggacgaagggagagaggaggaggaagggaggaggaggaagaggaggaagggagagaggatgaggaagggagagaggaggaggaagggagtgaggaggaggaagagagaggaggaggaagggagtgaggaggaggaagagagagtaggaggaagagaggaggaggaagggagagaggaggaggaagggaggaggaggaagggagagacgaggaggaagggaggaggaggaagaggaggaagggagagaggatgaggaagggagagaggaggaggaagggagtgaggaggaggaagagagagtaggaggaagagaggaggaggaagggagagaggatgaggaagggagagaggaggaggaagggagtgaggaggaggaagagagaggaggaggaagggagagaggaggaggaagggagaggaggaggaagggagagaggaggaggaagggagagaggaggacgaagggagagaggaggaggaagggagagaggaggaggaagggaggaggaggaagaggaggaagggagagaggatgaggaagggagagaggaggaggaagggagtgaggaggaagggaatgaggaggaggaagagaggaggaggaagggagcgaggaggaagggagagaggaggaagggagagaggaggaagggagagaggaggaagggagagaggaggacgaagggagagaggaggaggaagggaggaggaggaagggagagaggaggaagggagagaggatgaggaagggagagaggaggaggaagggagagaggaggaggaagggagagaggaggaggaagggagtgaggaggaagggaatgaggaggaggaagagaggaggaggaagggagcgaggaggaagggagagaggaggaagggagagaggaggaagggagagaggaggaagggagagaggaggacgaagggagagaggaggaggaagggaggaggaggaagggagagaggaggacgaagggagagaggaggaggaagggaggaggaggaagggagagaggaggaggaagagagagaagaggagaaagagagaaattgacctcatcagaattattactattattattattattgagacagggtcttgctctgttgcccaggcttgagtgcagtgaggcaatcatggctcactgcagcctcgacctcctggattcaagtgatcctcccgcctcagcctcctgagtagctgggactacaggtgcgcaccaccatacccagctactttaaaaaactttttgtagaaacaaggtattgctatattgcccaggctggtctctaacttctgggctcaagcagtccttccacctcagcctcccaaagtgctgggattacaggcatgagccacttcgcctaaccaagtccctgttttatttatttttctttatatttttaacctttttttttttttttttgagacagagtctcactctatcacccaggctggagtgcagtggtgcaatcttggctcactgcaacctttgcctctcgggttcaagcaattctcctgtctcagcctcccaagtagctgggattacaggcgcccgccaccatgcctggctaatttttgtatttttaatagagacggggttcaccatgttggccaggctggtctcaaactcctgacctcaggtgatccgcccacctcggcctcccagagttctgggattataggcctgagccaccgagcccggcctatttttaacaatttttaatctttaatttttgtgggtacataagaggtgtatatatttatggggtccacaagacattttgctacaggcatgcaatgtgaaataatcacatcacggcaaacacggcacccatcacctcaagcgtgtatcatttctttgtgttacaaacaatccaattacactcttttagtcatttttaaaagtacaataaattattgttgacaatcgtcaccctgttgtgatatcaaatactagatcttattctattctttgtacccattaaccatccccactctacccaccctgagactctgttttaaagaaagattcagaccctgggagggaagagcccgggggggtgactctgggtgactctgggcgatgtcttaaggcctctggtctctgggagagtgacctcggaagcccttgaaggcactgaaatagaaggtggccttctgatttgagtatgctgaagctctgcagggtgggctcacgtgtgtgtgtgcaggtgtgtgtgtgtgcaaggagatggggagctagagtcccccagcttccagtaaggaaaaagcctggctggtccccaaggcagaagggacgggctgacccttctgttttccaagtgactgaggctgggctgggcaggcctccttctcaggctgctgggcatggccaggcctgcctgcatccgcaaatgcccgcagcccatcggagcgaaaaggggcggggcacgcactctcagagaccctccagggtggaaggggagctggtctgacgccccagcagcagggcccccacaggcccacgcaggagagcctctccctaagcctgggctgcactccagctgccccctccactcggggcactgaaccgtggggccggccctgcttctgcccaggaccacttctgggttcccaggactgtggaattttgttccaaaatgcctggagtcagcacccacggcctgtggtgcccccagaacaggccacaaggccagcaggtacacactgggcccaagggtggctgcggcatggctgatgtggtggtggagatggaggccctgagtgccaggtagggaggggcaggcccaagactccagcccccacaaacatccaggctgccagcaccgtctccaggagctgcctcctcctcggagactgcccacagccgccagcctctttccctccctcctcccagccccgctctgccctacctgctcccaggacgcccgaggtgactcagccgctcaccatttccggagctggtctggggcacctgggcaggtgagccacattcttgggcggagccaggtggggcaggcgtgtcccccaagccaaggcccaggctggggcacgggcagggggaagggcagtagccaagcctgtccatggggccctggttccagagacctttgccctcccaaggctgacgcacagcagagatcccggtgcaatcgaggccgggtcggacgctggctctggccaggcacgcaaggggcagccgaggtgggagggtgcccttgtgggaggggctccggtgcccaccacccttcacagctacgtgaggcggggcatgcatggctgtcccccaggcacacagcgccccactccacgccgggacccaggcccctctccaactccacctccgtgttttccgcatattgtctcccagttacccgtggcccctgcagggatccaggctcccgccggccggtggatccccgccagcgaggcagtgacctctggagctcctgggtgggctggcagcccacacctcctctctgcccaggccccaggcccccaccccctcacgctcacgggcggccctggtcccattccattgtgaggatggggccctttggccctaggaacaggccctcccaggacccagatccgctggcccctacccggttcagggacacgtgcccctctcctggatccctcccagcagcacccggacacagtccattttcagaatcccccaaggacccccccaggctctgccctgcccctgccctcccaccctctctccctcctggtgctccagccccatggcgggaaccccacggtgaaggccatccccagtcctccctggacccaccctgtcccggagcccactctctggggcccccaccccgcgcccctccaggctcttcagggccctcgcacagggccgactcctctgcggcccatctggtctctcggctgtcaaaccctatttttggaactcgatagaggtggtggttgaacagcatggagaacgttctaaatgccactgaattttcactttgaaatgattaacctgaggccgggcggggcggctcacgcctgcaatctcagcacctggagaggccaaggtgaacgggtggagcccaggagttcgagaccagcctgggcaacatagtgagacaccgcgactccatccttatgaaaaaattttctataaaaggttaatttttcatatgaattgcacctcgatttggggttttgttttttttcattttttgtttttttgagagagggtctctgtcactcaggctggagtgcagtggtgcgatcatggttcactgcagcctcggcctcctgggctcaggtaatcctcccacctcagcttcctgagtagctgggaccacaggtgtgcaccaccacatctggctgattttttggttttttttttttttttttttttttagagatgggggtctcaccatgttggccaggctgaccttgaactcctgggctcaagcgatcctcccacctcagcctcccaaagtgctgggatcacaggtgtgagccaccgcgcctgaccttgattttttaaaaaaggagagagagagacattcacacactggcaacccctgtctctagctcgggcctctttcctccagagctgcccgtgtggctgctgtccccttggcggccaccgtggcttctcacagagcctcacatcgcaggtccctcccgtctccctggacactgctcccacgtccccatctcagcgagtgaggtgctggctaaaggcctgggtgtcctcatttctactctgcccaccacaccccgacatcccgttgaccagcaaatatttcaagacgatccaggctttgaccctccccacactcacgccccgccctaggcctagccaccgccacccggcctcggtcactgcagcagcgtcccacccacctcccgctcccgcgctggccctacctgccctggaccctgtgacgcgtcgtgggctgggacactcctcggctcacagaattcccatggttcccatcccacccagagtcacagcgcagccctcacagcgtcccccagccctgcgtggccccacagccacctctgaccccgtctccccgccctggctctgggcacactggcctcctcacttctcctttgacctgcgaggtgcacggctgcccccacccccgacaccaccagcccctttaccccactccctcttccggaggggccgtctccctcctccactgaggacccagcactgccagggacctgctcctcaccccacaccttggccaccccacggcctgccctgaacccactctattccagctgggcctttcccgctcacaacccccgagccccagcccccatcctgaatctgcaggacctgacccagccatgtctgtccctccctcctcaccccatgtctgccctgcccccttgcccgggtgggatcccaggtccatcagtgcaatgccctctgcccctcacccctccctccatcattcagtacggagcccacctctgtacccaccgaggcctgagcttctgaatggaacacgccctgcagaagcgctccatggtggcccgagtttcggcttccctccccgccggcccatggcctggcctcgtcctccatgaggaaaacagtcacttccaaagccttcccctgcatgccaggccgagctcccactgcaggtctctgccccggtcttagcagaacttctccaaatgcatctccaggtgccacgaccccctctggcctctcattcacctgctaagctgtcaccaccaccacctggacagtgacttggggtcagtttgctcctccctgacccacctcccagcccataggtgcccagtactccttcctgaaaacgcgtttacggggcatccaagattctatgtccttttttttcccttgagacagaatatcactctgtcgcccaggctggagtgcagtggcatgatctcggctctctgcaacctctgcctaccaggttcgagcgattctcccacctcaacctcccgagtagctgggatttcaggcgtgcgccaccacacccaactaatttttgtatttttagtagagatggggtttcaccatgttggccaggatggtcctgatctcctgacctcgtgatcctcccgcctcagcctcccaaagtgctgggattacaggcgtgagccaccatgcccggccctcactcacaattcttactctaccctcggccctccttgtccccaagtgccatcaccatctgtctgtctttgtctccccaccaaggaacagcagatgctgagagtgaagggctggtctggtccggtctccctgagccccagtgactgcacagggcaaggcactcagtacatgtgtacggagcgggggaaggcgccttgcccgccggagtccagggtagggcaccactggagttcagcccttgtgggcctctgcctcccccgggcacatgagcacatacctcctggccacacggccacgtgccgcctcagccaccttggaccccaccaagtcccttcctgtttcctcattctttcatttgcaaaatggggccatgcagccacctgacgggtgacgggagaagcagttgggggatgcggctggctgccaggtactgccacagagcaaggcttggcaaatgcctctgagtctaaaacggcagtggcctaggagcacagggcctgggggcaggggcagtgccacacctaacctgagatatgtccagagctgaggtctagctcacagcatctgtgcgtcggggaagctggctgcggtcacggccggtgcagcggcccaaggagtccacaaagcagtaaccttggcccacaccacagctccggtgcatttggagagggggcccagtctaggctgaagttggggggagcctggccagggcctgggcaccaggcgcaggacagagggaaaggcctccagctcctcccagccctgagtgccagctcatcgcccacaccagccctccacacccggccacagggcaggcaggcaggaagcgggcacgtgtgacctgctggcctgtggcttgagtgcaaaaccaggtaggggaaacctgtctagcagttcttcaaccaggtaagcacagaatcgccatgggacccagcaactccagtcctgggtacagacccaaacaacctgaagcagggtctcagagagacatctgcaccctcgtgctcacacagcacgattcacaatggcccagaggtgggagcagcccagtgtccgcctgcagacaaacgggcacacgcaatgtggttcatccacacagggtatgatcctgtcttcacatgcagggaaatcccagcactcgccacagcatctgtgaacctggagacgttagactgagtgaaactggccagtcacaaaagaatgaataccagccgggcatggtggctcacacctgtaatcccagcacttcgggaggccgaggcaggcggatcacaaggtcaggagatcgagaccgtcctggctaacgtggtgaaaccccatctctactaaaaatacagaaaacttagccgggcatggcagcgtgcacctgtagtcccagctactcgggaggctgaggcaggagaatggcatgagcctgggaggcggagcttgcagtgagctgagaaggtgccactgcactccagcctgggcgacagaatgagactctgcctcaaaaaaaaaaaaaaaaaaaaatgaataccgcatggtttcacttctacaaggtccctggagtcataagattcatagagacagaaagttgaggccaggcacggtggctcatgcctgtaatcccagccatttgggaggccaagttgggaggatgacctgaggtcaggagttcaagaccagcctggccaacatggcgaaaccctgtctctaccaaaaatacaaaaattagccgggcgtggtggcaggcgcctgtaatcccagctactcaggggactgaggcaggagaatcgtttgaacccaggaggcagaggttgcagtgagctgaaatggcgccactgcattccagccttgggaacagagcaagactccgtctcaaaaaaaaaaaaaaaaaaaaaacaagaaaagaacagaaagtagaagggtgggtgccccggactggggagaggaaatctggagcccttgttgaatggggacagagcttcagtttgggaagatgaggaggttctggagatgatggtggtggcggtggcacggtgagtgaacgtgctcaaggccctggaactgtgcactcgaaacgatgagaaggtaaattattatttttaaaaaatccggccaggcgcggtggctcatgcctggaatcccagcactttgggaggccgaggcgggtggatcacgaggtcaggagacggagaccatcctggctaacatggtgaaaccccatctctactaaaaatacaaaaattagccgggcgtggtggcgggtgcctgtagtcccagctactcgggaggctgaggcaggagaatggtgtgaacctgggagggggagcttgcagtgagccgagatcgcaccactgcagtccagcctgggcgacagagcgagactccgtctcaaaaaaaaaaaaaaatatccaacctagttgggccgctgccctggggagtgaagggagtctgcccagcccccgcccacactcagcagccttcttcccaccctcacccatgccggccaaattcctgaaactgggagttggaggttgcagtgagcaaagatcgtgccactgcactgtagcctgggtgacatcgagagactgtcttaaaaaaaaaaattagctgggcatggtggcatggacctgtagtcccagctactcaggaggctgaggtgggaggattgcttgagccctggaggcagaggctgcagtgagttgagattgtgccactgcactccagcctgggtgacagagcgagaccttaaaataaaaaagaacaaaagagctcagacaccccagtccttccggggcatcagatggcctgagactcaaagaggacaatatgtgtgtttttcttttttttgagacagggtctcactctggcctaggctggagtgtgatcacgactcactgcagcttccaactcctggcctcaagcgatcctccggcctcggcctcccaaagcactgggatgacaggcgtgagccaccgcgcccggtccctgtcctctgttgcgatttggcaggcagctccaaggatgtacggaggcacccctccctcccccaaaccccctcccaggatggagacccccacccaggtcttgcaggacagtgggtccaggcacgccccaactcgcccacctgccaggcctgtgatgagaggctctctgggtggccgccgggcgaggccccctgttcaaggtcaggggaacccactattgggacaggtcatggggagagtttggaaggagcttctgtgggggccgcagagaccccaagactgcttccctcacacggccagtgggtggggcctgctcaagtccaccccagcagccccaggcctggcgttcagaggtggtctgggactgtctgatgtcctgctgtccaggtctgcaccagccgtgagccagtgcacaggacagtgttctgaggacagcagaacgtgccggccaccccagcccccatcaacactggagtggctccttgtcacccagctgggccacagcaggtacaggaggccccagagcagctggcatcagcaaagaagcatagagccctcgaggatgtgccatggggtcatggggggctaggcagccccctgcctcctgacaccgcctgttccaccctgtggccacagccctggggagcggggacaagcagagcggccgggtgtggctcatgtcccctaagacagctccctttgcaaggcatcagtctccctggctgtgaaatgagagtgacccctcacagcatcccccctccatggcgctacccgacagaggagacaagtcactgaggtcatggacagattctgaaaaggcagaggtgcggccctctacccggcagcgtggggcatagggacggcagagaacagctctggggtgggccaggctggaagcttagcaggtgggccgggcacacctgcacccactgggaggggaggaccactgaaggacccagcaggccaaggaagtcccctcttcctcctgccaggctgggaggcccagagggtggctggagaggggccctgagtccagagaagctgctcccaggctgtgtggccagggagcagaggttcagaggcaagcccagctcccctccccgtgggccacagcaaccccaggccaggccctttcccactggctttcctggggagcgttcctgaacaggaggaaataaaggtctgtggggagagagtctggggtgagccagcccagggggccacccccaccgcaccattgcacgggccctgtggccgtcctgtctcaggagaccatggaaaggccgtgggtcctgcctgtctttccagaaaagcctgtcgctatctcccctctgggacaccccagctcctgatgtggcctttccagccaagctgcagctggacagcagccaccacgacctggggctggcagagacccctccatcccagagggaatcagggacacccctttttccggggtgtccagaggctgcatattgaaccagggccttggaggcaaatgcaaggtcttcccccgctccctccctgggggcctcagtcaccgccggtgagtttaggtttgttccgaggggcttagactattggcccccagattcctggtgtgcctggggtggcgggactgctccggcggggccctctctccacagcgcagccggcctggggctgacagctcccgagcagggcagggctggtctgggcactgggccccgttcacagccaatgacaggaccagtggcctggaagggagggactccgcgcctttggactccagtgccttgaaatcccacagacctgggaaccgagtcagggatgcccggccgggagaacgaacgggcgggcggcgggtggaccaggacctgcgtccggccccacctctcccaggcctcacctgtcctggcgcccgccccggagcagccaggtgcagggagccgggggcaggggcgccgctcacacccagggacccccggtgcgggaagggcgtgaggcatgacgtcaccgcgaggtggccccggagccggcggaccccacagcccagcagagacctccggctggcggcccgttccctccctgcagccagccccgatttcctctttctgtgcggagcccgagctttcgcaacgcggcgggcggtccgcggtgaccgtcagacacgcagcccggcgggggaggggaggcggcggcaggggggtttccgggggtcctggggggggcccccccgctgcgcccggcccgggaagacgtccgggtccccgcaggtgcagccgggcccgggccccatccgagcgccgatccggcccggtgttcagcgccgccacccccgcggcgtccgcgctcccccgcccggtccccggggcccacccaggacccgcccggcaccccgcgcccaccccggacccacccggccccgcgcccacccggcccacacctacccggggcgccgcagtccgcgcagcgcgcgttccccggccgctgcagcagctccaggaccgccctgcgccgctccttggccatggccgcgatgcgcccgcgatgccgatgccggggccggggccgggagcgtcagcccggctcgctagggccccgcgcaggccgcccgccgccgccgcccctgcgccatcccgggcggcctcagcccgcgcgccggttccgcattcccgccgccctctgggctccgccgccgccgctcgtgtctccgccgcggtcgctgagcgagtgccggcgcggggcccggggcgcacgctgcgctctggccggcaaggcccgcgggcggcccccagcggtgcaggcgagcgtgtggcctcctccctcggccgctccggcgcccccagcccgcgcgccccggggacccgctgggaaatcgcccgccgcccgccccccatccgcgcgcagcgtcccacctgcctgggcgcgaggccgccccaaaacctggccggcctgggtgggaccctcgtgccgccgcgcgcggtgggacggagcctgcacacctactgtggggggcgcagagggcacctgccgagccccccaagaccatgggcatgggggtgctcgtggaaggcaggtgtgcgtgtcctgtgctacggaccaggaccagggcgctcacaccatccctgcctgggagggacggccagcaggtgacaaacgccagcgtcccagggcactgaggccacacagggcccagacggagcctccccctgagacccacccagccgcccaccacccacagatgggaccctctgggcacccctcccactcaggcaggctgaggggccccgccgggacttcacatgggggctgacaggtaacaaaaaacctggttttgatgtcacctcttcccagactcgcgtgcccttcctctgcacaccccagcatcttttcaaattctttccattttcttcttgcagggttaatgcagtacacccggctgaaaacaaactggaacataccagactgaaaaaggaaagcctctccccactgccttccaggcaaccactgttaacaaattcctcagcaccttccagaacgaacgtctgaggcctgtagcctttcatttatttattcatttatcgagacagggttttggctaacggctgtaatcccagcactttgggagaccctggcgggtggatcacttgagctcaggagtttgagaccagcctgggcaacatggtgaaaccccgtctctactaaatacaaagaaattagccaggcgtggtggcgcatgcctgcaatcccagctacttgggaggctgaggcaggagaatcgcatgaacctggcaggcagaggttgcagcgagccgagattgcgccattgcactccagcctgggcaacaagagtgaaactccgtttcaaaaaaaaaaaaaaaaaaaaaaaaccaaacaaacaaactcaaaaaaagttcaactgaaccatgacttgttcacccagtcactggcataggggcatttagagcgttttgaatgcatcagaatcatggtctacacaacctcatctttgcaagagttttgtgtggatttgctctcaaagtggcatcgctggggcaatggtatgggatgtggaatggcctccactgaggctgcactgaccagagtgccaccaaaggcacctgtccccctgctccagctgacgtggaatcatggagctgcttaagctctgtctacctgaagcagaacgcggcagcccgttttggtctgtgtctctgggatctgtgccctgtggcatcacgtgctctttccctgtaaccgttcacatgggtctttctcctccctcagacgggaggctttggggtggggacaggtggcattttcatctctgggtccctgtgccaggctcggaatccaccacacagcatgagcttggagaatgtcgcctaataaagacagacgaacattaatagcagcagctcagcactaggtattaggagaaatagcctgacttgtattttctattttcttttcttttcttttctttttttcctttttttttttttttttttgagacagggttttggtctatcgcccaggctggagtgcagtggcacgatcttagctcactgcaacctctgcttcctggggtcaggtgattctcctgcctcagcctcctgagtagctgggaccacaaatgggtgccaccatgcccaggtatatttttattttttggtagagacagggtttcaccatgttgcccaggctggtctcgaacacctgacttcacgtgatctgcctgccttagcctctgaaagtgctgggattacaggcctgagccaccacgcctggcttgacgtgtattttcttttttcttttttttttttttgagacagagtcttgctctgtcacccaggctggagtgcagtggtgcgatctcggctcactgcaagctccgcctcccgggttcacaccattctcctgcctcagcctccccagcagctgggacacaggtgcatgccaccacgcccggctaatttttttgtatttttagtagagacggggtttcaccgtgttagccaggatgctctcgatctcctgacctcgtgatccgcctgcctaggcctcccaaagtgctggtattatgggcgtgagccaccgcgaccggccgacatgtattttcttattaaagccccattttcagctgggaagactgaggcacggggaggttcgatcccttgctggaggccacacacctcaggactggaatatacataaaacaatataacaaagcaacacaccctaacatagccgttacccaaggtccccaaagacggggaggccatcatacaaggcatagaaaaggcagcagggaggcctcaggggagccggtgcccaacaggccagagcttctggcacaagccaaggccatctactgcctgctgttccggggcctcctccgtggggcagctccagcaggcgagtgtcttgccttctctgtccttccatcccgctaagggaactgtggcattgctcggggggcaggagggcgtgaggatgagacagaggtgggtgaacccctcagatctaagagatgtcggggctgagggggattcttccaaagaggaggctgctcggccgagatccgcaagtacctgcctggacccgagagcacagccgtcctgaaatcctgctgccctgactggctgtggtcagggctgggcggggcagtaggggagcccaggactcaggaagaatccggaaggtaacagcatagctcagggtgggccagacccagaccagtgctggtcaaaggctgttccggccagcagcggctggcgaggggcctggacaggcggcgtgcctgctcctgctcatgcccagtggcctacaagctctgctgagcctcctgccccttgtcactgacccgcacggctgcaccagggaccctgacactcattcctcaagcatctccctagtccagagccagaggcagccgcctcctgtgaccactcggccacacagcagaccctgggcagggggctcccgagagcagggcgagctcctccagcgtcgcctgagcacggggcctgcgcgtggtaggccagcaggcgactttgattggaagctggctcctcccaggcctgcagctgcggggtgagcctctcagcacaggcagaagggggctctccccagggaaggggcaaatggactgaggccgggctgggccctgctctgcgaccatcttgcccaacagtcagccgagcttgctcaaccaggagctgcctcaggggaggggaggttgtggaggttcacttccctctccaccccgcacaggggccggggctaattcagtcaaattatctgcgcttggacagcgtctggcacgatggccgtgagctcagtaggccgccctgcaggccaagcaggggtcttcaacatccaggctcagcgcaggggtgggcagctcctggctctacctgcctcagctgtggggatgggggtcgtgatgttgggcccccatgtggccggccccctccagggaagcccactggctgtggcctccaccctgtcctctgcgacgtgcctttggccctgcgcagaccctcctgggccttctgactgctgtgagggccctgccacccctcgggcacctggagtaggagttcagcagcatacggatcctaaagagctctgctcaggaggggcttaaaggagacacggggattccccagaaatagccactgcgccccgaaatgcaggggagggggaaccacccaggaccagaggtggtctcagcacaattttctgggagtggggctagacccggaaacccctttggaagcagggagtcctcaggggcctccccaagatgtcatgccatggaggtgggtgcacaggttggcttactgggttttggggcatttgaacatatcttttgcttggttttccaaacagagatggggtctggagtggagaagggggtcgtggagagccatccccataaccgacgggctggtggcaggtatggagctgggcactgcgcccgggcaggggtcctgccatcccccatcctttcctcccccgctgggcagctggcgttggcctgaccactccagtcacggcccaggtctccccaggtgccatctcttgaggcctgggaccagcagggtcctcgggggccggaatcacgcacttggcatggggaggaggcccagacccgacgcgggaccccgggaggtgctctccccccaccagccctggcccctttccgcagcgaccatcgggcagtagtgtttacaaagttcacacccaaattggtgcagcctcaagaaacagttacttataaagaacccatacaactcagtaataaaaaggcaagtgaaccaattaaaaaaagggcaaagaatctgaacacatgcgtccccaaggaagacacacagccgccgacaggcaccggaaatattcagcaccattagtcgtcagggaaatgccaatcacaccaggatgcagtgaagacctggcggggggccggtggagggaggctaggatttacagacagacagtgccggtgaggatgggggaagctggcccctcccacagcgctggtgggtgtaagacggtgcagccgccctggaaaacgggctggcatggagctaccgtgggaccctggaaacccactccccggtatgcgcccgaggacggcagaaaacacgtccacacgcacctggacacgagtgttctcagcagcatctttccttttctcttttttttttttttcttttgagatagagtctcgctctgtcacccaggctggagtgcagtggcatgatctcggctcactgcaacctccgctcccgggttccagcaattctcctgcctcagcctcctgagtagctgggatcacaggtgtgagccaccacgcctggctgatttgtgtatttttaacagagacgagggtttcaccatgttgatcaggctggtctcaaactcctgacttcgtgatccgcctgcctcggcctccccaaatgctgggattacaggcatgagcgcccagccttattatttattttttcaagatagggtcttgctctgtcacccaggctggagtacagtggtgcaatcatagctcagtgaagcctcaacttcttaggctcaagccaattctccctcctgagcctcccgagtagctgggaatacaggtgtgcgccaccatgcccagctaagttttgtggggttttttgtatcttgctacgttgcccaggcagatcttgagctcctggcttcaggtgatccacctgcctcagcctctcaaagtgctgggattacaggcgtgagccgtcatgcccagccagcgttactcagaacagtcccaaatgcagacagcccacacgtccaccagctgatgggtggataagccaagagtggtctgtgcccaccacagagtgcgactcagccgtgaacacaccacgacacaggcgaagcttgaacacactgtgcgaagtgacaggagccaggcacagaggagcagatggcgggtgctgtgggaactgacaggagccaggcacagagggtcggatggcgggtgctgtgggaactgacaggagccaggcacagagggtcggatggcgggtgcggtcacacgcaggacgtgccgttcacgtgagccctgtgcatgcaagttctcgttctgaaggccgtttctctcgtgtttaaataagaatccagcagctcttcctgcttggctgcatgggctgagcccatgaggccgactctgccccagagtgtagggtccagccctccggggcttagcgggtgttttccccgtgtgcagagacaagaagagattgtaataaataaagacacaaaacaaagagatgaagagaaaacagctgggcccgggggaccactaccatcaagacgcggagaccggtagtggccccgaacggctgggcgcgctgatatttactgcatacaagacaagggggcagggtaaggagagcgaatattctaagtgattgacaaggtgaagcaagttacgtgatcacaggacaggggtcccttccctcttaggtagctgaagcagagagagaaggcagcacatgtcagtattttcttctctgcacttacaagaaagatcaaagactttaagactttcactatttcttctaccgctatctactacgaacttcaaagaggaaccaggagtacgggaggaacgtgaaagcggacaaggagtgtgaccactgaagcaccatagggaggggtttaggcctccggatgactgcgggcaggcctggagaatatccagccttccacaagaagctggtggagcagagtgttccctgactcctccaaggaaaggagactcccttccgcagtctgctaagtaaagggtgccttcccagacactggcgttaccgcttcaccaaggagccctcaagcggcccttatgcgggcgtgacagagggctcacctcttgccttctaggtcacttctcacaatgtcccttcagcacctgacgctatgtccgccggttattcctaggttatgttagtaatacgacaaagagtaacattaaaagctaatgattaataatgtttataatcatgattgataattgtccatgatcatctgtatgtctaatttgtattatgactattcttattctaactatcttctttattatactgaaacagtttgtgccttcagtctcttgccttggcacaatcctccgcccacaccagagtccactccagccctcagtgcccaggtgggctgataccatatgcctttagcagacagggagactgaggctgagtcacccatctcatgggggcttttgcgggaatgagccacaggcctgtctgtgccagaggcaggcacgagatagcaccctgcagaatcaggctggagaagccccagagcctcacagagagggaggcatggacgcgcgtcaggaaaggcggaggccatctcagacggggcttgcctttcccaaggggccctgactgtgccccccaccctcaccatccccctctgtccatccgtccatcatctatctgtgcgatctgtcatctgtgtctatatgacctgtcatctctgtttatccatcatcaagcagtctccctgtcctctacctctgtcatctgtttatctgccatctgagaatcatccgtcaccgccatcatctctatccaccatctacacacctatcctctcccagcggttccctcccctccctgacccctcctccccacagtgtgtttaacacgtaacactcctctcactcccacctgcgatgtgggccgcgaccgtcccggcccgtgcaggaccactgtgggcgaggggctgtgaccgtcccggcccgtgaaggatcaatgtgggcgaggggccgtgaccgtcacggcccgtgcaggatcaatgtgggcgaggggccgtgaccgtcccggcccatgcaggatcaatgtgggcaaggggccgtgaccgtcccggcccgttcaggacagtggcctataacctctgacatggggtcacaggagaatgcggcttctgccttgcccctcgcccctgccccgcctccacatacacagctgggctcccctgcagcctccacgctgcagaggccacgcggacagagcccaggaacagaggtgctgggcccagccctgcccgcccaggggctggaaccctcccctggagggcagagctcagtgactccagcctcgtcacacctgcgtgtgaaccacccggctgagcccagctagccccagagccacaggcgtgggagccggaagatgctgtgtgaggccctgagtttggggtggtctgtgacacagggattggtatctgcactggtggcctcagagaatcccagccccgccagccccacgtccagaccactcctctcaaagagaggcctcggtgaagcaggatccctaccccagtggacgcccatgagggctggtgccttcccatgtctgccttgagaagagctgggaccccagggagggtctgtggctctgctgcccaaagggcccctacctggaccatcctcccccagcccctattgcagcccccaccagtggggagacccctgccccgcccaggctctggctcagcccctgcagcccctcactgaaccccacacttcataggcacctgccctgtgctgccctgatctgggccacatctgggtctccaaatcccctgaaaactgagctggcaggagtggtggggggtgagtggggggctgagggtggtggcaggcaggtgtcagcgggggcaggtggtaggagggggctgcccagtcaggaaggcctggggcttcaggacacacgaccctggccctagcctgtggctcctggagcacgggcctaactgcacaagccaccgtccccgccgcagccgacagctacgcacctggctctgcctctgcaggtggccctcaagggtgctgtggtgctgccgccgtccagcaggggccacccccaccacagcgcttctgcttcctccaaagtcacgcagcccctgcaggctaagaggggtgggctcccatcccccgccccaaagcaccacgctctgcccggccctgtctggcaggcggccccgcagaccaaccccactgccctcagggatgacccccaccccctggctcttcagagtggatgagtaagcccccagcctcccctctgagtggggacacctgtcgaggtgcacagccgtccagagccacgaggggaccagacggacctgctgccgccccgggccaagactgagccacccacacgcctcacaaccaggtgtggggacagaaaaatgtccctgaagatgtctgcatcctggcccctgggacctgtgaacatggtgcttacatggcagatcgggttgtttgggaccaagatggggatggtccaggattagcaggacccggtgcagttccaagggtcctgagagggaggcaggaggtcagagccggagagacaggagcacaggggcaggggtcggagggatcggaaggggctgcactgctggctggaggttggaccaagtcccagccctgactgaggtgcctcgactccagagaaggcaggaaacggttccccccagagcccgctgaggcgggaaacggttccccccagagcctgccgaggcgggaaacggttcccccccagagcctgccgaggcgggaaacggttccccccaagagcctgccgaggcgggaaacggttcccccccagagcctgccgaggcgggaaacggttccccccagagcctgccgagaggacacagccctgttgggctgtttgtaatttgagacagggtcttgctctgtctcccaggcttaagtgcagtggtgccatcattgtgcactgcagcctctatttcccaggctcaggcgatcctcctgcctcagcctcctgagtagctgggactacaggtgcatgccaccacaccagggtaagttaaaaacaattttttttttttttttgtagagacagggtttcactacgttgccagggctggtctcaaactcctggcctcaggcgatcctcccaccttggcctcccaaactgccgggatgacaggtgtgggccatcgtgtctggcctgggccccttttggactctgctctggaatagtaatgcttgcggtgctctaagccgctgggcttgcagtaatctgttactgtggccagaggaaccgatacaggaagcttcaggaagtcagtttgccttgctcctgaggcctgggtggaacaggtccagtgctgagctccggctacacggcctgtgcaaacgaggccccacgcatcagccctggggctgcagacccgaccctggggggctctgggcctccaggtcagacctcccctcctgggaagccacaccgggccaggcctgcgaccacgctacctccggtttacctcatgcccaaggggaacgcttggaaaacgctctggaaattgtctttgggagccttttaataatgtggccagcttatcatgacaagcgtctatgagaaataaaatgcattctgcacatgacagatgatgaactacccgaaatatctcgtgagaagctctggcaggaacagcagggtgtctggattcttaagcacaacacattcagcagccagacccagaaactcaaaggcagcgttgggaagccactgcacgtttgtaaggagtcaggttgtccacacaatggcgctattttgggattccggcttctctgcctgtgacctgctgaaaccattcatgccggcatcatgcctattggaaactctttgaagttattttgaaacgagaatcgactcaatctattccactatagtttcatacaaagcatatttttttttcctcatttcaaaataatttcagacttccaaaaagttccaaaaataggacagtgtgtatacccagttccccaaataacatcttacataacactgggccagcgctgccagtcacttcgcagaaggctcctcggtttgggtctgtgtctgttgtctccttgggattacattcaggccatgcagttgggcaagagaaccagaggtgataccgtgccctcagggacgcccccaggtcggtgcggcctgtgcggctcgctcccggcggtggctgctgggttaagggggcctctgccgttctgctgtgacatgactgtctcctcggaggtcactatgcatctcaaggggaggtttctcaagacagcagacacctgcttcacaccacacgtcaccactcacttcatggcaactgagggtttttttttgagacggttctgtcgcccaggctggagtgtggtggcacaatccctgctcactgcaacctctgcctcctggtttaggcaattctcccacctcggcctcccgaatagctggattacagacacacgccaccagccccagctaatttttgtatttttagtagagacagggtttctccatgttggccaggctggccttgaactcctggcctcaagtgatctgcctgcctcggcctcccaaagtgttgggattataggcgtgagccactggcgcccggcccgctgagggttgtaatctgcacctgtcaccagtgtggtttctgcaacagcagtttcctgttcccaaaatttcttctatattagaattaataagacagacctggccctccccccactgacatgtttacgcaattacttatgttgtatagactactggatatcaatttattctgagggctacagcccattacaataattaattttctttcttttttttttttttttttcctttggtgagacagggtccggctctgttgctcaggctggagagcagtggtacaatcacagctcactgcagacttaacctctggctcaagtgatcctcctgcctcagcttcctgagtatctgggactacaggcgggtgacaccacgcacggctgacttttcttgttttgtagagacagggtttcacccatgttgcccaggctggtcttgaactcctagcctcaagtgatcctcccaccttggcctcctaaagtgctgggattgcaggcgtgagccgctgcgcccagcctgtttattttcacgctcacactgccccaggctggccaccgtatgctccttcccatccccccgtgttatttcaacaagtctcctcatcccagcaattcttcactctctagctcatctgataccttctctggcctggccctggttccttttggttacagaatggtatcagggccaagaccctgagtgcacgggtcactgcgtccaggccctcaagagctgggaagcatttgtgcacagcccctcacaacagtcccccttacacacggtttcagttacccatggtcagccacagtccaaaaatactaagtggaaaattccagaaataaaccattcatgagtttttgattctgtgctgttctgagcagccgtcctgctgggacatcaaccctccctccgtccagcgtttcgggccatccacgccaccctgcctgccaggcactcagcagccatctcggtgatcagaccagccatcgcggtgatgtggtgcttgggtccaagaagcttttattttacttaagaatggccccagaacaggacagtggtgatgctggcgactcagacacaccagagaagctgccaagcgcttcctttaagggaaaaggtggaaactcctgacctataagaaaaacagccagatgccgaggttgctaagatctccggtaagaacacagtgcccatctgtgacattttgaagagggaagcacgaagttctgtcagttttgctgtcacatctcagacttgatcacaggtacgtacaggaaaaaacagcgcctggacaggattcgggactatccgcggcttcggcacctgctggggtctcggagcacaccctggccaggtgagggcgatcactccgtttctctatccagttacctttgtacgttagaaaagcaggagctcaatacacctggctctcatcatccacgatgtatttctagttttttccatctcagaatacagcttcagaatcgtccacgcaaaaaacaaatttactacttagagtacagtatctttcctgtctttcccacctttggtcgacggtcttccccacactcctgatgggtcagagtgcggtgcgagcggagaggcaggcccgacccgggcctgggcactcccgacggcgggtcagagtgcgatgggaagaggccccggcctgggcccgggagctccgtcggcatggtctgttgtgttcccctcacatcctggttcatttactttttgagcatgtgaaaccctctgccatgacagccagaactccacccccaggggtgctcggaggggctcgcggccggctatgcccatcagttccagctcctccttctggatttctcgtttgtagtttcctgtgtccctgcctgtctccacttcacagtaagctcacgccatatagccgcactgcgttggcgagctcatggcacgtggccacaccgcgtcagcgccgactccacggtggctgcgtcacggctcacgtgcactgtgaggacactggggttgctatagacagtgctggggcaaccctgtgcctacacaaaatattctaataagaaacaagactttccatcaactagcggtgatatgttaatacgtctgcagcactcagaggcctgacagagagcagacacgggacagtgacagctgcagccgaatcaggtggtagaacccatgggtgcagggacacagggttgggcgggcccagccgcaataggtactcagttctgtgaggaggggagggacatgtttaaccacgaaagggccaccactgcaggacagaaacagagggcctttgctgcagagaactggagatgggctgagaagcctcagccacccacaggcagagggacgagggcagcctgtttcccagagcctgaggtcggccagattggtggccccacctgccagggcttggagacagccactgccagggtcgccagagaggacgtgacctggacctccccaccccgtctcctgccaccatcctccactgaccagacaggagccagcccctttccgacagtgcctgggaaggagacaggtctggctgaggccccagatgtgcaggcacctgcgttccatccacaggtttgggccccggctggcctgacgcactgggtggacatgggccgggtttccgtgacccacaggcaactttggcacagacgggctgagaactccgctcagggcccctgccaggctgtgctgcgaacacgtgccggggtctcgggcggagctggctggcacagaagacagctggtgcgagcctaactgcaaatggactcaggttctttttctgttaaaatacaggagttactgaaattaaatacccaactttataaatacggtctgatacaaaccagaaaatgaccctttgattatgaagctacttggtgttcaggcaaaaccagaaagaatcaagaagtcaatccatccaaagccagaaggttctcacttcctactttgtgttccactttttttttttttcaaaggaaatgaactggcatatacgaatacaaacacgaaaacatcctgctagggaatggtaattttctgatgatatctgtgttcagaagggactgaaatttatacatttacctctttgaactcagaaagtatcagaggactgcaacataaaaccgccccatacaagagggccccggcctcgaagacaagggtcttgccccacgctcgcctccctcccagctttgccgggaacgccgtcccactcaggggttcaatgcagaaacaccctcctaagggaaaacgggacgccactccctacccaggagacgcccccacagggctggagcttctattcgaagcccatttctaaggacaccacacgcaggcctcagccaacctaagaggcaggatgatgccctccgtcctgagagaccactgaacacggcccaggggtcttctcatcaaaattcatttttttgcctctgattttccactagtcaagtctccaaatcccagtttctccaatggttcttgtagcatcaattttctaaaagaaaaagagaaagctgtcagggcgggaggtgcagagaggacaagaggggcccttgcaggccccggcttcccagcgcacaccgggggtcctgccacctccatctatgtcctgggagagtggcccctcggcctcggcccccgtggctggtgccttccccactgggcagagtcaacaggacaatgaggacatgaccacctagagacgtcccagggcctaaggcctggggcctcttgggatgtccaagaggggcagctgcacccgctgcccccagcacacaacacttccccacgtcctgctaaaagatgtatctggacaacagcttcacgctgacctctcagacttgctttgattggcaggataatgacccctaaaggtgttcacgccctaatccctggaacctgtgcctgccactgtccacggcaccctggcagaggagacctcaggctgccatccacccagatggaactgagactcaggtcgacagttatctgatatttttttttttttttttttttttaagagacagggtctcattttgtctcccatgctgggatgcagtggtgtgatcagcggttcaagttcactgcagccttgaactcctgggctcaagtgatcctcccgcctcagcctcctgggtagctgagactgcaggtgtatgccaccatgcgtggctaatttctttttttctcccagaaacagtctcgctctattgcccaggctggagtgcagtggtgcaatctcggctcactgcagcctccgcctcccaggtccaggtgattcttgtgcctcagcctcctgagtagctgggagtacaggtgcacatcaccacgcccggctaattcttgtagttttagtagagacggtgtctaaccatatgggtcaggctggtctcgaactcctgacctcatgtgattacccaccttggtctccaaaagtgctgggattacatgcatgagccacactgcccggccagctaatttttttttttttttttttttgagacggagtcctactctgttgcccaggctggagtgcagcggcatgatctcggctcactacaacctccacctcccgggttcaggcaattctccctgcctcagcctcccaagtagctgggattacaggcgcctgccaccatgcctggctaatttttatatttttagtagagatggggtctcaccatgctcgtcttgaactcctgacctcaagtgatctgcccacctcagcctcccaaagtgttggaattacgggcgtgagccactgcacccagccagctaatttttaaagaaattttttgtagatatagggtttcactatgttgcccaggctggtcttgaactcctgagctcaagtgattctcctgcttggcctcccaaagtgctggggctacagatgtgagccaccacttcttgccaaaataattcccagctgtttatcgcagtgcagccatcttagaggcgtgaaattcacgactcagcatgactctttatgggccccacactcttcaggacaatagttactatcatgtgaactcacacatggtgacatgccccccgatcctgctgcgtgctttacagggatggcctcctttaccctccccacaacccagccaggaggcccagccatcatcccatgaggcccccacacactctgtgctgctggcagcgggtgaggctgctccaccagcgtgtctgtgtgacacccggtgcctaaaactaatccccaggacaggacgcgaacttctaaatcactgcagaggcttgagatctcagaccacggtggtcatctgaaacacacctatattttcacatcaggcaaacatggtccaagtcctcaagagacaactttaaaaactccattgtggtccattttctaaaacagacacacacggaggttggagtaaagtaatgtctaggattttagcacagaaggaaaaaggaagaaaagaaatggctaaaaacagggtgggtgccggttatgggaggtagactcctcaacttctgtatgtttgatgattttcatgaacaaataaccaatcgaaattctacggtcaaaataactgaccttatagaaagagcagaatttttaaaaagaaaaaaaaaacaacaaaaacaaaaaacaacaaaagcctgggcgtagtggctcacgcctgtaatcccagcactttgggaggccgaggtgggcggaccatgaggtcaggagatccagaccatcctggctaacacggtgaaaccccgtctctactaaaaatacaaaaaattagccgggcaaggtggcaggcgcctgtagtcccagctactcgggaggctgaggtgggagaatggggtgaacccgggaggcggagcttgcagtgagctgagatcatgccactgcgctccagcccaggcgacagagtgagactccgtctcaaaaaaataataaaaataaaaataaaaaaacaatgaaatcagaacacatgccctgaacatttgcctaaacattgcctggccaatctgcgagaccacctgaggcactggccctggctcccttcctcccagctgtcctcgggagtctcaaggggaagctgagcatgaggcaggaggcctgctcacaccctcacatgaacacagctcaattcacaccgtctctaaagcttggcagcgccatggagaaattctcagttccagttttctaaagatgcattaatttaagactaatttaaacacgtggctccagccagcgtgctacttaaacaatcagagatggtaagagacagattcagtgtggcccttggacaaatgtgagccctggaagagcctaagcgacagccccaaaggcagaacgtggcctcactgaagtccacaccgctgagaaggcccatcatccttacggccccaaatgtccacacgaataaccccgaacacacctgtgtggccggaaaggcacaacggatttttgccttgagggaaatgtctgagtatgttatgggaacatgaaaagagctcaaagtggatgggcgaattgtcaacctgtcttcagcacagacacagctgtctgaagaccgaaatgcttagtgagctaatcatggcctcgaaaagcaaaaataaaagctcccactaccatcatataattatatgctacatgcttttttttgcataagggattagaatagctcttattttaagtaaaagctgtggggatcatagctacgaacgcgtttgttttgtttaaaagtgagcgttggtgagttctggttacgacaggaagaatcattcatctttcatcatttcttaaatagtctattgtcctccctgtcacattcccaacaactcaaagtggagcaaaacaacccagtgcatatttttaacatctgagaatgcaaaagaaagaaactgtgtacccacgaccttcggtcttttgactcagtcagcattatgatgcacgatttggaaaccttaatgacaggtcggtagcaaccaaatgtggaaagaaaatgctctcaggaatgcaatcatgtataacgctatcgctcaagaagaggttcccaagattgtgaaacagcttttggtacaatttcatctgataagcctccatttcaagggcaaacaattccacagtcctgaaaaacacaagctgaatcccaggcctgaaaaagacacactgtaatggcctttcaggaaaaaaaaagttttaattggagtagtgggaggtggaaatcataacgcttagctgtactcacctctccatcctgcattctaaatattcttttgattcctttctgcacaaagcattttcaaaattattgttatgaagacacttcatgaatttctctttaaagcttttacattcacctagaacgagaaagaaaacagcatgttcttttcagagtgaaaagtgatgcaaccacacaacagccttaagagctgcgctgcttttcctttcaaaacttgtttcctcaggccgggcgcagtggttcacacctgtaatcccagcactctgagaggatcacctgaccccaggagtttgagaccagcctgggcaacacagtgagacccccatctctaccaaaaaaaaccttaaaagttagctaggtgtggcgcacgtctgtggttccagctactcaggaggctgaggcaggaagatcgcttgagcaaaggatttcgaggctgcagtgagcttgatcacaccactgcactccagcctgggcaacagatcaagaccttgtctcctaaaaataatccaggctgggcacggtggctcacgcctgtaatcctagcactttgggaggcagaggtgggcggatcacctgaggtcaggagttcaagaccagctcggccaacttggtgaaaccccatctctattaaaaatacaaaaattagccgggcatggtggcgggtgtctgtagtcccagctactcgggaggctgaggcaggagaattgcttgaacccaggaagcggaggttgcagcgagtcgagatcacaccactgcactccagcctgggtgacagagtgagactccatttcaaaaataaataaataaatgatccagcctggtcaacatggggaaactccatctttaaaataaaaaataaaaaaacaataataattagctgggtgtggtgttgcacacctatagtcccagctactctagaggctgagacaggagaatcgcttgaacccgggaggaggaggttgcagtgagtcgagatcacaccactgcactccagcctgggcgacacagtgagagtccatttcaaaaataaataaataaataatccagcctgggcaacatggggaaaccccatctctaaaaaaaaaaaaaaaaaaaaaattagctgggtgtggcgctgcacacctatagtcccagctactctagaggatgagacgggagactcccctgagtccagaaaggaaggaggccgaggcttcagtgagctatgattgcaccactgcactccagcctaagcaacagagtgagaccgtgtctcaaaacaaaaacaatcaaacccaaacttgtttccttcataaacatgctgaaaacaacaatggaaaaaacccacaacgaggtaaagtcagcccagtacgaggttaaggtgcaatgtaggtgatggtcacaggagagttcagggtaaaaattctttcatttttgctatttgaaaatgtcacaataaaatgttgggaaagaaacagtaattcacagatcgtttttcttttcttttctttctttttttttttgagacgttgtctcactctgtcgcccaagctgcagtgcagtggcccgatctcggctcactgcaacctccgtctcccggttcaagcgattctcctgcctcagcctcccgagtagctgggactacaggcgagccacatcacacccaactaatttttgtatttttagtagagacggggtttccccatgttgggcaggctggtctcgaactcctgatcttaaatgatccgcccacctcggcctcccaaagggctgggatcacaggcatgagccaccgcgcccggcccacagatcctttttcgaagccttcatgtccacggaggcaaccccaccggaatacggccagaagcactgggagatcagcagcacttttgagctttgcagcagacatcgctaggcaccgtctaagcctcggctgcagctgcgggcttcgtggccgccgaatgccccgctctgcaaacccggggcacagacagcccgccgggtgagtcagggcggagccggagacccggaagcctgacgcaaggagtcggtgtcgctgccctcgtcacgggggccacggtcctgccagctcccacctgcagggtccatgccgcagcagtgacccagggctgggtggggcggaggggcgggccgccgtgcctcagtttccccgcctgcaccgcgctagatgccggcaccccatagggctccgcgctgggccgcgacccagaccccccatcgcagacccctgcccgccgaccttccgccgctcacctaagtgatccagcgggaagctgcccttgtccgggggccgcggctggaagctcttggtcccgaaattcatggcggtcgacatgttggcgactccggagtctgcgagcgccttgcgagcgtacgcagggcggccggcggagcgccgagcaggcaccccgggagcgccgagcacgtcgagcaggccccgtccccgccccttgcccaggcccggccccaccccttgcccaggcccggccccaccccctcggagtcagcaagctcctgagccccggtgggcgctgcccgggctttcccgtcccctgggaccctcacagcagtcggtagcagaatcagcttttggaattggtgagaaaccgaggcccagaaaggttcagcgaattgcccgaggacacatagcggtgccggcgtctctgcatcccaggtgtctgctggggcctcggtttccccatcgcacagagacctcatcacggggcccatccggggaggaaggcggcacaggagggacaggagagcctgtggactcgctgcctgcggctgctggagccaggtgacaccaccgagcagcttcaacaacaggaatgtgctctcagtcctggggtctggagtccaagcagtgggcagagtgggggccctggggacagtcccagcctcccagcttctgggggccgccagcctgtgaggttccttggcttgcagaagcaccacctggtctcggcccctacgttcccatggtgtcctgcgcatgtccaaatttactatttttatttatttatttgagacagggtattgctctgttgcccagactagagtgcagtggcatgatcacgactcactgcagcctcgacctcctgggctcagcgatcctctcaccccagcctcctgagtagctgcgaccacaggcacacaccaccacaccaggctaatttttctatttttcgtagagatggagtctccctatgttgcccaggctggtccttaactcctgggctcaggcaatcctcctgcctcagcctccctaaatgccgggattataggcatgagccaccatgcccaacctaaatttcctctttttataaggatagcagtcatgttgggttaggggccgtcccaataacttcatcttaactaattatatcagcagcaaccctatttctttgttttgttttttaaaacggagtctctctcttgttgcccaggctggagtacagtggctcgatcctggctcattgcaacctccacctcccaggttaaagcaattctcctgcctcagcctcccgagtagctgggattacaggcgccaccaacacacccagctaagctaatatattttttttcaacacagagtctcactttgtaacctagacgggcgtgcagtggtacgatctcggctcaccgcaacctccacttcccgagttcaagagattctcctgcctcagcctccagagtagctgggattacaggcatgcgccaccacacccggctaattttttgtacttttcgtagagacggggtttcaccatgttgcccaggctggtctcgaactcctgaccttaagtgatccgcccacttcagcctcccaaagggctgggatgacaggcgtgagccgccgcacctggcacctatttccaaagcaaaggtcacatcctaaggtcctagtggcttcaccatacgaatgggggtggagggtagttacagctcaacctagggcagaatgaaagtgtactgtaagcggtaaggcccggcctttgcgtgtcgatgttgtcatggcaacagcagtcgtgggaaagggctcagtgaagaccccagcgtccacccgcggagtctctcccagccctgaacctgtacccacatgccctggccatgaggaagtcgaacactggcccgtttagagcaccatgggcggcccaagctccagcctccactcactcatccatcccacccatttcacaatcactggccaggccttcgtggtaagcacagtgggtccccagcaacaccgagttcgctgcctggccccccgctctggaccctggtgacacctgaacccactgttgcttaggagccagttcctgcaacttcccaggggttgtttgcagacaagggcttggtcaaggcggcccaacagcccccaccccggggcctgcccttcttcaagcctggcccaggcgtcccctccccatgaggcttccagctctcccccttgctgggccctggtgagcccgcccctctgcaacgcacaccattagattcctgcgcccaaggtcggactctgctctgtgtgcccagagctgagccgcgagtctggcactgaatgtttctgaaccgcagggcagtggggacctcaggccaaggccccaaggagggcccagggctgcagaaatctagttgtcagggccttgattggcctgtggctctgggcccactgaaaactacaaaaggaaaattgcagcttgttcctgtcatataaacaactctttttacccggttagaaccaggggcccacacactgggttactcattaatacgtttccagggctgtgaacaatgggagcttttattacccagcaaagaagcgctcactcacttgactgcagcagccgcttagcagtggaaggacagcccctccgttggggctgccctaagcctctctgtctgggagccccgagggacctgtggcctggaggtgacaaccggcttttaattgactgtcacctggcaaatgattattgctttatatgagatctttagctgggagagaagaggaaagaatttttatgagcagttcaccggtgtaaaaagtggggaaggggctgggggcggtggctcacacctgtaatcccaacactctgggaagccgaggtgggcggaccacttgagcccaggagtttgagaccagccttggcaacatgctgaaaccctacgtctataaaaaatacaaaaattagccaggtgtaggggcgcacacctgtggtcccagctaccccagaggctgaggtgggaggatcgtctgagccctgggaggtcgaggctgcagtgaaccaagatcgcaccactgcactgcagcctgggcaaaaaagtgagaccttgtctacaagaaaaaaggaaaagctcagaggagggaacgaaccttgtttgagccccagcaatcaaggtattgcaggtatttccacccggatccccagagcctggagcagtgcctggcacgctggacatgctccgtttatagctcgaagatgaatgaatccagctaagtaggtcttattatcctcatctgacacagagaaacagactcaaggggtcacagcccttgctgaagcttacgtggccgtgttcgcgttatctgccgctgcataacaaagcaccccaagcttagcagcagtcattacccgtgaacttgcacttgcacttgggcagggcttggtggagacattccacgaggcctgggccgggttagctccacttcggactggaggatccactcacgatggcacacgtggctggcaggcaggtgggctcagtgtcatgtggaagcctggccacggttccgtgggcctgggttcccctccacatgggcccctctacgagctgctgggcttccccacagcatggcggctgggtccacggcacaggtgtcctggggaaccaagaggaagctgcatcactgtttgggatccagcctccactgtgaccacaagtgcccacccagattcaaggagtcaactaaaggaaaattttgctttcgaagaagtaaagttagttttattcagattcctattgagcattgcaatccgggagggtgtttcagagtttcagttcgactgctccaaagcaatgtttcgggcctcagcttatcaacgggtggcggaggctctgcacctgctcagggggtaccttagagccaaatgcgatcgtcaaagtttcggtgcaagagtccatctcgtcatagattacagccactgatcctgtcagacgttatcttatgtgcaggaggagacaagggctaggatcgttgaccttatctttttaaaaaacgcagtgatttggccgggtgtggtggctcacacctataatcccagcactttgggtggccacggtgggcggatcatctgaggttaggagttcaagaccagcctggccaacatggtgaaaccccgtctctactaaaaatacaaaaattagccgggcgtggtggtgagtgcctgtagtcccagctactcgggaggctgaggcaggagaatcgcttgaacctgggaggcggaggttgcagtgaactccagcctgggcaacagagagggaaactccgtctcaaaaaaaaaaaaatgcagggattcaagcaagagccgtgggcacctgcgccatgcctgctcagtctctgggacattcttccagggggcgcgcccagtcactgaggcagggcgcacacttttttccgcacagttgcaaggatgccttaaactaaatttctggaaaccgggtcactgagccaagggacagttcctttttgatgtctggtggatctctagtacactgcaaaagcgtcccgtgaaactccactggtggaatgagcaaacacggcttctggcctctgctactttgtgtcacaaagggggaagaccacaagccctcctgtggagggagaagcgcagtgttacgttacgcaaagacagtgtggttgggagaatctgtgcggccatctctggaagccgccatctgccacagccgacaggtgcgggcctgagtttggaaccaggtctgtctgactccagcccctccgggggagcccacctacccacccacctcacccaccctgcctctgatgtcaggggcaggcaggacctgagccagccaatcacagcactgcattcctcctggtcacagagatggctcaggcctgagtatctgtctggcaggcctggaccaatcaggtctgatcagaacagtcccaagttccacttgagcagccaggctgcagttggcacacgtctctgcagggcgctggaggtccaccggacatcagaaagcaacaatgccttggctcagccacctggtttctaggaatttattttaagatatccttgcaactgtgcccatccttgcaagtatgcacacagatatttttatcaggatgttctgtgacagcctaagattggaaacaatgtaaatatgcaaatatgcacacccggccatgccccccagagcaagcaacacgtacaggcagccgttagaggagtactgttaattccccagtaacagttactttttttttttttttttttttgagatggagtctcactctgttgcccaggctggagtgcagtggtgccatctcagctcactgcaagctccgcctcccgggttcacgccattctcctgcctcagcctcctgagtagctgggactacaggcgcccgctatcacgcccggctaattttttgtatttttagtagagatggggttttgccgtgttagccaggatggtctcgatctccatctcctgacctcgtgatccgcccacctcagcctcccaaagtgctgggattacaggcataagccactgcgcctggccaacgagttacttttaaaagagggctgggcactgtggctcacgcctgtaatcccagtgctttgggaggccaaggcaggtggatcacttgaggtcaggagctcgagatcagcctggccaacatggcaaaaccccatctctactaaaaatacaaaaattagctgagtgtggttgcatatgcctgtaatcccagctacccgggaggctgaggcaggaggatcacttgaacacgggcgatggaggttgcagtgagccaagatcatgccactgcactctagcctggatgacgagagtgagactccatctcaaaaaaaaaaaaaaagaaaagaaaagaaaaagaggccggcccggcattggctcatgctagtaatcccagcgcttttggaggcggccacagtaggatcgcttgagcccaggaggagttgaagaccagcctctagactccgtctctaaaagaaacttacaaaattcccagcactttgggaggccgaggcgggcggatcatgatgaggtcaagagatcgagactgtcctggccaacatggtgaaaccccgtctctactaaagatacaaaaagtagccaggtgtggtggcatgtgcctgtaatcccagctactcgggaggctgaggcaggagaatcgctccaacccgggaggcggaggttgcagtgagccgagatcacgccattgtagtccagcctggcaacagagtgagattccgtctcaaaaacaacaaaaacccaaaacttaaaaaattaggccaggtgcagtggcttacacctgcaatcccaacactttgggaggtcgaggcaggaggattgcttgaacccaggagtgtgagaccagcctgagcaacgtagcaggacctccatttctacataaataaataaataaagttagctaggctgggtggtgcatgcctgtagtcccagctatttgggaggctgaggtgggaggatggcttgagccccggaggtggaggctgcagtgagcctgtactcacagcacggtaatccagcctgggcaacagagcaagaccctgtctcaaaaacaaaacacacaaaaatcaaaagtattccctgaattaattaagtaagctttgctaggcaggtaggacccccagcctacccagtagtggccctggtatgaggcactccctccccgccgcagccgcccagccctggccctgaggctgtacagcaggatagaggtagtgagctccccaccacaggaggcgtgcaactgtgcctggggctttccctcccagctaatcaagggggaccaacccgggaggggtgagggcctcaggctgcctcggctctgcggttcgtacagggcatgggcaacgccccagagacaggctgtggggagggtcccgcctgctggcaccagctccagctgcctcatgtcagggcaggctgtgcaaagcaggcccggccgcttctgctgccagcagccctcccacccctcacagcggtttaggagacagatggacagggctcgtaaaccccgccacctgccacccggcagcccttccatgttcctgtcctagctcagagggttgcatcccctgcccgggcggcagccctcgaaaaggagcaggctgctacctggggcgagaacaccctcagggagatgcaggtggtggcctgtgaggactgtgggatttgagtcccctcatgggcctccctgaggtctcactcctgtcccttgccccgtggggagctgatcagagaagagctgggtgcacacggatggggtttgggagaggcctgcaaaccctgagctccacagggtcccactcagagcctgtgaaaggcagggggctcccctccaagccagggcctcggccccaaagccctgtgaatcccagttctgaggggtggccaaggccaaggggcctggactggcaggttcagcagaaccaggggcagaacagtccacgtgccacatccttctctctggccaggagacatggatgcctgaccccagagggcccccgtcctgccctcccctccccggccccaccagggcctcagccccacattccacgcaggcctgcagctttaagccatccgctgggggctgccctgcaaactgctcgttccacattctcggggtggtggggtgggtggggtgcgggcacgccctcccgcggaggcctataagggtgcgggggggacggggcccaggaggggagtggagcctcaccagccacgtcctcatggccctgctgctcttgctgttcctgggcctcctggggctctgggggctgctctgcgcctgcgcccaagacccctccccagctgcccggtggcccccggggcctcgcccgctgccgctcgtcgggaacctgcacttgctgcgtctgtcgcaacaggaccggtccctgatggaggtaagtcagggagcccgggcagctcttgccgcttggacagctgccgtgtcctggcccccctcctggggaagcccagcccggtttccactgcctgttcccacatggtgccgggcccagcggggcacgcaggaggccggagggcccaccctgcttttgggaggggaaagccaccccgtcccacagggaagcagacacactgagtacgaagaagccctggaggtgccacccagggcccggcgcgggagcaggagtctggagggctccctggaggcggtggcgtctgctgagagcccgcaggaggccaggcttagggacagcagaggggaggagggcctgggcctgtcggctgcctgctcagcccccctcggccctcaggtgttcaacagcaaacacttcccacttttgagcctctgtttcctcatctgtgaaatgggaagaggacctgtgcctcctgggcagggatgctgaggacagagggtacggggacacggcacggggcagcctgactagcactgtcgctctgtcctcgctcaggcaggcagggtcagtgggacagggctgaccagtcaggcccgggccctgcctccggctgcccccaactgcgggctggggtgctgctggccgggctgtagccgaagacggatcagggtctctgggggccgagcctgtcaggccctcactctgtgcagactctggcctttcagaatgtgccaccccagcagacagccagcccccgaccttgccgcctggactcagggcttgagccccccaggaattttggctaaaaagaaaatcccacaacagccatgccaagcaccagatggcgagagggctctgtgggctccaggctgggcgctgccccctccaccctgcaccactggctttatggcatctaggcgtgccccctccacctgcccccattttccctctgtccccacccctacccagcacaggcccggcctgaggggacctaaggggggtcttgtgggtgagggctgcccgggtgacccccgccatccctgccagctctcagaacgctacgggccggtgttcaccgtgcacctggggcgccagaagacggtggtgctgacggggttcgaggcggtcaaagaggcgctggcgggccccgggcaggagctggccgaccggcctcccatcgccatcttccagctcatccagcgaggtggaggtcggtgtgtggccggcgctacggggcctactgggtgtgggggcacctggaccccaggaggggtgggggctccagcagcgggagaggctcccagggtggcctggggaagagcaggcaggagctgggcctccgggctggtgctgagaagggccaggtgtccacacagcaggtcaggtgctcagaaccagcaagcggggttcttttcatcccaagtcaagaggggccagggccagcccagggggacgggagtggggatgggggtgagggcccagccagtctcgctgccctgtcagcctgctcacttcctgcccacttgcctggcggggctggctggggtgggaacctgggctcaccacgcactgtatctgcctacaggcatcttcttctcatctggggcgcgctggagggctgcccgccagttcacggtgcgtgccctgcacagcctgggcgtgggccgggagccggtggctgacaagattctgcaggagctgaaatgcctctctgggcagctggatggctacagaggtgagcagggggccggggacgcccctccccgggcctggacgtgcctgaggcccgtctccctcgcaggccggcccttcccgctggccctactgggctgggctccctccaatatcaccttcgcgctcctcttcggccgccgatttgactaccgggaccccgtgtttgtgtccctgctgggtctcatcgatgaggtcatggtcctcttggggtcccctggcctgcaggtgaggagctgcctcccatccttggggtggggtggagcctggagtgatgggcgtgcagcaggaggacgggggccccagtgctgtcccctctcagcttctcggccctcccaccttgcaaaaggaatcagatgcaagggcctgaatcaggacccatccgacgttagctggtcatgtggcctcaaaccagcccctgtgctctccaggaaatgggggatccccatctcccccacgccctcatcagtaacagatgcatctgctcaagagtggaggtgggaggcaggcatggcttccccagaattgcggggggctccatgtctgccccccaagccctgacagtccaggcaccttcctgctgacgccctttacagtgccccaaaaccacaaaagttcacccctctgatgtttggaactcgtgtatttcatttagcttcaaaatgtaaagatcccctctttttggtcctcatggtatttgatgtgcgtttaccgaagcctgcatctcaggtgtgatacctgcatcacggctggacccagagaccacgggtcctagtcctcctggcttcccagaggcagggccgtgatgggagtgagtgagcagcggtgggttcacaagcgagagcagggggcacagcttccacccgtcctagccagggtgagaccaccctgcctgctggtggagccaggcaggcccagagcccacctggaagatggaggctgcacggcactgcgtggtccggggcagccccagggcagcagagcattccctggccttccctgctggtgccagctccttaccacagagacgccgcgtggaactcactactggcgatcgcggacgccccaggaaggcgagtggcacgaggtgtggcggggctgccacaggcagtcacggagcacccgctggtgccaggctcacccctcagatacagcctcgtgggctggctcagtggcggccctgactctccaaggtctgaggtctgctctgtcttccattccatgactcacaggaggttcaaggttacttccactgtggcagctgcctccctcctccctctccaggatggaaggaggtcctgctgtgcaaacctgcctggctgtccccttccgggacacggacagggggtttcctggcagttcctggtcctcccttgagggctaagggtccccccgttctgtggccgcctccagcacatccacccagagtccctgggcgtgaacacagccgctggggacgatcaccgcctgcccagcgtgcagccctggggctgcgtccttatctccgctcctcctgcctcctgcccagctgttcaacgtctacccatggctcggggccctgctccagctgcaccggcccgtcctgcgcaagatcgaggaggtccgtgccattctgaggaccctcctggaggcgcggaggccccacgtgtgcccgggggaccccgtgtgcagctatgtggacgccctgatccagcagggacaggtgtgtcgggacccaagacctccttgaaggcctgtaggggtcctgagggacaccccaggggagcacggctggggaggggtccataccggtcctgcaccaagctccctacctcctggctgggggcctctcagagcctcagtctccttgtctgtgaaacggggcatccatagtgcctgcctcggggacgctgagggatggctgagcccagatcaccggggcatccatagtggagcccagggctgggctgggtctgtggggtgggggacagaccccagatcatcccacgagccctgccccacgcctctgcagggggatgaccccgagggcctgtttgctgaggccaacgcggtggcctgcaccctggacatggtcatggccgggacggagacgacctcggccacgctgcagtgggccgcacttctgatgggccggcacccggacgtgcagggtgagaccccggcgcctggcgagacggctccttctgcccccgggggacccccagggacgagggatggcgctgccacccaagcggcccaccctttgccccaggccgggtgcaggaggagctagaccgcgtgctgggccctgggcggactccccggctggaggaccagcaggctctgccctacacaagcgccgtgctccacgaggtgcagcggttcatcacgctcctgccgcacgtgccccgctgcaccgcggccgacacacagctgggcggcttcctgctccccaaggtggggctgggcctcccttgccccttccatctcctctggggtaggcctcggcgggggtccaggggcactgggacggctgagcgtctggtgggtgcctgatggcccagcgctccccgaccggaggcaataaagggcgtccagccaggagcaggagggaggcagccctctcaccccacagcccagcacttggcctggccagcagacctggcgcctccctgcatgtcctggggtcccctccgtgtgtcctgtgggcatccctgtccatgtgtcctaggcgatcccctcggtgtgtcctgggggtcccctctgtatcctgggggggtcccctctgtgtgtcctggggatcccctgtgtgtcctgaggggtcccctctgtgtgtcctggggatcccctgtgtgtcctgggggggtcccctgtgtgtcctgggggtcccctctgtgtgtcctgggggggtcccctctgtgtgtcctggggggtcccctctgtgtgtcctgggggtcccctctgtgtgtcctggggcgtccctctccatgcatcctgggtgtcctctctatccttggggcccctggctgtgtgccctggggtcatgtggcctccctgggtttgggtgcctcacctgcaaggtgggctaacaccaggtgtggtgggtgtggcaggagctacatcctggaatgaaacttcccaacccaggccccatcccatcttccccggggcccctctctctgtgccccggctgcccccacagggcacgcccgtgattcccctgctgacctcggtgctcctggatgagacacagtggcagaccccaggccagttcaaccccggccatttcctggacgcgaatgggcactttgtgaagcgggaggccttcctgcctttctctgcaggtcagcagccctcggggccggggtggggcggcacctccagggctccaggggtgggacggccccagctccgcctgccgcctctgcacccacctcctgatctcaggttctgaaggcggctgtggtggctgctcctgtgctcccctggggaggtccccacccctcccctccaggagcaggcctggtgcagcccactctgtgcctggacatcccccgcaggccgccgcgtctgtgttggggagcgcctggccaggaccgagctcttcctgctgtttgccggcctcctgcagaggtaccgcctgctgcccccgcctggcgtcagtccggcctccctggacaccacgcccgcccgggcttttaccatgaggccgagggcccaggccctgtgtgcggtgcccaggccctaggagctcccccagcccccaggtcctcctgaccactcccctcccagccctgggtcctcccaccctctctcctcccaccccacagctcggactgctctgggagggccctgaggactcccaccctcacccccacccccacagggtcagcaactgcttccggttacacccaggactacccctgcccgaccctgtgggacccccacccctctgatgctgtctgcagctcagtccctgccagcccccaggagcgcctccagggccccgcccactctcccacccctgaagctgcactcccacccacctagctccccccagggccccccagcacctacagctggggctgcagggagacaacgggtggctgcatccagccagagacaggcgcaggtgggtgtcctcagcgtgcgagccctgcaccccccaggtcctgggactcctgcagaccccactccattcccgctcctggaacacttcctgcagctgtgcctggaggcagtcggcctgcagtgccagactctgagccaagccactggggccatgcgtatgactggtgcagggaggcaaggcccacattctccttcagagacaggcactggcgccagaggcttccttggggcggggggagggcacctcagcccctgaagacaagcagcactgcagtggcaaaaatggaaacactgacccggtgcggtggctcatgcctgtaatcccagcactttgggaggccgaggcaggcgaatcacgaggtcaggagttcgagaccagcctgcccaacatggtgaaaccctgtctctactaaaaatacaaaaaaattaaccgagcatggtggcacgtgcctgtaatcccagctactcaggaggctgagacaggagaatcgcttgaactgggaggcagaggttgcagtgagccgagttcgcaccactgcactccagcctgggtgacagagcgagattccatctcaaacaaacagaaggaaatgtcgagccacacagtggcacatgtgctgggggcggtgtggtcggcagtgtgacagtggcacgtgtgctgggggcggtgtggtcggcagtgtgagtggcgcgtgtgctggtggcggtgtggtcggcagtgtgagtggcacctgtgctggtggtggcgtggtcggcagtgtgagtggcgcgtgtgctgggggcggtgtggtcggcagtgtgagtggcgcgtgtgctgggggcggtgtggtcggcagtgtgagtggcgcgtgtgctgggggcggtgtggtcggcagtgtgagtggcgcgtgtgctgggggcggtgtggtcggcagtgtgagtggcgcgtgtgctgggggcggtgtggtcggcagtgtgagtggcgcgtgtgctgggggcggtgtggtcggcagtgtgacagtggcgcgtgtgctgggggtggtgtggtcggcagtgtgacagtggcgcgtgtgctgggggcggtgtggtcggcagtgtgacagtggcgcgtgtgctgggggtggtgtggtcggcagtgtgacagtggcgcgtgtgctgggggcggtgtggtcggcagtgtgacagtggcgcgtgtgctgggggcggtgtgatgggacacctgctgtctgagacggccctggcccccgtcccagcgctgaccatgcacgtgagcggcaggcggcccctcaccgagaagagaggataagccgtggcacattcccacctgcgccacgtgaagcggcgccccaaggccaggacgtggtggccgggacacacgggtgcacggcaggcctcacactcccaggtggggccgcagggtccctaagtcatgggtggggctgctgctacggaggctgctgcaaggacgagaaggggacggagggatggaggctcggctgcgtccgctcacctatttctggggaagagcagggagcgagtgctggcttcactattctcccctcagacatttgaaatatgacactgaaaacttgaagggcagaaagaacggggtcctggcgccaggtggggccacgcgacgccccgcccagtgtgactggtgagcccctctggagctctggagggtgcggaacattccagggcaggggctgtggaaaatccctcccagctgttgtcagggctcccgggtggggtggaggactcaggcccagccctcccaaccctacaggggacccttgtgcccccaacttagtcggcctcctgggtggggtgaaggactcaggccccgccctcccggccctgcaggggaccctcgggcccccgccttacattggcctggggtattcatctccttgggcccaggggagcccgaggcccacgtggggaggaaacgctgaccctgtcccgccccgcttcatggaggcccagccgacatgagggacaccaagtacagggtgatgagtgacggctgagcttcccctcaaagcactaagacaataaacataaccccaaccccaaccctgaccccgctcgcccgactcacgcgacctctcaccccatctcacccagcgagatgacagggctcctccacggggtacagccagggccccccaagttaggcggtccaaatgcctcatcctcaagtgtaaacaagcctagcttagtgaaaacagagtaagcaggtatcaaatctcctttaactcatgcccatgggcgtgggcaggggtgtggggcgggcaggagggaaggggggggccgagtgagacccgaaccccggaggtgccaccttctcgaccgggcagacctgggtgcccgtttctcacagctgcactcaggctgccggccaggtgcacaggcacgtcccgggggcttgctgggaaatgccggggcttggcacggatgggacaggccactgtctacgcctcatttccaagtgtttctttgttgctatttttaaaaatggtgataaaatacacgtagagaaaattcaccatcagccatgttgaagtgcacagttcaggggcacaggcacattcacggccaccactgtcacctccagagcctcctcatcttcccgggctgaagccctgcttgcgttaaacacccactccgagtccctccccagtccttctccttcctggtctgtgggtgtgagggctctggggacctcgagtgagtggggagtggtggatttgtcctgctttatccacgcacctgccaatgggtccttttcaagggtcccagagaactcaggcagcaggcagcccgcgagcccagcttcctcacgcatggatttcgggagatgcctctgtggacatccaccccatggtgcccagatgtcatgggagaggcagacaccccgaaccccatggctcacgacgtggctgaactgctccccggacgtctgtcctcagcagcaggagggacgccgaccccggcccctgtacccccccgcctctaggtctccagggaaggaccctcctggggtgggcaggctgctcttggccccagatctgcttctgggtctgaggctggccctcagccgtgctgcacgtgcctcccattaccatcctaagtcaaggcaaaacaaaaccacacagtccccgagccatctgcaaacctcccgcagcaagctcggagggtgaggcccccccacagtaagctcggagggtgaggccatctgagtgccgggctggccgggcttaggaggagaccaagctttacccacggagacgccgtttaatggaacccaagtgacccaagggagctgccacgccaccgctcaaccacacccacagggaacacacatgtaggggcctggggccttgtccagtgcgggggtcctggttcagatgtcctgaagctcctccctcggagtgggcagcccccattaggaaacttctggaacccagaggctggaagccggcctcactctgggacctgggccctccccgtgattgctggggctccctcagagcgggggtcttcttcccaccttggatgctgagggggaaggaggtaagacactggagggctgggtgggctcaccaggcttcagagggcagtgggacttggacagctcagcctagcgcccagctgtctctccgggcaggtctcacaaatgccccgaggcaggcccatcgctggccaaattccccacccctcctgcctggactcagtcccttgtgaaatctaagcaaagtcctagggtgtctgaggtcagagtgcatcgggtagctcctgctgcataacaagcgggggagggggtggcccatggctctggactgctgggccacaggggtgatgaggtgaggcttggaggagtctgtaggatcctaactttgatgactattcagggtttgaggctgcactcagctttatcgcctgtgaaacccacgcagccagcaggggtacgagtgcttctgaggtcccagtgggcggggggtggcctgacccagacagggcagggggacggcagctggagcgcgtggagagagcgcccgttccctgggagccactggagggcgcatggcacacggcgcccggccagggcctgactgtgactggagcaagagccgagggactggacggacggcaggtggcgccacctgatctgtgcagggacaggcagggacaggcaggcagagaaagagcccagctggaggctgccagcgctccagggagaggagtggggagcggacagcgccggcccgggctggaaggtcagcttgaggctctgggatgtgaacactgggcaccgagtcatccagaaataaatcccaggcacggtgggaggccggggtgggaggatcgcttgagcccaggagtttgggaccagcctgggcaacacagtgaggccccccgcttctacaaaaaatacaacaattagccgggtgtagtggtagcaactgtgttcccagctactcaggaggctgagggaggagaatcgcctgagcccaggggggtggaggtggcagcgagcagagctcatgccaccgcactccagcctggacgacagagcgggaccctgtctctaaaaagatgccactctcttgggggcttggagggaggacagtgctctgagggtgattcagcggccgaacctgtgtcactcttggtggagtgagcagacggcacttggtgtgagctgtttctcagacatcaatcctgcaagaaactgctcagaggccgcttcctcagagggctgggtgggctggggcaggggacggtttccggaagccgcacagtgctgtgagaggtctccagcctgctctgcgtgttcgcacccccaatgtcagggagggccctttggcctggaaagcccagtctccacaggtctgctcacccccacaggcaatgtgattctgacttccaaacaatgctccctttgattatttaaaaacattacttttatttttatgtatttattttttcagacagagtctcgctctgtcacccaggctggagtgcaatggcacgatctcggctcactgcaacctctgcctcccgtgttcaagcgattctcctgactcagcctcctgagtagctgggatcactacaggcgcccaccaccacacccagctaatttttgtatttttagtagagatgggctttcaccaggttggtcaggctggtctcgaactcctgacctcatgatccgcccaccttggccccccccaaagtgttgggattacaggcgtgagccactaaatttagtgctcaccgcactaaattttttttagatgccggctcttgctgtggtgtaatcactgctcactgcagccttgacctcccgagctcaagccatcctcccacctcagcctcccgagtagctgtgacttcagacacatgccaccatccccggattttttattttttgttggggggcagtttagccatgttgcccaggctggtctcaaactcctgagctcaagcgatcctcccgcctctgcctcccaaagtgctgagttacaggtgtgagccgccgcacccggcatgagttatcagtgtgtggcttggtgtttctgacacctgcacacacttgcatatctactgcaggggacatttctccctgagctcagcccagatgtgctttgtcacagcccatcgcttatggccttggacacatgtaaagagaaaagcacgcatgcccacgctaagtttggcccctcacttggcacaaatcttgatccgccacctggcttgctgcaggtcagcagctgccctctccacagtgaggagcacttgccgagggacagacgtgagggacggacgtgagggtcgtctccaggctaaggcggggcttccttagccggaggcttcctggctcaggagtcccgcacaagcgccagctgagacagggactcacagcccgttctctggcacacacagccccagcacatgtggcccgacgtggctggcccctggggttgcatgggcgggtgccctcactgacctgcacggggtagggagaccccagggcattgcgtggttctctgagccacattccaggtgacttctctgcgccacccttcggcatctttcgccagggagccctgaggagcttggcaccttacgatccacagaggtgggaacccctgacaggagagcactccccagccggtgtcaggggagcatctgtgctgtcgaagggaggcccaggtcttcaagaccttttcccactaatggtcggtgaggactcagcagtacccaccccatttccctttctgactcggctgctgggaagctcagagctctggttccggatccatcagccctgccttctggaggctgctgccccttctttgcactttgcgggtctccctctgctgtttgtttaaggtgcctcacagcataggggccactgagcaccctgactgctgggctgaacacggacccttcccagcagcttctctgcaaagctgtgctgccagcagccggatcgcaggcaccgcctggcagatctgaaggttcgtgctgtgcaacgtgccttcctgtttgtgagttcacctgaccgacaccaggaggggctggactcctcgcctgccgccggcaaggcccgccctgggccagtggggtgcacacagcaggccctgtgtcgtgggtggtcagaattttggccacaacagttgggggacggggctggatgctggccaaggaagagcaaacagcctcatctccttcccacagcttggtgggtgcagagaggacctggggacacggtccttagggtctcctccggccccgagtctcgatgtccacatagagatggcctgagtggtcagccagacctgcccagaccacatcttagcccagccctgggtgtcttcagggagctaaggaggaagtcacagtagacgtccgtgggtcacagtgaggccagtcaccgctctgaggtgcaggccccgagggccacggctgaggaggcccagccaggccagcagtggccacgggaagccccaggccctccccacaacaacagcccggagcctgtgactctgctctcacagggtgaggccggccgtgctcccgcctcccggcttgcatgcagctctgtgcgttggtggcagccaaggcaggcagcctgacctcagcctcaggagctctgttggggggcggggcacccaagggggcactcagggcctgagccagcgtcccaaggctgccaggtggcggctgcagctacttttctgctccaccgtcagttgggggagcactgagacggcagcacaaacccccaacctgggctgggcaggaggcggccccggggcagcagcacctgggtgtctgctctgtggaggcccagaccggagccactggcctcagccctccccggggcccatcctggaacagacttggcctcacagtggggcacggacctcctcgccatggccacccggccctccccggcagctgcccagagctctgcggtcaccacggccttcccggtgccccgcggcacctcacatcacttgggggcagggaccacgtgctgctgggaacaggaagtgtgtggcttgattctgtgcggcctaagtgtgtttcctggcagggtcagtcactgccagcgctgggaaacggccggcctgaacgcaagcgatcagccagcacccactgccaggaacgccacacacagagcgcccccaatgccatcctggggtggccaccaagggcggggtctcagcctggcgtgcgctggcatgggggacacgaattccgcagtccacaggcaggggacgcaccgcacagagcaggacccacggctgctgctgggtcgggttttatttcaaatgcagccacagaggcggtttctgcacaggtacgtgatccgactccacaagctcccaccaggggctccccatgacccgcaatgacgctgtgtggggtcaaaggaaaacaggccacagccaggcccctcgatggacgcaggcaggggaccaggaatgcggcccacgcagggggatcgggaatcaggcggaaggtgcaggtttgcagctggcgggaggagccagcatgccccaatctctaaaatattcccggtagaaaaatagacatttccctccaaagcagattcctggggctggagggtccctccaaggccaggggtccgggtgattccagagcatccacgctctgcgctgaaggcactgaacctgccatcactgtcacagccgtcaccggccaaggagggtctggaggagggaaggggcccttgcgaggctctggtgctggtgatcccggcccccaccaccggaggagctgaaagcccttgctcagccgctgccctgctggtgaacccggcccccaccgccggaggagctgcaccctgtgtggtctgaggcagccctgcactgggcagcggccccgccccgcgctgaacccactaggagagcagctgcagcacctgtcggatgcgctgggccctccccggcagggggggatcagagccctcctcatccagctcccgcatcagggcttccgccttctgcaccgtcagctctcgggcccggccctgcagcccctccaggtaggccagcagggtggagaagtgctcatcgggaacctggacgggaaagagatcgagttggtcaggggcggggcagggaggggccgctgaacagcaggagacaagacgctgcagccaaggacacccgggcctcaggcttccgtcctcccactgcggcccgagggtgtgggcggcctggccacagaccctgccccagacactggaagaagaaatgcctgtttagaggcagacaaaacactcaattaacctgggtaaaacagaagccatgtggggagaacatttcacaaacatcgtaaaaatcctcagaaaaacgagaaaatacgacacaaattaaacaggggtaggcggtgatgaaaggtgcattcagaacaaaaagccaagtacgcgccggcacttggcacgctcgcctttctgctcagggcggcagtggccaggtggcatctcttggtggcctgacccttgtctgctggtggtgggggcacctctgcacaggctcaccagtcacctgacaatctgctctgcaggcacgtttcctcaggtccctgtggcagcggctttcaacactgggctctttttattattgagttagcagagttctaattccatgtgggacttgcaaacgtctcccattctgtagcaaccatttcactttccagctagttctgtatgcagtgcaaagtcttagattctttttttttttttttttttttttgagccggtcttgctctgttgtccaggctgcagtgcagtggcacgatctccacctcccaggttcaagtaattctcctgcctcagcctcccaagtagctgggattataggcaagcattaccacacctagctaatttttgtatttttagtagagacggggtttttgccatgttggccaggctggtctcgaactcttgacctcaagtgatccacccgcctcagactcccaaagtgctgggattacaggcgtgagccaccacgcccggctacacagtcttagatcctgatgaaggcactttctcgactttttcctttgtcgcttgtgcttctagagtcacatttaagaaactgttgcctcacccaagggcacgaaatggactcttaggctttcatctaagagttttggttttagcttttaaatttaagtctctgatccattttgagttaatttttgcatatggtttgaagctgggggctgcgtgcattaagtatcggcatgttcgtaaatatgtacggttacatatgaaataaaaatgtataagtaaaatgcacatttgtgtgtttgtaaatatgtttaattacaatagtgtaagataactttgcgtgtgtgtgtgtgtgtatacacacatataaatggctaaccaaggatttgcctaaccaaggattatttttctgtataaaaagttttgtataaagtttacttttggtaatagtaacatataaataaaaccacgtaaactttggttaaaacaaaacaaaacgtggaaacccagttgtccctggctctgttaagaagaggtttcctccacatggaacaggcttggcccgcatgctggaatcagctgcgccgggcgtgagggtttccaggcttcccaggctgttccagtcaattctacttccacccttgtgcctgccccagtgtcgcgattaccggagcttgcaatagggcttacaatcggcttgcaatagggcttgcaatgtgtatgtcctctaactctgaggagggagagtggcttgagcccaggaggtcgaggctgcagtgaactgtggtcgtgtcactgcactccagcctgggcggggccctgtctcaaaaaacgcccacaaaaaaacaaaaacaacaaaataatgaaagcaagtggctgcaggctatgcctggaatgtcccagggctgtgagtggggctggtgggcctgccccgctctgagagtctgcaaacacaggcacggcaggagaaaaccgccccctgcccccagcgcgcacgcaaagcctggcctctagtaacgtgggcgcaggagagccagcgaggcaggaggcactgcccaccggatgcagctcagcagaaccgtgggctcctccagcctcacaggacatgggcagctccagcccgccgcccagcagccgggtttagttcagaattctgtctcgtttcatcctgtgaaactctatctcacatcctgcccctgaaaagggcagctgcccacacgtacatgtgtagacacacatcacgcgcacacacgtgtagacacacgtcatgcacacacgcacgtgtagacacacgtcacacgcacacgtgcaggtggcctccctaaggttaccccgtgttggtttctgccctgaaggccggcggcagggctcctggaggaggcaacgctgctcatctcttagaacaaaggcaccgggagacctgccctgcagtggggccctgagggcggccccttcagactgtggcttctgctgtcagggccccagtgtgcacaggagacgtgcctgccccgccgtgcacagacctcagcccccaggagacgtgcctgccccgccgtgcacagacctcagcccccgggagacgtgcctgccccgccgtgcacacacctcagcccccgggagacgtgcctgccccgccgtgcacacacctcagcccccggggggacgtgcctgccccgccgtgcacagacctcagcccccaggagacgtgcctgccccgccgtgcacacaccttgtcactgtcatacatgtgcagcaggagccacgtctgcctcgtcttctgaaacctccagttcttgtgcttttgggcccatctagggaaagaaagacaaacatctcagggtgctgggcaaaagactcccgggcagctgtctgccctgagagatcggggacgcctcatcttaggagagggagccccaccaggtactacacaccaaggcctcatgtgaagagggtttttggagcccctcccctgcccgaggcgaggagccgcaaccccacgccctgctgccgcctgaccaggaagcatgtggacgccactcttaccttggccaccccggggctcaccactgaccccaagtccccgtgggctcccccaccccatgggtcacacggattttacagggggggaaggatgaagtgagtttctctgggaggaaagggagcgatgtaattgatgatgtattttctacatgaaaatgggaagactgtaaggaacaataccctgagtcccaggccctcacaacaaggggccagtgggcctggggacgagcagctcccggctgctagaggggccgctccccagggccacaaccaccccgcccccgtcccgctcctgcactagggcctgtggacatcaggcagctgcggatggagcgaagctggcgctgtgggtagggcagggccaggcagggtttactccagagacctcctcccagatacgtgtttccagggccaggcagggcagccgagcccagaagcctccatcagtgtgcaagcccaggtctgaagactgtaggctcagcacgggggagggccctgtgcctgttgtgagcgctgcccagcttgcctgaccccactgccatgtgggtgagtggagtcctgtgggtgtggagccctgtgggtggggagtcctgtgggtgagtggagtcctgtgggtggggagtcctgtgggtgagtggagtcctgtgggtggggattcctgtgggtgagtggagtcctgtgggtggggagtcctgtgggtgagtggagtcctgcgggtgagtggagtcctgtgggtgagtggaatcctgtgggtggggagtcctgtgggtgagtggagccctgtgggtggggagtcctgtgggtgagtggagtcctgcgggtggggggtcctgccggtggggagtcctgcgggtggggagtcctgtgggtgagtggagtcctgtgggtgagtggagtcctgcgggtggggagtcctgcgggtggggagtcctgtgggttagtggagtcctgtgggtggggagtcctgtgggtgagtggagtcctgcgggtgagtggagtcctgtgggtggggagtcctgtgggtagggagtcctgtgggtgtggagccctgtgggtggggagtcctgtgggtgagtggagtcctgtgggtggggagtcctgtgggtgagtggagtcctgtgggtggggagtcctgcgggtggggagtcctgcgggtggggagtcctgtgggtgagtggagtcctgtgggtgagtggagtcctgtgggtggggagtcctgtgggtgagtggagtcctgtgggtggggagtcctgtgggttagtggagtcctgcgggtggggagtcctgtgggtggggagtcctctgggtgagtggagtcctgtgggtgggaagtcctgtgggtgagtggagccctgtgggtgagtggagtcctgtgggtgagtggagtcctgcaggtggggagtcctgtgggtggggagtcctgtgggtgagtggagtcctgcgggtgagtggagtcctgcgggtgagtggagtcctgtgggtgagtggagccctgtgggtgagtggaatcctgtgggtggggagtcctgtgggtagggaatcctgtgggtgtggagccctgtgggtggggagtcctgtgggtgaatggagtcctgtgggtggggagtcctgtgggtgagtggagtcctgccggtggggagtcctgtgggtggggagtcctgtgggtgagtggagtcctgtgggtgagtggagtcctgtgggtggggagtcctgtgggtgagtggagtcctgtgggtggggagtcctgtgggtgagtggagccctgtgggtgagtggagtcctgcgggtgagtggagccctgcgggtgagtggagccctgtgggtgagtggagtcctgcgggtggggagtcctgtgggtggggagtcctgcgggtgagtggagtcctgcgggtgagtggagtcctgcgggtgagtggagccctgtgggtgagtggagccctgtgggtgagtggaatcctgtgggtggggagtcctgtgggtggggagtccttgggtgagtggagtcctgtgggtgagtggagtcctgtgggtggggagtcctgtgggtgagtggagtcctgcgggtggggagtcctgtgggtgagtggagtcctgtgggtggggagtccttgggtgagtggagtcctgtgggtttggagtcctgtgggtagggagtcctgtgggtgagtggagtcctgtgggtgagtggagtcctgtgggtgagtggagtcctgtgggtggggagtcctgtgggtgagtggagtcctgcgggtggggagtcctgtgggtgagtggagtcctgcgggtggggagtcctgtgggtgagtggagtcctgtgggtgagtggagtcctgtgggtggggagtcctgtgggtgagtggagtcctgtgggtggggagtcctgtgggtgagtggagtcctgcgggtggggagtcctgtgggtgagtggagtcctgtgggtgagtggagtcctgtgggtggggagtcctgtgggtgagtggagtcctgcgggtgagtggagtcctgcgggtgagtggagtcctgtgggtgagtggagtcctgtgggtgagtggagtcctgtgggtggggagtcctgtgggtggggagtcctgtgggtgagtggagtcctgtgggtgagtggagtcctgtgggtggggagtcctgtgggtgagtggagtcctgcgggtggggagtcctgcgggtgagtggagtcctgtgggtgagtggagtcctgtgggtggggagtcctgtgggtggggagtccttgggtgagtggagtcctgtgggtgagtggagtcctgtgggtggggagtcctgtgggtggggagtcctgtgggtgagtggagtcctgcgggtggggagtcctgtgggtgagtggagtcctgcgggtggggagtcctgtgggtgagtagagtcctgtgggtggggagtcctgtgggtgagtggagtcctgcgggtggggagtcctgtgggtgagtggagtcctgtgggtgagtggagtcctgtgggtggggagtccttgggtgagtggagtcctgtgggtttggagtcctgtgggtggggagtcctgtgggtgagtggagtcctgtgggtgagtggagtcctgtgggtttggagtcctgtgggtggggagtcctgtgggtgagtggagtcctgtgggtgagtggagtcctgtgggtggggagtcctgtgggtgagtggagtcctgtgggtgagtggagtcctgtgggtggggagtcctgtgggtgagtggagtcctgcgggtggggagtcctgcgggtgtggagcccagctgtgcagctgctaggtgacacgccagccccatgtgcagtgacgcagggctcctgcgtgatgagaggggcctccagggaggtttaattttagttcctttctttttagacagcccctcgtcgtctgctccccggatggccgacccggccagcgtgggcccctgtgaggtgcacccgggccagccaggccatggcatctgcctgctgggggctgttttactgctgcagggcagaagttggcatctcagctctgccactttctaactctcccaccttgagctgctgctttccaccagcctgtttcctccttgggaacgtggagtggtgaccccacttcacgggtgttcccagagcccacgcacggtgagtcagtcacagctgctctgcacactgccctcccatggggcccgaggccacagcactgctggcccacggcactgctgcctacggccctgaaccaagggcgcctctctctcaccgagcccccaccaggcagacttcaagccaagcccatctccctcaccaccgaagatgagccgtcccgctcttggcctcgtcactgggggagcagacacagcgaggggcgggtggggtgctcagtgtcgcctgtggctcccacggcactgggcgaggcctcctgagtgatggccgcctctgccacccaccctggggactggtccctgcgcaactgcagacagttcacaggatgcctgacacaatcctctttctctctgcctaggtggggcagcagacaggagatcccatcttggaaaaggaaagagagcagtcagagggaagtcccttgtccccagcatagcgggggccacggccactgcagggtcatctgtccagagctgcctagagcgtcccagagagaagaacctcctcccctgtggtactcaccagctgggtggagaaccagagtccctgctagcctactgggctttaaaaaaaagcaggggcggctgggaacagtggctcatgcctgtaagctgagcactttgggaggccgagggcggatcacaaggtcaggagactaagaccatcttggttaatacggtgaaaccctgtctctactaaaaatacaaaaaattagccgggcgtggtgtcggatgcctgtaatctcagctactcaggaggctgaggcaggagaatcgcttgaacctgggaagcagaggttgcagtgagccgagattgcaccactgcactccagcctggcgacagagtgagactccatctcaaaacaaacaaatcaacccacaaaaagcggggacttcttgctgaacccccttccaaaagaaaactgagcgagggtctctattgagacagcatctgaagccggcccgcagccacagcgtccacgtgctcgtaactgcaccaggaactgccagccgcggtgagctgacgacccaatgcccatgaaggcccgagcggggctggcgcggacaggggccgggtctcatgggttggccgctcctcccatacttggagctgggctgtccctcggcagcggggcagtgggcctgggtagggaaggacacccaagaagtgtcccctaagctccgctgatgggacagtttggcctctgctctggcctgtcattccagggttccaggcccggaggccacggggtgcaaggaagctaagacaccatgctgtgctgggcaacatggggacctgaggagggtgggcttgggctgccccacggcaggagtgacttgctctcgtcctcccgcaggtgagtgggggaccccgtcccggctggatccgtgtggcccgggtcccagttcctctctctcctgtgggggcaccacctcccgcctcgagcagacccaggcagggggccggcccccgaaaatggtgagctaaagcctgcagggaccagggaggcagcggtcacctcgccacctgcccccaacgtgaacacccagcaggcgtgttcttttcttttttcagttatatggaaagtaaatcttcaatttcctgcgttaagcatataaataaactcttttttctctttaatgaggaacaggcttgcccgctgacagccgtgtgcctcgctcgtgtaactaccacagtggacccagcaacccagagacccacagaaagtggagggactgcttccagcactcccaaacttccgcctatgcaaggccagagcaacacggctctgagctggaaaaacagagctgggtgaccaagcctggggcccctgctgggaaaacgaggggacagtcacagcacagggaaagagcacacgacggccacagtgcagggaaagagcacacgacggtcacagcacagggaaagagcgcacgacagccacagcgcagggaaagagcgcacgacggccatactgcagggaaagagcgcacgacggccacagcgcagggaaagagcacacgacggccacagcgcagggaaagagcacacgacggccacagcgcagggaaagagcgcacgacggccatactgcagggaaagagcacacgacggccacagcgcagggaaagagcgcacgacggccacagcgcagggaaagagcgcacgacggccacagcgcggggaaagagcgcacgacggccacagcgcggggaaagagcgcacgacggccacagcgcggggaaagagcgcacgacggtcatactgcagggaaagagcgcatgacggtcacagcgcagacaaagagcgcacgatggccacattgcagggaaagagcacacgacggccacagcgcagggaaagagcgcatgacggccacaatcccccatcccaccctctggaccctcctctccccagccctgctgcacagctcccaccagccccagcttcacacaaagccaggagcaccccaggaaggctgcagagaagatgaacccttgagatgcaagaacagcccagggtatggtcacaggtcacaggccaaggtcaccgtgtggggatgctgcccagcctgcgacccgggttctagggacggggcatgaagaccccacgggcgctgggtcgacgtgtcttgggggcagagccttcgggggatctggaggggagacccccagggcatcctggaatgggtggggtgcctcccagagagatgagaggaaactctccaccatgagggctcaccagaacaatccggggtctcctccacacacagcccacctggctgcagatggctttgcctccaggaggaacccctcgtggaaagcccataagatggccatgggccacggcggccagcatgtccttcctgagtcccagtccagggcctctgcagggggaggacaactcgggcacctaccttcaccgctgcccctgggctgggggctctgtggtgggccagccgtggcgtggccagtggtccccagcagcccccaggctgaggacactcccctgtgggaatccccgggctccctctctttggaaaaccctgagttgccagggataccgtgggctccacacatccacgtgggctctgatctggcagccagatgcggctgggcttgcgggcacccttggagacccccaggaaagaggagaagctcctgccggggtgcccgtgtctgtggctggcctcctggtgtgtggatcccaggccttggaagaagctcctgtgggaaggggctgctttgtttctgagaagtaagagtcccccctcctcccacacgcaggacacagtgcctccgcacaggacttgccgctggggggttgcgagtagcatgagggggccacgggaaggtcccagagggggagaggagggtgaggctgaggacaggagcagggcatgctcccaggcactcaggaccttcccgtggggtgctcggagggtgtgtgatcatgggaggggagtcagggcgcctccagggtgaagaccagggagtggcagccacgtggtggagcggatggtcccccctctcctgcccacgtccccaacgtcccccttgtctaacagtccacactgagagcagcggaaggtgctggcaggcctggggaggaaggcggacaaaccccgcccaccccaggcatcagggcaggtccagaacagccacgccccgaggcacctgccagtggagacaggcaaggtgcaggtggcacagctgcaggccccagtcgccctctgcctcccacagggagcctacagtccattcctgcggctggcaccttccaggcctgtgtccacctgctctgcccagcccacaccttgcccacacctgggctcgagacgctgcactaacggcctctgaagccctctctgtctacagcgactgaccctgcggtggcttgtggccctgcctgcagagaagcaggagcaagcgcaggaggcagcagcctcacaccacagccagcaccttgggtcccctgggccaagcgcgttcttggagcaagaaagagctcttggggtagccctgggcacctggcctcagccccacgttcctcagggagagggaagctaagcctcccagagaactgaccagtgcagagacccatgatgactcaactggttccagccggatccacccaacgctatggctgagctcattctatgacccccaaggcagcctcagctccgagaaacaggaggcttgcccccatccccatttcctgagcccggcgctgagcctggtgcagggcagccaagagcccctctcctgtaggtggaggaccgtcctggggatgcacacacaacacacatgtccacacataaacacacacgtgcataaacacacacgcacacacgcacatgtacacacgtgtatacatgtacacacgcacatatacacaacgtgtatacacacgcacacacgtaaacacacgcacacacgtgtacgcaacactcgtatacacatgcacacacgtacacaacacacgtatacacatgcgcatacatgcacacgcacaccccctcatgcgaacacacatgcgtgcacacacacccatgcgaacacacatacacgcacacacacacacatacacatgcacacacgtacacacatgcacacacgtacacgtgcacgtgcacgcagcgtgcacatccacgcaggccccgctgctcattccgctagctgcacagccacactgggggtcatctctccgaggaggtcatctctccgagggcctcccacagaggtgcggctggagcctgtcctcccaggcagggccagtgcccccacaggacaccctgcccctcagggtgacttcatcacagggacccagttcttgtctctgaggaagccagccacgtggacgccgtgacaccccttattctaggtgcagtcagcactcagggagacggacaggaacgggcagcagcctgtgctggtaagcgccggggaggtggccacagcccgcaaggctgtggagcccacgccaaagcctctgacccctgcaatcctgcaccgtccacccagccacgccgcaggcaccatccacccagccacgccgcaggcagccagggagatggcaccctgtgcccagcaccccacaatccccgtcttcccgctcttgggtggcagtggcgcctcctgtcactgtgagtgagggctgaaggccaccacgagttccctcctcctcacccccacgtccaagcagcacccaggcccgtggattcttcctgtggtgacgtacacctggggcaaggttcaccatcagacccatttctaaaagcacagttccaggcaacccctgcacgggcagcggctctgctgccgcccctgaaagttcctgaaggctctgggccattcagacagaaagggcagcggcggcagcacaggcctggcatgaacgctcatacctcactgctctagaaccttccacacagtgtgtgcgagtgaaggcttcgaccccctcaggcaggggttgagacgcacgtccgggacagaaccgcgtggggcagggccgctcacctgcagaggtagtccagggccagttcggcccccgagcgcctggcaggcgggtgctgggccacaaggcctgcctcccgcagacgctgcctctcctctttcttccgttcctttttcagcttcctttccaggaccctctgctcctctggggacagctctggctctgcgtccagcccgggcctgagcacagcttcgccctgaaagtcaaggaacacattaggcttcggggatggagggtatccagagacgggtgcgctgcctggggcttcccgagcccaccctcacctcagtagtggccaaaagctctgtccccaagcactgggtctggccccagccgacaccatcctgctaagcctgtgctacgacgaagagacagacaggtcacatgaggatggccagtcctgcgggcaggcgccaggtgccaggattaactgcccagaggccccttccccacccccgtggtccacctcccacagactcccggctcccgaggctctgtgaggtcctgccccaccccacacagtgtgggaagatccgggccggcccccagggagggctgaaggtgtgctggtgtctgttatcacacaggccaggtgctggccccagacccgacccaaatcccgaggacggtgcctggggcctgctgctttctggcaggtcactggctgcctggctccccctgggctgcaggctggggacagggagggggccaagacccccaagggagggagagggcccgctgtgacaatgtgtccccagccacatcttgccaggaaagcacaggccctccgtcttgcccaccctcccctcattaacaaaagaagaggacggtgcaaagcgtcaaagccggggcagccgggccacggggtgaccatgaggtgaccccagccgctgtccacatgcttcgtcttttttggaaaagtcagaacatcagtgtagactccgagcaaggccaggagcccagggaaacagctaggggcgtgggaatcaaccaggcagcgcccaccgggagcttccaggaagggaggccgggcacagcccacgcctacgcatcccaccccagggagagcaaagaagagtgaggcaagcccccctacatacagatatatatgcaacccacccccccgacacagacacacacattcacagacttcacccctacagccaaacacagcccccaaccctcacccatgtacccacaccgacccacacccacccacacccaccatacacccacactcacccacccacacccacagacacggacacatacccacacccacagacacccacatacccacccacccatacccaacacacccacacccagatacccacacatagaccaacacccacacgcccagacacccccacatgccacacacccacatacaaccacacacaccgacacccacacacatccaacaaatccacagacacccagacacccacagatacccaaacacccacagacacccagaaacacccacacacccaaacacgcccacagacacccagacacacccacacccagacacaccttcacacgcccagacaccctgacacacccacacccagacacctgcagacacccacaaacacccagatacccaaacacacccaaacacatccacacaccatcccaacatacccacagccacacacgcccacacccagatacacacccagacacccacaaacacccagatacccaaacacacccagagacacccacagatgcccacacacccagacacccacagatacccacacacgcccacagacacccacaaacaccatacacacacgccaacacacccacagccacacccctagagcctgccaggggtccgcagcaagagacagggcctgggggacgaggagcaccgtccctggcagatgcctggcacagagaggctgaagagtcccggtgggaaggcctgggccgcccacacacacaggcaggaagctctgagacccaccaggccaggcacgcagggctctggggagcaagattttcatggtggggatgtggacacccccaggggagcctggtgactgaccggctccaaccagctccatgctcacctcctaggcccacagccgcccacagccgccaacggggcaggcacaacaactggccctgaaaggtaaaaaccctcctggcacaggcccagcctcggcccccactccatgggccacgcagtccacggctgatcacacgggccaaagtccataacactgttagggctgcacaggggccgccgcggtggccgagggtctgtcagcccacatgcgtggggcacccggacaggccctgagcaggtgggcagggccctgtggtagggtcccaggttggtggccagagtggtgaggacggggccaaggctgtgggcaacagcaacgtgggcgctggcctggggtggatggcgaggggaacccaggccgtcccatatcggggccaagccggggacccgcaggcccagctgtttcccgcaggcttcctgagcgcccggtgcagggctgcttcttgtgtcaggcaccagcctggatgctgcggaaacagcttccttcacacccactcttgcttctggccaccagcgctcccgacctggcaggagtcacagcccaattcccgtgtccccgagggagccactgtggccacgaggtgggaagctcaggagtgccacccactgccctgggctgtgcctgacactggccggtgcacctttaacttttccaaactcctgctttctgtgctgggcctcaacagaagacgcaggggcagctggagtgagggtgctgcgcccggagagccaggggccttgggggtgcccggctccactctgcgccatcggctgccccgtgctcgcgctcgctgccttgggggtgccggctccactccgtgccatcggccgccctatgctcgcgctcactgtcctggaccaggcaattccccaaggctgtgaccacgacgtgagcgacctcagctatcttgggatgacacaggcgggccccgggagggtgtgcagctggagggggcgggaggcagcaggtccaggcaggctggagggcacaggccgcggcaggtcgtggggaatgggagcagcgcgggagggtggtggggccgccaggatcgggcgtggtaatgccgggacctgctgcttgctctgcttggcctgcacagtgtctggcccggagagcacaggtgagcaaggtaggacggctgtcacccacagacgagcagcggagaactttctctaactgccacccacgtgccgaccctccaaagaacgcgggtttcactagcaaataagaacgtgggtttaattagcaaatacttttaacaaacttgacactatcacatagagctcccaaagttgccccaagggcttgtaaattacctaagtacacaccaaaaaatttttaaaagaatcccagtgtcaaataaaagcctgtgagggccccttctagggacttgagtttcaggcctaggatgcaaagcctggcgcagcagccaggggtgactccaggctcctgccggctccaggattctgggaaggggcagactttgggccccctggagcccctctaggcccagtgtccaccccacagaccctgcagtctggggccactcttctgggagaggtgttgtgggcggctttgagccgacgctgtcagggcaagagtctctctcctaacatgaaggctttccaacacagatgggacgggcctcatggaaggaagcaccggcctgtcacagacgtgtgatgtctaaagccggcgtctctggaccatcccagggcaagcagaaagtcgtacaaaagtacagtcaccctatctcaatgctaggacagtcttcagcatttcagacttgatgaaatacagacactcatatgaagtacagaaaaataaaaattccagccacacattctgtctcctgcggttagatgtcttgaggattacatttgaaaatgcataaaacaattcaatgtgcatgaaaagctttacgcaggacctgcacccacacagataaactgcgaccccgaggactgggatggggaggcggccagcgggcctgtcactgcacctacgctgtgtatttcacaacatggacacttccacaggctacctcaagcaacgcggcactcgtgcagtcaaacatgaaatccttaccatcaggcaatgaaaatgttcactgtactcaacaaatacactgaaaacaaaccacaaagccaaaccctacaggctgggaggagcaaagtggatcagctgccacagcaccttccagagaagagagccgtgagattcttacagagaagctgacactgtttgagaaaatacaactgcatgaaaactatgtttttcctttatgcctttaactcctggcaagagacacagagaagaaatgacactaatttgattttaaaaggttcccgtagactcgactgaggcagcagagacccaacagctcctttccgcccaccccacccgtccatctgtgtgacacgggccctgtgtgagccgtgctccccagcgtgaggcttcgccacaacacactggctcttcctggagatgtccaaggtgggtaacgtgctagtcataccccaacaaaaagaactggcaaaaataaaaaccatcatcatttcccaggtttgtgaatttaagccaacaggaaacgtgggacaaacacgcggggctccataaagccagcctggcactggctctggcaccaggtccctggactggacaccaatctccacggttccgaattttcctaagtgatgactgtgaacaccacatgcacagaaacgagccctgagaagggggccacattggccgccggccacggcagcgatccggccacggcagcgttccggttggtgtcaccctgtcccggcctgagcccagctctcagatccgagcaccaattctgtctacagccccttcctgcctgcgggccctccctgccctgcagggacggtgcagtgagcacaggtgtgcaggctgtgcggtgagagccccagggctgggaggagcaggcagccttggtccagcaggtcctgtgtgggggccgcgttgccgggcagggcctgacacggctgctgtgggctctgagaacagcccaaaaggacagtgacccctgcccagtctctccagcctgggaatgaccttccccagccgtggcctcacaccagggaggtgtgatgggaagtgacctgggtggctcctgggtccctgctaaattgtttgggggggctgaggggtgaggaaagagaggtggggtcgggaagaggcgtggcctccctgtccctgaggcatgagagcaggtgtgggctccccgtcccccgggcatgagagcaggtgtgggctccccatcccccaggcaccagcaggtctttgtcaacctgaggctcagggaggaatgtgcaagtccctgtctcatgaacagggtttgggccatcggagcccctgcagaggctgggttcatgcaagctgtcagctgccagtgcccacaggcatccacgccgctctggactcagggtttagaaaaagtcatctgttccagccagaccttcctgtttccccccaagtctaagggagcagagccttcacctaagaaaggagggcagagtggggagacccagggctgacccagagggcagggaggacccaggactgacccaggagagcagggaggacccaggactaacccagagggttggtgggcggggccaggactaacccagagggccagagggactgaagctgcggcctgtgggcccctaatgcccagccctcgtggaggcaccaaggcaggagcgcatcccaacacccagcccgggcttcctcggtgggcagctcctcacccacggtgaagcgaggccaaaggtcatcgagtgctgaccgccactctccttcctccaggggcccctctgtgagcaatgcccatgtgacactgatgtcacctgaccagacggaaatactcctgacgcccctcagtgaccctggaccggcgtctgtctgggcaggaccatctagtgtctgaggtgtcacatgaggatcccagacaaaacacgctccccctccggtgtcacgccagcacacagcagcacacgccagcgcacagcagcacacagcagcgcacagcagcacacagcagcgcacagcagcacacagcagcacacgccagcacacagcagcacacgccagcacacagcagcacacagcagcacacgccagcacacagcagcgcacgccagcacacagcagcacacgccagcacacagcagcacacagcagcgcacgccagcacacagcagcgcacgccagcacacagcagtgcacgccagcacatagcagcacagcagcacacagcagcacagcagcacacagcagcgcacgccagcgcacagcagcacacgccagcacacagcagcacacgccagcacacagcagcacacagcagcgcacgccagcacacagcagcgcacgccagcacacagcagcgcacgccagcacatagcagcacagcagcacacagcagcgcacagcagcacagcagcgcacagcagcacacaccagcacaaagcagcgcacgccagcgcacagcagcgcacaccagtacacagcagcacacagcaccacacagcagcgtacagcagcgcacgccagagcacagtagcgcacgccagcgcacagcagcacacagcagcgcacagcagcacacgccagggcacagcagcgcatgccagcacacgccagcgcacagcagcacacagcagcacacgccagcgcacagcagcacacgccagcgcacagcgcacagcagcgcacagcacacagcagcacacagcagcacacaccagcgcacagcagcgcacagcagcgcacgccagcacacagcagcacacagcacacagcagcgcacagcagcacacaccagcgcacagcagcacacgccagcgcacagcagcacacagcagcacacgccagcacacgccagcgcacagcagcacacgccagcacacaccagcgcacagcagcgcacaggagcacacagcagcacacagcagcgcccacccacgcaccctggtcctctgctgtgcatgtcattgagggtgtggggtctgcggagagcccggaccactcggaagccttggtctgagggccacgcgggcccagagcacctcaggctgtgggcattgggcctctgcctctggccccaggctcaggctccctggggaagtggtggccgtgctgcatcaaagaatggcagagaaaggacccactgtctctcagagggaagataaggagctccctgctcaaattggagctgcggggagggacgccaccagcccagcctgccgtgccctgctgtgcgggtgaaggggctgcagggaaggacaccaccagcctggcctgccgtgccctgctgtgtgggtgaaggggtcacctggcctggaccccgtccagtagccatgcaggggatggtgtgcatgaggcaggcgctgctgtcctacgatgtcagccgggtgacggggccagggctccagtgtcgctgccatcgggcccttggggagcatccctggatggcaggggaggtggggagggctccacacagcggggtggaggacagcgagacagctacacaccctcctgcaccttcttctggggaaacaagacacggactcaatgcttacgaactttcctggaggggagacttctccagtgggatcaccccaacacgtgaactgactgccgcgggcagccgcggacggcgggataaggggcagccgcggacggcggggaaggggcagctgcggacggcagggtaaggggcttctctgcccccgccccccactgcacacaaggccaggcgctctggggcccagcatccagggctcaaaaccacacccgccttggtgggatggcgcctcgcctttcccatgctgcggtaggaacaccccatgtttgttcggtgcattttattttctaaaatgtggcagaaagaaaataactccagcaggtgacagaagacctagtagaacttagcccaaatttaaataatgacccaaagtccccacggggctcagacgttcccacaccctaggcagaaatccccactcacacgagcacgcatacacacgcgtgcacacatacccctgccatcatctctgctctctccattgagccctggctaagcaccagctcccaccaggcaggaagcacaggcccaggacacgcacactagggcttggcgggtggggtccaagtcaggaagtgagagccgaggccctacaggaacagggggttgccccttcaacaggtaacccatataaaaagtgactaacgaaagcacgtctttcttctcactccacagctttgaaaccagtgtgcattgtacacgtggagccatctcagacccagactcaaagtccgcctggctccggccatcagcctgtggcgtctcagagcctggccccgagcagagaggagatgtgatgaggagacatgacgagggagtcgtgatgggcacacccatggccagcaggcccagcctgtgcctctcggaggtccgcacttcctgcccagtgggagcaggcacctggcgtgggctccagctgaggtgtgcacgcgggcgggtggggtccagcccagagtgtggtctttatgtaaatcagggtggagctcaggtgcactggctcacacatgttaatctgagtgctttgggaggccaaggtgggaggctggcttgagcccaggaattcgagactgcagtgagctgtgatcacaccactgtaccccagcctggatgagagtgagaccctcaaaaaaaaaaatcaatagatcagggctgcgccccaggtcttctacctcgggggtgggtgataggctcgtccagctggtggtcccaggacaagctctctgtccctcccaggtgttccatagtctaggacagtgcaggaactgcagacttgtcaagaactggggagtggtccgggcgtggtggccgaggtgggtggatcatctgagtcaggagtccgagaccagcctggccaacatggtgaaaccccgtctctactaaaaatacaaaaattagccgggcatggtggtgcacgtctgtaatcccagctacccgagaggctgcggcagaactgcttgaacccgcggggggcagaggttgcagtaagccaagatcgcgccattgcactccagcctgggcaatagagtgagactctgtctcaaaaaaaaaaaaaaaaaacgggggggggggagtgaaagggagggaggaagagggtgagggtttggaggggcccaaccaggcccacctgtggttgcatggatctctagccacctgcaaagccagaaggtaccttgagctgacaggtccgtgttgttcaatggttctatagggaaaatcctgcccccatgccaggttgttgatcacccacggtcaatgctggaaggggcagctgctgctctgggccagaaggagaggcttcctggcaccgtgaggaggctggcctggctgctcacagctcccggcagaaatgcggagcggggtcctcactgcagcacccgggcttttcaaaacagtgaagccctgagtccaagaaggcaaaccacagagcggaggtggccgtggggctgccctgtgggcagaggggaggctgcgcaggcctagtggctgcaggaagctccagtggccgccaagaacctccaggcccatcaccacgaggagggcccccactgcagccagggcttccagatgggcagaggcggtgggaagcaagcaggaagcggagactggttggctgtggccctgacccctcttctcctgatggagtgtggggggctggggctgcctggggtcacccactcaggtctctcactcctgccctcccaggaagtggacagttcccacagcctggacccagaggcccgcaggcactgaccgcggcccggaggccaagctacagtgcgtttcccgagggaggccacggggcacctggacgagggcagcttaggacgctcggctctcgtctcaggaatcccagggcagaagtccagcgaggcggcaggtggccggcccgtccccagcctctcttcccactgaggagagatccaagggccggcggagtggtggtgtctgctgctctgcgggaacacgggcaccaggcccagcagtgcatggatgcggcaggccccgcgtgcgatgccctggtccccacgactgtttcttcccacgccgcggagtgcattccctcatgctcccgtcctgtgaacacgcgctgcagcctctgccctcctggggctccctgagagagggggcaccccttgctcccatcacagtcctcctgcagcctggggccccgcccagcacgcccaccaaaggggaggcccgctctcctgtcagagccaggacagcctctccaagaactcttcctgggccccagagaggagccccctaggttcgcttctcgccagccgctgccccatcttgcgtctccacccaagggcccctcggcgcccccacctggagcccccactatcgagatatgtctggcggataccacacacaagggggctgtagcgggttggggaacaggccaacccctcccagactgcagggacagctagtctcacacagctaagaccaagatgatgtaaggaaatgaatgacagtaaacccagcccttaccgaacccacctcccgagcagggaggacctgcaggaaacagaggctcccattcctgttcacctctgatctcaggggcagacaccagcccatcactgccgtctgctgaccacccctgcagacggcctgggccttggatgtcctgctcctctgggctccccaggctgtgtgaaacggaggagtgcaggggtcaggacccctcgactttcttgggggcaatcaggccgtttgtaacgtcctgatacagcagagctggggactggggttacgaaagacaccaccaggtggagccttcagatgggacctcccagcagggcctccaggctggggaccttcaggcaggtacctccaggctggggaccttcgggcaggtacctccaggttggggacctcccagcagggcctccaggctggggaccttcaggcaggcacctccaggctggggacctcgcagcagggaactccaggttgggaacctccaggctggagacattccagtagggaccttctggttggggatcaccaggttaagaacctccaggttggggacctccagactggggacctcccagcacggacccccaggttgggaacctccaggctgaggatctcccagcagggacctctagtgtggacctccaggctggcacccatggggcaggtttctttcttcttccccagaagccttttcatcgtggcagtaggacctgaccttgacagagatgatattgcggatttcagtgactgaaccagagtcggaagctgaagggctctgcccagccttcctgtcttcatccattaagtctgatcggcagtggtggccacctgggctgctgctttgacaagatgccagtagttggagctgggaccccagggagcactgtccatccaccgcacaggagtagggcttcagcagctgtcagtcatcaatcatttccccatgctgttggatgggagagggacaatctgccagccccagtgtgactggctttgctgccaccgcagtgccctctgggaaccccggcaggtcggctggaggcaggacaccacccccctaacccctccctacttgtcaccagacagaaaccacagctcaccaacaaccatacacaaacagcgcactaaaagaaagcatgcggcaaaaatacccgcccagcgagaactgtgcctggcacagggaaacacactgggatgcgcgcccttccgcggggcgaggctgggttttggggtataaactgagtcctggggagcaggagcctgaggggcacccagggcagctccctcggcctgggtcacaaggttcgatcctccgcgggggtggcaggaaagcccaagtccctcggcacacccaggtgacagcgatgacggcccacaggcgtggcgcctgggccacatcggacaggccatgtcctggagcactcgaggccccctggacagggcaaccaaccctgagtccaccggagtctgctcgcagaccaccaggagccccgctgcactgttcgtgagccacggtggtgacggcagctcaggagggaaggaccagaagcctggccccgtcccacgaggacagacgcaggcctgcggggaggcatgtccccacccacgggcgcctcctggaccacgagctcctctttctcctgcatcgcctcagaccccacccaggggttccaaggtcaggagcctggtcccacgtcccacagccctctcatggggcacccgtacggattctgttccaatttaaagaaaaccttaatcagatttgtcacctcaattattagaatcccacggcgttaactcacagtagtaaactgctttttctgagaaacaagagttttttaaattaagccttttattttaacgcaatcgcaggtgacggccgtcgtgagggaacacgggacgcccacgtgccctccaccacctcctcccacggcaaccctcgcagagtagccaggaccacattcggatccggactctgaaatgacagtcccacaaccaacagcatcgatgtggtcttcttaatgatcagaaacacacaggggattaatttactcttattctgcaaaataaacaagctctcaaattctttaaaggtgaatcccccccaaccgtcctccctccccagttacttcctccctaggacaccagtggaacatcccatgctctcccccttccatgttgataccgaataaaacacactgcatcagaagacccacgcatacgagtccaggggctgctacaaagcgcggcgacgtctccgcggcgagctggccactccatggtcccggcgcagacactggggcaggcgccggctctgtcgtcgaggcgctcacaggcaggcacacgtgagctcctggaggacagggagagcggccgccccgcccctgcggagcacaggacgcttcctgccacccctgcagagcatgggacgcttcctgccatggtgccgtggcaatgggtggcacctgcctgtggccccttctcagaaggacgttttaaacgcgtgaggtctgatgcacagccacagggagacacagacgagcagatgtgggcatccgagtatttacaaggtctggtggctcctgcagccgcgacacgggctgagcgcaagtgatgtgtgaggtgtccccaacagatggcacggggagcgcccacacccgccaccgcggggtctgcggaagctcgtgtcagctggaggttagggaagacgcacaggggctcattctccacccaggttcaaactccctgaactcacgagacccaggctgaagacatggacagactcccagccccagcgcccctctgacagcacacccgaggcctcctgggaacctgggaccccttgcagggtcccccgcaggctccttcctaggaaggaggttcctgccctactccacatttctctccatccaacttccaactccttcccactgggccctggagaacatgacgaagactgtgggggcctcctcccaggaaacacacacacacacacacacacacacacacacacacacacacacacacacacaccagcaacgcgcccagaccccaatccctggcagcccaactcctcgttctacagagaaggggtgaggatctccaggggtgactgctcacatctcggcacatgtagaaggcagggctggagcagcggcccctgcagcccaggccaggggactggaggcccttccacagcagccctttccttttgatacacggtcttgctctgtggcccaggctggactgcagcggcaccatcacagtttactgcagcctcgacctcctgggctcaagcaatcctcccacctcagcctcccaagcaaccaggactataggcatgtgccaccatgcctggccagtttttaaattttttatagagataggaccaaggctgctctcacactcctgagctaaagtgatcctccttgccggcctcccaaagtgctgggattataggcatgagttttcaaactcctgggccacagcgatccttccggccagcctcaaagtgctgggattataggtgtgagcccacggtgcacagcccccactttcttcctaaaaaccctgcagcttggctgacagtgggacctgcctccctactcccgcctgcaggagctcagaagcctcagctcaagaggagcccttgccagagacgtgggaccaggaatgggccccacagagccctgcccgtgggcttcctaaacagcctgagttcagctgcacacaccaacgatgtaggcgggttgcaataaacagcaagaaacggacccatgggacaggagagacgcaggagccccaggggacaagagagacgcagaagccccaggtgcctttggtggccagtgctgaatttcgcaagcaacattagaaactttttatttctgcttttctcttaacccaagggtgaaaatggaaacctgtggtgtggggcagtctgctgaacgctaggatgagggcggggaacaagagccaccctccccgctgccacctggcccacaagagccttcccaggggctctccagtttcagcccctccttgccgagccagacagcgctgctggcctggacgggagggacacccaggctgactgagtgggggagcactttttataaagatcagatcaggtttctgaaaacaaaaagcccccagatcctgcgagcggctatttagacttgatgcacactccaccatttcacaggtatccattccccagcgtcacacatgcccatccccagctggagcacactcttcagtgtggaggacagaaggagcaggcagaagacgaccccggggcgctgggggagcaggtgcgggctcccacatacctgctctattctggttggaggtgtcacaagggaaggcgtggagatgctgccaatggggacatgcggcaaatgcgcccccttcgccccacacctgtcagcagctagcaacaactcagtcctgactgctggagacccagccccaggggcaggcaggttgggatccgctgctcaggaggcagctctgggccagggtggaggccggagaagccaggcacctccccgcagagctctgcagaggccagtctgctaacggcttcatcactggagtggcggctgctaagagcaaagcctgtgcttggtacctcagggacctaaataagaaccactccctgttccaaggatcccaagtggggggaggacgagaagcagctgagcctatagggcacaggaaactcactggggtgtgggtgctggggacttgggttgggtgctgtactgagcaagcctgttgtggggggacactgctggcggtgggtgacgaggtgcacaagtcaggtcagggagggggactctgccctggcaccactggggcacaggacactggctcaggtgtcagctgaggaccccccagtgtaggcgaggggctcacagccgctgaggcctgtggacacagctccgtgtattctgaggtcgcagagcacaagaggttgggggtgtgatacggaccgacctcgcccatccaggaggaatggggctggtaacagagctttgtaacccctcagcaccggtagaggttgcgtggctccccagtggagctgagggctggcgtttcttgctctgtatttccctgcgctctggaaactcctgcatcaccagccttgggcccttcactgcctcccctgggcctccagaggcaggctggggtcctctcctttctcagcctctgtggccagtgtctggcacaggacagacacccaccaggacgctgcctgggacggagggctcccttctgcctccggccaggtgggccccgcagcagctcaagcttcagctgcagtggagggaagagtgaactgctgggcagaagcagaagcaggcctcctggaactcgtgaccccagatcccagaggccctgcccccttgccacctcccagaatgttggcagggcctctgggtgattttcatccacatgcaggggtgtcacataggcccctcggctccagctccccactgaacggcagctttgtatccacaggtagggctgggcggctccaatttgaacttaggcatatggacgctgtctaacgattcacgacactcctcgtttctttcgctactattggaccagttgagggtggagcccagctgcacctggcattgctgaagagccctcgcagagccagcgctccagctgctgggcgcgcaacagctgtcccgggaggcaagggttcgcacggcagtgcttctgtgtcgacagaggctgtgctgcagctccctgccaggcccgtgtccttcctagctgtgagaacactaacctgcctgggcttcacctgtggaagccaccgctaactagcagtcggaggcggcgttgtttctaagacaggcaggcagctccggcaggagcagccgctaagcacaaggacggccaaccacacaaagcctgcttctcaaccactcgcaggaaatcggctcacagttgctccccattcttcaatgctgatcctgccactttactgaaaatcacattattaacgtttaaaaaacgtatgggaaccatgcaaagacatccacgcccctcagttgctgccgctccagcacctggctccgaagtctgtgcagaaggcgaggctcccgtagtgctcgagagggccttgcacgctccccgtggagggaacaaagctacgtggattttccagcgcgccgcccacttcaccaggcagcctctgggcttcatcctgctcgcgtgacaggtgtgctctgtgctgtcaggaaactgcagggctttgagtataaactgaaaacagcaaaactatgttctcatctcagaggccccttccttctcaccccgaggtcacctgggcccggagacacagcacccgccgtgcaggagcctggaagatgtttgagcaacgagaaaaaaaccagaacatgttaccgcctctaaaatgaggaaatggcaggatcaaaattaaatgtccgtgaagtgtatcatcatgtcaattttgttagttgttacacatagtttaaaacactcacatctgggaactaagtacgggttaggttttctcactttgccacaactcctcagtgaacacaacgaattcataaaaatcactgtcaattcggtgagttactcataagcaaaggactctgaagcaacggtcaccattcccgccgtctctgcagccgtgaggcccagcagcaggctaggcagcccggttcgcgtgtccgtgcggacgcctggtccacggcccccggctgtgcgcgcagcagctgaggacgctccgaggacacagcgcaggaccagcacggggccgccacctgcccgggccaccgcctcctcagaagataaaagacctactccaagcacctctgcgctctccaacccaaacccggctgagccccgacgcagcagcggcagagcctcttgaaggagctgccggcctgctccgccgtcctcaggaaggctcccgagtgaagctatcccaattgttttcatcatgagtctttgctttagttgacacagcttcgcactagcacccaccgcgccagtgggggccgccttgcctcaggcaagaaagcccctggcttccgagcgcccgacggggtcctctaccccggcacagggaggggcgaggcggcgctgcgggcgacacaggccctctcaagggtcacgcggcggccgacgactccagcccacccagggtcctcagagctcgtgtctcccccgggggctgctgcatcgcggctgggggcccggtccgcccccacagcgcaggggagcctggccggttgcggggccgctcgagggctcgcagtgcctgcagggcctggacccccaccctgccacccgcggcccggccctgcgcccccaggagagtaagtgggaggagggcgcgggaccgtccagggcctgctcggggaccggcgcgcagcggagccccgagaagcgcggctcggccggcccggggtctgcagggcggagcggggcccgcagcagcgtctggacgccacggcctcccacgggagcgcccacctgtcgccgcggggcggggctggggcagggcccctcccatcccctccctgcagcccgaatgcgggccctggccccgcgcactgacctctgacccggctgtcaggtcccgcgggcacggacggcgccggggcagggatgcgcccgcagtgcgcaccgcccggcccgcgcctccagcagcctcagcgcctccacactgcgcatgcccggccggcccggcgcggctggaaccagatccagccggctggaccgtgacgccagcgcgcctgcggggactggacctcggcgcgggggtgggagagccggggcggggcctcggcgctgagaaggcgggggtggaccctgctgcagcccccactgctcctccgacgaggctctctggccgcccgggtctccggtccacctgcgcgaactgcggggagggcgcacccccgcccgcgcagggcccctgccagctgcacgtacttgcctctgaccgcccccacccagcccctatcgtccccctacttccctcatctcccccatctgcctccattatcctctcatctcccccaatctgcccccatagacccatctcccccagtctgtccccatctcccctagtctgccgccatctgtccctatcgctcccccaatctgactccatggcccccatctcctccagtctacccccatcttcccagtctgccatctccccaatctgccccatctctccccagtctgactccatgtccccccatctcccccagtctgcccccatctcccctgcccaggccctgcaggcagaggcctagccctcccatggcatccacgtgtccagcaaaggggaggtgctcaataccggctggctgaatacgtggatgagtcacgcagccttttcctcaccacacagagggcgtctagatctcttctggcttgtgagttccctgccctgggactaagactgcagatcagtttttttgcttgtttgtttttggagatagggtcttgctctgtcgcccaggccagagtgcagtggcaccattgtagctcactgcagccttaacttcctgggctcaagtgattttcccaccttggccttccgagtagctgggaccacaggagtgcaccactgtgtccagctaatttttaaattttttgtagagacagggtctcactacgttgcccaggctgttcacgaactcctgggcccaagtgatcttcctgcctcggcctcccaaagtacggggattacaggtgcgccaccatgcccagcctaatttttaatttttggagagatggggtctcactatgttgcctggcataggaggctggaactcctggactcatgtgattctcctgccttggcctcctaaagcactgactgggattacaagcatgcaccacggtgcctgccttgcagatcaattttatggtgcgagggagccgcactggccacccagagcacagtgagcccggaggctgggccggttccagggcgaggagaggcttagactccctctcaaggcgtcagggtccaggacacactgagctctggtcgcggggccctgctctggaggccacccctgcctggggagatggtgtgggcaccccagtcccccgctgaataaggatctgcaaggccggagtcaggcctggggctggctgttttcctcctagctgtttcaccatctccaggcctgttcccatgcagaggcctgctctgtggtggcagcggggcaggcaagggtggactggaggcctcctggagctggtggacacgaggaggccaggaagggatgtcctgcccactgcctgcagcggctatgcagcagtgcttccgatttagtgtctctgccactggtgtccgcaccccgctttcagagaaggaagtgtgtgctcagtcaccggtttcatgggagggactcaattctgggccttctgagccccagtggccttgtgtgctgactccccagtgtgtccccagcatgaatgcgccagcccacacagtcacctgcctccatgaacgcgccaactccgcgcatccacctgtcttcccgaacgcaacgacccacgcatccacccacctccataaacgcgccaatcctgctcatatgccgtcttcaccaacgcaccaactcgcacagccacccgcctccacacctgagcccggggaccaggtttcttctccccgtagttctgtgctggtggctctgggctgctttgggagacaaggacaagagcaggagcgggagaagctggcctggcagtggggtgcagtcccccgggctgggccactagccccacagggatggggacgtggtgccggctgaggacccaggggcttcactccactccatctcacgcttccagctaccctgctctttgcacttcacagagggggaaccaagcccagcaaaggtggacacataccagctgctatgccttcctaggtcctgcccagtgacccacctgtagggctgtgctgtccccagacccatgaaggcacacataggcttgtggctttgggcaggtgggactctctccccaggcaggcggggactctctctctcccggggcagggggggactctctctctctcccggggcacgcggggactctctctctctccccgggcaggtgggactctctgggcaggtggggctctctctgggcaggtgccaccatgcctgctttcccacagcccacaccccgggccccaacacagagcagcaggtgcaaggctgcttactgcaccggcacatccaaccacctccacaaacgcaccagtccatgcatccacccaggctgagctaagggccccttgtacctagtacagtctcaagggtggagtggccaggagggagccctcccactgccttggcttgaagcatcagtggccagtggtccacactgagaccctagagccccctcccccagcaggtcggcaggtcctggtcaggaggaggggccaagaggaaaggaagggcccccagatagctggtgaactgcttccgtacacgggatgccgttccacacgctacatatctggacagaacaagacaaagatgcgtgcccattgagaggctatgctgaggctgtaataatactgaggagaaaagttaagggtaggctgtttttctgagatttaaaatggttgggggaggaaggaaatcagcgaaacagcaccacaggcggctcctgggcagagaccagagacctgcagggagggcagagtggtccgggggggcagagacctgcagggagggcagagtggtccgggggggcagagacctgcagggagggcagagtggtccgggggaccagagacctgcagggagggcagagtggtccgggggggcagagacctgcagggagggcagagtggtccggggcggcagagacctgcagggagggcagagtggtccggggcggcagagacctgcagggagggcagagtggtccggggcggcagagacctgcagggagggcagagtggtccggggcggcagagacctgcagggagggcagagtggtccgggggggcagagacctgcagggagggcagagtggtccggggcggcagagacctgcagggagggcagagtggtccggggcggcagagacctgcagggagggcagagtggtccggggcggcagagacctgcagggagggcagagtggtccggggcggcagagacctgcagggagggcagagtggtccaggtgcggggggcggccagcaaacaccacccggggaacgggccgagagtgcggggtggctgcgaggacgtgccgagggggtgagagcaacctggtcaccaaaccttccccaggtaatgagaattcgccttcgaaacctgggcccaggcaccccaagaccatgtggaacattctgagtcattcaaaaaagacataactatgtctgacgcattcttttaattaactgccctgctcaggtggtcagtgggccccacttcccagacacaccacgccccagagaatgcagggtagatgggccccagatccagagaactgctcccggcccacggggctgccccaacacgtccctcgcactcggcccgcctgaaaataaagtctctgaaatggaacctggggctgaggatgaagccccccgcaggtggatctgctgttggccagagcagagaaggtgccgtggaggcctcggcgaggctcctgtctgccaaatcactttcgtggcatcacgagaatccactgtggaggtgtgagggccaggaggccctgagaagtggaggccctggccctaagaggccacagccacccacccacaccgccaggcacccccaggtgacagtggccctcagaggccgcagccacccgcccacaccgccaggcacccccaggtgacagtggccctcagaggccgcagccacccgcccacaccgccaggcacccccaggtgacagtggccctcagaggccgcagccacccgcccacacctccaggcacccccaggtgacagtggccctcagaggctgcagccacccgcccacaccgccaggcaccctcaggtgacagtggtgcccttgggctgctccagtctgtccatgacaggcaggggcaggaaaaccacccaatcagcaggtccccaagagaaaacactggtcggagtcgccagaaagcacgatgctagggtactgcacgtatgtgggcagcgcctctgccccaagcatccttcctccagcccagtcacactgtctgcctgaccaatcctgcacgctcctcactaccctgcccggataccccctctctgggaaacctgtgagccccaggttgctggagggcctctcctctgtgcactcgcggtgcggacccaggccacggacagtctcggcaagtccgttacacactgaaagaagacctaccactacctcgatacgagggaacacagcccagctcggtctgtgaagctagtagaaccctgatacccaaaccagatgaaaacaaaactacggactaatatccctcataaatacagacgcaaacatcctactcagcaacatggaaaaagagtcacacaccatgcatgacaagtggagttcactccagaggtggaaggctcgctcactatttaaaaatccattgggccgggcgcagtggctcacgcctgtgatcccagcactttcggaggctgaggcgggcggatcacctgaggtcgggagttcaagaccagcctgaccaacacggagaaaccccgtctcaactaaaaacacaaaaattagccgggtgtggtgggttatgcctgtaatcccagctgctcaggaagctgaggtaggagaatcatttgaacttgggaagtggaggttgcagtgagccgagatcacgccatttgcactccagcctgggcaataagtgcgaaactccgtctcaaaaaaaaaaaaaataccatcgatataatgcaccctatcaacaggctacagaagaaatacgacgtggtcaaaccaactgatgcagggggcatgtgacgaagttcaagacccacgcgtggtaaacacccaggaaatcggtaacggaggtgcctcctgagtgaggtgaggagacacccaggaaatcggtaacgggggtgcctcctgagtgaggtgaggagacacccaggaaatcggtaatgggggtgcctcctgagtgaggggaggagacacccaggaaatcagtaatggaggtgcctcctgagtgaggtgaggcacatctgcaggtgctcctccaaggccaggagcagggcaagggtgtccaccctcccactgtccccactgctatccagcatcaggctggccgggggccacaggcaggaaaaggaccagaaaggaagaaatataactctctgtatttgcaagcggcatgaccgttgatgcaggaaatctcagagaatctacgaaataaatcctgggactgtgagtgggttcagaaggcatgggaccgcagacagtcagggagctggcaatgggctcgcggaaactggcattcccgtgctaccaccacctacgactgctcagcaacaaggaaatgcttagctgtaaatttaacaaaacctgtatgggacttaaatgctaaaaaccacaaaacactgaagaaacaaactttgaagaatccaaataaatggagagacacattgtgttaatggacagaacagacagcagagtcaagacataaattctcccaaaattgaggtacatgtttcatgcaactcctaccgtaaatttcagggtttttttttttgtaggtaaagacaagattattctaaaacgtatatggaaaagcaaagtaacaaggatagctaaaacagcgctgaaatacgatgactagaaggaactgcatgcctgatttcgggacatcggacagctgtggtgcagcacgggcagtgggagacacacagaggaacagagaacagtgaacccaggaggtgccccatgttagcaatgtgattttttcaaaattttattttaggtttaggggtgcctgtgcaggtttgttatacaggtaaacttgtgtcatgggggtttgttgtatagattatttaatcacccaggtactgagcctagtacccagtagttatttttcaccttctctccctcctcccaccctccaccctccttttcctcctccctcctcccacctccaccctcaaattcactcctgtgtctgttgttcccttctttgtgctcatgagttctcatcattcagctcccacttatagtgagaacatgtggtatttggttttctgttcctgccttactttgctaaggataatggcctccagctccatccatgttcccagaagacatgatctcactctttttaatggctgcatagtattccatggtgtatatgtaccacattttcttcatccaatctgccattggtggcatttcagctggtgtcatgtctttgcgactgtgaacagtgctgggctggtgtcatgtctttgctattgtgaacagtgatgggctggtgtcatgtctttgtgactgtgaacagtgctaggctggtgtcatgtctttgctattgtgaacagtgctgggctggtgtcacgtctttgtgactgtgaacagtgctgggctggtgtcatgtctttgctattgtgaacagtgctgggctggtgtcatgtctttgtgactgtgaacagggctgcagtgcacatttgcatgcatgcatctttatggtggaaagatttatattcctctgagcgtaaacccagtaatggagcaatctgatttctgacaaagtttgtaaaggcaattccatggggacagaaatcttttcaacaaatgctgctggaacaacgggatgtccacaggaaaacaataaacctcgaaccaaagaagatttacagatggcaaataagcatggggccagacgcgcgacatcctttgacgtgagggagcgcccatgaaaatagcagtgaggcaccgcacacctgtcacgatagcccaaatccagagaccgacatcaccaaatgcgggtgaggatgtggagcaacaggaatgcactcatcgctgtgggaagtgactgcaaaatggcacagtcacttggaagtcagctggtggtttcttaccaaagttggcaaaatcctgccacaggattcagcaaatgtgccccttggtattcacccacatgaactcaaaatttaggttcacatgaaacctgcacacagatatttcgagcagttttattcataactgccacagattggaagcaaccaagatgcccttcagtaggggactggataactgtagtccatccagacagcacaagaaaggctctgtcatgccgtgaaaagacgtgagcaagtttaacagcattctgcgaagtgacaggtgccggctgaaaaggctgcacgctgtggggtcccaccgcatgacattctggaacaggtagcaccatggagccaggaaagggttccgtggtggtcagtggtggtggggagggagggatgaatgggtggtgcacagggcccttttggggcagtgaagctgctgcgtgtggtgctgtaacagtggatgcacagtggatattcatttatcaaaacccacagagcatgccataccaagaggggccccccataagctgcggacttcgggcggttgtgctgtggccatgtagggttcgggtgctcgtgctgtggccgtgcagggttcgggtgctcgtgctgtggccgtgcagggctcgggtgctcgtggtgtggccgtgcagggctcgggtgctcgtgctgtggccgtgcagggctcgggtgctcgtggtgtggccgtgtagggttcgggtgctcgtggtgtggccgtgtagggttcgggtgctcgtggtgtggccgtgtggggttcgggtgctcgtggtgtggccgtgtagggtcatcactgttgtaaatgcactattcagggtgcagggtgttgataatgggggaggccgtgcatgtgtgtcaggagggtgtacatgcaaactccatcatctgctcaattttgctgtgaacctaatattactcttaaaaaaaagtctattaaaaaaaatccgataggggaagatggcggctgctcctttggaggagcgggattgagaggatcggggtggggagaccaaacaagagagacatttctggctctgaaggcgaacgcttcgctggccatttaggagctctgctcaaagccagacggccaagcgtggtggctcatgcctgtagtcccatcactttgggaggctgaggcaggagtatctcttgagcctaggagatggagaccggccggggcagcatgtcgaaaccctgtctctactaaaaataaaaaaaattagccaggcgtggtggcatgcgcctgtagtccaagctattctggaggctgaggtgaaaggattgcttgagcctgggaggcggaggctgcagtgagccgagatcacaccactgcactccagcctgggtgacagagtgagaccctgtctcaaaaaaaaattttttttagcaaaccagtaatagaaaggatttacttcagtctgataaagggcatctacaaaaaatctgtagttatgtcatacttaatagtaaaggagaaatgcctcctccctaaagtttggaacagtacaataaggcaagaaaaagaaatgaaaaggcttttagaacagaaaggaagatgcggagccgaagctggactgtactgctgccatctcggctcactgcaacctccctgcctgattctcctgcctcagcctgccgagtgcctgccattgcaggtgcgcgccgccacgcctgactggttttggtggagacggggtttcgctgtgttggccgggccggtctccagcccctaaccgcgagtgatcccgccaacctcagcctcccgaggtgccgggattgcagacggagtctcgttcactcagtgctcaatggtgcccaggctggagtgcagtggcgtgaatctcggctcactacaacctacacctcccagccgcctgccttggcctcccaaagtgccgagattgcagcctctgcccggccgccaccccgtctgggaagtgaggagcgtctctgcctggccgcccatcgtctgggatgtgaggagcccctctgcctggctgcccagtctggaaagtgaggagcgtctccgcccggccgccatcccatctaggaagtgaggagcgcctcttcccagccgccatcacatctaggaagtgaggagcgtctctgcccggccgcccatcgtctgagatgtggggagcgcctctgccccgccgccccatctgggatgtgaggagtgcctctgcccggccgagaccccgtctgggaggtgaggagcgtctctgcccggccgccccgtctgagaagtgaggagaccctctgcctggcaaccaccccgtctgagaagtgaggagcccctccgcccggcagctgccctgtctgagaagtgaggagcctctccgcccggcagccaccccatctgggaagtgaggagcatctccgcccggcagccaccccgtccgggagggaggtgggggggggtcaaccccccgcccggccagccgccccatctgggagggaggtggggggtcagcccccccgaccggccagccgtgccatccgggagggaggtgggggggtcagccccccacctggccagccatgccgtccgggagggaggtgggggggtcagccccccgcccggccagccaccccgtccgggaggtgaggggtgcctctgcccggccgcccctactgggaagtgaggggcccctcagcccggccagccaccccgtccgggagggagatgggggggtcagccccccaacccggccagccgccccgtccgggagggaggtgggggggtaagccccccgcctggccagccgccccgtccgggagggaggtgggggggtcagccctccgcccggccagccgccccgtccgggaggtgaggggcgcctctgcccggccgcccctactgggaagtgaggagcccctctgcccggccagccgccccgtccgggagggaggtgggggggtcagccccccgcctggccagccgccctgtccgggagggaggtgggggggtcagccctccgcccggccagccgccccgtctgggaggtgaggggcgcctctgcccggccgcccctactgggaagtgaggagcccctctgcccggccagccgccccgtccgggagggaggtgggggggtcagccccccgcctggccagccgccctgtccgggagggaggtcggggggtcagccctccgcccggccagccgccccgtctgggaggtgaggggcgcctctgcccggccgcccctactgcgaagtgaggagcccctctgcccggccagccgccccgtccgggagggaggtgggggtgtcggccccccgcccggccagccgccccgtccgggagggaggtggggggggtcagcccccctgcccggccagctgccccgtccgggaggtgaggggcgcctctgcccggccgcccctactgggaagtgaggagcccctctgcccggccagccgccccgtccgggagggaggtgggggtgtcagccccccgcccggccagctgccccgtccgggagggaggtggggggggtcagcccccccgcccggccagccgccccgtccaggaggtgaggggcgcctctgcccggccgcccctactgggaagtgaggagcccctctgcccggccagccgccccgtccgggagggaggtgggggtgtcagccccccgcccggccagccgccccgtccgggagggaggtgggggggggggtcagcccccccgcccggccagccgccccgtccgggaggtgaggggcgcctctgcccggccgcccctactgggaagtgaggagcccctctgcccggccagccgccccgtccgggagggaggtgggggggtgtcagcccccccgcccggccagccgccccgtccgggaggtgaggggcgcctctgcccggccgcccctactgggaagtgaggagcccctctgcccggccactaccccgtctgggaggtgtgcccaacagctcattgagaacgggccaggatgacaatggcggctttgtggaatagaaaggcaggaaaggtggggaaaagattgagaaatcggatggttgccgtgtctgtgtagaaagaagtagacatgggagacttttcattttgttctgcactaagaaaaattcctctgccttgggatcctgttgatctgtgaccttacccccaaccctgtgctctctgaaacatgtgctgtgtccactcagggttaaatggattaagggcggtgcaagatgtgctttgttaaacagatgcttgaaggcagcatgctcgttaagagtcatcaccaatccctaatctcaagtaatcagggacacaaacactgcggaaggctgcagggtcctctgcctaggaaaaccagagacctttgttcacttgtttatctgctgaccttccctccactattgtcccatgacccagccaaatccccctctgtgagaaacacccaagaattatcaataaaaaaataaatttaaaaaaaaaaaaaaaaaaaaatccaggctgggcatgctggcacatacctgtattcccagctgcttgggaggctgaggtgggaggattgcttgaggccaggagtttgagaccagcctgggcaacacagtgagaccccccatctctgcaaaaaaactttaaaaattagacaagcatggtggcacgcacctgtactcccaggtactcaggaggctgaggagtgaggattgcttgagcctgggagttcaaggctacagtgagctatgattgcaccactgcattccagcctgcatgacagagtgagaccctgtctcaaaaacaaaaaacaaaaccaaaacaacaacctaaacctcacaaaaaatgaactcaaatagatcatctatttaaatgtcaaatgtaaaactataaaactttgataagaaaatgtaggagaaaatgtttgggacctgggagtcattgaggaattcttaaccatgacaccaaaagcagatccttaaaattaaaaagaacaaaaaaaaaaaaaaggataaattagacttcaccaaaattaaaaagaaaacagccttttctctgcaaaaccctcattaagaggatgtaagagataagctatggactgggacaaaatatgtacaaaccacacatacacaaaagcctagcgtctagaacacataaacaacgagaaggcgcagctcggagcagggctgggaggatgagcaaaggcagacgtgccggctgcagccgcgctccgtgagtgagtggctgctgcatgccgggcctccagctgacagtgcctcccggggtcaacccgaagccagaggctcggcagtatgctggagacccatcctgggcacctcccacgttccctgtgggaacagctgcttctgcagaacatggaaaaggtccaggctggaagctgtgcagacggaatttaggaagagggaatggagagactgcaggagggagggaggcccctgcacggcaaggtggtggccacatgctccagaataggacttgggctccgcactgcagggaacgggacctgggctccgcgctgcggggaaggggacctgggctccgcgctgcggggaaggggacctgggctccgcgctgcggggaaggggacctggggtccgcgctgcggggaagggggcctggggtccgcgctgcggggaagggggcctggggtccgcgctgcaggggaacgggacctggggtccgcgctgcggggaacgggacctggggtccgcgctgcggggaaggggacctgggctctgcccagggactccacaattccttactaatgttactgccgtttatgggggctctgaacaacaccacgttttactaacacatgcacagagtgtcagatgatgacatgctgtctcacttatgctaaggctcactctcattttatcatctgtaacgtcagggtgcgccttataatcaacaggccacaaagaaagtcacaagccatactcacaagaactaggaagttcgttgcaaaatattccttgtccgtgatcagcacggcacacactatcagactcactctgccaacacaatcactagagaaggttcatctccgaggggaacgccagccagcttttcccaccactccccgcccccccgagaaagcccacgtgcagacagacccaggccagctgggcccctctccagctcctgaccagcagccccggatccctgggtggcgtgcagctgaccttccggaccgccactcagaataagcaagaaagcctgccgacccgcctgaatgcggcaggggaaggggaagggcggccccaacatctccgtgagacacgcggctgcatcacagcagcctcggcagtgcgcggggctgcagaatcaggtgcacaactagggcatcccccacccgcaacacacacggcacacaggagcctcggcagtgtgcggggctgcagaatcaggtgcacaactagggcaccccccacccgcaacacacacggcacacaaccacagaggcacggccccaggccaggagcgcacaactgccctcaaatgccctcagcagccacggcacacagccacaaacacatgaccccaggccaggagcacacaactgccctcaaacaccctcagcagccacacaggggaggccacgtccacgtgacttcaaccatcaacactagactagggtgcaaccgcgaagctgagtcagggccacacgcccccctacgccccaatctgtgcccacccttccccccaccaactgagtcttgtacaggggctggccaggacccttggatacgcagctgcagcctctggagagtgtttacacctccagctacgagtacttattgtggttcaggtggagggtggttccctctgacgatctccccaggacacacacgagctttctgccattctccctggcaggactggcctgcttggcacaggaagtctctgcagataaggcggtctgcagtgacctgaaagctcagtggctcagaggaagctaacctctagcattccccagccacagagcacccctgtgggtaggacccacagagcctggcattacagggaccagacagcacggtgcatactctgaccagggattctgaggtcttgggccatttataccatggagataacgaagcttactcttaggcgggctgctaggagggcccaaagtcccaaaccctgatctaaaaagccagaatacccctgggttcaaatcctggctccacggcctgcagtgtgacctgggctaagttgcttaagttttcacaattaaatttaaaatgcatggccgggcacagtggctcacacctgtaatcccagcactttgggaagcccaggcaggcggatcacttgaggtcaggagttcgagaccagccggaccaacatggtgaaaccccatctctactacaaaaaaaaaaaaaaaaattagctgggcatggtggcacgcacctgtaatcccagctactcaggaggctgaggctgcagtaagccaagatcgtgtcattgcactccagcctgggcaacatgacagaaactctgtctttaaaaaaaaaaaatattaaaatgcacatggtgacctatctcattgacgctaaggcgtactctcactttatcatctctaatgtgaggctgcgccttaaaatcagcagtatgtcggaatgcagtgggcagagggccttctttttagtaatccataaagtaatggcatagctaacactaatcaatcgtgtcttaggtttgatgaaatacggacgtatctacctcaaaacaatggttgaggctcagaacagaacctgacacagaatacgccctcaggtatcggtgttggctgcttgtgactgactttggcaagtaagatcagtacccggcacctctgggcacacagggcccgggctgcatccactgtgccagggacctggtgccgggctgggcagcaagtcagtatcacatcagcaggcgtgggccgtggtgagcctctccctgccctcccgctggaatagaaagctgatcccgacgtcccactccgacccacggtcaggaagccgtcctgtccaggaaggtggctgaggtacatccctgaacacagtaggccacagcatcatgcagcggagggccagtggccgctattcttcctgctggtgttccactcggcccgcagctcacagcaagtctatgcggaactgtgcaacccacatgctttgattttcagaaatgttcttagatcctgaaccagaatgtgctgcaggggggactgtttcacctcaaaaattacagcaaagtaaatgtccttgtttcaacttttccaatgtttaacttgtaacaatagctacaaaagtaatcaggcagcaatgtgtttctttaaacggctttcctgtgagtgttccgtttacaaagaaaatcctaaaaccaggaagtaattggtgactttggaaacagcaagcaaacttcggtgaagaaagaggggcaggtgtacctggaaggactaacttgcagccactcatttcctttggagggcggtggggcctgcaccctgcagattagggtatattctgactcagcaaaataggcaccagcgcctgtaaaaagaaaccgcgctgggcactaagagaattctcaaaaactcccagagccctgggtgtgtgtcctcccacagggaatctcgcacgagttcgtcaaatggctggtctcaggttacacaaccagcactgcctccaccgccaccgccccccaacctcactggtccgagcggttccccgcggtctagccgcgagggcggggccggggggcggaggccagggcgggaccaaggctgggggcggggccaggcatggctgccgctcgccggccgggggcggggccgacggtcacgtgtgtacacagggcccgggcggcgtgcgcgccgtgagccccgccgcctccgccagcccgagctgcccgcccggcggcgactgcgccggccgccgcccagcaagccggtgagtggggcccgggagcctgggtcgggtcggtggggcacaccccagtctccgcctggggagggagccagccccaagctgctgtggctcccacggcgggcgctgcgcttactgggacccgggtcctctggccgctgcccactgcggggctccgtgcccttgccaggaggggaccgctgtcctccggccgcgctccgagctgagccccgggggcgggctttcggtcattaacctggtgatggcagctgaatctgctgctggcgtaggcggcacagcgccgccaccggctctcccatgtccgacagggcaccactgatcctgctctcctgggccactatggtgactgctttcggccccttctgtgattagtattttaaagtgagttttggatctgtgtctacatggaggtgtcggatctgctgtcagttgtcttggacagttggcagggcatgggacggctctttcaaagcttcactggaaggcagtgctctccagccgccaaaccaccactaccagcagcagaaaacccactattaagttagtattacaaacctggcaagtcagttctctaaattaggcataagtctatgaatcttgatgtgactggtcacagtggcttatctctgcatgcatgctacgtggtggaccagaggataattctgtattcgtcgtccacagcctttgatctcttccctgcccatgaagatgtacttgagtcctccattccactttaaatagccagctcctgtccacacggaacagcacgacatcttttatcttttgctacctgcagcacctagcagaggccagcattcaataacagagggccaagaggcttgctttaaaggcctctgcaaacttgtatgagtaaaatggttctgaccatgttcccaggctcctcacgcatagtggtgatgagatagtctaccgtcctaactggggcaccgttgagagtgaacggaggcactgttaataattagacgaagtgcacactcacagtacccatgacctgctgtgcagctccttcaagaactactgtaaggcctggctccccaaccagaacccctgttgtcttcctcaatgaagaaggcctctgaagacaagtgacttaagaacatagtttatatgtattgaatactgttgtgccttttcacagagaagaggtcggggccagagcagctcactgcccgacgccacacacatccgtgctggcgtccacgtgtcatcctgcctctctagccagcatggacatatcctgaggatggaggacagaggaacacagatttagggcagaatgtaaatgtgaaagttgacttggagtccctgcaactcataagccccagtaggctgatgaagagagaagggcaaagggcgcgcacctcatgtgggtcttaggttaggatgtaaaggctgattatgtctatcatacaatgaatcaatggcatactgcacagatcgctgcatggggaaatatgctcgtgtaatttacagtcggaagagggacatgctgctcactggggcagggagaagaaggggctcctcagggagctgcagctctcacagcccctaggatgtcacctggaatcctgaaaaaataactgccgcctggcctttaatgacccaaccacagcgaggtcttgcctttgcagaaggaaattatcacccagagaacccggccgaccgtgtcatgcacacgacgatcccacggtgctgttttctgtcaacagtgtttcccagccattccctcctgcctgccagctcctctgctctccagggctttcactaacccacctgcccaactgaccagcattaggctgtccagttagcccacatctgtccaggggtctcaaaggctcaggaccccaagacaccatccaggtagccaagaggacagggaacccagtctggagagtagaataacgtttcgctccaatttgagatcccaaaatgagatgctgcccgggaccagaagtcagtttcttaggagcgtgctgtctacccaggagcctccgtgtgcaaacaaaatacccccttgagcagcagagggacaaggtggaggtggggcaggtgtctgttcatccccacagagccacacccaggtcccttcctgcaaaggccaaggactttccccattttaattggaaccaaagcaagccttgcagaccactgtcgctaccacagctagtgtgatgaggagaaataacctgctggctcctccctgggaaacaaccaaggtggtgataagatctgcggctcagagcacttccctgcaggttaaggccacgcctagtaaaccggggctcgactgaggagccccggcaggtgccaagcctgcccagagtctgcagtagagactctctgttcttggccatgtctttgaatttctgaggaagtcttttttgtttgtttttgtttttccctggtctgatgtacctggagagggaattagcctcacacccagttagggaacaaaaagccaagtaaacaaaagaatgctgttccaccttcctccagggaactggcgcccatagctgcaggcacaggtgcccagcccgctcactggcccagcaaggtgcttggtctcaggcccccagagcccgtcctggagccgcaaacatgctcgctattatccatggaaaaagagaggcattctctttgtttatagtctaagccaagtcacctgattcactctcactactctgcagtttggaattgtaaaaagcaaaaggctacaaaaatcaaaaccatccacctatgtaatgaaccatttcttatcacagcccaggaaggcacagataggaatcagtgtgcgtaagctctccacgtggaaaccctgtgaaaacagcagcctttcctaagcatctggctcctaggagctggatgttaattgttgctaaagcctgggttcattactacaaaagaaaataaacattccagagtcattttgaaagcattttgattgttctagaatattcacccaaggttgagcgtctcatggtgcatggcaggttcggccaggagctggccgtggcctttccactcctttcctgccatcctgaggatgcagtgcctggggcctcgcagttctctttccactcaaatctaaggctctaaagcttcaacaactgtccggggacaataagaagcgctgagtccccatcctgcctgctctcctttctactcagtggggaaattggatcactcgccttggtggatcactcgccttggggaaaagccttttacgtcactgaatacatccccttctacggttttcatccaacagtcagccaggctgctgcagcacacataaaaacttcccctgcctgcaaagagctccaagtaggtgggaaagagatggatgtgcggacaggcttaacccagcaaagactggcggaggggtgtggagacagtggtcaggggtgggttcccgggggtactgatgcccaaaataaaatgtcaaggacgagccagtgtttgctgagagaacgaggggaagcgagaaagggtgttctaggcaagggatcaacatccgcaaaagacagtgtgaaagagagcacccggaaacttcgtggggatcaccgggcgtgggacttgccggccccagctctgacagggccctaagcaacagaggaaagaacttcccgactaacagggacagcaacatcaaaagcgatggcttttgtgaaactaattcagccaccttggagcagaggattcctctgctggtttctgtttctgccagctctgtgcagtgcaggcgctatgatgctctctctctctcacacacacatacagggacccgggtgcctctcaggttgagaggggtggtcaccatccccacactcaggccccctctgtctccaggcctgctgcttctgaacaggcctggggcagtccatttctattgggaaaccctatcaaagttagccccgggtttcagccaagttcacctcactgctcacagacattcattattaacagactgtttgatagcactttgggctctttttaaataagacataggaagaaagatgccaacgtctcacttgaaatgctcctgacagctagcctgacggggtcctgtcgcccctgtccacaccctgctatggccatggtctccacagccgcacatgggctccagacattttcacgagttatgaaccaaagagaaccaagacccctggagcaggttctccttctcagtcccatgttcctttgaaagcaatgaatagaggccagggaagcaagtgtttatactccagtttagtactagtgaaacatcgggaaattcattgtgttggtatcaaagccccaactcagaacataaaaaccataaaactgcaaagcctctgcagtctgagcgtgcgctccccaggcctccgctgtctgagcgtgcgctccccaggcctccgcggtctgaccctgtgctacccatgcaagcgccacccacaggtgcgtcctgggggctgcctcctcctccacgttggccagcagtgtggggtgtgacggggcccatgttgttgtcacccccaccctgccccacgtttcccctgcacacgtgtccgttcacaaagattctgagcaaaaggtcacagaacagcttcctctgccctttatatgtggggaggaggccaaagatctgcaggcgtcatcattccaggccagggctggtaacagggagcaagtgccggcaccctcttgtgcttcacatgggttggtttacttaaccctcctgtctcatgaagatttttactaagcctggggaaaccgaggcacacagcaaggatgtcacctgcctgcaaaggcacctgtgtgcaagccaggactggaaaagctcagcctgcagtgcacatgcttgctgcccgctccagagcccaggcccttaaccacttccttcctaggaagacggagggcgagggagtttcttacagcgctttatgtcaactcctgctgctgcccaccactaatccaggcccctgcagacagcagggcacggtccttcctattagcaggcttactacgaagccctgtctaacattccctcatgtcaggtagaaggaattagaccagaggtcaaggcagcggctggaatcagtggtgctttctcaaagtaggaaaacagttttaggactgttttcggtcaaattttgggcaccactgtactgggttctatgacagagaagccaccaaggtcccgtgcgaggagccctgggcagggacagctgaagctgtgcacctgggccctcctgattgcctcccaccagccaggcctggcagcagccagcagccaccccaccccaccccaacaaggagagctgccccacactgtcggcccccaaagctcaggacacacgggccctcccaggtctctctggcgggctaggccttggcagggccccctcagcgggccccactctgaaccgcaagcacagggtcttctgcagtgtgagacctgctgtcagcacaggcctacgggctcagggctgtgcagggacacaggggccagcaccactgggcaccctgtcctttcccagcctgcctgtgcaccctgtgggcagtgactcctcaggcacagtggcccaaagtcacaggtggtctgtcaggggctctccagggagctgcacacactcaggtttagatcaggaacagaacagatccccagggcatttgctcaggctcagagcagagcagggagccgtccacctgcggcgggcacatccatggacttccaagccaacagggacagctgcccagacgggctgcccaagccaaccaaggatcctgtcccatcggtccctgtggcttgggagtctggcactttttggctttcgtttctcaagctgccccagcttctccaagtcctgagcctggggctcccagctccgagcggatgctctgcaagtggcgtccctgcaccctggtgcaggcggacggacgcgggagagcctgcaaggaagtaaggcccagcagccttcccgggcttggaacgccgcagcctaggaagcatcttcccaggaaatcgcagtcggagcccagcacggggtcttggaagaaaagaccccaccttctatcctcacgagtacgcattaggagtgagcaccgctcagtagatgacacagaggcacagctgaggacccagagacgggcacaccggaaacagcgacatccacaggcgctcggcagggaccaggaacccagaaggcttgacgcgcagggggtctcggagaaactctacccctgtccttcccagcctcctgcttagctgggcctcaccctagcctgctggagatggcggccatgccgtcagagagggggatggacagattggctgaggtcaccccacaggccagcagaggcctgggtgggagtgcgcagccccgcagatggctgcttatggggcccagaggccacccctgcgtcgcagagggcaccagcacacccggacttctcggaggcttcatacagctcccaggccctacaagcagcgtgtgggcagaggcccaggccccttcctgtcactcagagaagctgctgctcccagaaaacggggcgccagcctcccatgaacactgtgtcccaaccccggtcctctcaggattcctgtgtggaaatgtcacttctgtctcagctctctagggtgcacctggtgtccagctctgccccctttctgcttaagcagctgctgggagaccctgagccaggccgccgaggttctgagctcgtaaggggtgcagcagtttcccgcacagcgtggggagcacatttgctcacgtctcccaggctggctgtacactctgaggccagccctgggctctgacactctcccgattccgctgggctttctaagccctcctctcctgtccactgctggcccatggcccaggcccagccttgaagctgtaaagacctctcaccccatccaaatgagactggtcagatatctcccgcatgtcagccgccactggattgggaaggacgggcagtaggtgcaaggctggccctttttatgagagttatttaaaaaacaaaaacaggtcaggcacagtggctcacacctgtaatcccagcactttgggagactgaggcaggagggtcacttgaacccaggagtttgagactagcctgggcaacatagtgagaccctgtctcaaaaaacgttaaatgaaaacattagccagctgtagtggcacatgcctgtagtcccagcttcttgggaggctgagatgggaggatcgcttgagcccaggaggcagagggtgcagttagctatgatggtaccactgccctccagcctgggtgactgagagagatcctggctccaaacagaaaaataaaaacagaaggaggaacagatgaatcccatttgtgacattaatgaattctcttatagggaaaataaaacgattgaaactgggccatcttggacagctgaggcccaggcagggccagctccttccaccttcccagccaagatgctgcctgcagcccctcccctcctggcccgagatgctgtcattgtccctaagagaccctgaggcctgctccaggctgagtgggtgaggagcctctgagcaggcgcctgcgtcgaggcgtgccctcctgagaggcaaggctgacctgcagcgtccagctcggctggggacactcgcctgagaagagcccagtcctgggtgcctccctaggggagggccttggggctgcttgtgctggcaccgacgtgggcctggggagggacctgtgtgtgggtggcaggtcttgaggagtcgtgggggagggtttcggggggagatgctggtggtaacagtgggaggtaggcagtgaaaccagcagtgtgactttggaggtgctggaggagcagggcctggcggaagaagtgggggagccaggaaggagcagctgccagggacggcctggcctgggagggtggccaggaactgggggagaagctggtggtcaagctggtggtctctcagtgcaagggtggggctggcctggaggagaggcccacagggtccctgctggctgagggtgcagtggcgggccccggaagctgaatatcacccccgccaccgggcaggcagtgctcagagtgaaacgggtgactggagggcatgtgccagctctgttcctcaagcctggcccaagagggacctgcagcatggggttggggggcaggcggcccttctctgtggtctctcctgccttctgagggagcctccagaatctttcatcgccgccacaacaaactcaggctttcgtgtaggaaaagagcccacttctgaagaggctcatctgggctttctctcccagcctctaagtctcccatcacctggctccccgttcctctttcccttgaaggaagtcagcagagtgcatggctgacgggaaagaaggcacagcgggcctgcccagtggggggagaagccaggccattgttctccagttccaggatgccctgcttccccagacagcccggcagccaggaaaaggaagtctctgggcacagaggcaagcgggagaccacggacaggcaaggagtcgaaggctccacccgcctgggccaggccaggctcccacagaacccctctgcgtccaggcaggcacatggggcccaggccatccccctcactcggacgcaggtgtggcgacggcgggggaggggcgtccacagtagagcccagaaccttgcgcaggacaggaccccggctaatgtgcccggaacagctcagtgacagggagctgggttggctgtgagggtcgggcacgcgtcacgagtctcctgtgaccaggagaggtgggaacactggctcacatgactcgccaggccctggcacgctgtggttctgtgggtgccccctgcagctcccagccttcaaaatgtacacctcgctgggcatggtggctcacgtctgtaatcccagcactttgggaggccgaggtgggcggatcacctgaggtcgggagttcgagatcagcctggccaacatggagaaaccttgtctctactgaaaatacaaaattagctgggtgtggtggcagtcgcctgtaatcccagctattcgggaggctgaggcaggagaatcgcttgaacccgggaggcggaggttgcagtgagctgagatcgtgccactgcactccagcctgggcaacagagcgagactccatctcaaaaaaacaaaaacaaaaccaaaaaaccatacacttcatgctcacttctctgggagccatggctaacagcctgcaggcaggagactctcaaaccagctcctggagggcactcccagggaccctgttcctaccacgagccagagaggaggcgggtcaaggagacttgtccacccaggcgtggccgaggaaggggtttcctcacgctctgctgacctgccttccttccatgcctctcagcccagctcctgacaaaggcgggaacctgagctcctcggctttaggaacgtcccccagagcccacggagcagcccgccccagctgccttccctcccaagatctccaggtaatgcctggtaaaaggagggctccagggagaatgttggggccaacacgcaaagcgcgtcaccttccaaccggcggagagtgaaggaaaacaggtacggctcggcttgtaaccttcactggaaacagaaaccatcctcatcattgatacctggtgaactgcacagagagcaacctgcagctttggaagtttataaatccttcagtatttgtcagctgccagtaaacaaaaatgcacacaggagaactttccacctgaaccccacctggaggccggcgttcccaggtcccggctggggtgacagcacgcactcgagagccggttcagggcgagtgggacgcacgtgagcgcagggaggaaatgccagcagatcccactccacgtgaccgcctcccaagaatcgagccattcttagccctgcagacgctggctgcctccctctgacttggcctcacggcggggcaggcattagcagtccaggcagcttctctctgactccctgcaaggaggggtgcggcgggcaagaggctggccccacgcggttccacctgcaggagagccctcgcaggaggctccgtgtgtggggccggctcagcttttctctggccatgcccatgtcccttctgtgcacggagacaccagatgcacgtgctcccctctctgcttccgacctgaacctgccctcggcctccgctgtgcctctgaggactcctgtgtcctgtgggctgagtgagcatggccaggccttcgctcgacagtagacccttctgttgatttcctcacggaaaaaagggttcgtaaccaacatctcaagcacactgaacaccaggggcggctgcatggacctggacacgtgtctgcctgacctgcccccaggctctcggtgggatgccccgcagcgatgcagacaggggcctgcgggcagaggccacggcctccatgagccaggctatcagaaaggtgggacaagaggccagctgtgctcagtttgcagtgcggtcccgtccaagcacggcccctcgtttctggaacccggctgtaaacagcaggagggaggggcggtggcgcctgccaagggaggcagaggccctgcggggaacttaccagaggaaaacaagtgcttgcatcctggggccctcacacgcacgcgcaggcggccaggcgcgctgctgataagagcctgcgggtttgcagagaagcgcagcagcgcgcaggtcctcaccagagctctggtggccacctctgtcccgccatgctgctcaccgacagtggccagggcccacagcaccaagaggcttgggccacaaagtaaaggtgagagcctgtggtgcccgcaggtgggagaggggacgtggggggctctgtgcagtacaggcaggagaggggacgtgggggactctgtgcagcacaggtgggagagaggacgcagggggttctgtacagtgccaccggtggacatctctaagtagggaatgaaggggtgccgagacagagcacactgaggggtcccggcccttggcaagctctgtggggtgaaggtggccggccgggtgtgccctcctgtcagtgactggaggcagcacacaaggcacacagtgagacagccctcccgctccgatcttctaatcacagaccctggacagatccgggggccctgggcgccccgtgaggccaggccaggctgctgactgacacccagtcacagtggcctgtgtttcctggggactcaagtggagtctggggggtgacgtccccaaactgggctgccactctgtgacagccaaagccacgcagcctcgcaggcagaagctggggtgtggaggaccacgatcctgaagaaccccgtcctctggaccctggcgcagcaggaggggagaagcccgcagaggcccggacgcccccctcaccggcccccgccactgtgcttggagcaggtgcatcctgtctggagcttgtgctggcccggcgagtggagcccagagcccatcccccactaccaacatcccccagattgctgcctgcctggccgaggaggtgctggtcaagatgtgttagtgaggagctagacaagatgagcacgtgagaagcaggggccagggagcctggacccaggacctgggggtgctgtggcagcgctgtggcggcactgtggctgggcacacccagcagcggcctagctctgcatgcctgtcaccacctgaccctcactgcggctgctgggcggcattcaccaggattgcacaatgacccacacctcccgtcctgcccaccagcctctggatgggaattcccacatctacccacccgcccatctgccagctgcctgcatgcctagccacagggccgtgtctcctgctgtggggcgcacactgctattggcactggggacctcgggccctggcccctccgtgctctccacctcccgagcctcctcttcccgccctgctgtggccctgtgtgtggtgtgggtcttgcagtggcaggactccagtggcgggtgaggacccactcccgcagccggcctcctcactgctcttgcctccgggcatgggaacgatgccgtctgctcgtctgcctgagacgctttctactccactccgagagtaggaggtcagccggggccaaatgacatggtggacatcaggcttcccgagatagctgtaggtccctgaggaatgcctgtgctttggcggggtgggggggtgggggtggcgaaattcttactctaggatggctccagccgccaggttctcattcagtgacgtcaagactggctatgtccccatgtccccagagcctcctgtgggctgcgacccacaggtcaccctccctggagcccaggctctgcaaggactccaaggcaccgggtcctgagctgccgggagggagggagggaggctcagggctgaccgtccacattgcctgtccctcccggcgctggcaagatgcgaacaccacagacatctagtaaacttcgggaatcactgtgcggggcccgtccaaccaactcaggacgaccacccaggcctcgcagcagcccgtgaagagtgggctctgagaatgtggggcacaccaggctccctctctggtccccaaccccagccgctgaaataagtgcagccacagcagcccccggtcacgacccgagacggccatttttcaacgctcaacttcactttcacagaaatgcaccaaaggcagcctcgctctgtggtctgcagcgattactgcaccaggagaaggcacccctgtacctgcagcacttctgggccagggctttgggacgggacgcgagctgaccacctgttccttctttccgcagggtcgcggagcctcgccggccgccatgtggagctgcagctggttcaacggcacagggctggtggaggagctgcctgcctgccaggacctgcagctggggctgtcactgttgtcgctgctgggcctggtggtgggcgtgccagtgggcctgtgctacaacgccctgctggtgctggccaacctacacagcaaggccagcatgaccatgccggacgtgtactttgtcaacatggcagtggcaggcctggtgctcagcgccctggcccctgtgcacctgctcggccccccgagctcccggtgggcgctgtggagtgtgggcggcgaagtccacgtggcactgcagatccccttcaatgtgtcctcactggtggccatgtactccaccgccctgctgagcctcgaccactacatcgagcgtgcactgccgcggacctacatggccagcgtgtacaacacgcggcacgtgtgcggcttcgtgtggggtggcgcgctgctgaccagcttctcctcgctgctcttctacatctgcagccatgtgtccacccgcgcgctagagtgcgccaagatgcagaacgcagaagctgccgacgccacgctggtgttcatcggctacgtggtgccagcactggccaccctctacgcgctggtgctactctcccgcgtccgcagggaggacacgcccctggaccgggacacgggccggctggagccctcggcacacaggctgctggtggccaccgtgtgcacgcagtttgggctctggacgccacactatctgatcctgctggggcacacggtcatcatctcgcgagggaagcccgtggacgcacactacctggggctactgcactttgtgaaggatttctccaaactcctggccttctccagcagctttgtgacaccacttctctaccgctacatgaaccagagcttccccagcaagctccaacggctgatgaaaaagctgccctgcggggaccggcactgctccccggaccacatgggggtgcagcaggtgctggcgtaggcggcccagccctcctggggagacgtgactctggtggacgcagagcacttagttaccctggacgctccccacatccttccagaaggagacgagctgctggaagagaagcaggaggggtgtttttcttgaagtttcctttttcccacaaatgccactcttgggccaaggctgtggtccccgtggctggcatctggcttgagtctccccgaggcctgtgcgtctcccaaacacgcagctcaaggtccacatccgcaaaagcctcctcgccttcagcctcctcagcattcagtttgtcaatgaagtgatgaaagcttagagccagtatttatactttgtggttaaaatacttgattcccccttgtttgttttacaaaaacagatgtttcctagaaaaatgacaaatagtaaaatgaacaaaaccctacgaaagaatggcaacagccagggtggccgggccctgccagtgggcggcgtgtgctagcaaggcctgccgggtgtgccgcagtcaccacagggttctgagaacatttcacagaagtgcctgagacgcggagacatggctggtgttaaatggagctattcaatagcagtgacgcgctctcctcagccaccaaatgtccctgacaccctccccagcccccacagataacatcagctgaggtttttttcagtatgaacctgtcctaaatcaattcctcaaagtgtgcacaaaactaaagaatataaataaacaaaagaaaggtgtgttaggatttctgactgaataccagagctgaagggcccccaggggacagagggctggaggctgtgtcacacggggtctggtgggagggccagggtggaaccttaggccagctccgaaggcacctgggaccagcatccgtatctccgcggccctcgcagcctcagatggggcccggaccggcctgttccacttgagcgaacaggtctgcagtgaggagggtccatagtggggcgggtccgtagtggggcgggtctgtagtgaggcgggtcttagtgaggtgggtcttagtggggcgggtcggtagtgaggcggttcctagtgaggcgggtcccgagtggggcgggtcagtaggtggttcttagtggggcgggtcttagtgaggcaggtcttaggggggcgggtccatagtgaggcgggtccgtagtggggcgggtctgtagtgaggtgggtcttagcggggcgggtcttaggggggcgggtccgtagtgaggcaggtcttagtggggcgggtccgtaatgaggcgggtcttaggcaggcccgtagtggggcgggtctgtagtggggcgggtctgtagtgaggcgggtccgtagtggggcgggtctgtagtggggcgggtccgtagtgaggcgggtcttagtgaggcgggtcttagtgaggcaggtcttagtggggcgggtccgtaatgaggcgggtcttaggcgggcccgtagtggggcgggtcttagtggggcgggtctgtagtggggcgggtccgtagtgaagcgggtccgtagtgaggcgggtcttagtgaggcgggtctatagtcaggggcaattgttcctcgtcagcctggctggagaccttccacccattgggattttgtaaaacatctactttaaaatgtggccgtgagaaaagcccccattcctccccaccatctgttcgcctctccccgattttctaggattaaagtataggcctgcaatcccctacctccaattctaaagcccacaaagctctgaaaacaggaagatgttccccccaagtttgcggcaaattcactgggcagcgatccctgacctgaaggggtgtgagagcttcatgtccatgaggaccccaccccttctgaagacgggcgatgtggctgtgggagctgccctgagcgccccggggttctgtggcacacttcctccccacagcctgaacgcgctggaattctgagtatgcccggccccagggacagtgggggtagggtggggggagtggcaccttgcagccatcgacgtggggacgtcacgctctggtcccagtctcctgggaattcagccacgaggtcactggcattattcatcacgtgctgcaagcaggtcagagtcggcctcgtgaggacgcggctctgggtggctcctgcggggagctcagacccgcgggacaggctcctaagccgtgggattagagaacagttttctgagctgtttctgtcagtgtttggttttcaggtgtgaacctttaaagccgcaggcctgacctccctcccagtccctctggagccggagctggtgtaagggccgcgtctctgatggtcctgggcttcccatctccatgcctgcgcggaaaccctggcatccgatgaggaagtgatcacacaagcccacagaaccctgcccagcggctggcgccgtcagcccaaccctcctacccctgcagcccagctctgcagggtggggtgtccgtgcttgaaccaggagaccctcttcctggccgatactttccagccccccttggctccacgctgagctcccaggaaaggggcggaccccacacacaggtgagggtggcactgggctgcctctgcccgggaattacaccatccaactatcctccccaaaggcaggaggggcctaaaatcactatttcaatttctcagtggctttgggtgcaaactggctcatttctgtgggtctgttgcagggactggcctgcattcaatacccctcatcccaccctgctgggtggccctgtggagggccccagggacaccctgctgcctggtcagctgtttgttcctggtgggccccacacagggcaggcacatgcctccagcgcagaccctcgcctgctacctgagacacttcactgtgcaccatggcctgccccaaccccctacccagcgagtcccctcctcatccccaggttcccgccagcctctgctgcctgagaccctccactgagcgccaaggcctccgtcctcatccccaggctcccgccagtctctgtggctgcttcccaggagctgcgtcgctgactgttcctgagacacctgctcatctatattctatgttgccttcacctgggaagattcggcacctcctgagcctcctgccgcccctgagagattcccaaagccttcacctcatcacacacagaacacaagaacttgagccactcggggcagaaaaaggagctacttgtaaaaagtcaaggtagtcatctgcccaactccccagttcttccaggcagccgtctccccaactccccaattcttccaggcagccatctccccgactccccaattcttccaggcagccatctccccaactccccaattcttccaagcagccatctccccgactccccaattcttccaggcagccatctccccaactccccaattcttccaagcagccgtctccccgactccctagttcttccaggcagccatctctccaactgcttaattcttccaggcagccgtctgcccaactccccaattcttccccagtttgtgagacaccaggatgcgagtttttcagcgcgtgacgtcccagggtcaatgatattgctaccaacacttcttgactactatagttcaaaaatcattcatcttatttttgtttatccacaaaaaaaccttgccctgcttccttaagtaagattccagaaagggagtttagttgtagattcacttcattaaagttctaagagttattgtaggaagctggatctagtgtgaattgtggcgaagagatcaggaaatctacacacgtcttctttcatacacagcacacgacagagggaaagcaggaatagtaaccgtaacagaaagtatcataaaattggttatttcccctcgaattgtacgtcttaatactttcggttacccctctgtagttcctctttgcagaaattctgtccacccaatcacccaggagtggttccacttcacacacgcccggcacgcaaaccatgtctccacacgtctcaaacggtagcagagggagtaccttggctgctggcgaaggagtaagccacgcgggctgaaggagcaggcctgccccacacaggcgttactcctcccagaaaaataagaataaagtcaccattgactacacccctgagggcttctgtggccctgaggatggaactgaggtgttggggtatcctgcagccctcccaaggattccggcagaagtgagagcacacgttcctgcctccgccctgctgcacagggcagcctggcaagagctgtatggcggtgcgcacggtccgcactccgtcaggttgggaagtgaaggaagagcagagaatctaggcaggaagcataacgtgaacatgcccatttcacatgtacccctactcctacccctaacccctaactctaaccaacttaaacctcacctaaacaccctattcctaaccccctaaccaccctaaccctaaccctcaacaccctcatctgaaccctaacccttacagaatcgctctgggcctgcctggctttgccaagcctgagcgtgtgagtgcatctgacctgccccctccgttctctctgggctcctccatcctgtgtcttctggcatcgctcacagacccacgccccatcctgctcagccaccttgactcgctgggggaccacaagatgaacacagctctgacactgccagctgaccccggccacatctggggcttccacgcgacgctgtccccaaacagcaaatctttgggtacagatgggaggtgtcctgaggctgggacaaggactgggcaggggctggcagccggtctggcaacataagcaagcccctggctttccctctgctcactgccttggctctcttcctcactctgtgactgatcgggtgctcctgagaaacgggctccctgtgctgctcctggggtgtgcgtcattttcccgtgtcccgtgtcctacgggttccaggcactacaattcactggtccactctgctgctgacaaacatggggactgtttccaacttcaggcgttcgtgcacgaagcggcgctgttctgccgtgtctcccagcgcccaccatgggcccctgcgtggggcactcctcgggcagacacagcctcagccgcgcacgctcaccaagtgtggtctcacgtcctcacctgcccttggtgccatgtgtctctggtattttgtgccaagccaggggaggattaaggcatttcccatcttaatgacgtcttggcctctccaaagaagcgcctctttagggattctgcccacgtttctaagtgatgtaaggtccttcctggtttgatctgaatgtgggcccttgccacggctccttaatctaactgtacccctgtacctgtgctgttctctttccacagaagggcgagctctaccacaccacagagaagtcagagctccgcaagtccccacctggaagatggaagacccctcagaaacagcctgttcttccctctgcagaggtggggggcagggggactagccacactcaaaactggcagcctctgaggggagggagtcacttcaacatctcgaactttagacagatgtgtgtgcctcaggcagcatacacacacacatacatgcgtgcaagcatgcacacatatgcaagcacgcatgcacacacacaaacacacacgtctgtctcctccccgggaacaccgtgaccggaagaggcttcacacgcgtcggctgcagggtaggagacccgacacagcgccgggtcctgatgggctgtgacgtgaagatggggagaccaggcccttagcagccacagaacaggccccggaaagacgcacgatggctccactcttggaagcagatggtggctctgtagggagacaaggcttaccttgctccagggactctgaggagacgctgtgcagggaggctgcccccagcaggagacatggaaggtgagggcagaaggaacagcggtggggagcaggggcactgacaggttccagaccctgaccctgagcttcccacagcgcctaaacgcaacaccctgactcctgagaggaagggagggaatcccgagctgacctcacacaggagaaccgaagctcaaagctcaagtgtcaccctgaaaggcaccggcagacaaaggctgagtctccagagccagccctgcatccacccaaactagcgtggtgcagggactcctccctcggccgagtcacctggccccagactgtcggatgtggggccagcagggaccctgggggacgccccatggacacgagggagactgaagcccagagggtcccacagcagtgagcagcccctgctgccaggctccagggctccagaagccccagcccaccccacactcagggcgcacagaaggccccaggagggagggtgggaaacgccgggtcaagcgagcagcatccgattcttttctgcaggacatctgagctgtgccacacacactaaggcttctaaagagaaaagacagcgttccccgctgtccaaccctaatcatcacaggacgctttctgcagagccccttggggcacgaagattcttcccagcaccagtctgagaaacgatgaggccctgggcaggaggaaacatggccccatcctgttctaggggccacggcaccccttggggcccctctccagacccaaggctggacaggctggcttcctgcccagctccagccaactggcatacactaagtgtgcagctgaggtggtgggaggaaggtaaaacgtaccccacttggcagccacttccaaaattaaacatggttaacatctgtccccgctgtcccaattccagacatttacccaagaaaaatgggaacggaagtccacacaagcacctgtatacaaatgttcacggcagctttgctcataagagcccaacactggaaacaacccaactgcacatcaacaggtggacagataaacagatgtggcctgcgcatcgaaggaatgctactcgtcagcgaaatggacccaccggggacaggcgcggcaacagggtgaacctcacaaaacatgcagctgcggggacacagctagatgtgagcacgtgcaaagtactggagcctcttcagcagctccagagcagggaacccacctcaccagcgacacagcggcgacgagggccgggtctgggagggcgtgggccagggaggggcgacggaggcggtctcccttgccggggtgctggtgacacagcggctgcacctgtcagaacacgccagggtggagacaggagatctgtgtgcttcccgagtacagatcacggctcagcatctcatgggaaagggacagggctctcttcaggacacgcagtaagatttcaagtgcgggcacttttaatactccgcgatccaaaggcagctccagggccagccgcggtttccggcctcaagggcaggctcggttctggagctccctccagtggccgtcggggtgccgtcactttcagggccccaccaggagagcaggggccccgccgaggaccagagcgcctggaccagagggagccctgcgcggccggcacggatgcctctcaataggcggcatggggccgacacgactcggtgagttcccgccacggctttcgccggcagccggcggctggaggacaaggagaatgcgccggttctgttcctggacaagctccatggcgctgcggggtcccggcccagaaagcccaccctcccccagaatttccccaggcccacagaaggggaccggaatgggaaaaataccgacaaacgcagcaacggtgcggccgtaggtgtctgcgcatccggcggggctcctacgggacccccacgccgcctggacgccgcctagcagatttggggccaggctaattggggcccatcgtggcccacagatgccagctccgggccatgctgagggacaggggagcggaggatactgcctgtttcccggcggggggccctgctcaacagcctttcccttccctacaaactgtcccaggatcccgggccattccttccagtaagttgggaagtccaggaccagacctcaacgtggaaaaagctggaggagagaaggggggacgaggggttctacctgccctctacctacctgccctcctacctgtctgtccacgggatgcccagaggctcccagaccaccagccccagacccttggtactgcgtccccagctgtctgccaggggcctgctggggaggccgatgcccatccctaagcctgagcctccagcccggcacgagggaaggccccacatgccccaaaggagagggttcggggcacaatcttcacaaaggctggagtgcaccccagaggtgagggtttggggcacagtctgttggcggaggcaggagtacaccccagaggtgagggtttggggcacagtctgttggcggaggctggagtgcacccagaggtgagggtttggggcacagtctgttggcggaggctggagtgcacccagaggtgagggtttggggcacagtctgttggcggaggctggagtacaccccagaggtgaggatttggggcagtctattggcagaagctggagtacatcccagaggtgagggtttggggcacagtctgttggcggaggcaggagtacaccccagaggtgtgggtttggggcacagtctgttggtggaggctggagtgcacccagaggtgagggtttggggcacaatcttcacacaggctggagtgcaccccagaagtgagggtttggggcacagtctgttggtggaggctggagtacacccagaggtgcgggtttggggcacagtctgttggaggctggaatacacccagaggtgagggtttggggcacagttttcacacaggctgcagtgcaccccagaggtgagggtttggggcacagtcttcacacaggctggagtgcaccccagaggtgagggtttggggcacagtctgttggtggaggctggagtacatccagaggtgcgggtttggggcacagtctgttggaggctggaatacacccagaggtgagggtttggggcacagtcttcacacaggctgcagtgcaccccagaggtgagggtttggggcacagtcttcacacaggctggagtgcaccccagaggtgagggtttggggcacagtctgttggtggaggctggagtacatccagaggtgcgggtttggggcacagtctgttggaggctggaatacacccagaggtgagggtttggggcacagtcttcacacaggctggagtgcatcccagaggtgagggtttggggcacagtcttcacacaggctggagtgcaccccagaggtgagggtttggggcacagtcttcacacaggctggagtgcaccccagaggtgagggtttggggcacagtcttcacacaggctggagtgcaccccagaggtgagggtttggggcacagttttcacacaggctggagtgcacaccagggaggcttcccgcctctggcagaatcaccgccatgctcagtcacaaacccagagctgcgtttggacgctgcagcacacgctgcggccccagcaacggtcctgcgcaccaggctcctctcccagtaaggtccgcttctctgtggagctcaggggtccctgcagtgcccaccttagcagagggcaaagccttgagacacggatgctttgtcctcaggtctccactggctcctcagaacagggcccctcagcgctgcagtgtgtcacatgtccccagtttcccctcgtggtgctcacgccacacccctggcacggaggctggaacccaggtgtcagtcctggctctgaccatgaccttggacaaaccacccctcagacctagagccctcatgcacatccccatggtcactgccacccggcagggagcaggacagccccgggggtctgtgactgtccccgggacatcagtctgagaaacagcgctgagttggacgctgcctggtgtggacactcgccctccacagtcacacaaactcggctttgcaaaatcagttctcagtgaaggaaataatgtacacagtggtccacttccaagacacagtgtcttgcttgcttaggtcagcaagctacagaagaccaggcccctgcctgggttgccgatgcctgcttgtcggcctccccatccccagtcttccccccaacgcttagttgccttcacctgacccaaaaacttttagtctatgacgaaagttttactcgtctgcaacatagcttgttttgtctgttcttatcagtctgcccagctacttaggtcataagtcaaatacctgaaaagcccctgagctgactaggattgcaacgcattgtgggctgcaacaaaatgcagcaggacaaccctaaaaaaacacctacagcccctgcccgacaaccaataggtgatgtccgggaagactgtgaccccagagtactcagcctgtgaggaaccgggggaggggcctgcgtactgggggataaatggcttgttgtgaccgtgctgggtgtgcctgcccaccagacacccgatcttgcaagaccgtcattaaaagtctcacgtttaggccgggcgcggtggctcatgcctgtaatcccagcactttgggaggccgaggcgggcggatcacctgaggtctggagttcgagaccagctttaccacatggagaaaccccgtctgtactaaaaatacaaaattagctgggcgtggtggcacatgcctgtaatcccagctacgaagaaggctgaggcaggagaatcgcttgaacctgggaggcagaggttgcggtgagccaagattgcgccactgcactccagcctgggcgacagagcgaaactccgtctcaaaaaacaataaatagctctccctctcccctctcccctctcccctctcccctctccccacggtctccctctcatgcggagccgaagctggactgtactgctgccatctcggctcactgcaacctccctgcctgattctcctgcctcagcctgccgagtgcctgcgattgcaggcacgcgccgccacgcctgactggttttggtggagaccgggtttcgctgtgttggccgggccggtctccagcccctaaccgcgagtgatccgccaaccttggcctcccgaggtgccgggattgcagacggagtctcgttcactcagtgctcaatggtgcccaggctggagtgcagtggcgtgatctcggctcactacaacctacacctcccagccgcctgccttggcctcccaaagtgccgagattgcagcctctgcccggccgccaccccgtctgggaagtgaggagtgtctctgcctggccgcccatcgtctgggatgtgaggagcccctctgcctggctgcccagtctggaaagtgaggagcgtctccacccggccgccatcccatctaggaagtgaggagcgcctcttcccagccgccatcacatctaggaagtgaggagcgtctctgcccggctgcccatcgtctgagatgtggggagcgcctctgccccgccgccccatctgggatgtgaggagcgcctctgcccggcgagaccccgtctgggaggtgaggagcgtctctgcccggccgccccgtctgagaagtgaggagaccctctgcctggcaaccaccccgtctgagaagtgaggagcccctccgcccggcagctgccccgtctgagaagtgaggagcctctccgcccggcagccaccccatctgggaagtgaggagcgtctccgcccggcagccaccccgtccgggagggaggtggggggggtcagccccccgcccggccagccgccccatccgggagggaggtggggggtcagccccccgcctggccagccgtgccgtccgggagggaggtgggggggtcagccccccgcccggccagccgccccgtccgggaggtgaggggcgcctctgcccggccgcccctactgggaagtgaggagcccctctgcccggccagcacacatgcacgcacatacgcacacgggcacacgaatgcacgcgcacacatgcacacatgcatacacatacaccacgtgcacagggacacacgcacacacgtgcacacacgcaaacacgcacacacggacacgcacacacatgcacacatgcatgcacacacgcacacaacacgcacacggacacacatgcacacacatgcacgcacatacgcacacgtgcacgatacacatgcacacgcgagcacatgaacacgcacacgcgcacacatgcacgcacatatgcacacaacgcacgcagacatacacatgcacacgcacgcacacggacacgcacgtgcacacatgcacacacatccacacgtgcacacatatacacacaacgcgtgcacacggacatgcacacgggtgcgcacacgtgcacatgcacacacgtgcacacacatgcacgcacacacacagcacgcacagacacgcacacatgcacacacacacagcacagacacgcacacgggcacatgcacacgggcacacacacacgtgcacacgcacatgcacacacatgcacacacatgtacatgcagccccgaggcagtgccaccggagccagcctcatggagtagcctcctccagggtaacaggagccatgggggggccatgagactggattccactcatagaggccccgcacgcggctgttcaagcaaagccacactgacatttctggtatcaaatttaagtcaatcttttaatgaccctcccgcacgtcagcagcagatcaggtgcaatggcaagaaaagcaagcgagaaacagctgtacaacaacataggcttaaaaattctttttgaaagaaactttaataaacgatgcattggtgagtggtgaaaccggaaggtgctgtggccccgggctctgtggcactagcactggtagtctcaggtgcgctgagaggaccagagctccttggagaaaagctgatgtgaggtgggcctgggcactggtgggggcagctggggggctcccactggccacacaatacattaaaaaccacaaaaactgatgagtccacagcgacactccaaaaagcaggcaggagagggaggaaaggggagagggaggagaagtaccactgtgagtcttcatagaagccactgtttccacagaccctgggcaggaacaggttcaggagacgcaaaggcaccgggtgggaggggtcaggagggcaccgtcgccacaggacgcattgccagggaagtcctatgatgcacgcccacctcagtcaagtgaccacattcggtgtcaccagcatgggacaaggtggaatggcatgcctgcgggcgacaccacatacaacaggcagccttgcccctgtgggactcacccacctgagcggagccagcctaggacaccttagaaggagggcttcggcacccaagactgcacccccaggcccaggccgcacggccgcagcacaggctctgcacctggtcggcacacgtggggaggctgcaggacagagccaagcagctccgggcaagtggcacccaacagtcacccagccacacaggggaagagcggggaggggcccagcgctcaggatggaagagccctgggcaaggcatccatagacaccttagactctggcctaagcggggatccaagatggccttccgatacccagaccctcccatgcacacagagtgcccccacctccccatggtcctggagccgtaagcatcctggctgcccacggtcagcacacactgacccgcccacggtgcctctgctgcccttcttctctcgccactccccatctgcagcccgtgtgctgaggagggcacggaaaacgtccaaagcacgcacgactgcagcgaagctcatgaatgcccctgcacacacgcagtggggcagacaccggttggccctgccaggccctgcacactcacgggaccacgactgctcctcggatttccagggtcaaatggggtccagtcaaggaagggctgtgcgttctgcttctgcgcttaggcatatgattaaacacatttcataattagatatgatcattgcatctgagacacagagctttccaaaacggtctgtgggctgaagttactgcttccctaaacgtctgaggggcaacaccagtgccgccagctaggcctgcagccctccttgtagaaaggctttaattatggagtcaatttcttttctttcctttttttttttttttttttttttgagatggagtctcgctccatcgtgcccaggctagagtgcagtggcgcaatctcagctcactgcaacctccgccccctgggtgcaggtgattctcctgcctcagcctcccaagtagctaggattacaggcgtgcgccaccacgcctggctaatttttgtatttttagtagagatgggatttcaccatgttggccaggctggtcttgaactcctggcctcaagtgagccaccgcgcctggcctggagtcaatttctttaatcgttacaggactgacagagccttcaaaagttcttcttgggtcagtatggtaatctgtatcttaaaacaatgtgcccagtattggcataaagttgttacaacgttcccttactgtcattctgttgtctctagaaggatctgaaaatgtcctaggcccaccgaccaaaaccaggtcctgaaaaccaggactggcagcaggagaggaaggcgctcctctggcctctttctcacccactctgcagcttcacacacaggaacaccttacaccctccttcggtgtgaatttcaaaatacatacaatgaccagagggcagcggaagggaagggcagagagctttcaagcaacactgccctccctcctcacctcctcaccgcattccactcccgtttcggagacaagtggccggcagctactccccgcgcaggcagctcctccggcgccccagctgagctcccgccgcccctcaggctctgcttcccaccccctaatcccgctggaatctgccacagcatcgacctgctgctcctcaacatcgtagagctttcttactttctcacaactccttgagacactttttttactgtggtttccttccccagcccatatttggttctcatgaaaacagaagctccgtggagaaagcgaagaaagtccttgaaagtcgcctgttcaaagccgccactgccgtgatgccccctgactccgcaccccgccgccgtgacgccccccacgaccccgcaccctgctgcagtgacgcccccgacccctcaccccgctgccgtcacacaccccgaccccgctgccgtcactccccacgaccccccaccccgctgccgtcacgctccgtgacccccgaccccgacaccctgctgccgtgatgttcccccaaccctgcaccccgctgccgtgacgcccgccccgaccccgcaccccgctgctgtcacgcccccgaccccgcaccccgccgccgtcacgcccgacccctcaccccgctactgtgacgccccccgacccctcaccccgctgccgtcacacaccccgaccccgctgccgtcactcccaaccccccaccccgctgccgtcacgctccgtgacccccgaccccgacaccctgctgccgtgatgttcccccaaccctgcaccccgctgccgtgacgcccgccccgaccccgcaccccgctgccgtcacgcccccgaccccgcaccccgccgccgtcacgcccgacctctcaccccgctactgtgacgccccccgaccccgcaccccgccgccgtcactcccccgaccccgcaccccgctgccgtgacaccccccggcccccatcccgctgccatcacgcctcccggcccccgcacccttgtctgtgtcccacagcacaggacctgtcagcgagcgtctggcgccttcctccctcccctgcagagacccgccaggcccctccaacaggtgctttctaaactgcaatttcctcatctacaaaaggacaatcacaacaacggaacccaggggacaggactgcatcaactacaaccagacggcgtagacggcagcacacggccctcggcaaatcagaggtgctcagtgaccacagccaccatgacagcagggcggggcggggggcaaggaagcagcgctgggagacttggagacaccccaaaaaacgccttcctcagaaacactgctgtgcaagaacctgcaaatccctaaggcgtctgtgtccgatggccccggccccatgctctgaggtcgcgcaggtgtccaaggccaggaggggcaccgaggagacgaagaagaaacagcacagcagctggggaggtgaggcccgcgcctgggatgtcatgcagcctgccgagcagccgctcgagggacccgcccatgtgtcatcagctctggccttcctcctctcacagatcagagagcagagggcaaggtgggcagagcccagggtgggtgcagggctgagcgcaggacagacgtccctccagcctggtggctgccccgtgtggtgagcgggaggacgtgtgagctggggggctcctccttgtgactgcagatgtgtgggccatgggagggcgcagaatggaccaagaaccgaaaggtctgtgcaggggccctgcagcatcccaggagcagccttgccgccacctccatccaacacaacccgtcctctggcctcactgaattc

SEQ ID NO: 18 - DNA, 332 bp, Homo sapiens
  source 1..332: mol_type = genomic DNA
gatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtcta

SEQ ID NO: 19 - DNA, 1616 bp, Homo sapiens
  source 1..1616: mol_type = genomic DNA
aggtctctgcgcagcccagcccgccggtccacgccgcgcaccgctccgagggccagcgccacccgctccgcagccggcaccatgcgcgagatcgtgcacatccaggcgggccagtgcggcaaccagatcggcgccaagttttgggaggtcatcagcgatgagcatgggatcgaccccacaggcagttaccatggagacagtgacttgcagctggagagaatcaacgtgtactacaatgaggctgctggtaacaaatatgtacctcgggccatcctggtggatctggagcctggcaccatggactctgtcaggtctggacccttcggccagatcttcagaccagacaacttcgtgttcggccagagtggagccgggaataactgggccaagggccactacacagagggagccgagctggtcgactcggtcctggatgtggtgaggaaggagtcagagagctgtgactgtctccagggcttccagctgacccactctctggggggcggcacggggtccgggatgggcaccctgctcatcagcaagatccgggaagagtacccagaccgcatcatgaacaccttcagcgtcatgccctcacccaaggtgtcagacacggtggtggagccctacaacgccaccctctctgtccaccagctggtggaaaacacagatgaaacctactccattgataacgaggccctgtatgacatctgcttccgcaccctgaagctgaccacccccacctacggggacctcaaccacctggtgtcggccaccatgagcggggtcaccacctgcctgcgcttcccgggccagctgaacgcagacctgcgcaagctggcggtgaacatggtgcccttccctcgcctgcacttcttcatgcccggcttcgcgcccctgaccagccggggcagccagcagtaccgggcgctcacggtgcccgagctcacccagcagatgttcgactccaagaacatgatggccgcctgcgacccgcgccacggccgctacctgacggtggctgccatcttccggggccgcatgtccatgaaggaggtggacgagcagatgctcaacgtgcagaacaagaacagcagctacttcgtggagtggatccccaacaacgtgaagacggccgtgtgcgacatcccgccccgcggcctgaagatgtcggccaccttcatcggcaacagcacggccatccaggagctgttcaagcgcatctccgagcagttcacggccatgttccggcgcaaggccttcctgcactggtacacgggcgagggcatggacgagatggagttcaccgaggccgagagcaacatgaacgacctggtgtccgagtaccagcagtaccaggacgccacggccgacgaacaaggggagttcgaggaggaggagggcgaggacgaggcttaaaaacttctcagatcaatcgtgcatccttagtgaacttctgttgtcctcaagcatggtctttctacttgtaaactatggtgctcagttttgcctctgttagaaattcacactgttgatgtaatgatgtggaactcctctaaaaattacagtattgtctgtgaaggtatctatactaataaaaaagcatgtgtagaaaa

SEQ ID NO: 20 - DNA, 789 bp, synthetic construct
  source 1..789: mol_type = other DNA
gttcgttgcaacaaattgatgagcaatgcttttttataatgccaactttgtacaaaaaagttggcatgaaccgactcttcgggaaagcgaaacccaaggctccgccgcccagcctgactggctgcattggcacggtggacagtagagcagaatccattgacaagaagatttctcgattggatgctgagctagtgaagtataaggatcagatcaagaagatgagagagggtcctgcaaagaatatggtcaagcagaaagccttgcgagttttaaagcaaaagaggatgtatgagcagcagcgggacaatcttgcccaacagtcattcaacatggaacaagccaattataccatccagtctttgaaggacaccaagaccacggttgatgctatgaaactgggagtaaaggaaatgaagaaggcatacaagcaagtgaagatcgaccagattgaggatttacaagaccagctagaggatatgatggaagatgcaaatgaaatccaagaagcactgagtcgcagttatggcaccccagaactggatgaagatgatttagaagcagagttggatgcactaggtgatgagcttctggctgatgaagacagttcttatttggatgaggcagcatctgcacctgcaattccagaaggtgttcccactgatacaaaaaacaaggatggagttctggtggatgaatttggattgccacagatccctgcttcatnnccaactttcttgtacaaagttggcattataagaaagcattgcttatcaatttgttgcaacgaac

SEQ ID NO: 21 - DNA, 10567 bp, Homo sapiens
  source 1..10567: mol_type = genomic DNA
acttccggggttgctctggatccgctggttccgtaacaacatcccgttggcttccctcaggcggcgggaccagtgcagccgccgcctcccaggatcgtccgccggtcagggcccttgccctccccggcacaggccaccatggccaccaacccacagccgcagccgcctcctccggcgccgccgcctcccccgccgcagccgcagccgcagccaccgccgccgccgccgggccccggggctggccccggcgcgggcggggcgggcggcgcgggtgcgggcgccggggacccgcagctcgtggccatgatcgtgaaccacctcaagagccaggggctcttcgaccagttccgcagagactgcctggccgacgtggacaccaagcctgcgtatcagaatctgagacagcgtgttgacaactttgttgcaaatcacttggcaactcacacatggagtccgcatctcaataagaaccagctaagaaacaacattagacaacaagtcctcaaatcaggaatgttggagtctggtattgaccgaattatttctcaggttgtggacccaaagatcaaccacacattcagacctcaggtagagaaagctgtgcatgagtttttggccacgctaaatcacaaagaggaaggaagtggcaacacagctcccgatgatgagaaaccagacacttcccttattacacaaggtgttcctactcctgggcccagtgctaatgtagccaatgatgccatgtcgatattggaaaccataacttctcttaaccaagaagccagtgctgctagggcttcaacagaaacatcaaatgccaagaccagtgagagagcgtcaaaaaaacttccatctcagccaaccactgatactagtactgacaaagaaagaacttcagaggacatggctgataaagaaaaatctacagctgactctggaggtgaaggactggaaacagccccaaagtctgaagagttcagcgacctcccctgtccagtcgaagaaattaaaaattacacaaaagagcataataatttaattctgctaaataaggatgttcaacaggaaagcagtgagcaaaaaaataaatcaacagacaaaggtgaaaagaagccagacagcaatgagaaaggagaaagaaagaaagaaaagaaggaaaagactgaaaagaaatttgatcactcaaaaaagagtgaagatacacagaaagttaaagatgaaaaacaagcaaaggaaaaagaagtagagagtttaaaacttccttcagaaaagaacagtaataaagctaaaactgttgaagggacaaaagaagatttctctttgatagattctgatgtggatggacttacagacatcacagttagctctgttcataccagtgacctttcatcttttgaagaagatactgaggaggaagttgtaacgtctgatagcatggaagaaggagagattacgtcagatgatgaagagaagaacaaacagaataaaacaaaaactcaaactagtgattctagtgaaggaaaaacaaaaagtgtacggcatgcgtatgtccacaaaccatatctttactcaaaatactatagtgattctgatgatgagcttactgtagaacaacgacgacagtccattgccaaagaaaaagaagagaggcttttaagaaggcaaatcaatagagaaaaacttgaagaaaaacgaaaacagaaagcagaaaagacaaagtcttcaaaaaccaagggtcaaggcaggagtagtgtggacttagaagaatcatcaacaaagagtttggaacctaaagccgccagaattaaagaagtccttaaagaacggaaagttttagaaaaaaaagtagccttaagcaaaaagagaaaaaaagattcaaggaatgttgaagagaactccaaaaagaaacagcaatatgaagaagattccaaagaaacccttaaaacaagtgagcattgtgaaaaggaaaaaatttcttcttcaaaggagctgaagcatgttcatgcaaaaagtgaaccaagtaaacctgcccggagactttcagagtctttgcatgtagttgacgaaaacaaaaatgaatccaaattagaaagagaacataaaagacggacatctacccctgttatcatggagggggtacaggaagagactgacacaagagatgtaaaaaggcaagtagaacgctcagaaatttgcaccgaagagccccagaaacagaaaagcacacttaaaaacgaaaagcatctaaagaaagatgattctgaaacaccacatttgaaaagcctacttaagaaagaggtgaaatcctccaaggagaagcctgaaagagagaaaactccatcggaagacaaattgtctgtgaaacataaatataaaggtgattgtatgcataaaacaggtgatgagactgagcttcactcttctgagaaaggtttaaaagtagaggaaaatattcaaaagcaaagtcaacaaacaaagctttcttcagatgataaaaccgaacgaaaaagtaaacataggaatgaaaggaaattatcagtattaggcaaagatggaaagccagtttctgaatatattataaaaacagatgagaatgttcgtaaagaaaacaacaaaaaagagagacgcttgtcagctgaaaaaactaaggcagagcacaaatcaagaaggtcaagtgattctaaaattcagaaagattctctgggttccaagcaacatggtatcacattacagagaagaagtgaaagttattcggaagataagtgtgatatggactccactaacatggatagtaatttgaaaccagaagaggttgttcacaaggagaaacgacgaacaaagagcttgttagaagagaaacttgtgttgaagtctaaatcaaaaactcaaggcaaacaggtaaaagttgtagaaacagaattacaagaaggtgccacaaaacaggcaaccactccaaaaccagacaaggagaagaacacagaagaaaatgactcagaaaaacagcgtaagtctaaagttgaagacaaaccttttgaagaaactggtgttgaacctgtattagagactgcttcttcttcagcacatagtacacagaaggattctagtcatagagccaagttaccattagcaaaggagaaatataagagtgataaagactccacttccaccaggcttgagagaaagttgtcagatggccacaaaagcagaagcttaaagcatagtagtaaagacataaaaaagaaggacgaaaataaatcagatgacaaggatggtaaagaagttgacagtagtcatgaaaaggccagaggtaatagttcactcatggaaaagaaattaagtagaaggttgtgcgaaaatcggagaggaagcttgtcacaagaaatggccaaaggagaagaaaaattagcagcaaacactttgagcactcccagcggttcctcccttcagagaccaaaaaagagtggtgatatgacattgatccctgaacaagagccaatggaaattgattctgagccaggtgttgaaaatgtgtttgaagtatctaaaacccaagacaaccgcaataataattctcagcaagacattgactctgaaaatatgaaacaaaaaacttctgccactgttcaaaaggatgaattgagaacttgcacagcagattcaaaagcaacagctccagcttataagccaggccgtggaacaggagttaatagtaattctgaaaagcatgccgatcatagaagcaccttgaccaagaaaatgcatatacaaagtgctgtgtccaaaatgaaccctggggagaaagaacccattcatagaggaactactgaagtgaatatagattctgaaactgttcatagaatgttactgagtgccccatcagaaaatgatagggtacagaagaatttgaaaaacacagctgctgaagaacatgttgctcaaggagatgccactcttgaacattccacaaatttagactcctcaccatccttaagttcagtgactgttgtgcctctgagggaatcgtatgatccagatgtaattcctctgtttgacaaaagaactgttttggaaggtagcacagccagcacctcccctgcggatcactctgctctccctaaccaaagtctgactgttagggaatcagaagtccttaagacaagtgacagcaaagaaggtggtgaaggtttcacagtagatacaccagcaaaagcaagcatcactagcaaaagacacattccagaagctcaccaggctactttattggatggtaaacaaggaaaggtaatcatgcctcttggaagtaagttaacgggcgtgattgtggaaaatgagaatattaccaaagaaggtggcttagtggacatggccaagaaagaaaatgacttaaatgcagagcccaatttaaagcagacaattaaagcaacagtagagaatggcaagaaggatggcattgctgttgatcatgttgtaggcctgaatacagaaaaatatgctgaaactgtcaaacttaagcataaaagaagcccaggtaaagtaaaagacatatcaattgatgttgaaagaaggaatgaaaacagtgaggtagacaccagtgctggaagtggctctgcaccctctgttttacaccaaaggaacggacaaactgaggatgtggcaactgggcctaggagagcagaaaagacttctgttgccactagtactgaagggaaggacaaagatgtcaccttaagtccagtgaaggctgggcctgccacaaccacttcttcagaaacaagacaaagtgaggtggctttgccttgcaccagcattgaggcagatgaaggcctcataataggaacacattccagaaataatcctcttcatgttggtgcagaagccagtgaatgcactgtttttgctgcagctgaagaaggtggggctgttgtcacagagggatttgctgaaagtgaaaccttcctcacaagcactaaggaaggggaaagtggggagtgtgctgtggctgaatctgaggacagagcagcagacctactggctgtgcatgcagttaaaatcgaagccaatgtaaatagcgttgtgacagaggaaaaggatgatgctgtaaccagtgcaggctctgaagaaaaatgtgatggttctttaagtagagactcagaaatagttgaaggaactattacttttattagtgaagttgaaagtgatggagcagttacaagtgctggaacagagataagagcaggatctataagcagtgaagaggtggatggctcccagggaaatatgatgagaatgggtcccaaaaaagaaacagagggcactgtgacatgtacaggagcagaaggcagaagtgataactttgtgatctgctcagtaactggagcagggccccgggaggaacgcatggttacaggtgcaggtgttgtcctgggagataatgatgcaccaccaggaacaagtgccagccaagaaggagatggttctgtgaatgatggtacagaaggtgagagtgcagtcaccagcacggggataacagaagatggagaggggccagcaagttgcacaggttcagaagatagcagcgaaggctttgctataagttctgaatcggaagaaaatggagagagtgcaatggacagcacagtggccaaagaaggcactaatgtaccattagttgctgctggtccttgtgatgatgaaggcattgtgactagcacaggcgcaaaagaggaagacgaggaaggggaggatgttgtgactagtactggaagaggaaatgaaattgggcatgcttcaacttgtacagggttaggagaagaaagtgaaggggtcttgatttgtgaaagtgcagaaggggacagtcagattggtactgtggtagagcatgtggaagctgaggctggagctgccatcatgaatgcaaatgaaaataatgttgacagcatgagtggcacagagaaaggaagtaaagacacagatatctgctccagtgcaaaagggattgtagaaagcagtgtgaccagtgcagtctcaggaaaggatgaagtgacaccagttccaggaggttgtgagggtcctatgactagtgctgcatctgatcaaagtgacagtcagctcgaaaaagttgaagataccactatttccactggcctggtcgggggtagttacgatgttcttgtatctggtgaagtcccagaatgtgaagttgctcacacatcaccaagtgaaaaagaagatgaggacatcatcacctctgtagaaaatgaagagtgtgatggtctcatggcaactacagccagtggtgatattaccaaccagaatagcttagcagggggtaaaaatcaaggcaaagttttgattatttccaccagtaccacaaatgattacacccctcaggtaagcgcaattacagatgtggaaggaggtctctcagatgctctgagaactgaagaaaatatggaaggtaccagagtaaccacagaagaatttgaggcccccatgcccagtgcagtctcaggagatgacagccaactcactgccagcagaagtgaagagaaagatgagtgtgccatgatttccacaagcataggggaagaattcgaattgcctatctccagtgcaacaaccatcaagtgtgctgaaagtcttcagccggttgctgcagcagtggaagaaagggctacaggtccagtcttgataagcaccgccgactttgaggggcctatgcccagtgcgcccccagaagctgaaagtcctcttgcctcaaccagcaaggaggagaaggatgaatgtgctctcatttccactagcatagcagaagaatgtgaggcttctgtttccggtgtagttgttgaaagtgaaaatgagcgagctggcacagtcatggaagaaaaagacgggagtggcatcatctctacgagctcggtggaagactgtgagggcccagtgtccagtgctgtccctcaagaggaaggcgacccctcagtcacaccagcggaagagatgggtgacaccgccatgatttccacaagcacctctgaagggtgtgaagcagtcatgattggtgctgtcctccaggatgaagatcggctcaccatcacaagagtagaagacttgagcgatgctgccatcatctccaccagcacagcagaatgtatgccaatttccgccagcattgacagacatgaagagaatcagctgactgcagacaacccagaagggaacggtgacctgtcagccacagaagtgagcaagcacaaggtccccatgcccagcctaattgctgagaataactgtcggtgtcctgggccagtcaggggaggcaaagaaccgggtcccgtgttggcagtgagcaccgaggaggggcacaacgggccatcagtccacaagccctctgcagggcaaggccatccaagtgctgtttgtgcggaaaaagaagagaagcatggcaaggagtgccccgaaataggaccatttgcaggaagaggacagaaagagagcactttacacctcataaatgcagaagagaagaatgtattgttgaactcccttcagaaagaagataagagcccagagacagggacagcagggggcagtagcacagcaagttattcagcaggaaggggcttagaggggaatgctaactcacctgcccacctgagaggaccagaacagacgtctgggcagacggctaaggatccctctgtcagcattcgctatttggcagcagtaaacaccggtgctataaaagctgatgacatgccacctgttcaagggaccgtggctgagcattcctttcttcctgctgagcagcaggggtctgaagacaacttgaaaaccagtaccaccaaatgtattactggccaagaatcaaaaattgctccttcccacacaatgatccctccagctacttacagtgtagctctgttggctcctaaatgtgagcaggacttgactataaagaatgattatagtggcaaatggactgatcaagcatctgctgagaaaacaggagatgataacagcacaaggaaatcattccctgaggaaggagacataatggttactgtgtcttctgaagaaaatgtgtgtgacataggcaatgaagagtctccattgaatgttttgggaggattgaaactgaaagccaacttgaaaatggaggcttatgtgccttcagaggaagagaaaaatggtgaaattctggcaccaccagaaagtctgtgtgggggaaagccaagtggaatagctgaactccagagggagcctttgttggtgaatgaatcactaaatgttgaaaattcaggcttcagaacaaatgaagaaattcatagcgaatcttataacaaaggagagatatccagtggtagaaaagacaacgcagaagccataagcggtcacagtgttgaagcagatcctaaagaggttgaagaggaagaaaggcatatgcctaaaagaaaaagaaagcagcattatctctcttcagaagatgaaccagatgataatccagatgtcctggattccagaatagaaacagcacaaaggcagtgtcctgaaacggagccacatgacacaaaggaagagaactccagagatttggaagaattacctaaaaccagttctgagacaaatagcactacctcaagggtcatggaagaaaaagatgaatatagcagcagtgaaactactggtgaaaagccagagcagaacgatgatgacaccataaaatctcaggaggaagatcagccaataattattaaaaggaaaagaggaagacctcgcaaataccctgtagaaacaacgttaaaaatgaaagacgactccaaaacagatactggcattgtcactgtagaacaatctccatctagcagcaaactgaaagtaatgcaaacagatgaatccaataaagaaacagctaacctacaagaaagaagtataagcaatgatgatggtgaagaaaaaatagtaacaagtgtgcgtcggagaggaagaaaacccaaacgttctctcactgtatcagatgatgctgaatcctcagagccagaaagaaaacgccagaaatcagtttctgatccagtggaggacaagaaagagcaggagtctgatgaggaagaggaagaagaggaagaggacgagccttcaggagccaccacaagatccaccaccagatcagaggctcagagatcaaagacacagctctccccttctatcaagcgcaagagagaagtcagccctcctggggcccgaacaagaggccagcaaagggtggaggaagcccctgtgaaaaaagcgaagcgataatcctgaccactgctgccctaggcttatggaggaacacggtggagaggaaagagacatgccttggtggccataggcttctctttaaccaggaaaaagatatgcatgtgctgtaagtccctaggtgcaagctttttcttgttatgttttaaacagctttataaactattgttcatagaagatattatgtacatttatttcagataaaggacaataagtttactttgtatctgaactcaaaacaaagtagttgtatattttaacattcaaaattgggatttcccaatgtgacacatcatgaatgcaaacccctccagcccatcagacgccaggctgcctactggtaatctgtgtatagtatataaacatgtaaaaataggttgtattttactctatgtatgatgctaatcaatgaacactttatttattttacagagaaaacttatctgtgaactttactatatatctgtttattttactttattttttttttaaataaaaagggttttaaatgctatgcagtcattagtagaaaattttttaggactctgcctgctctgtaactatcttaatatgatctggcagaaactcgcatgtatccaagtaaagtagtttagctaaagaaaggttcttcattgcttttctgttcacagttgtggctctgttttttaagaatgtaacttgtttttagattatacttgcatctgtgactttactaccagccacgttgacacaaaacaggttctggttcaggtaaagttgcgtcagtcacctgcagcagaaatccctcttcattcctcttctctgtgttcattcctcttctgtgctgttctgaagcttctaccaatactctttccatattgtctttttcagtgaagagaaatgcattcaagattaggtccctcctgtctatccagtttcaggattttatgttgttttatacacagttatttcagtatagaaactggctttattgccaagtgtttttttaaacatgttttaactctcatatgagcaaactgtccaacttcagtttttcataagattaaacttcttacgatcaaatttgtctcttgcaatgatgtgatgagttgccaaataattgagattattttaaaatgttttgttcatattcttgttttataattaaaatttacattcagtgtgtatgggtttttttttttattttgactcttaatgtaaggtggatatttctgtcattttacatggtttcttactgagattttatatataaattataaaatgtttaccaaaa

SEQ ID NO: 22 - DNA, 3765 bp, Homo sapiens
  source 1..3765: mol_type = genomic DNA
gcaaccctcgacatggcgctgaggcggccaccgcgactccggctctgcgctcggctgcctgacttcttcctgctgctgcttttcaggggctgcctgataggggctgtaaatctcaaatccagcaatcgaaccccagtggtacaggaatttgaaagtgtggaactgtcttgcatcattacggattcgcagacaagtgaccccaggatcgagtggaagaaaattcaagatgaacaaaccacatatgtgttttttgacaacaaaattcagggagacttggcgggtcgtgcagaaatactggggaagacatccctgaagatctggaatgtgacacggagagactcagccctttatcgctgtgaggtcgttgctcgaaatgaccgcaaggaaattgatgagattgtgatcgagttaactgtgcaagtgaagccagtgacccctgtctgtagagtgccgaaggctgtaccagtaggcaagatggcaacactgcactgccaggagagtgagggccacccccggcctcactacagctggtatcgcaatgatgtaccactgcccacggattccagagccaatcccagatttcgcaattcttctttccacttaaactctgaaacaggcactttggtgttcactgctgttcacaaggacgactctgggcagtactactgcattgcttccaatgacgcaggctcagccaggtgtgaggagcaggagatggaagtctatgacctgaacattggcggaattattgggggggttctggttgtccttgctgtactggccctgatcacgttgggcatctgctgtgcatacagacgtggctacttcatcaacaataaacaggatggagaaagttacaagaacccagggaaaccagatggagttaactacatccgcactgacgaggagggcgacttcagacacaagtcatcgtttgtgatctgagacccgcggtgtggctgagagcgcacagagcgcacgtgcacatacctctgctagaaactcctgtcaaggcagcgagagctgatgcactcggacagagctagacactcattcagaagcttttcgttttggccaaagttgaccactactcttcttactctaacaagccacatgaatagaagaattttcctcaagatggacccggtaaatataaccacaaggaagcgaaactgggtgcgttcactgagttgggttcctaatctgtttctggcctgattcccgcatgagtattagggtgatcttaaagagtttgctcacgtaaacgcccgtgctgggccctgtgaagccagcatgttcaccactggtcgttcagcagccacgacagcaccatgtgagatggcgaggtggctggacagcaccagcagcgcatcccggcgggaacccagaaaaggcttcttacacagcagccttacttcatcggcccacagacaccaccgcagtttcttcttaaaggctctgctgatcggtgttgcagtgtccattgtggagaagctttttggatcagcattttgtaaaaacaaccaaaatcaggaaggtaaattggttgctggaagagggatcttgcctgaggaaccctgcttgtccaacagggtgtcaggatttaaggaaaaccttcgtcttaggctaagtctgaaatggtactgaaatatgcttttctatgggtcttgtttattttataaaattttacatctaaatttttgctaaggatgtattttgattattgaaaagaaaatttctatttaaactgtaaatatattgtcatacaatgttaaataacctatttttttaaaaaagttcaacttaaggtagaagttccaagctactagtgttaaattggaaaatatcaataattaagagtattttacccaaggaatcctctcatggaagtttactgtgatgttccttttctcacacaagttttagcctttttcacaagggaactcatactgtctacacatcagaccatagttgcttaggaaacctttaaaaattccagttaagcaatgttgaaatcagtttgcatctcttcaaaagaaacctctcaggttagctttgaactgcctcttcctgagatgactaggacagtctgtacccagaggccacccagaagccctcagatgtacatacacagatgccagtcagctcctggggttgcgccaggcgcccccgctctagctcactgttgcctcgctgtctgccaggaggccctgccatccttgggccctggcagtggctgtgtcccagtgagctttactcacgtggcccttgcttcatccagcacagctctcaggtgggcactgcagggacactggtgtcttccatgtagcgtcccagctttgggctcctgtaacagacctctttttggttatggatggctcacaaaatagggcccccaatgctattttttttttttaagtttgtttaattatttgttaagattgtctaaggccaaaggcaattgcgaaatcaagtctgtcaagtacaataacatttttaaaagaaaatggatcccactgttcctctttgccacagagaaagcacccagacgccacaggctctgtcgcatttcaaaacaaaccatgatggagtggcggccagtccagccttttaaagaacgtcaggtggagcagccaggtgaaaggcctggcggggaggaaagtgaaacgcctgaatcaaaagcagttttctaattttgactttaaatttttcatccgccggagacactgctcccatttgtggggggacattagcaacatcactcagaagcctgtgttcttcaagagcaggtgttctcagcctcacatgccctgccgtgctggactcaggactgaagtgctgtaaagcaaggagctgctgagaaggagcactccactgtgtgcctggagaatggctctcactactcaccttgtctttcagcttccagtgtcttgggttttttatactttgacagcttttttttaattgcatacatgagactgtgttgactttttttagttatgtgaaacactttgccgcaggccgcctggcagaggcaggaaatgctccagcagtggctcagtgctccctggtgtctgctgcatggcatcctggatgcttagcatgcaagttccctccatcattgccaccttggtagagagggatggctccccaccctcagcgttggggattcacgctccagcctccttcttggttgtcatagtgatagggtagccttattgccccctcttcttataccctaaaaccttctacactagtgccatgggaaccaggtctgaaaaagtagagagaagtgaaagtagagtctgggaagtagctgcctataactgagactagacggaaaaggaatactcgtgtattttaagatatgaatgtgactcaagactcgaggccgatacgaggctgtgattctgcctttggatggatgttgctgtacacagatgctacagacttgtactaacacaccgtaatttggcatttgtttaacctcatttataaaagcttcaaaaaaacccaaacattgcttcattctttgttatttgctcttacgttgggtttgtctcttcttcctagcatttcagtggttaggcatgcacactaaaaatgctatcaatcacccatccaccagggaagtcagtgctgggcaggaggtcagcctgtgtgctcaagagcagtgctgcgcctctccgccaccgagcccacctgtgttgcggttcccacttgggatggcagtgggcagatctcagtggcatcccatcccccaaa

(19) United States
(12) Patent Application Publication
(10) Pub. No.: US 2026/0009080 A1
(43) Pub. Date: Jan. 8, 2026

(54) Genetic Biomarkers for Identification of PTSD and Major Depression

(71) Applicant: Trugenomix Health Inc., Manassas, VA (US)

(72) Inventors: Tshaka CUNNINGHAM, Woodbridge, VA (US); Jesus Enrique HERRERA, Columbia, MD (US); Anne NACLERIO, Fairfax Station, VA (US)

(21) Appl. No.: 19/243,989

(22) Filed: Jun. 20, 2025

Related U.S. Application Data
(63) Continuation of application No. PCT/US2023/085806, filed on Dec. 22, 2023.
(60) Provisional application No. 63/434,605, filed on Dec. 22, 2022.

(51) Int. Cl. C12Q 1/6883; G16B 25/10; G16H 50/20; G16H 50/80

(52) U.S. Cl. CPC C12Q 1/6883; G16B 25/10; G16H 50/20; G16H 50/80; C12Q 2600/158; C12Q 2600/178; G01N 2800/52

(57) ABSTRACT

Disclosed herein are assays and methods of identifying a subject with post traumatic stress disorder. The methods developed to identify post traumatic stress disorder may then allow a subject with PTSD to be treated for the disorder.

### CROSS-REFERENCE

This application is a continuation of PCT/US2023/085806, filed Dec. 22, 2023, which claims benefit of U.S. Provisional Application No. 63/434,605, filed on Dec. 22, 2022, which are incorporated herein by reference in their entirety.

### INCORPORATION BY REFERENCE OF SEQUENCE LISTING

The instant application contains a Sequence Listing, which has been submitted via Patent Center. The Sequence Listing titled 210536-010500_SL.xml, which was created on Dec. 22, 2023 and is 269,686 bytes in size, is hereby incorporated by reference in its entirety.

### STATEMENT REGARDING FEDERALLY FUNDED RESEARCH

No federal grants were used for this application.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

To the extent publications and patents or patent applications incorporated by reference contradict the disclosure contained in the specification, the specification is intended to supersede and/or take precedence over any such contradictory material.

### SUMMARY

Provided herein is a method for detecting post traumatic stress disorder (PTSD) in a human subject, the method comprising: obtaining a cell sample obtained from a human subject; measuring with a first computer program, executed on a computer, a test gene expression level of one or more PTSD-associated genes from the cell sample, wherein the PTSD-associated genes are selected from the group consisting of: TSPAN5, HIST1H2AE, UBE3A, GPX4, EPB42, SLC4A1, NDUFA1, HIST1HEH, ELOVL7, ALES, COMPT, PDZK1IP1, ITGA2B, CYP4F3, EPB41L3, PRDM1, RP-11-449P15.1, FAS, TUBB2A, and JAM3; with a second computer program, executed on a computer, comparing the test PTSD-associated expression level(s) to one or more control gene expression level(s); determining the differential expression of the one or more test PTSD-associated gene(s); and determining the positive detection of PTSD based on the differential expression. In some embodiments, the test gene expression level(s) and/or the control gene expression level(s) is of one or more, 2 or more, 3 or more PTSD-associated genes. In some embodiments, the test gene expression level(s) and/or the control gene expression level(s) is of 4 or more, 5 or more, 6 or more PTSD-associated genes. In some embodiments, the test gene expression level(s) and/or the control gene expression level(s) is of 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or all 20 PTSD-associated genes. In some embodiments, the PTSD-associated genes are selected from the group consisting of TSPAN5, HIST1H2AE, UBE3A, GXP4, EPB42, SLC4A1, NDUFA1, HIST1H3H, EVOLV7, and JAM3. In some embodiments, the PTSD-associated genes are selected from the group consisting of TSPAN5, HIST1H2AE, UBE3A, GXP4 and EPB42. In some embodiments, the test PTSD-associated gene expression level(s) is differentially expressed with a p Value of at less than 2.5×10⁻⁶. In some embodiments, the test PTSD-associated gene expression level(s) is differentially expressed with a p Value of at less than 9×10⁻⁷. In some embodiments, the test PTSD-associated gene expression level(s) is differentially expressed with a p Value of at less than 1.6×10⁻⁷. In some embodiments, the cell sample is obtained from a blood sample, a saliva sample, a buccal smear sample, a cerebrospinal fluid sample, saliva, skin, cerebrospinal fluid, or any combination thereof. In some embodiments, the test gene expression is a messenger RNA (mRNA), small interfering RNA (siRNA), a MicroRNA (miRNA), a small nuclear RNA (snRNA), a U spliceosomal RNA (U-RNA), a Small nucleolar RNA (snoRNA), a Piwi-interacting RNA (piRNA), a repeat associated small interfering RNA (rasiRNA), a small rDNA-derived RNA (srRNA), a transfer RNA derived small RNA (tsRNA), a ribosomal RNA derived small RNA (rsRNA), a large non-coding RNA derived small RNA (lncsRNA), or a messenger RNA derived small RNA (msRNA), a gapmer, a mixmer, double-stranded RNAs (dsRNA), single stranded RNAi, (ssRNAi), DNA-directed RNA interference (ddRNAi), or any combination thereof. In some embodiments, the expression level is assessed on a transcriptional level. In some embodiments, the expression level is assessed on a translational level. In some embodiments, the first computer program and the second computer program are the same. In some embodiments, the first computer program and the second computer program are different. In some embodiments, the method is performed more than once. In some embodiments, the method is performed weekly, monthly, bimonthly, three times per year, four times per year, twice per year, or annually. In some embodiments, the method further comprising the step of treating the subject for PTSD. In some embodiments, treating the subject comprises administering to the subject a pharmaceutical therapy, psychotherapy, or a combination thereof. In some embodiments, the subject is administered a pharmaceutical therapy. In some embodiments, the pharmaceutical therapy is an SSRI, a tricyclic antidepressant, an MAOI, an antipsychotic, a beta-blocker, a benzodiazepine, a psychedelic, or a combination thereof. In some embodiments, the pharmaceutical therapy is the SSRI. In some embodiments, the pharmaceutical therapy is the tricyclic antidepressant. In some embodiments, the pharmaceutical therapy is the MAOI. In some embodiments, the pharmaceutical therapy is the antipsychotic. In some embodiments, the pharmaceutical therapy is the beta-blocker. In some embodiments, the pharmaceutical therapy is the benzodiazepine. In some embodiments, the pharmaceutical therapy is the psychedelic.

Also provided herein is a method of screening PTSD in a subject, the method comprising: detecting PTSD in a subject comprising any one of the methods of detecting as described herein; and determining said subject should be clinically diagnosed for PTSD based on the positive detection of PTSD in the subject. In some embodiments, the control population is a PTSD negative population. In some embodiments, the method further comprises clinically diagnosing said subject with PTSD using one or more clinical PTSD diagnostic criteria. In some embodiments, the clinical PTSD diagnostic criteria is CAPS-5 or PCL-5. In some embodiments, the clinical PTSD diagnostic criteria is CAPS-5. In some embodiments, the clinical PTSD diagnostic criteria is PCL-5. In some embodiments, the detection step and the clinical diagnosis step are performed by two different entities. In some embodiments, the method further comprises evaluating one or more co-morbidities related to PTSD. In some embodiments, the one or more co-morbidities comprises generalized anxiety disorder, alcohol use disorder, anxiety related insomnia, childhood traumas, or any combination hereof. In some embodiments, the one or more co-morbidities are diagnosed with one or more clinical criteria comprising Generalized Anxiety Disorder 7-item Scale (GAD-7), Alcohol Use Disorders Identification Test (AUDIT-C), Pittsburgh Sleep Quality Index (PSQI), Adverse Childhood Experiences (ACE), or any combination thereof. In some embodiments, the detection step, the clinical diagnosis step, and the co-morbidities diagnosis step, or any combination of two thereof are performed by two different entities.

Also provided herein is a method of selecting an eligible subject for the treatment of PTSD, the method comprising: detecting PTSD in a subject comprising any one of the methods of detecting described herein; and determining said subject is eligible for PTSD treatment based on the positive detection of PTSD in the subject. In some embodiments, the subject was screened for PTSD according to any one of the methods of screening described herein.

Also provided herein is a method of evaluating treatment efficacy of a subject undergoing PTSD treatment, the method comprising: detecting PTSD in a subject comprising any one of the methods of detecting described herein; and determining the efficacy of the PTSD treatment based on the detection of PTSD in the subject, wherein a negative PTSD detection indicates the PTSD treatment is efficacious. In some embodiments, the PTSD treatment is a single PTSD treatment or an instance of PTSD treatment that is part of a series or ongoing PTSD treatment. In some embodiments, the PTSD treatment comprises Cognitive Behavior Therapy, Cognitive Processing Therapy, Cognitive Therapy, Prolonged Exposure Therapy, Eye Movement Desensitization and Reprocessing (EMDR) Therapy, Narrative Exposure Therapy (NET), Group Therapy, Brief Eclectic Psychotherapy, selective serotonin reuptake inhibitors (SSRIs), serotonin and norepinephrine reuptake inhibitors (SNRIs), anti-anxiety medications, anti-depressants, sertraline, paroxetine, fluoxetine, venlafaxine, ketamine, a tricyclic antidepressant, an MAOI, an antipsychotic, a beta-blocker, a benzodiazepine, a psychedelic, co-morbidities treatment, complementary and/or alternative therapies comprising acupuncture, yoga, animal-assisted therapy, meditation, or any combination thereof. In some embodiments, the method comprises detecting PTSD in the subject prior to treatment comprising any one of the methods of detection described herein. In some embodiments, the method comprises screening PTSD in the subject prior to treatment comprising any one of the methods of screening as described herein. In some embodiments, the method comprises selecting the subject as eligible for the PTSD treatment prior to treatment comprising any one of the methods for selecting an eligible subject as described herein.

Also provided herein is a method of determining prevalence of PTSD in a population of interest, the method comprising detecting PTSD in each subject in a population of interest comprising any one of the methods of detecting described herein; and determining the prevalence of PTSD in said population of interest based on the positive detection of PTSD in each subject of the population of interest. In some embodiments, the population of is a military population.

### DESCRIPTION OF THE DRAWINGS

FIG. 1 is a flow chart depicting sequencing of a subject's RNA and amplification and analysis.

FIG. 2 depicts machine learning to determine the possible number of genes that may be associated with PTSD.

FIG. 3 depicts twenty highly relevant genes associated with PTSD and their P values.

FIG. 4 depicts the demographics evaluated for the generation of an objective molecular detection assay to screen for PTSD.

FIG. 5 depicts the sensitivity of the molecular detection assay, or the percentage of subjects correctly identified as having the disorder, is 90%.

FIG. 6 depicts the Area Under the Receiver Operating Characteristic Curve (AUC) of a previous model with different thresholds using the same genes as achieving 85%. FIG. 6 also depicts the Receiver Operating Characteristic (ROC) Curve for the overall sample (all_in) and for a cross validation with a size of 1 or k=1 (k1_Cross).

FIG. 7 is a flow chart that is a schematic of an exemplary method that is consistent with some aspects of the present disclosure.

### DETAILED DESCRIPTION

### Overview

PTSD is on the molecular level characterized by a disturbed regulation of the primary stress-hormone system, the so-called HPA (hypothalamic-pituitary-adrenal) axis. The hypothalamic-pituitary-adrenal (HPA) axis is the major constituent of the neuroendocrine response to acute and chronic stress, resulting in the release of corticotropin releasing hormone (CRH) and vasopressin (AVP) from the parvocellular neurons of the hypothalamus into the portal vessels system to activate the synthesis and release of ACTH from the anterior pituitary. In turn, ACTH stimulates the adrenal cortex to synthesize and release glucocorticoids, in particular Cortisol (de Kloet et al., Nat Rev Neurosci 2005; 6: 463-75). In PTSD, the fine-tuned regulation of this HPA axis is disturbed, which is indicated by reduced levels and an exaggerated responsiveness of ACTH and Cortisol in these patients. Enhanced responsiveness of the glucocorticoid receptor (GR) appears to underlie these disturbances. These findings contrast with observations of greater Cortisol levels and reduced GR responsiveness associated with major depression (Ribeiro et al., Am J Psychiatry 1993; 150: 1618-29; Holsboer F, Neuropsychopharmacology 2000; 23: 477-501). Though initially interpreted as reflecting enduring consequences of trauma exposure, there has been reason to suspect that HPA axis alterations in PTSD reflect pre-traumatic risk factors. Recent studies of infant and adult children of parents with PTSD also support the idea that both low Cortisol levels, and glucocorticoid responsiveness are risk factors for PTSD. Indeed, it was recently observed that adults who experienced child abuse and have genetic variations in the FKBP5 gene (involved in the regulation of the GR) appear to be at greater risk of PTSD symptoms as adults (Binder et al., JAMA 2008; 299: 1291-305). There have been two other studies examining gene expression following trauma exposure. In a study of persons exposed to severe trauma encountered in the emergency room who either did (n=8), or did not (n=6) meet criteria for PTSD at both a 1 month and 4 month follow-up, gene expression changes associated with several interesting molecular categories related to the stress response. Interestingly, the expression of FKBP5 was shown to be upregulated in this study. Given the proximity to trauma exposure, however, the findings may have reflected biological changes associated with recovering from the effects of trauma exposure, rather than with the development or persistence of chronic PTSD.

Indeed, genetic factors are known to be important in determining responses to environmental events and have been previously linked to PTSD, depression, schizophrenia and other mental health disorders. Duncan L E, et al., Mol Psychiatry. March 2018; 23(3):666-673; Petra Zimmermann, et. al., Am J Psychiatry. 2011 October; 168(10). For example, twin studies estimate PTSD heritability to range from 24 to 72% following trauma, suggesting that an individual's genes and epigenetics impact whether or not they experience PTSD after a trauma or stressor. Sartor C E, et al., Arch Gen Psychiatry. March 2012; 69(3):293-9; Stein M B, et al., Am J Psychiatry. October 2002; 159(10):1675-81; True W R, et al. Arch Gen Psychiatry. April 1993; 50(4):257-64; Wolf E J, et al., Psychol Med. May 2014; 44(7):1499-509. A study of Holocaust survivor families was the first to show that epigenetic changes in specific gene loci can be passed on from parents to offspring leading to an increased risk for PTSD. Rachel Yehuda et al., Society of Biological Psychiatry Sep. 1, 2016; 80:372-380. Gene expression levels which control for glucocorticoid receptor regulation were found to be dysregulated in 9/11 World Trade Center first responders with severe PTSD and have also been found altered in brains of patients with PTSD. Casey Sarapasa et al., Disease Markers 30 (2011) 101-110; and Sophie E. Holmes et al., Proc Natl Acad Sci USA. 2017 Aug. 1; 114(31):8390-8395.

It is found that over 90% of adults in the United States have experienced at least one traumatic event in their life (Breslau, N. et al., Soc Psychiatry Psychiatr Epidemiol. August 2016; 51(8):1137-48) and about 8%, or an estimated 13 million Americans, will develop posttraumatic stress disorder (PTSD). Kessler, R. C., et al., Archives of General Psychiatry, 52, 1048-1060. PTSD is a highly debilitating condition characterized by intrusive and recurrent memories of the trauma, avoidance of trauma related stimuli, numbing and/or negative changes in mood or cognitions pertaining to the trauma, and changes in reactivity and arousal.

Early treatment has the potential to reduce adverse outcomes, as PTSD may be reversible if caught early. Wlassoff V., brainblogger.com/2015/01/24/how-does-post-traumatic-stress-disorder-change-the-brain/, Accessed Nov. 19, 2021; van Wingen G A, et al., Proc Natl Acad Sci USA. Sep. 18 2012; 109(38):15508-13; Shalev A Y, Ankri Y, Israeli-Shalev Y, et al. Arch Gen Psychiatry. February 2012; 69(2):166-76; Bisson J I, Olff M. Eur J Psychotraumatol. 2021; 12(1):1824381. Several studies have found early interventions can decrease the development of chronic PTSD by as much as 50%. Rothbaum B O, et al., Biological Psychiatry. 2012; 72(11):957-963; Shalev A Y, et al., Arch Gen Psychiatry. February 2012; 69(2):166-76. Chronic PTSD has been linked to increased risk of cancers, metabolic syndrome (Cohen B E, et al., JAMA. 2009; 302(5):489-492; Weiss T, et al., General Hospital Psychiatry. 2011; 33(2):135-142) and increased inflammatory response (O'Toole B I, et al., Journal of Psychosomatic Research. 2008; 64(1):33-40.) Psychosocial impacts can include homelessness, poverty, and incarceration (APA CPG for PTSD).

However, early treatment is hindered by the fact that PTSD is underrecognized by healthcare providers (Joneydi R, et al., Med Care. Jun. 1 2021; 59(6):557-564) likely because there are no objective tests that can be ordered to aid in diagnosis. Lack of prompt diagnosis leads to delays in treatment which result in maladaptive coping and the development of disorders such as alcohol and substance use. Studies have repeatedly shown a very high rate of comorbid disorders. Kessler, R. C., et al., Archives of General Psychiatry, 52, 1048-1060, 1995; Pietrzak R H et al., Am J Geriatr Psychiatry. May 2012; 20(5):380-90; Walter K H, et al., J Trauma Stress. December 2018; 31(6):837-844; Smith S M et al., J Psychiatr Res. November 2016; 82:16-22. A meta-analysis found that 52% of people with current PTSD had co-occurring major depressive disorder, Rytwinski N K, et al., Journal of Traumatic Stress. 2013; 26(3):299-309.

The typical PTSD diagnostic journey can take months to many years with delays introduced by both patient and provider characteristics. On the patient's side, very often, patients don't associate their current experiences to a traumatic event and therefore have little insight that behavioral health care is needed. In other cases they see the connections but want to deal with it on their own; often due to the stigma that otherwise they appear “weak or not resilient”. Many coping behaviors begin at this stage that are negative and cause subsequent disruptions in their personal and occupational life which eventually prompt them to seek care from their provider.

On the provider side, PTSD and other mental health diagnoses are generally made by subspecialty trained, behavioral health providers. However, early diagnosis depends on the ability of the primary care provider considering the diagnosis in the differential and appropriate referral being made. Primary care providers generally lack the time, training and resources to do lengthy clinical interviews so they depend on self report surveys as screening tools to help identify the patients who should be referred.

There are several open-source psychometric screening tools available for use; however, they generally all suffer from low precision and also vary significantly based on the population to which they are applied. Spoont M, et al., VA Evidence-based Synthesis Program Reports, 2013. While many studies have been done to determine the precision of various tools they are often hard to compare directly as different “cut offs” are used. For example, in one study a lower cutoff may be used in order to maximize detection of PTSD cases. However, this decision is associated with more false positives. While a higher cutoff can be used to minimize false positives, at the expense of increasing false negatives.

The most frequently used self-reported screening instrument is the 17-item PTSD Checklist (PCL) (Scott D. McDonald, Patrick S. CalhounClinical Psychology Review 30 (2010) 976-987) which has been used widely for clinical screening and tracking symptoms of PTSD. Berlant, J., et al., The Journal of Clinical Psychiatry, 63, 15-20 (2002); and Blevins C A, et al., J Trauma Stress. December 2015; 28(6):489-98. The preliminary version of the PCL-5 recommended a cutoff for diagnosis of 31 to 33. Weathers F W, et al., ptsd.va.gov/professional/assessment/adult-sr/ptsd-checklist.asp, Accessed Aug. 2, 2022. In a sample of 273 trauma-exposed mental health service users, at a cutoff of 33.5, the PCL-5 had a sensitivity of 95%; however, this resulted in a specificity of 52%. Roberts N P et al., Eur J Psychotraumatol. 2021; 12(1):1863578.

In addition to precision issues, relying on self-reporting alone is problematic (Althubaiti A. J Multidiscip Healthc. 2016; 9:211-7) and the current standard of care has issues with stigma (Johnson H P et al., Psychiatr Danub. November 2018; 30(Suppl 7):508-510; Mellotte H et al., Eur J Psychotraumatol. 2017; 8(1):1389207; Wilk J E, et al., Psychiatr Serv. Aug. 1 2016; 67(8):878-82), malingering (Hall R C, Gen Hosp Psychiatry. November-December 2006; 28(6):525-35; Ali S, Jabeen S, et al., Innov Clin Neurosci. January-February 2015; 12(1-2):12-20) and underreporting (Johnson H P, Agius M., Psychiatr Danub. November 2018; 30(Suppl 7):508-510). For example, reporting bias have been well documented; patients may not be truthful for fear of public discrimination, fear of hospitalization, embarrassment, shame, or a myriad of other factors that underlie public, self, or structural stigma. Corrigan P W et al., Psychol Sci Public Interest. October 2014; 15(2):37-70.

This can be especially seen in those who have served in the military. PTSD affects US Service Members and Veterans (11-20%) disproportionately more often than their civilian counterparts (6-8%). The US Military currently uses survey questions imbedded into their pre-deployment, post-deployment and periodic health assessments as well as in the post-deployment health reassessment to identify members who should undergo further assessment by a trained clinician to make appropriate diagnoses—including diagnoses of comorbid conditions, such as depression or traumatic brain injury—and to acquire additional information that is required to plan treatment.

Example of a Primary Care PTSD Screen Used in DoD Assessments:

In your life, have you ever had any experience that was so frightening, horrible, or upsetting that, in the past month, you:
1. Have had nightmares about it or thought about it when you did not want to?YES/NO
2. Tried hard not to think about it or went out of your way to avoid situations that reminded you of it?YES/NO
3. Were constantly on guard, watchful, or easily startled?YES/NO
4. Felt numb or detached from others, activities, or your surroundings?YES/NO

Concerns about how answers may affect current or future assignments, promotions, compensation and benefits and/or security clearances often affect how these questions are answered by the respondent. Missed identification and delays in treatment can lead to maladaptive coping strategies, increased social dysfunction, and decreased medical readiness. See e.g., Sarapas C, et al. Dis Markers. 2011; 30(2-3):101-10; Le-Niculescu H, et al., Mol Psychiatry. 2020 May; 25(5):918-938; and Cer R Z, et al., Gigascience. 2014 Oct. 13; 3:20.

Indeed, one study found that PTSD diagnoses were consistently not reported in military medical records to reduce stigma or protect the service member's career prospects. Wilk J E, et al., Psychiatr Serv. Aug. 1 2016; 67(8):878-82. Another study used anonymous surveys to obtain more accurate reporting from a single infantry brigade combat team (n=1712). They found that 20.3% of soldiers who screened positive for depression or PTSD reported that they were uncomfortable reporting their answers honestly on the routine post-deployment screening. Warner C H, et al., Arch Gen Psychiatry. October 2011; 68(10):1065-71. Understandably, a disorder with symptoms like avoidance of reminders of trauma may not be reported accurately on a systemic level.

These findings also extend to studies of civilian patients. A South African study of a psychiatry inpatient unit randomly sampled 40 participants who did not previously have a diagnosis of PTSD and found that the prevalence of PTSD in the study group was 40%, up from the unit's original prevalence of 5.5%, obtained using standard of care methods. van Zyl M, et al., Afr J Psychiatry (Johannesbg). May 2008; 11(2):119-22.

This result demonstrates that individuals with other comorbid disorders may not be screened or diagnosed properly for their PTSD. Further support of this trend of underdiagnosis comes from another study of a US population of children and adolescents. Researchers interviewed patients using trauma-focused interviews at 2 sites and identified PTSD in 47.7% of patients in one site, up from their original prevalence of 2.3% (n=44), and 44.6% in a second site, up from their original prevalence of 5.4% (n=56). Miele D, O'Brien E J., Journal of Traumatic Stress. 2010; 23(5):591-598.

Less often discussed is the bias introduced by the observer. A study comparing the rates of PTSD in hospitalized male and female veterans found that only 1 woman was diagnosed with PTSD, compared to 7 men (n=62), which demonstrates that women may not be adequately diagnosed with the disorder due to bias introduced by the provider. Grossman L S, et al., Psychiatr Serv. March 1997; 48(3):393-5. In addition to the aforementioned shortcomings, these self-report tools are not widely used in primary care offices. They are most often used in high risk primary care settings such as military or veteran clinics. In other settings, an individual would have to voluntarily report their symptoms or request a referral to BH for definitive diagnosis.

Collectively, the findings noted above explain the many reasons that PTSD is woefully underdiagnosed in the current standard of care. They also highlight the profound need for objective diagnostic tools that can be more broadly applied without adding time or administrative burden to the primary care provider while earlier identifying the many individuals who otherwise would go undiagnosed and untreated.

Once the patient does arrive at a specialty care provider, diagnosis is usually made by clinical judgment after an unstructured interview, as the Clinician-Administered PTSD Scale for DSM-5 (CAPS-5) structured interview is often unpractically time consuming. Due to a shortage of these providers, there is often significant delay in receiving care even after being referred due to waiting times or failure to follow through on referrals because of social stigma.

Considering PTSD's negative impact on overall health and functioning and the availability of effective evidenced based treatments, there is a clear need for an objective test that can be used for early identification of affected individuals. Scott D. McDonald, Patrick S. CalhounClinical Psychology Review 30 (2010) 976-987; Department of Veterans Affairs, Veterans Health Administration (2004), (VHA Directive 2004-015).

Unfortunately no single treatment, even ones that have substantial evidence bases, has been demonstrated to be effective for everyone who has PTSD. Therefore the current treatment approach is often one of trial and error to find the right treatment for an individual patient. The most recent American Psychological Association Clinical Pathway Guidelines recommends several therapies to include cognitive behavioral therapy with a trauma focus (CBT-TF), cognitive processing therapy (CPT), cognitive therapy (CT), and eye movement desensitization and reprocessing (EMDR). Bisson J I, Olff M., Eur J Psychotraumatol, 2021; 12(1):1824381. However, about 33% of people in the general population who have PTSD are resistant to first and second line treatments; the non-response rates for cognitive behavioral therapy may be as high as 50% and for selective serotonin reuptake inhibitors about 20-40%. Green B. Advances in Psychiatric Treatment, 2013; 19:181-190.

An objective biomarker for PTSD is needed to meet the challenges associated with diagnosis, such as stigma, symptom overlap and observer and reporter bias. Behavioral health is the only area of medicine still lacking objective measures to aid in diagnosis and treatment monitoring. Moving forward diagnostic and treatment monitoring categories that link to the biological dysregulation underpinning the disorder are needed.

### Definitions

As used herein, the singular forms “a”, “an”, and “the” include plural references unless the context clearly dictates otherwise. Any reference to “or” herein may be intended to encompass “and/or” unless otherwise stated.

As used herein, the term “about” may mean the referenced numeric indication plus or minus: 5%, 10%, 15%, or 20% of that referenced numeric indication. In some instances, “about” may mean the referenced numeric indication plus or minus 15% of that referenced numeric indication. In some instances, “about” may mean the referenced numeric indication plus or minus 20% of that referenced numeric indication.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

As used herein, the term “adjustment disorder”, may relate to a stressor for situations in which the response to a stressor does not meet the criteria for post-traumatic stress disorder (or another specific mental disorder) and for situations in which the symptom pattern of post-traumatic stress disorder occurs in response to a stressor that is not extreme (e.g., spouse leaving, being fired). Symptoms may include avoidance, numbing, and increased arousal that are present before exposure to the stressor and may not meet criteria for the diagnosis of PTSD and require consideration of other diagnoses (e.g., brief psychotic disorder, conversion disorder, major depressive disorder), these diagnoses may be given instead of, or in addition to, PTSD.

The terms “administer,” “administering”, “administration,” and the like, as used herein, can refer to methods that can be used to enable delivery of compounds or compositions to the desired site of biological action.

As used herein “clinical diagnosis” and grammatical equivalents thereof in reference to PTSD can refer to the diagnosis of PTSD by informal or formal clinical interview with a trained behavioral health provider.

As used herein “complementarity” can refer to non-covalent bonding of nucleobases through hydrogen bonding, Watson-Crick base pairing, Wobble Base Pairing, Van Der Waals interactions, or any combination thereof.

As used herein, “control” as used herein refers to a control subject, a control population, or control data, such as from a baseline or a database.

The phrase “control gene expression levels” as used herein can refer to expression data of one or more PTSD-associated genes as derived from a baseline, a control, a control population, or datasets as described herein.

As used herein, the term “detect” and grammatical equivalents thereof describes the identification of the presence or absence of PTSD in a subject. A positive detection of PTSD, or a positive PTSD detection as interchangeably used herein, describes that PTSD is identified or detected in a subject. A negative detection of PTSD, or a negative PTSD detection as interchangeably used herein, describes that PTSD is not identified or detected in a subject. Likewise, a subject or group (e.g., a subject or a population of interest, or, a control or control group) that is negative for PTSD comprises a subject that was not detected for PTSD, such as, by methods and assays described herein, and/or by clinical diagnosis described herein.

As used herein, the term “differential expression” and grammatical equivalents thereof describes the statistically significant difference in gene expression level(s) between two experimental conditions (e.g., test group and a control group, such as a group clinically diagnosed with PTSD and a PTSD negative group). Examples of calculating differential expression are further described herein.

As used herein, the term “expression level” may relate to the degree of gene expression in terms of preprocessed or post process mRNA (before and after removal of introns) expression level of the gene. In certain instances, the term “expression level” may refer to a translated protein that is the product of the gene. Comparison or Assessment of expression levels, i.e., determining the degree of expression, may be the decisive factor in the process of detecting PTSD. Likewise, assessment of expression levels can also indicate the degree of PTSD progression in a subject. Comparison of expression levels can include arithmetically, algorithmically or mathematically calculating a differential value. In some embodiments, the differential value informs the analysis of more complex quantitative information, such as the correlation of certain PTSD-associated gene expression levels or a subset thereof, to an occurrence or likelihood of the occurrence of an outcome of interest (e.g., identification, monitoring, and/or progression of PTSD) as described herein. Such calculations

The term “fragment,” as used herein, may be a portion of a sequence, a subset that may be shorter than a full-length sequence. A fragment may be a portion of a gene. A fragment may be a portion of an oligonucleotide sequence. A fragment may be less than about: 20, 30, 40, 50 amino acids in length. A fragment may be about 10%, about 15%, about 20%, about 25%, about 30%, about 35%, about 40%, about 50%, about 60% or about 70% of the total length of an amino acid sequence or a nucleotide sequence. A fragment may be less than about: 20, 30, 40, 50 oligonucleotides in length. The term “homology” can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein.

The phrases “nucleic acid” or “nucleic acid sequence,” as used herein, can refer to an oligonucleotide, nucleotide, polynucleotide, or any fragment thereof, to DNA or RNA of genomic or synthetic origin which may be single-stranded or double-stranded and may represent the sense or the antisense strand to any DNA-like or RNA-like material. Nucleic acid molecules can be naturally occurring, recombinant, or synthetic. The term “nucleotide” may be further abbreviated as “nt”. The abbreviation “NA,” as used herein can refer to a nucleic acid.

As used herein, the term “peptide” may describe a chain of up to 30 amino acids. As used herein, the term “protein” may describe a chain of greater than 30 amino acids. Peptides and proteins may further form dimers, trimers and higher oligomers, i.e. consisting of more than one molecule which may be identical or non-identical. As used herein, the terms “peptide” and “protein” (wherein “protein” is interchangeably used with “polypeptide”) may also refer to naturally modified peptides/proteins wherein the modification is affected e.g., by glycosylation, acetylation, phosphorylation and the like.

The phrase “pharmaceutically acceptable excipient” as used herein may refer to a pharmaceutically acceptable material, composition or vehicle, such as a liquid or solid filler, diluent, excipient, carrier, solvent or encapsulating material.

As used herein, “post-traumatic stress disorder” as used herein may relate to a condition characterized by the development of characteristic symptoms following exposure to a traumatic stressor such as direct personal experience of an event that involves actual or threatened death or serious injury, or other threat to one's physical integrity; or witnessing an event that involves death, injury, or a threat to the physical integrity of another person; or learning about unexpected or violent death, serious harm, or threat of death or injury experienced by a family member or other close associate. The person's response to the event may involve intense fear, helplessness, or horror. In children, the response may involve disorganized or agitated behavior. The characteristic symptoms resulting from the exposure to the extreme trauma may include persistent reexperiencing of the traumatic event, persistent avoidance of stimuli associated with the trauma and numbing of general responsiveness, and persistent symptoms of increased arousal. In post-traumatic stress disorder, the stressor may be of extreme nature.

As used herein, the term “predisposition for a disease” is established in the art and used herein analogously.

As used herein, the term “sample” may refer to a biological sample, such as, for example, cells, tissues (from any organ including post-mortem brain tissue), or fluids (including serum, whole blood, cerebrospinal fluid, lymph, saliva, milk, pus, urine, faeces), which has been isolated or obtained from an individual or from cell culture constituents of a cell culture comprising a subject's cells. A tissue or liquid sample obtained from a patient and/or subject that comprises cells may be used for the assessment of expression levels of the one or more genes or proteins.

As used herein, the term “trauma” may refer to a direct personal experience of an event that involves actual or threatened death or serious injury, or other threat to one's physical integrity; or witnessing an event that involves death, injury, or a threat to the physical integrity of another person; or learning about unexpected or violent death, serious harm, or threat of death or injury experienced by a family member or other close associate.

The terms “treat,” “treating” or “treatment,” as used herein, may include at least partially: alleviating, abating or ameliorating a disease or condition symptom; preventing an additional symptom; ameliorating or preventing the underlying causes of symptom; inhibiting the disease or condition, e.g., at least partially arresting the development of the disease or condition; relieving the disease or condition; causing regression of the disease or condition; relieving a condition caused by the disease or condition; or stopping a symptom of the disease or condition either prophylactically, therapeutically or both. Treatment may include treatment of a condition associated with PTSD such as reduction in feelings of anxiety, depression, intense fear, helplessness, or horror, disorganized or agitated behavior, reexperiencing of the traumatic event, persistent avoidance of stimuli associated with the trauma and numbing of general responsiveness, and persistent symptoms of increased arousal. In some aspects, a method described herein may comprise administering a therapeutically effective amount of a PTSD treatment to a subject, who can be a human or animal subject, who can be a mammal.

### PTSD Molecular Detection Assay and Methods of Use Thereof

Provided herein is a PTSD molecular detection assay or a use thereof. In some embodiments, a PTSD molecular detection assay is useful in methods of detection, diagnosis, screening, patient selection, treatment, and/or monitoring as described herein. In some embodiments, a PTSD molecular detection assay and methods of using thereof as described herein can detect PTSD in a subject. In certain embodiments, the detection of PTSD in a subject as described herein can diagnose PTSD in a subject. In some embodiments, the detection of PTSD in a subject as described herein increases the likelihood that the subject is clinically diagnosed with PTSD. In some embodiments, the detection of PTSD in a subject as described herein can predict and/or establish the efficacy of a PTSD treatment in a subject.

In some embodiments, a PTSD molecular detection assay as described herein measures a test gene expression level of one or more PTSD-associated genes. In some embodiments, the one or more PTSD-associated genes are a plurality of PTSD-associated genes, such as one or more genes set forth in TABLE 1. In some embodiments, a PTSD molecular detection assay as described herein measures PTSD-associated gene expression level(s) of a subject to generate a test PTSD-associated gene expression level(s). In some embodiments, a PTSD molecular detection assay as described herein compares the one or more test PTSD-associated gene expression level(s) to a control gene expression level(s). In some embodiments, comparing the one or more test PTSD-associated gene expression level(s) to the one or more control gene expression level(s) determines the relative expression of the one or more test PTSD-associated gene expression level(s) compared to the one or more control gene expression level(s). A person of ordinary skill in the art would understand that the compared control gene(s) are those that correspond to the one or more test PTSD-associated gene(s).

### PTSD Related Genes

In some embodiments, a PTSD molecular detection assay as described herein determines the relative expression of one or more PTSD-associated gene expression level(s) of a subject (i.e., the one or more test PTSD-associated gene expression level) to one or more control gene expression level(s). In some embodiments, one or more PTSD-associated genes any be any gene that is associated with the occurrence of PTSD in a subject. In some embodiments, one or more PTSD related genes comprise one or more of TSPAN5, HIST1H2AE, UBE3A, GPX4, EPB42, SLC4A1, NDUFA1, HIST1H1E, ELOVL7, ALAS2, COMT, PDZK1IP1, ITGA2B, CYP4F3, EPB41L3, PRDM1, RP-11-449P15.1, FAS, TUBB2A, CHMP5, BOD1L1, OSM, JAM3, or a variant thereof (e.g., a splice variant), or any combination thereof. In some embodiments, one or more PTSD related genes comprise one or more of TSPAN5, HIST1H2AE, UBE3A, GPX4, EPB42, SLC4A1, NDUFA1, HIST1H1E, ELOVL7, ALAS2, COMT, PDZK1IP1, ITGA2B, CYP4F3, EPB41L3, PRDM1, RP-11-449P15.1, FAS, TUBB2A, JAM3, or a variant thereof (e.g., a splice variant), or any combination thereof.

In some embodiments, the expression level of one or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or up to all of TSPAN5, HIST1H2AE, UBE3A, GPX4, EPB42, SLC4A1, NDUFA1, HIST1H1E, ELOVL7, ALAS2, COMT, PDZK1IP1, ITGA2B, CYP4F3, EPB41L3, PRDM1, RP-11-449P15.1, FAS, TUBB2A, JAM3, or a variant thereof (e.g., a splice variant), or any combination thereof, are measured.

TABLE 1 lists the exemplary PTSD related gene mRNA transcripts in DNA form with T substituted for U.

TABLE 1
EXEMPLARY PTSD RELATED GENES

| | | NCBI Accession |
|---|---|---|
| SEQ ID NO: | Gene mRNA | Number |
| SEQ ID NO: 1 | TSPAN5 nucleic acid sequence | XM_005262680 |
| SEQ ID NO: 2 | HIST1H2AE nucleic acid sequence | AY400521 |
| SEQ ID NO: 3 | UBE3A nucleic acid sequence | KR710362 |
| SEQ ID NO: 4 | GPX4 nucleic acid sequence | NM_001367832 |
| SEQ ID NO: 5 | EPB42 nucleic acid sequence | KR711753 |
| SEQ ID NO: 6 | SLC4A1 nucleic acid sequence | KR711958 |
| SEQ ID NO: 7 | NDUFA1 nucleic acid sequence | NM_004541 |
| SEQ ID NO: 8 | HIST1H1E nucleic acid sequence | AF531302 |
| SEQ ID NO: 9 | ELOVL7 nucleic acid sequence | AB181393 |
| SEQ ID NO: 10 | ALAS2 nucleic acid sequence | KR710694 |
| SEQ ID NO: 11 | COMT nucleic acid sequence | CR456422.1 |
| SEQ ID NO: 12 | PDZK1IP1 nucleic acid sequence | KJ898169 |
| SEQ ID NO: 13 | ITGA2B nucleic acid sequence | NM_000419 |
| SEQ ID NO: 14 | CYP4F3 nucleic acid sequence | NM_000896 |
| SEQ ID NO: 15 | EPB41L3 nucleic acid sequence | KJ902290 |
| SEQ ID NO: 16 | PRDM1 nucleic acid sequence | NM_001198 |
| SEQ ID NO: 17 | RP-11-449P15.1 nucleic acid | AC073957 |
| | sequence | |
| SEQ ID NO: 18 | FAS nucleic acid sequence | KC863989 |
| SEQ ID NO: 19 | TUBB2A nucleic acid sequence | NM_001069 |
| SEQ ID NO: 20 | CHMP5 nucleic acid sequence | KJ906176.1 |
| SEQ ID NO: 21 | BOD1L1 nucleic acid sequence | NM_148894.3 |
| SEQ ID NO: 22 | JAM3 | NM_032801.5 |

In some embodiments, one or more PTSD related genes can be any one of the genes described in TABLE 1, consisting of the corresponding nucleic acid sequence set forth in TABLE 1, or a variant, such as a splice variant, thereof. In some embodiments, the expression level of one or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, or up to all PTSD related genes as set forth in TABLE 1 are measured.

In certain instances, a gene expression level may detect a portion of an RNA sequence of agene associated with PTSD to determine the level or amount of the entire RNA sequence. The portion detected with sequencing or hybridization techniques may be about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% 100% of the RNA sequence or any number in between. In certain instances, a contiguous region within the sequence is detected by sequencing or hybridization techniques. In such cases, the sequence may be about 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, or 1000 nucleotides in length or any number in between. In certain instances, multiple regions within the sequence are detected by sequencing or hybridization techniques. In such cases, about 2, 3, 4, 5, 6, 7, 8, 9, 10 or more regions within a RNA sequence can be detected.

In certain aspects of the disclosure, gene expression levels may be determined from any form of RNA such as messenger RNA (mRNA), a small interfering RNA (siRNA), a MicroRNA (miRNA), a small nuclear RNA (snRNA), a U spliceosomal RNA (U-RNA), a Small nucleolar RNA (snoRNA), a Piwi-interacting RNA (piRNA), a repeat associated small interfering RNA (rasiRNA), a small rDNA-derived RNA (srRNA), a transfer RNA derived small RNA (tsRNA), a ribosomal RNA derived small RNA (rsRNA), a large non-coding RNA derived small RNA (lncsRNA), or a messenger RNA derived small RNA (msRNA), a gapmer, a mixmer, double-stranded RNAs (dsRNA), single stranded RNAi, (ssRNAi), DNA-directed RNA interference (ddRNAi), or any combination thereof. In certain aspects, the expression level is determined by measuring the expression of pre-processed mRNA. In certain aspects, the expression level is determined by measuring the expression of nucleic acids which interfere with expression or translation of PTSD related genes.

Expression levels may be dependent on, at least roughly proportional to the number of RNA molecules or peptide or protein molecules for a given gene, thereby allowing a quantitative assessment of gene expression. Depending on the means and methods employed for said quantitative measure the determined level of gene expression may be in the form of a variety of readout parameters, e.g., the intensity of a radioactivity signal (e.g., Northern blot with a radioactive probe), of a fluorescent signal (e.g., DNA microarray) or the mass-to-charge ratio (e.g., mass spectrometry).

Accordingly, in some embodiments, the PTSD molecular detection assay and methods of use thereof comprises measuring the one or more test PTSD-associated gene expression level(s) and/or the one or more control gene expression levels. Measuring gene expression levels as provided herein include methods based on hybridization analysis of polynucleotides, methods based on sequencing of polynucleotides, and proteomics-based methods. Exemplary methods known in the art for the quantification of RNA expression in a sample include northern blotting and in situ hybridization (Parker &Barnes, Methods in Molecular Biology 106:247-283 (1999)); RNAse protection assays (Hod, Biotechniques 13:852-854 (1992)); and PCR-based methods, such as reverse transcription PCR (RT-PCR) (Weis et al, Trends in Genetics 8:263-264 (1992)). Antibodies may be employed that can recognize sequence-specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS), Other methods known in the art may be used.

### Reverse Transcription PCR (RT-PCR)

Typically, mRNA is isolated from a test sample, such as a biological sample described herein. The starting material can be total RNA isolated from a biological sample, such as cell sample obtained from a subject, a control, or population. Optionally, a baseline sample from the subject, control or population can be used as a baseline control. mRNA can be extracted from a tissue sample, e.g., from a sample that is fresh, frozen (e.g. fresh frozen), or paraffin-embedded and fixed (e.g. formalin-fixed).

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker. Lab Invest. 56:A67 (1987), and De Andres et al, BioTechniques 18:42044 (1995), in particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, WI), and Paraffin Block RNA isolation Kit (Ambion, Inc.), Total RNA from tissue samples can be isolated using RNA Stat-60 (Tel-Test), including, for example, by cesium chloride density gradient centrifugation,

The sample containing the RNA is then subjected to reverse transcription to produce cDNA from the RNA template, followed by exponential amplification in a PCR reaction. The two most commonly used reverse transcriptases are avian myeloblastosis virus reverse transcriptase (AMV-RT) and moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction.

PCR-based methods use a thermostable DNA-dependent DNA polymerase, such as a Taq DNA polymerase. For example, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction product. A third oligonucleotide, or probe, can be designed to facilitate detection of a nucleotide sequence of the amplicon located between the hybridization sites the two PCR primers. The probe can be detectably labeled, e.g., with a reporter dye, and can further be provided with both a fluorescent dye, and a quencher fluorescent dye, as in a Taqman® probe configuration. Where a Taqman® probe is used, during the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, high-throughput piatforras such as the ABi PRISM 7700 Sequence Detection System® (Perkin-Elmer-Applied Biosystems, Foster City, CA, USA), or Lightcycler (Roche Molecular Biochemicals, Mannheim, Germany), in some embodiments, the procedure is run on a LightCycler® 480 (Roche Diagnostics) real-time PCR system, which is a microwell plate-based cycler platform.

5-Nuclease assay data are commonly initially expressed as a threshold cycle (Ct), Fluorescence values are recorded during every cycle and represent the amount of product amplified to that point in the amplification reaction. The threshold cycle (Ct) is generally described as the point when the fluorescent signal is first recorded as statistically significant. Alternatively, data may be expressed as a crossing point (Cp). The Cp value is calculated by determining the second derivatives of entire qPCR amplification curves and their maximum value. The Cp value represents the cycle at which the increase of fluorescence is highest and where the logarithmic phase of a PCR begins.

Real time PCR is compatible both with quantitative competitive PCR, where an internal competitor for each target sequence is used for normalization, and with quantitative comparative PCR using a normalization gene contained within the sample, or a housekeeping gene for RT-PCR. For further details see, e.g. Held et al, Genome Research 6:986-994 (1996).

The steps of a protocol suitable for use in the methods of the present disclosure use fixed, paraffin-embedded tissues as the RNA source. For example, mRNA isolation, purification, primer extension and amplification can be performed according to methods available in the art. (see, e.g., Godfrey et ai. J. Molec, Diagnostics 2: 84-91 (2000); Speeht et al., Am. J, Pathol. 158: 419-29 (2001)), Briefly, a representative process starts with cutting about 10 μm thick sections of paraffin-embedded tumor tissue samples, The RNA is then extracted, and protein and DNA depleted from the RNA-containing sample. After analysis of the RNA concentration, RNA is reverse transcribed using gene-specific primers followed by RT-PCR to provide for cDNA amplification products.

### Design of Intron-Based PCR Primers and Probes

PCR primers and probes can be designed based upon exon or intron sequences present in the mRNA transcript of the gene of interest. Primer/probe design can be performed using publicly available software, such as the DNA BLAT software developed by Kent, W J., Genome Res. 12(4):656-64 (2002), or by the BLAST software including its variations.

Where necessary or desired, repetitive sequences of the target sequence can be masked to mitigate non-specific signals. Exemplary tools to accomplish this include the Repeat Masker program available on-line through the Baylor College of Medicine, which screens DNA sequences against a library of repetitive elements and returns a query sequence in which the repetitive elements are masked. The masked intron sequences cars then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. See S. Rrawetz, S. Misener, Bioinformaiics Methods and Protocols: Methods in Molecular Biology, pp. 365-386 (Humana Press),

Other factors that can influence PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases, and exhibit Tm's between 5° and 80° C., e.g. about 50 to 70° C.

For further guidelines for PCR primer and probe design see, e.g. Dieffenbach, C W. et al, “General Concepts for PCR Primer Design” in: PCR Primer, A Laboratory Manual, Cold Spring Harbor Laboratory Press, New York, 1995, pp. 133-155; Tunis and Geifand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol 70:520-527 (1997), the entire disclosures of which are hereby expressly incorporated by reference.

### Mass ARRAY® System

In MassARRAY-based methods, such as the exemplary method developed by Sequenom, Inc. (San Diego, CA) following the isolation of RNA and reverse transcription, the obtained cDNA is spiked with a synthetic DNA molecule (competitor), which matches the targeted cDNA region in ail positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proe. Natl. Acad. Sci. USA 100:3059-3064 (2003).

### Other PCR-based Methods

Further PCR-based techniques that can find use in the methods disclosed herein include, for example, Bead Array® technology (Ilumina, San Diego, CA; Oliphant et al, Discover)’ of Markers for Disease (Supplement to Biotechniques), June 2002; Ferguson et al., Analytical Chemistry 72:5618 (2000)); BeadsArray for Detection of Gene Expression® (BADGE), using the commercially available LuminexlGO LabMAP® system and multiple color-coded microspheres (Luminex Corp., Austin, TX) in a rapid assay for gene expression (Yang et al, Genome Res. 3 1: 1888-1 898 (2001)); and high coverage expression profiling (HiCEP) analysis (Fukumura et al., Nucl. Acids. Res. 31(16) e94 (2003).

### Microarrays

Expression levels of a gene or microArray of interest can also be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g. formalin-fixed) tissue samples.

For example, PCR amplified inserts of cDNA clones of a gene to be assayed are applied to a substrate in a dense array. Usually at least 10,000 nucleotide sequences are applied to the substrate. For example, the microarrayed genes, immobilized on the microchip at 10,000 elements each, are suitable for hybridization under stringent conditions.

Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After washing under stringent conditions to remove non-specifically bound probes, the chip is scanned by confocal laser microscopy or by another detection method, such as a CCD camera. Quantitation of hybridization of each arrayed element allows for assessment of corresponding RNA abundance.

With dual color fluorescence, separately labeled cDNA probes generated from two sources of RNA are hybridized pair wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et at, Proc. Natl. Acad. ScL USA 93(2): 106-149 (1996)).

Microarray analysis can be performed by commercially available equipment, following manufacturer's protocols, such as by using the AlTymctrix GenChip® technology, or Incyte's microarray technology.

### Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculeseu et al, Science 270:484-487 (1995); and Velculeseu et al, Cell 88:243-51 (1997).

### Gene Expression Analysis by Nucleic Acid Sequencing

Nucleic acid sequencing technologies are suitable methods for analysis of gene expression. The principle underlying these methods is that the number of times a cDNA sequence is detected in a sample is directly related to the relative expression of the RNA corresponding to that sequence. These methods are sometimes referred to by the term Digital Gene Expression (DGE) to reflect the discrete numeric property of the resulting data. Early methods applying this principle were Serial Analysis of Gene Expression (SAGE) and Massively Parallel Signature Sequencing (MPSS), See, e.g., S. Brenner, et al, Nature Biotechnology 18(6):630-634 (2000). More recently, the advent of “next-generation” sequencing technologies has made DGE simpler, higher throughput, and more affordable. As a result, more laboratories are able to utilize DGE to screen the expression of more genes in more individual patient samples than previously possible. See, e.g., J. Marioni, Genome Research 18(9): 1509-1517 (2008); R. Morin, Genome Research 18(4):610-621 (2008); A, Mortazavi, Nature Methods 5(7):621-628 (2008); N, Cloonan, Nature Methods 5(7):613˜6!9 (2008).

Methods of isolating RNA for expression analysis from blood, plasma and serum (see, e.g., Enders, et al., Clin Chem 48.1647-53 (2002) (and references cited therein) and from urine (see, e.g., R, Boom, et al, J Clin Microbiol. 28, 495-503 (1990) and references cited therein) have been described.

### Immunohistochemistry

Immunohistochemistry methods are also suitable for detecting the expression levels of genes and applied to the method disclosed herein. Antibodies (e.g., monoclonal antibodies) that specifically bind a gene product of a gene of interest can be used in such methods. The antibodies can be detected by direct labeling of the antibodies themselves, for example, with radioactive labels, fluorescent labels, hapten' labels such as, biotin, or an enzyme such as horse radish peroxidase, or alkaline phosphatase. Alternatively, unlabeled primary antibody can be used in conjunction with a labeled secondary antibody specific for the primary antibody. Immunohistochemistry protocols and kits are well known in the art and are commercially available.

### Proteomics

The proteome is the totality of the proteins present in a sample (e.g. tissue, organism, or ceil culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics.

### Control Gene Expression Level(s)

In some embodiments, the one or more control gene expression level(s) is determined, for example by using the PTSD molecular detection assay and/or methods of use thereof as described herein. In some embodiments, the one or more control gene expression level(s) is predetermined, for example through the use of the PTSD molecular detection assay and/or methods of use thereof as described herein, or from a pre-existing database.

In some embodiments, a control gene expression level(s) is derived from a baseline control, a control, or a control population.

A control or a control population can be any appropriate comparator control or control population. In some embodiments, a control or a control population does not have PTSD. In some embodiments, a control or control population tests negative for PTSD, e.g., by clinical diagnostic methods such as those described herein. In some embodiments, a control or a control population has been exposed to a trauma, but did not develop PTSD, for example within 6 months from exposure to the trauma. In such embodiments where a control or a control population is not afflicted with PTSD, such a control or a control population can be described as a negative control or a negative control population herein.

In some embodiments, a control or a control population is afflicted with PTSD. In some embodiments, a control or a control population is afflicted with PTSD but is not receiving PTSD treatment. In some embodiments, a control or a control population is afflicted with PTSD and is receiving PTSD treatment. In such embodiments where a control or a control population is afflicted with PTSD, such a control or a control population can be described as a positive control or positive control population herein.

In some embodiments, a control or a control population is obtained from database entries, which can be negative or positive controls based on the database entries selected.

In certain instances, a control is selected from i) a control that has been exposed to a trauma, but did not develop PTSD, ii) a group of subjects negative for PTSD and representative for the investigated population and iii) database entries. Alternatively, the range of expression levels obtained from a group of subjects negative for PTSD and representative for the investigated population may be used as normal values. Such normal values can be used to normalize data collected from subjects when performing methods as described herein. A person of ordinary skill in the art would understand how to select an appropriate control depending on the methods performed, the data to be captured, the information being analyzed, and the like.

In certain instances, a sample of a control subject may be assessed more than once or that several samples of said control subject are obtained in order to increase the reliability of the data relating to the expression level. The data may further be pooled to calculate the mean or median and optionally the variance for each control subject. In certain instances, the expression level(s) are compared to the expression level(s) of corresponding gene(s) in samples of more than one control subject such as at least 2, 10, 20 or more control subjects. In certain instances, the expression levels of the samples of the control subjects may be pooled and the mean or median and optionally the variance is calculated. In such embodiments, said mean or median expression levels may be referred to herein as the mean expression level or median expression level. These values may, e.g., be deposited into a database as a standardized value for each gene and if required retrieved from a database, hence making the need to also experimentally assess the expression levels in a control sample every time the expression level in a patient sample is assessed dispensable. Accordingly, a control may also be a database entry. Moreover, by using the variance of the expression level of the control sample, the statistical significance of deviations from the mean of controls in the sample to be assessed may be determined. Finally, and where deemed appropriate, age- or gender-specific controls may be used.

In control samples from a database of subjects without PTSD, RNA or protein levels are expressed at certain ratios contributing in concerted action to normal body functions. Disturbances, such as an upregulation and/or downregulation of one or more genes, in gene expression levels may lead to a medical condition if endogenous rescue mechanisms do not exist that can compensate for said disturbances. Several steps in the gene expression process may be modulated to regulate the expression level of a gene such as, e.g. the step of initiating the transcription process involving the amount of transcription factors, the presence of or enhancer/inhibitory sequences, the step of translation involving the half-life of the mRNA to be translated, the posttranslational modification of a protein having an effect on the half-life, the secretion, the folding of the latter. Gene regulation gives cells control over structure and function and is the basis for events like differentiation, morphogenesis or adaptability of a cell or a multicellular organism.

In some embodiments, a control subject or control population that test negative for PTSD can be determined by one or more of clinical diagnostic classification schemes such as those described herein. PTSD clinical diagnostic classification schemes include: Clinician-Administered PTSD Scale for DSM-5 (CAPS-5), PTSD Checklist for DSM-5 (PCL-5), or both. Likewise, a control subject or control population that tests negative for PTSD may also test negative for one or more PTSD co-morbidities as determined by one or more PTSD co-morbidities clinical diagnostic classification schemes. Such PTSD co-morbidities clinical diagnostic classification schemes include Generalized Anxiety Disorder 7-item Scale (GAD-7), Alcohol Use Disorders Identification Test (AUDIT-C), Pittsburgh Sleep Quality Index (PSQI), Adverse Childhood Experiences (ACE), or any combination thereof.

### Clinician-Administered PTSD Scale for DSM-5 (CAPS-5)

The Clinician-Administered PTSD Scale for DSM-5 (CAPS-5) is a structured interview and is considered the gold standard for assessing trauma severity and diagnosing PTSD. This interview is conducted by a trained clinician, covering all DSM-5 criteria for PTSD. It assesses the frequency and intensity of various PTSD symptoms, as well as their impact on daily functioning.

To score the structured interview, the assessor combines information about frequency and intensity of an item into a single severity rating. CAPS-5 total symptom severity score is calculated by summing severity scores for the 20 DSM-5 PTSD symptoms. Similarly, CAPS-5 symptom cluster severity scores are calculated by summing the individual item severity scores for symptoms corresponding to a given DSM-5 cluster: Criterion B (items 1-5); Criterion C (items 6-7); Criterion D (items 8-14); and Criterion E (items 15-20). A symptom cluster score may also be calculated for dissociation by summing items 19 and 20.

For a diagnosis of PTSD to be made, patient must have: at least one Criterion B symptom; at least one Criterion C symptom; at least two Criterion D symptoms; at least two Criterion E symptoms; Criterion F is met (disturbance has lasted one month); and Criterion G is met (disturbance causes either clinically significant distress or functional impairment).

### PTSD Checklist for DSM-5

PTSD Checklist for DSM-5 or PCL-5 is a self-report questionnaire specifically designed to assess symptoms of PTSD based on the criteria outlined in the DSM-5. It is widely used in both clinical and research settings. It takes approximately 5-10 minutes to complete.

The PCL-5 consists of 20 items that measure the severity and frequency of PTSD symptoms across four symptom clusters: intrusion, avoidance, negative alterations in cognitions and mood, and hyperarousal. Individuals who have experienced a traumatic event are asked to rate the extent to which they have been bothered by each symptom in the past month, using a scale ranging from 0 (not at all) to 4 (extremely).

Interpretation of the PCL-5 should be made by a clinician. The PCL-5 can be scored in different ways:
A total symptom severity score (range—0-80) can be obtained by summing the scores for each of the 20 items.
DSM-5 symptom cluster severity scores can be obtained by summing the scores for the items within a given cluster, i.e., cluster B (items 1-5), cluster C (items 6-7), cluster D (items 8-14), and cluster E (items 15-20).
A provisional PTSD diagnosis can be made by treating each item rated as 2=“Moderately” or higher as a symptom endorsed, then following the DSM-5 diagnostic rule which requires at least: 1 B item (questions 1-5), 1 C item (questions 6-7), 2 D items (questions 8-14), 2 E items (questions 15-20).

By summing the item scores, an overall severity score for PTSD symptoms can be obtained. The PCL-5 provides a quantitative measure of PTSD symptomatology and can assist in the assessment, monitoring, and diagnosis of PTSD. It is not intended to provide a formal diagnosis on its own but can be used as a screening tool or as part of a comprehensive evaluation conducted by a healthcare professional.

Generalized Anxiety Disorder 7-item Scale

The Generalized Anxiety Disorder 7-item Scale (GAD-7) is a self-report screening tool for generalized anxiety disorder. The GAD-7 score is calculated by assigning scores of 0, 1, 2, and 3, to the response categories of ‘not at all’, ‘several days’, ‘more than half the days’, and ‘nearly every day’, respectively, and adding together the scores for the seven questions.

Scores of 5, 10, and 15 are taken as the cut-off points for mild, moderate and severe anxiety, respectively. When used as a screening tool, further evaluation is recommended when the score is 10 or greater. Using the threshold score of 10, the GAD-7 has a sensitivity of 89% and a specificity of 82% for GAD. It is moderately good at screening three other common anxiety disorders-panic disorder (sensitivity 74%, specificity 81%), social anxiety disorder (sensitivity 72%, specificity 80%) and post-traumatic stress disorder (sensitivity 66%, specificity 81%). Wilk J E, Herrell R K, Carr A L, West J C, Wise J, Hoge C W. Diagnosis of PTSD by Army Behavioral Health Clinicians: Are Diagnoses Recorded in Electronic Health Records?Psychiatr Serv. Aug. 1 2016; 67(8):878-82. doi:10.1176/appi.ps.201500292.

### Alcohol Use Disorders Identification Test

The Alcohol Use Disorders Identification Test (AUDIT-C) is an alcohol screen that can help identify patients who are hazardous drinkers or have active alcohol use disorders (including alcohol abuse or dependence). The AUDIT-C is scored on a scale of 0-12 (scores of 0 reflect no alcohol use). In men, a score of 4 or more is considered positive; in women, a score of 3 or more is considered positive. Generally, the higher the AUDIT-C score, the more likely it is that the patient's drinking is affecting his/her health and safety.

### Pittsburgh Sleep Quality Index

The Pittsburgh Sleep Quality Index (PSQI) is a self-rated questionnaire which assesses sleep quality and disturbances over a 1-month time interval. Consisting of 19 items, the PSQI measures several different aspects of sleep, offering seven component scores and one composite score. The component scores consist of subjective sleep quality, sleep latency (i.e., how long it takes to fall asleep), sleep duration, habitual sleep efficiency (i.e., the percentage of time in bed that one is asleep), sleep disturbances, use of sleeping medication, and daytime dysfunction.

Each item is weighted on a 0-3 interval scale. The global PSQI score is then calculated by totaling the seven component scores, providing an overall score ranging from 0 to 21, where lower scores denote a healthier sleep quality.

### Adverse Childhood Experiences (ACE)

The Adverse Childhood Experiences Questionnaire (ACE-Q) is a 10-item measure to quantify instances of adverse or traumatic experiences that the client has had before the age of 18. The ACE-Q checks for the client's exposure to childhood psychological, physical, and sexual abuse as well as household dysfunction including domestic violence, substance use, and incarceration. The ACE-Q was used in the Adverse Childhood Experiences (ACE) Study (Felitti et al., 1998), which found that the ACE-Q score is correlated with later life mental health challenges as well as health risk behaviors (including substance abuse) and serious health problems. These include increased risk for depression, suicide attempts, alcoholism, drug abuse, smoking, 50 or more sexual partners, physical inactivity, severe obesity, sexually transmitted disease, increased risk for broken bones, heart disease, lung disease, liver disease, and multiple types of cancer (Felitti et al., 1998). Each positive response is summed to provide an overall ACE-Q score (out of 10). Scores of 4 or more are considered clinically significant. A minority (5%-10%) of the general population score 4 or more, where the general long-term health consequences become most pronounced (Hughes, K., et. al. (2017). Lancet Public Health, 2(8), e356-e366).

### Differential Expression

As described herein, in some embodiments, the PTSD molecular detection assay and/or methods of use thereof comprise determining differential expression of the one or more test PTSD-associated gene(s) compared to the one or more control gene(s) and determining the detection of PTSD based on the differential expression. In some embodiments, the PTSD molecular detection assay and methods of use thereof measures one or more test PTSD-associated gene expression level(s) in a sample obtained from a subject and compares the one or more test PTSD-associated gene expression level(s) to one or more control gene expression level(s) to determine differential expression of the one or more test PTSD-associated gene(s); and determining the positive detection of PTSD based on the differential expression.

Any suitable method of determining differential gene expression can be utilized in the context of the present disclosure. In some embodiments, differential expression can be calculated by normalizing expression data between two experimental conditions (e.g., a group clinically diagnosed with PTSD and a PTSD negative group) and identifying statistically significant genes correlated with the condition (e.g., PTSD). In some embodiments, differential expression can be calculated by gene expression ranking, which involves rank ordering of normalized expression data between two experimental conditions (e.g., a group clinically diagnosed with PTSD and a PTSD negative group) and identifying statistically significant genes correlated with the condition (e.g., PTSD).

Examples of normalization methods suitable with the present disclosure include, but are not limited to, global normalization, Lowess normalization, trimmed mean method (TMM), quantile normalization, scaling normalization, variance stabilization (VSN) and invariant method (IN). In some embodiments, normalization of gene expression can be conducted relative to a set of control genes, including a database or a predetermined discrete set of genes.

In some embodiments, gene expression levels can be normalized using Normal Quantile Transformation (NQT). In some embodiments, gene expression levels can be normalized using NQT as follows: random numbers are drawn from the normal distribution based on the number of observations (e.g., the number of mRNA species) and sorted based on Equation 1. Then quantiles are assigned according to each rank using Equations 2-3. All the equations included herein are R language commands unless or otherwise specified.

$$rn = sort ( rnorm ( no\_of \_mRNAs ) ) \qquad Equation 1$$

$$qt = rank ( x ) / length ( x ) \qquad Equation 2$$

$$nqt = rn [ rank ( qt ) ] \qquad Equation 3$$

In some embodiments, upon normalizing gene expression level(s), differentially expressed genes correlated with PTSD can be identified. Any suitable method of identifying differentially expressed genes can be used in the context of the present disclosure. Examples include a regression model, such as a linear regression model or a logistic regression model. For example, using a regression model (e.g., linear or logistic), quantile transformed data can be analyzed to identify any mRNA species which display either increasing or decreasing expression. Using NQT data over different time points, the corresponding P-values, intercept, and slope values can be calculated using Equations 4-8, and any mRNAs with P<0.05 are considered to be statistically significant:

$$l mod = lm ( y ∼ x ) where x = time , y = NQT data \qquad Equation 4$$

$$s = summary ( l mod ) \qquad Equation 5$$

$$p = as . character ( pf ( s\$fstatistic [ 1 ] , s\$fstatistic [ 2 ] , ) ) ( s\$fstatistic [ 3 ] , lower . tail = FALSE ) \qquad Equation 6$$

$$intercept = s\$coefficients [ 1 ] \qquad Equation 7$$

$$slope = s\$coefficients [ 2 ] \qquad Equation 8$$

A discussion of the above analysis can be seen in (Cer R Z, Herrera-Galeano J E, Anderson J J, Bishop-Lilly K A, Mokashi V P. miRNA Temporal Analyzer (mimaTA): a bioinformatics tool for identifying differentially expressed microRNAs in temporal studies using normal quantile transformation. Gigascience. 2014 Oct. 13; 3:20. doi: 10.1186/2047-217X-3-20. PMID: 25379175; PMCID: PMC4212236.), the entirety of which is incorporated by reference herein.

In some embodiments, one or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20, up to all, test PTSD-associated gene(s) is differentially expressed with a p Value of less than 0.05, 0.01, 5×10⁻³, 1×10⁻³, 5×10⁻⁴, 1×10⁻⁴, 1×10⁻⁵, 9.9×10⁻⁶, 9.8×10⁻⁶, 9.7×10⁻⁶, 9.6×10⁻⁶, 9.5×10⁻⁶, 9.4×10⁻⁶, 9.3×10⁻⁶, 9.2×10⁻⁶, 9.1×10⁻⁶, 9×10⁻⁶, 8.9×10⁻⁶, 8.8×10⁻⁶, 8.7×10⁻⁶, 8.6×10⁻⁶, 8.5×10⁻⁶, 8.4×10⁻⁶, 8.3×10⁻⁶, 8.2×10⁻⁶, 8.1×10⁻⁶, 8×10⁻⁶, 7.9×10⁻⁶, 7.8×10⁻⁶, 7.7×10⁻⁶, 7.6×10⁻⁶, 7.5×10⁻⁶, 7.4×10⁻⁶, 7.3×10⁻⁶, 7.2×10⁻⁶, 7.1×10⁻⁶, 7×10⁻⁶, 6.9×10⁻⁶, 6.8×10⁻⁶, 6.7×10⁻⁶, 6.6×10⁻⁶, 6.5×10⁻⁶, 6.4×10⁻⁶, 6.3×10⁻⁶, 6.2×10⁻⁶, 6.1×10⁻⁶, 6×10⁻⁶, 5.9×10⁻⁶, 5.8×10⁻⁶, 5.7×10⁻⁶, 5.6×10⁻⁶, 5.5×10⁻⁶, 5.4×10⁻⁶, 5.3×10⁻⁶, 5.2×10⁻⁶, 5.1×10⁻⁶, 5×10⁻⁶, 4.9×10⁻⁶, 4.8×10⁻⁶, 4.7×10⁻⁶, 4.6×10⁻⁶, 4.5×10⁻⁶, 4.4×10⁻⁶, 4.3×10⁻⁶, 4.2×10⁻⁶, 4.1×10⁻⁶, 4×10⁻⁶, 3.9×10⁻⁶, 3.8×10⁻⁶, 3.7×10⁻⁶, 3.6×10⁻⁶, 3.5×10⁻⁶, 3.4×10⁻⁶, 3.3×10⁻⁶, 3.2×10⁻⁶, 3.1×10⁻⁶, 3×10⁻⁶, 2.9×10⁻⁶, 2.8×10⁻⁶, 2.7×10⁻⁶, 2.6×10⁻⁶, 2.5×10⁻⁶, 2.4×10⁻⁶, 2.3×10⁻⁶, 2.2×10⁻⁶, 2.1×10⁻⁶, 2×10⁻⁶, 1.9×10⁻⁶, 1.8×10⁻⁶, 1.7×10⁻⁶, 1.6×10⁻⁶, 1.5×10⁻⁶, 1.4×10⁻⁶, 1.3×10⁻⁶, 1.2×10⁻⁶, 1.1×10⁻⁶, 1×10⁻⁶, 9.9×10⁻⁷, 9.8×10⁻⁷, 9.7×10⁻⁷, 9.6×10⁻⁷, 9.5×10⁻⁷, 9.4×10⁻⁷, 9.3×10⁻⁷, 9.2×10⁻⁷, 9.1×10⁻⁷, 9×10⁻⁷, 8.9×10⁻⁷, 8.8×10⁻⁷, 8.7×10⁻⁷, 8.6×10⁻⁷, 8.5×10⁻⁷, 8.4×10⁻⁷, 8.3×10⁻⁷, 8.2×10⁻⁷, 8.1×10⁻⁷, 8×10⁻⁷, 7.9×10⁻⁷, 7.8×10⁻⁷, 7.7×10⁻⁷, 7.6×10⁻⁷, 7.5×10⁻⁷, 7.4×10⁻⁷, 7.3×10⁻⁷, 7.2×10⁻⁷, 7.1×10⁻⁷, 7×10⁻⁷, 6.9×10⁻⁷, 6.8×10⁻⁷, 6.7×10⁻⁷, 6.6×10⁻⁷, 6.5×10⁻⁷, 6.4×10⁻⁷, 6.3×10⁻⁷, 6.2×10⁻⁷, 6.1×10⁻⁷, 6×10⁻⁷, 5.9×10⁻⁷, 5.8×10⁻⁷, 5.7×10⁻⁷, 5.6×10⁻⁷, 5.5×10⁻⁷, 5.4×10⁻⁷, 5.3×10⁻⁷, 5.2×10⁻⁷, 5.1×10⁻⁷, 5×10⁻⁷, 4.9×10⁻⁷, 4.8×10⁻⁷, 4.7×10⁻⁷, 4.6×10⁻⁷, 4.5×10⁻⁷, 4.4×10⁻⁷, 4.3×10⁻⁷, 4.2×10⁻⁷, 4.1×10⁻⁷, 4×10⁻⁷, 3.9×10⁻⁷, 3.8×10⁻⁷, 3.7×10⁻⁷, 3.6×10⁻⁷, 3.5×10⁻⁷, 3.4×10⁻⁷, 3.3×10⁻⁷, 3.2×10⁻⁷, 3.1×10⁻⁷, 3×10⁻⁷, 2.9×10⁻⁷, 2.8×10⁻⁷, 2.7×10⁻⁷, 2.6×10⁻⁷, 2.5×10⁻⁷, 2.4×10⁻⁷, 2.3×10⁻⁷, 2.2×10⁻⁷, 2.1×10⁻⁷, 2×10⁻⁷, 1.9×10⁻⁷, 1.8×10⁻⁷, 1.7×10⁻⁷, 1.6×10⁻⁷, 1.5×10⁻⁷, 1.4×10⁻⁷, 1.3×10⁻⁷, 1.2×10⁻⁷, 1.1×10⁻⁷, or 1×10⁻⁷.

In some embodiments, one or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20, up to all, test PTSD-associated gene(s) is differentially expressed with a p Value of less than 2.5×10⁻⁶, less than 9×10⁻⁷, or less than 1.6×10⁻⁷.

In some embodiments, determining the differential expression of the one or more test PTSD-associated gene(s) can establish if there is a significant relationship between the one or more test PTSD-associated gene(s) and the detection of PTSD. One skilled in the art will recognize that there are many statistical methods that may be used to determine whether there is a significant relationship between an outcome of interest (e.g., detection of PTSD) and expression levels of a marker gene as described herein.

Likewise, one skilled in the art will recognize that many co-expression analysis methods now known or later developed will fall within the scope and spirit of the present disclosure. These methods may incorporate, for example, correlation coefficients, co-expression network analysis, clique analysis, etc., and based on expression data from RT-PCR, microarrays, sequencing, and other similar technologies. For example, gene expression clusters can be identified using pair-wise analysis of correlation based on Pearson or Spearman correlation coefficients. (See, e.g., Pearson K. and Lee A, Bioraetrika 2, 357 (1902); C. Spearman, Amer. J. Psychol 15:72-101 (1904); J. Myers, A. Well, Research Design and Statistical Analysis, p. 508 (2nd Ed., 2003),)

Moreover, the values from the PTSD molecular detection assay and/or methods described herein, can be measured and/or compared by an algorithm, a model and/or a classifier. Similarly the values from the PTSD molecular detection assay and/or methods described herein can be used to train an algorithm, model and/or classifier for use in the PTSD molecular detection assay and/or methods described herein. Accordingly, in some embodiments, the PTSD molecule detection assay and methods described herein comprises an algorithm, a model and/or a classifier. Without intending to be limiting, a classifier as described herein can be a logistic regression classifier, a support vector machine classifier, or other classifiers. The classifier may be any one of many methods for classification or regression known in the art. The model may be an artificial neural network, a Bayesian graphical model, a Gaussian process, a logistic regression, a support vector machine, a decision tree, a hidden Markov model, or k-nearest neighbor. K-fold cross-validation may also be used.

An ensemble of models may be used via boosting or another ensemble method. The model may be any one of many methods for classification or regression known in the art. The model may be an artificial neural network, a Bayesian graphical model, a Gaussian process, a logistic regression, a support vector machine, a decision tree, a hidden Markov model, or k-nearest neighbor. K-fold cross-validation may also be used.

In some embodiments, the accuracy, the sensitivity, the positive predictive value (PPV), the negative predictive value (NPV), the area under curve (AUC) or any combinations thereof, of the assays and methods described herein, such as the assay or method for detecting PTSD, may be greater than about 50%. In some embodiments, the accuracy of the assays and methods described herein are greater than about 50%, greater than about 55%, greater than about 60%, greater than about 65%, greater than about 70%, greater than about 75%, greater than about 80%, greater than about 85%, greater than about 90%, greater than about 95%, greater than about 97%, greater than about 98%, or greater than about 99% accuracy. In some embodiments, the sensitivity of the assays and methods described herein are greater than about 50%, greater than about 55%, greater than about 60%, greater than about 65%, greater than about 70%, greater than about 75%, greater than about 80%, greater than about 85%, greater than about 90%, greater than about 95%, greater than about 97%, greater than about 98%, or greater than about 99% sensitivity. In some embodiments, the PPV of the assays and methods described herein are greater than about 50%, greater than about 55%, greater than about 60%, greater than about 65%, greater than about 70%, greater than about 75%, greater than about 80%, greater than about 85%, greater than about 90%, greater than about 95%, greater than about 97%, greater than about 98%, or greater than about 99% PPV. In some embodiments, the NPV of the assays and methods described herein are greater than about 50%, greater than about 55%, greater than about 60%, greater than about 65%, greater than about 70%, greater than about 75%, greater than about 80%, greater than about 85%, greater than about 90%, greater than about 95%, greater than about 97%, greater than about 98%, or greater than about 99% NPV. In some embodiments, the AUC of the assays and methods described herein are greater than about 50%, greater than about 55%, greater than about 60%, greater than about 65%, greater than about 70%, greater than about 75%, greater than about 80%, greater than about 85%, greater than about 90%, greater than about 95%, greater than about 97%, greater than about 98%, or greater than about 99% AUC. Methods of calculating the accuracy, the sensitivity, the positive predictive value (PPV), the negative predictive value (NPV), and the area under curve (AUC) are standard to an ordinary person in the art.

In some embodiments, determining the differential expression of the one or more test PTSD-associated gene(s) can be used to: predict the likelihood that PTSD is identified and/or detected in a subject; identify and/or detect PTSD in a subject; predict the likelihood that a subject will be clinically diagnosed with PTSD; diagnose a subject with PTSD; predict the likelihood that a subject would respond to a PTSD treatment; establish the efficacy of PTSD treatment in a subject; or any combination thereof.

Accordingly, the PTSD molecular detection assay and associated information provided by the practice of the methods of the present disclosure facilitate diagnostic and treatment making decisions in diagnosis of PTSD, patient selection for the treatment of PTSD, and monitoring the efficacy of a PTSD treatment. For example, such a molecular detection assay would enable primary care providers to identify patients who have a high likelihood of having PTSD, thereby prompting the primary care provider to refer the patient to a specialist who can clinically diagnose PTSD in said patient. Likewise, such a molecular detection assay would enable PTSD treatment specialists to identify patients who have a high likelihood of responding to PTSD treatment. Moreover, such a molecular detection assay would enable PTSD treatment specialists to monitor patients having PTSD treatment by evaluating the efficacy of PTSD treatment.

Thus, in some embodiments, provided herein are methods of detecting PTSD in a subject. In some embodiments, a method of detecting PTSD in a subject comprises: obtaining a biological sample (e.g., a cell sample) from a subject, analyzing the sample using the PTSD molecular detection assay described herein to determine differential expression of one or more test PTSD-associated gene expression level(s), and determining the detection of PTSD based on the differential expression of the one or more test PTSD-associated gene(s) compared to the one or more control gene expression level(s).

In some embodiments, a method of detecting PTSD in a subject comprises: obtaining a biological sample (e.g., a cell sample) from a subject; measuring one or more test PTSD-associated gene expression level(s) in the sample; comparing the one or more test PTSD-associated gene expression level(s) to one or more control gene expression level(s); determining the differential expression of the one or more test PTSD-associated gene(s); and determining the positive detection of PTSD based on the differential expression.

In some embodiments, a method of detecting PTSD in a subject comprises: obtaining a biological sample (e.g., a cell sample) from a subject; measuring with a first computer program, executed on a computer, one or more test PTSD-associated gene expression level(s) in the sample; with a second computer program, executed on a computer, comparing the one or more test PTSD-associated gene expression level(s) to one or more control gene expression level(s); determining the differential expression of the one or more test PTSD-associated gene(s); and determining the positive detection of PTSD based on the differential expression.

In some embodiments, the one or more control gene expression level(s) is derived from a control or a control population. In some embodiments, the control or a control population is a negative control or control population. In some embodiments, the one or more control gene expression level(s) is derived from a database. In some embodiments, the first computer program and the second computer program are the same. In some embodiments, the first computer program and the second computer program are different.

In certain embodiments, the detection of PTSD in the subject by methods and assay described herein can diagnose or indicate a diagnosis of PTSD in a subject. In some embodiments, the diagnosis of PTSD by methods, systems, assays, devices, kits described herein can be a laboratory diagnosis of PTSD, which is distinct from, and/or supplemental to the clinical diagnosis of PTSD. In some embodiments, the diagnosis of PTSD by methods, systems, assays, devices, kits described herein can replace the clinical diagnosis of PTSD.

In some embodiments, methods of detecting described herein further comprise determining said subject should be clinically diagnosed with PTSD. In some embodiments, methods of detecting described herein further comprise clinically diagnosing said subject with PTSD. Clinically diagnosing a subject with PTSD comprises a PTSD diagnosis by informal or formal clinical interview with a trained behavioral health provider. Formal clinical interviews include published PTSD clinical diagnostic criteria comprising Clinician-Administered PTSD Scale for DSM-5 (CAPS-5), PTSD Checklist for DSM-5 (PCL-5), both of which are described in further detail below.

In still further embodiments, provided herein are methods of screening PTSD in a subject. In some embodiments, a method for screening PTSD in a subject comprises: detecting PTSD in a subject as described herein, determining said subject should undergo evaluation to be clinically diagnosed for PTSD based on the positive detection of PTSD in the subject.

In some embodiments, a method of screening PTSD comprises: obtaining a biological sample (e.g., a cell sample) from a subject; measuring one or more test PTSD-associated gene expression level(s); comparing the one or more test PTSD-associated gene expression level(s) to one or more control gene expression level(s); determining the differential expression of the one or more test PTSD-associated gene(s); determining the positive detection of PTSD based on the differential expression; and determining said subject should be clinically evaluated for PTSD based on the positive detection of PTSD in the subject.

In some embodiments, a method of screening PTSD comprises: obtaining a biological sample (e.g., a cell sample) from a subject; measuring with a first computer program, executed on a computer, one or more test PTSD-associated gene expression level(s); with a second computer program, executed on a computer, comparing the one or more test PTSD-associated gene expression level(s) to one or more control gene expression level(s); determining the differential expression of the one or more test PTSD-associated gene(s); determining the positive detection of PTSD based on the differential expression; and determining said subject should be clinically evaluated for PTSD based on the positive detection of PTSD in the subject.

In some embodiments, the one or more control gene expression level(s) in methods for screening for PTSD is derived from a control or a control population. In some embodiments, the control or a control population is a negative control or negative control population. In some embodiments, methods for screening for PTSD further comprises clinically diagnosing said subject with PTSD using one or more informal or formal interviews. In some embodiments, the formal interviews comprise the use of PTSD clinical diagnostic criteria. In some embodiments, the one or more PTSD clinical diagnostic criteria comprises CAPS-5 or PCL-5. In some embodiments, the PTSD clinical diagnostic criteria is CAPS-5. In some embodiments, the PTSD clinical diagnostic criteria is PCL-5. In some embodiments, the detection of PTSD as described herein and clinically diagnosing PTSD in a subject is performed by the same entity. In some embodiments, the detection of PTSD as described herein and clinically diagnosing PTSD in a subject is performed by two entities, such as by a primary care provider and a specialist. In some embodiments, the first computer program and the second computer program are the same. In some embodiments, the first computer program and the second computer program are different.

In some embodiments, other clinical criteria can be used to evaluate the co-morbidities of PTSD (e.g., Generalized Anxiety Disorder 7-item Scale (GAD-7), Alcohol Use Disorders Identification Test (AUDIT-C), Pittsburgh Sleep Quality Index (PSQI), Adverse Childhood Experiences (ACE), or any combination thereof). Accordingly in some embodiments, methods described herein further comprise analyzing the subject for one or more co-morbidities consistent with PTSD, comprising: anxiety disorders (e.g., generalized anxiety disorder), substance use disorders (e.g., alcohol use disorders), sleep disorders (e.g., anxiety related insomnia), prior traumas (e.g., childhood traumas), and the like. In certain embodiments, methods of providing a differential PTSD diagnosis is provided herein. In some embodiments, the detection of PTSD, clinically diagnosing PTSD in a subject, and evaluating co-morbidities, and/or providing a differential diagnosis, or in any combination thereof is performed by the same entities or two or more entities.

In some embodiments, methods of screening for PTSD further comprises selecting an eligible subject for the treatment of PTSD.

In yet further embodiments, provided herein are methods of selecting an eligible subject for the treatment of PTSD. In some embodiments, a method of selecting an eligible subject for the treatment of PTSD comprises: detecting PTSD in a subject as described herein, and determining said subject is eligible for PTSD treatment based on the positive detection of PTSD in the subject.

In some embodiments, a method of selecting an eligible subject for the treatment of PTSD comprises: obtaining a biological sample (e.g., a cell sample) from a subject; measuring one or more test PTSD-associated gene expression level(s); comparing the one or more test PTSD-associated gene expression level(s) to one or more control gene expression level(s); determining the differential expression of the one or more test PTSD-associated gene(s); determining the positive detection of PTSD based on the differential expression; and determining the subject is eligible for PTSD treatment based on the positive detection of PTSD in the subject.

In some embodiments, a method of selecting an eligible subject for the treatment of PTSD comprises: obtaining a biological sample (e.g., a cell sample) from a subject; measuring with a first computer program, executed on a computer, one or more test PTSD-associated gene expression level(s); with a second computer program, executed on a computer, comparing the one or more test PTSD-associated gene expression level(s) to one or more control gene expression level(s); determining the differential expression of the one or more test PTSD-associated gene(s); determining the positive detection of PTSD based on the differential expression; and determining the subject is eligible for PTSD treatment based on the positive detection of PTSD in the subject.

In some embodiments, the one or more control gene expression level(s) is derived from control or a control population. In some embodiments, the control or a control population is a negative control or control population. In some embodiments, the first computer program and the second computer program are the same. In some embodiments, the first computer program and the second computer program are different.

In some embodiments, methods of selecting an eligible subject for the treatment of PTSD described herein further comprises determining said subject should be clinically evaluated for PTSD. In some embodiments, methods of selecting an eligible subject for the treatment of PTSD described herein further comprises clinically diagnosing said subject for PTSD. In some embodiments, determining said subject should be clinically evaluated for PTSD and/or clinically diagnosing said subject for PTSD can be before, or after, being determined as eligible for PTSD treatment. For example, in some embodiments, a method of selecting an eligible subject for the treatment of PTSD comprises: detecting PTSD in a subject as described herein; wherein upon detecting PTSD in a subject, said method comprising determining said subject should be clinically evaluated for PTSD; clinically diagnosing said subject for PTSD; wherein upon clinically diagnosing PTSD, said method comprises selecting the subject for PTSD treatment.

In some embodiments, methods described herein further comprise treating the subject with a PTSD treatment. Examples of PTSD treatments encompassed by the present disclosure include: psychotherapies (e.g., Cognitive Behavior Therapy, Cognitive Processing Therapy, Cognitive Therapy, Prolonged Exposure Therapy, Eye Movement Desensitization and Reprocessing (EMDR) Therapy, Narrative Exposure Therapy (NET), Group Therapy, or combinations thereof), psychodynamic therapies (e.g., Brief Eclectic Psychotherapy), medicaments or active agents (e.g., selective serotonin reuptake inhibitors (SSRIs), serotonin and norepinephrine reuptake inhibitors (SNRIs), anti-anxiety medications, anti-depressants, or combinations thereof, including sertraline, paroxetine, fluoxetine, venlafaxine, ketamine, a tricyclic antidepressant, an MAOI, an antipsychotic, a beta-blocker, a benzodiazepine, a psychedelic), co-morbidities treatment (e.g., treatment of substance abuse), complementary and/or alternative therapies (e.g., acupuncture, yoga, animal-assisted therapy, meditation, or combinations thereof), and any combination thereof. Methods of treatment, e.g., with one or more medicaments formulated as pharmaceutical compositions, are further described herein.

In some embodiments, provided herein are methods of evaluating treatment efficacy of a subject undergoing PTSD treatment. In some embodiments, a method of evaluating treatment efficacy of a subject undergoing PTSD treatment comprises: detecting PTSD in a subject as described herein, and determining the efficacy of the PTSD treatment based on the detection of PTSD.

In some embodiments, a method of evaluating treatment efficacy of a subject undergoing PTSD treatment comprises: obtaining a biological sample (e.g., a cell sample) from a subject undergoing PTSD treatment; measuring one or more test PTSD-associated gene expression level(s); comparing the one or more test PTSD-associated gene expression level(s) to one or more control gene expression level(s); determining the differential expression of the one or more test PTSD-associated gene(s); determining the positive detection of PTSD based on the differential expression; and determining the efficacy of the PTSD treatment based on the detection of PTSD.

In some embodiments, a method of evaluating treatment efficacy of a subject undergoing PTSD treatment comprises: obtaining a biological sample (e.g., a cell sample) from a subject undergoing PTSD treatment; measuring with a first computer program, executed on a computer, one or more test PTSD-associated gene expression level(s); with a second computer program, executed on a computer, comparing the one or more test PTSD-associated gene expression level(s) to one or more control gene expression level(s); determining the differential expression of the one or more test PTSD-associated gene(s); determining the positive detection of PTSD based on the differential expression; and determining the efficacy of the PTSD treatment based on the detection of PTSD.

In some embodiments, the PTSD treatment is efficacious based on the negative detection of PTSD. In some embodiments, the PTSD treatment is inefficacious (i.e., at that point in time) based on the positive detection of PTSD. In some embodiments, the one or more control gene expression level(s) is one or more control gene expression level(s) derived from a control or a control population. In some embodiments, the control or a control population is a negative control or a negative control population. In some embodiments, the one or more control gene expression level(s) is derived from a baseline. In some embodiments, the baseline is the subject prior to PTSD treatment. In some embodiments, the first computer program and the second computer program are the same. In some embodiments, the first computer program and the second computer program are different.

In some embodiments, the detection of PTSD in a subject can be compared before and after receiving PTSD treatment. Accordingly, the detection of PTSD in a subject can be compared to a baseline detection of PTSD. In embodiments, efficacy of PTSD treatment can be determined relative to the detection of in the subject before and after treatment.

In some embodiments, the PTSD treatment can be the initial PTSD treatment, or an instance of PTSD treatment that is part of a series or ongoing PTSD treatment. In some embodiments, methods of evaluating treatment efficacy of a subject undergoing PTSD treatment can be repeated once, more than once, twice, after each instance or some instances of PTSD treatment, or as many times as necessary for a treatment provider to monitor the efficacy of the PTSD treatment. In some embodiments, methods described herein can be used to provide data to the individual, healthcare provider, or both which can be used to assess the efficacy of the PTSD treatment over time or at a specific time point.

In some embodiments, provided herein are methods of determining prevalence of PTSD in a population of interest. In some embodiments, methods of determining prevalence of PTSD in a population of interest comprises detecting PTSD in each subject of a population of interest and determining the prevalence of PTSD in said population of interest.

In some embodiments, a method of determining prevalence of PTSD in a population of interest comprises: obtaining a biological sample (e.g., a cell sample) from each subject in a population of interest; measuring one or more test PTSD-associated gene expression level(s) in each sample; comparing the one or more test PTSD-associated gene expression level(s) of each sample to one or more control gene expression level(s); determining the differential expression of the one or more test PTSD-associated gene(s); determining the detection of PTSD based on the differential expression in each subject of the population of interest; and determining the prevalence of PTSD in a population of interest based on the detection of PTSD in each subject of the population of interest.

In some embodiments, a method of determining prevalence of PTSD in a population of interest comprises: obtaining a biological sample (e.g., a cell sample) from each subject in a population of interest; measuring with a first computer program, executed on a computer, one or more test PTSD-associated gene expression level(s) in each sample; with a second computer program, executed on a computer, comparing the one or more test PTSD-associated gene expression level(s) of each sample to one or more control gene expression level(s); determining the differential expression of the one or more test PTSD-associated gene(s); determining the detection of PTSD based on the differential expression in each subject of the population of interest; and determining the prevalence of PTSD in a population of interest based on the detection of PTSD in each subject of the population of interest.

In some embodiments, determining the prevalence of PTSD in a population of interest comprises evaluating the number of subjects that PTSD was detected in. In some embodiments, the first computer program and the second computer program are the same. In some embodiments, the first computer program and the second computer program are different.

In some embodiments, the one or more control gene expression level(s) is derived from a control or a control population. In some embodiments, the control or a control population is a negative control or control population. In some embodiments, the population of interest is a military population or a female population.

### Subjects

In some aspects, a subject can comprise a mammal amenable to receive testing for PTSD as described herein. Examples of such mammals may include humans, non-human primates (e.g., apes, gibbons, chimpanzees, orangutans, monkeys, macaques, and the like), Mammals may be any age or at any stage of development, for example a mammal can be neonatal, infant, adolescent, adult or in utero. Mammals may be male or female. In some cases, a human can be from about: 1 day to about 7 days old, 1 week to about 5 weeks old, 1 month to about 12 months old, 1 year to about 6 years old, 5 years to about 15 years old, 14 years to about 30 years old, 25 years to about 50 years old, 40 years to about 75 years old, 70 years to about 100 years old, 85 years old to about 110 years old or about 100 years to about 130 years old.

The subject may be a patient, such as a patient being treated for a trauma that may result in symptoms of PTSD. In certain cases, the subject may be predisposed to a risk of developing a condition or a disease such as PTSD. The subject may be in remission from PTSD. The subject may be healthy.

In some aspects, a method can further comprise diagnosing a subject as having the disease. In some aspects, a diagnosing can comprise employing an in vitro diagnostic. In some aspects, the in vitro diagnostic can be a companion diagnostic. In other instances, the diagnosing can comprise an in vivo diagnostic.

A diagnostic test can comprise an imaging procedure, a blood count analysis, a tissue pathology analysis, a biomarker analysis, a biopsy, a magnetic resonance image procedure, a physical examination, a urine test, an ultrasonography procedure, a genetic test, a liver function test, a positron emission tomography procedure, a X-ray, serology, an angiography procedure, an electrocardiography procedure, an endoscopy, a diagnostic polymerase chain reaction test (PCR), a pap smear, a hematocrit test, a skin allergy test, a urine test, a colonoscopy, an enzyme-linked immunosorbent assay (ELISA), microscopy analysis, bone marrow examination, rapid diagnostic test, pregnancy test, organ function test, toxicology test, infectious disease test, bodily fluids test, or any combination thereof.

In some aspects, a diagnosis may comprise a physical examination, a radiological image, a blood test, an antibody test, or any combination thereof. In some aspects, a diagnosis may comprise a radiological image and the radiological image may comprise: a computed tomography (CT) image, an X-Ray image, a magnetic resonance image (MRI), an ultrasound image, or any combination thereof.

### Administration and Treatment

In some aspects, disclosed herein are methods of administering a treatment (e.g., pharmaceutical composition) as described herein to a subject who can be a subject in need thereof. In some cases, a method of treating or preventing a disease can comprise administering a medicament as described herein. In some embodiments, provided herein is a pharmaceutical composition comprising a medicament (e.g., a PTSD treatment) and a pharmaceutically acceptable carrier, excipient and/or diluent, for the treatment of a subject in need thereof.

In some aspects, a subject can be a subject in need thereof. In some aspects, a subject can have a disease such as post traumatic stress disorder. Treatment may include a reduction in fear, a reduction in a feeling of helplessness, a reduction in disorganized behavior, a reduction in anxiety, a reduction in depression, a reduction in psychosis, a reduction in panic attacks, a reduction in substance abuse, a reduction in suicidal ideation, a reduction in insomnia, an increase in appetite,

Delivery of a pharmaceutical therapy for the treatment of PTSD may include direct application to the affected tissue or region of the body. Delivery can include a parenchymal injection, an intrathecal injection, an intraventricular injection, or an intracisternal injection. A composition provided herein can be administered by any method. A method of administration can be by inhalation, intraarterial injection, intracerebroventricular injection, intracisternal injection, intramuscular injection, intraorbital injection, intraparenchymal injection, intraperitoneal injection, intraspinal injection, intrathecal injection, intravenous injection, intraventricular injection, stereotactic injection, subcutaneous injection, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration. Delivery can include topical administration (such as a lotion, a cream, an ointment) to an external surface of a surface, such as a skin. In some instances, a subject can administer the composition in the absence of supervision. In some instances, a subject can administer the composition under the supervision of a medical professional (e.g., a physician, nurse, physician's assistant, orderly, hospice worker, etc.). In some cases, a medical professional can administer the composition.

The step of selecting a therapy may comprise the additional step of applying and monitoring a therapy and based on its outcome select a therapy. For example, subjects that are diagnosed to be at risk for developing PTSD and put on a prophylactic therapy can be monitored and, if necessary, their therapy subsequently be adjusted or changed depending on whether PTSD symptoms occur and to which extent or not. Also, a therapy may be applied to a patient having acute PTSD and routinely the effect of the therapy is monitored and recorded. The recorded data provides the basis for the assessment whether the therapy applied is beneficial to the patient or not. Based on said assessment the person skilled in the art, in this case likely a clinician, will be able to adjust the currently applied therapy, e.g., by increasing/decreasing dosage regimen or dosage amount of the therapy, or decide to completely switch to another therapy.

In some aspects, a pharmaceutical composition can be administered to a subject at a suitable unit dose. The pharmaceutical composition can be in unit dose form. In some cases, unit dose can be meant to refer to pharmaceutical drug products in the form in which they are marketed for use, with a specific mixture of active ingredients and inactive components, diluents, or excipients, in a particular configuration, and apportioned into a particular dose to be delivered. In some instances, unit dose can also sometimes encompass non-reusable packaging, although the FDA distinguishes between unit dose “packaging” or “dispensing”. More than one unit dose can refer to distinct pharmaceutical drug products packaged together, or to a single pharmaceutical drug product containing multiple drugs and/or doses. In some instances, the term unit dose can also sometimes refer to the particles comprising a pharmaceutical composition, and to any mixtures involved. In some cases, types of unit doses may vary with the route of administration for drug delivery, and the substance(s) being delivered. In some aspects, administration can comprise intravenous, intraperitoneal, intraarterial, intertumoral, subcutaneous, intramuscular, intranasal, topical, oral, or intradermal administration. In some cases, administration can comprise inhalation administration. In some aspects, a dosage regimen can be determined by an attending physician and clinical factors. In some aspects, a dosage for a subject can depend upon many factors, including a subject's size, body surface area, age, sex, general health, a compound to be administered, a time and route of administration, other drugs being administered concurrently, or any combination thereof. In some aspects, a range of a dose can comprise 0.001 to 1000 μg. In some aspects, a dose can be below or above such a range. In some aspects, a regimen as a regular administration of a pharmaceutical composition can be in a range of 1 μg to 10 mg. In some aspects, a regimen as a regular administration of a pharmaceutical composition can be in a range of 10² units to 10¹⁰ units per day, week or month. In some aspects, if a regimen comprises a continuous infusion, it can also be in a range of 1 μg to 10,000 mg of pharmaceutical composition or engineered polynucleotide or DNA encoding the engineered polynucleotide or vector containing or encoding the engineered polynucleotide per kilogram of body weight per minute, respectively. In certain instances, the range is from 1 mg per kilogram of body weight to 1000 mg per kilogram of body weight. In some aspects, progress can be monitored by periodic assessment.

In some aspects of the disclosure, when a pharmaceutical composition is a liquid it may be administered in a liquid dose form such as about 1 ml to about 5 ml, about 5 ml to 10 ml, about 15 ml to about 20 ml, about 25 ml to about 30 ml, about 30 ml to about 50 ml, about 50 ml to about 100 ml, about 100 ml to 150 ml, about 150 ml to about 200 ml, about 200 ml to about 250 ml, about 250 ml to about 300 ml, about 300 ml to about 350 ml, about 350 ml to about 400 ml, about 400 ml to about 450 ml, about 450 ml to 500 ml, about 500 ml to 750 ml, or about 750 ml to 1000 ml.

In some aspects, a composition described herein can be administered one or more days to a subject in need thereof. In some aspects, administration can occur for about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or about 31 days. In some aspects, administration can occur for about: 1, 2, 3, 4, 5, 6, 7, 8, 9, 10, 11, 12, 13, 14, 15, 16, 17, 18, 19, 20, 21, 22, 23, or about 24 months. In some aspects, administration can occur for about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 or more years. In some cases, administration can occur for life. In some aspects, a pharmaceutical composition described herein can be administered on 2, 3, 4, 5, 6, 7, 8, 9, 10, 11, 12, 13, 14, 15, 16, 17, 18, 19 or 20 or more days. In some cases, a composition described herein can be administered on consecutive days or on nonconsecutive days. In some cases, a composition described herein can be administered to a subject more than one time per day. In some instances, a composition described herein can be administered to a subject: 1, 2, 3, 4, 5, 6, 7, 8, 9, or 10 or more times per day.

In some aspects, disclosed herein are methods of use for compositions as disclosed herein. In some aspects, a daily oral dosage regimen can be from about 0.1 milligram per kilogram (mg/kg) to about 80 mg/kg of total body weight, from about 0.2 mg/kg to about 30 mg/kg, or from about 0.5 mg/kg to about 15 mg/kg. In some aspects, a daily parenteral dosage regimen can comprise from about 0.1 mg/kg to about 10,000 mg/kg of total body weight, from about 0.2 mg/kg to about 5,000 mg/kg, or from about 0.5 mg/kg to about 1,000 mg/kg. In some aspects, a daily topical dosage regimen can be from about 0.1 mg to about 500 mg. In some aspects, a daily inhalation dosage regimen can be from about 0.01 mg/kg to about 1,000 mg/kg per day. In some aspects, an optimal quantity and spacing of individual dosages of a composition can be determined by a nature and extent of a condition being treated, a form, route and site of administration, and a particular subject being treated, and that such optimums can preferably be determined by a method described herein. In some aspects, a number of doses of compositions given per day for a defined number of days, can be ascertained by those skilled in the art using conventional course of treatment determination tests. In some aspects, a dosage regimen can be determined by an attending physician and other clinical factors. In some aspects, dosages for any one subject can depend upon many factors. In some aspects, factors affecting dosage can comprise a subject's size, body surface area, age, a particular compound to be administered, sex, time and route of administration, general health, other drugs being administered concurrently or any combination thereof. In some aspects, progress can be monitored by periodic assessment.

A pharmaceutical formulation may be administered a daily oral dosage regimen can be from about 0.1 milligram per kilogram (mg/kg) to about 80 mg/kg of total body weight, from about 0.2 mg/kg to about 30 mg/kg, or from about 0.5 mg/kg to about 15 mg/kg. In some aspects, a daily parenteral dosage regimen can comprise from about 0.1 mg/kg to about 10,000 mg/kg of total body weight, from about 0.2 mg/kg to about 5,000 mg/kg, or from about 0.5 mg/kg to about 1,000 mg/kg. In some aspects, a daily topical dosage regimen can be from about 0.1 mg to about 500 mg. In some aspects, a daily inhalation dosage regimen can be from about 0.01 mg/kg to about 1,000 mg/kg per day. In some aspects, an optimal quantity and spacing of individual dosages of a composition can be determined by a nature and extent of a condition being treated, a form, route and site of administration, and a particular subject being treated, and that such optimums can preferably be determined by a method described herein. In some aspects, a number of doses of compositions given per day for a defined number of days, can be ascertained by those skilled in the art using conventional course of treatment determination tests. In some aspects, a dosage regimen can be determined by an attending physician and other clinical factors. In some aspects, dosages for any one subject can depend upon many factors. In some aspects, factors affecting dosage can comprise a subject's size, body surface area, age, a particular compound to be administered, sex, time and route of administration, general health, other drugs being administered concurrently or any combination thereof. In some aspects, progress can be monitored by periodic assessment.

### Systems

Provided herein are systems comprising the PTSD molecular detection assay described herein or a use thereof. Systems provided herein further comprise the materials for use in the molecular detection assay, methods, compositions, kits, devices or any combination of the foregoing of the present disclosure as described herein. For example, systems provided herein comprise agents, which may include gene-specific or gene-selective probes and/or primers, for quantifying the expression of the disclosed genes for predicting prognostic outcome or response to treatment. Such systems may optionally contain reagents for the extraction of RNA from samples, such as fixed paraffin-embedded tissue samples and/or reagents for RNA amplification. In addition, the systems may optionally comprise the reagent(s) with an identifying description or label or instructions relating to their use in the methods of the present disclosure. In some embodiments, systems comprise a collection container to obtain the biological example. Such collection containers can be any sample collection device that is capable of collecting a suitable quantity of human blood, tissue, or saliva including specific cell types such as peripheral blood mononuclear cells (PBMC), epithelial cells, or other human cell type containing nucleic acids from which total genomic ribonucleic acid samples can be isolated for testing. Examples of sample collection devices include the PAXgene® Blood RNA Tube (Becton-Dickinson Biosciences Cat #:762165), OMNIgene™⋅SALIVA DNA and RNA device OMR-610 (DNA Genotek Cat #: OMR-610), Oragene Discover OCR-100 saliva collection device (DNA Genotek Cat #: OCR-100), DNA/RNA Shield SafeCollect Saliva Collection Kit (Zymo Research Cat #: R1211), DNA/RNA Shield Blood Collection Tube (Zymo Research Cat #: R1150), SpeciMAX Saliva Collection Kit (ThermoFisher Cat #: A50696), Tempus™ Blood RNA Tube (ThermoFisher Cat #: 4342792), Buccal Swab Collection & Stabilization Kit (CanVax Cat #: SWC001), TAP® Micro Select (YourBio Health-FDA 510k cleared), or combinations thereof.

The systems may comprise containers (including microliter plates suitable for use in an automated implementation of the method), each with one or more of the various materials or reagents (typically in concentrated form) utilized in the methods, including, for example, chromatographic columns, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present disclosure (e.g., appropriate length poly(T) or random primers linked to a promoter reactive with the RNA polymerase). Mathematical algorithms used to estimate or quantify prognostic or predictive information are also properly potential components of systems provided herein.

The values from the assays described above, such as expression data, can be calculated and stored manually. Aspects of this disclosure also provide a system for completely or partially carrying out the methods of the disclosure. The present disclosure thus provides a computer program product including a computer readable storage medium having a computer program stored on it. The program can, when read by a computer, execute relevant calculations based on values obtained from analysis of one or more biological samples from an individual (e.g., gene expression levels, normalization, standardization, thresholding, and conversion of values from assays to a score and/or text or graphical depiction of PTSD progression and related information). The computer program product has stored therein a computer program for performing the calculation.

The system can comprise, for example, i) a computer; and ii) non-transient storage medium comprising computer executable instructions which are performed by the computer and which cause the computer to carry out any one or more steps of the methods described herein. The system can comprise, for example, i) a computer; and ii) non-transient storage medium comprising computer executable instructions which are performed by the computer and which cause the computer to carry out the steps of a) receiving at least one test gene expression sequence from a tissue sample (e.g., an RNA expression sequence) and b) determining the gene expression level of the at least one sequence. Ins some embodiments, the non-transient storage medium comprises computer executable instructions which are performed by the computer and which cause the computer to carry out the steps of c) obtaining a control gene expression level of at least one sequence (e.g., from a control) or the mean control gene expression level of at least one sequence (e.g., from a control population); and d) detecting the relative expression of the at least one test gene expression sequence relative to the control gene expression level.

The system also generally comprises an output device capable of presenting results obtained by the computer during or as a result of (e.g., in) expression steps. The non-transient storage medium may be on the hard drive of the computer, or may be located on a portable device such as a disc, CD, DVD, thumb drive, flash drive, laptop, portable computer (e.g., a PC or other type), or other such device. Alternatively, the non-transient storage medium may be at a location such as a remote location or a database that is accessible via the internet, or stored in a cloud, or in or on another computer or computer system that is accessible by the computer of the system. The non-transient storage medium may also include instructions for causing the computer to receive, as input, at least one genomic DNA sequence from a nucleotide sequencing apparatus or from a database. The database may be downloaded from a remote site (e.g., via the internet), and/or may be located (stored) on the computer or may be located on another computer or computer system that is accessible by the computer of the system, or may even be located on a portable device as described above. In other aspects, the data is downloaded from a gene sequencing apparatus, and the system may also include such an apparatus. If present, the apparatus is operably electronically linked to the computer in a manner that allows data gathered or measured by the expression level testing apparatus to be outputted and transmitted to and received as input by the computer.

The output device may be of any suitable type, including but not limited to a printer, a display (e.g., a monitor that displays the results as a list, as a graph, or in some other suitable format), or a modem that sends out information (e.g. to another output device, to another computer, or to a storage device such as a DVD, CD, etc.).

Such a system may allow for a computer with non-transient storage medium. The computer may be operationally linked to (or connected to, functionally connected to, or in electrical communication with) output device. In some instances, the computer is also operationally linked to nucleic acid sequencing apparatus, and data (e.g., a genomic nucleotide sequence, generally a DNA sequence) from nucleic acid sequencing apparatus can be output and transferred to and received as input by the computer for analysis by the methods of the disclosure. In other instances, the computer is operationally linked to a database and information and/or data can be output from the database and transferred to and received as input by the computer. Non-transient storage medium may contain computer executable instructions (e.g., code, computer program, etc.) which are performed by said computer and which cause said computer to carry out the steps of the methods described herein.

### Devices

In some embodiments, provided herein is a PTSD detection device or a use thereof. In some embodiments, a PTSD detection device comprises the PTSD molecular detection assay as described herein. In some embodiments, a PTSD detection device can comprise any one or more of components or materials of systems, compositions, devices and/or methods described herein, such as a molecular detection assay as provided herein. In some embodiments, a PTSD detection device comprises one or more assays which quantify the gene expression levels as described herein. In some embodiments, a PTSD detection device comprises a collection container which collects the biological sample from the subject. In some embodiments, devices comprise a collection container to obtain a biological sample wherein the container comprises one or more fixative or preservative reagents. Collection containers can be any sample collection device that is capable of collecting a suitable quantity of human blood, tissue, or saliva including specific cell types such as peripheral blood mononuclear cells (PBMC), epithelial cells, or other human cell type containing nucleic acids from which total genomic ribonucleic acid samples can be isolated for testing.

Examples of sample collection containers include the PAXgene® Blood RNA Tube (Becton-Dickinson Biosciences Cat #:762165), OMNIgene™⋅SALIVA DNA and RNA device OMR-610 (DNA Genotek Cat #: OMR-610), Oragene Discover OCR-100 saliva collection device (DNA Genotek Cat #: OCR-100), DNA/RNA Shield SafeCollect Saliva Collection Kit (Zymo Research Cat #: R1211), DNA/RNA Shield Blood Collection Tube (Zymo Research Cat #: R1150), SpeciMAX Saliva Collection Kit (ThermoFisher Cat #: A50696), Tempus™ Blood RNA Tube (ThermoFisher Cat #: 4342792), Buccal Swab Collection & Stabilization Kit (CanVax Cat #: SWC001), TAP® Micro Select (YourBio Health—FDA 510k cleared), or combinations thereof.

In some embodiments, upon quantification of the gene expression, a result report is generated. In some embodiments, the methods of this disclosure can produce a report or summary of information obtained from the herein-described methods. For example, a report may include information concerning expression levels of one or more genes, detection of PTSD, or the patient's risk of recurrence, the patient's likely prognosis or risk classification, clinical and pathologic factors, and/or other information. The methods and reports of this disclosure can further include storing the report in a database. The method can create a record in a database for the subject and populate the record with data. The report may be a paper report, an auditory report, or an electronic record. The report may be displayed and/or stored on a computing device (e.g., handheld device, desktop computer, smart device, website, etc.). It is contemplated that the report is provided to a physician and/or the patient. The receiving of the report can further include establishing a network connection to a server computer that includes the data and report and requesting the data and report from the server computer.

In some embodiments, upon quantification of the gene expression, a result report is generated to indicate whether the subject screens positive or negative for PTSD. Positive results for the detection of PTSD warrant referral to a specialty care provider for further evaluation and diagnosis.

### Kits

The present disclosure additionally provides kits for testing for PTSD in a subject. Kits provided herein can comprise any one or more of components or materials of systems, compositions, devices and/or methods described herein, such as a molecular detection assay, and instructions of use thereof. In some embodiments, kits comprise one or more collection containers. In some embodiments, kits comprise a collection container to obtain a biological sample wherein the container comprises one or more fixative or preservative reagents. Collection containers can be any sample collection device that is capable of collecting a suitable quantity of human blood, tissue, or saliva including specific cell types such as peripheral blood mononuclear cells (PBMC), epithelial cells, or other human cell type containing nucleic acids from which total genomic ribonucleic acid samples can be isolated for testing.

Examples of sample collection containers include the PAXgene® Blood RNA Tube (Becton-Dickinson Biosciences Cat #:762165), OMNIgene™⋅SALIVA DNA and RNA device OMR-610 (DNA Genotek Cat #: OMR-610), Oragene Discover OCR-100 saliva collection device (DNA Genotek Cat #: OCR-100), DNA/RNA Shield SafeCollect Saliva Collection Kit (Zymo Research Cat #: R1211), DNA/RNA Shield Blood Collection Tube (Zymo Research Cat #: R1150), SpeciMAX Saliva Collection Kit (ThermoFisher Cat #: A50696), Tempus™ Blood RNA Tube (ThermoFisher Cat #: 4342792), Buccal Swab Collection & Stabilization Kit (CanVax Cat #: SWC001), TAP® Micro Select (YourBio Health—FDA 510k cleared), or combinations thereof.

In some embodiments, kits provided herein comprises a sample collection container, instructions and/or supplies for said container to be sent by mail to a processing facility for processing by methods, devices, and/or assays as described herein. Processing facilities can be any suitable one or more laboratories that can carry out the methods described herein, or utilize the molecular detection assay or device as described herein.

In some embodiments, kits provided herein comprise a sample collection container and instructions for said sample to be processed at a point-of-care facility. A point-of-care facility can be a patient care facility, such as a doctor's office, clinic or a hospital, or it at be any location the subject is receiving care, such as at home. In some embodiments, kits provided herein contains all assay components necessary to undertake the methods described herein at home. Suitable point-of-care test systems can be used in kits described herein, and include, for example, membrane-based test strips.

In some embodiments, kits provided herein can include a kit housing containing assay components operable to detect a differential expression level of RNA or protein from a tissue sample from the subject, instructions describing how to perform an assay using the assay components, and a control pattern of an expression level of RNA or protein to compare with the differential expression level of RNA or protein.

A variety of kit housings are contemplated, and are sufficient to contain the assay components, the instructions, and the control expression levels of a subject without PTSD. Such a kit can comprise an assembly of the required elements and any helpful instructions to perform such methods and tests, for assaying the expression level of genes associated with PTSD or proteins corresponding to these genes to identify PTSD. In some aspects, the kit housing can contain a receptacle for containing and/or transporting a tissue sample.

Additionally, in some aspects the kit can contain a genome array, PCR materials, or the like. Furthermore, the assay components included in the kit can be used to perform any assay capable of differential RNA expression of genes associated with PTSD. Non-limiting examples can include a real time PCR, northern blotting, a nucleic acid microarray, western blotting, immunoassays, quantitative PCR and the like including combinations of techniques; and tools (e.g., computational tools) for data analysis and/or data interpretation.

The kit may also include a sample collection device that is capable of collecting a suitable quantity of human blood, tissue, or saliva including specific cell types such as peripheral blood mononuclear cells (PBMC), epithelial cells, or other human cell type containing nucleic acids from which total genomic ribonucleic acid samples can be isolated for testing. Examples of sample collection devices include the PAXgene® Blood RNA Tube (Becton-Dickinson Biosciences Cat #:762165), OMNIgene™⋅SALIVA DNA and RNA device OMR-610 (DNA Genotek Cat #: OMR-610), Oragene Discover OCR-100 saliva collection device (DNA Genotek Cat #: OCR-100), DNA/RNA Shield SafeCollect Saliva Collection Kit (Zymo Research Cat #: R1211), DNA/RNA Shield Blood Collection Tube (Zymo Research Cat #: R1150), SpeciMAX Saliva Collection Kit (ThermoFisher Cat #: A50696), Tempus™ Blood RNA Tube (ThermoFisher Cat #: 4342792), Buccal Swab Collection & Stabilization Kit (CanVax Cat #: SWC001), TAP® Micro Select (YourBio Health—FDA 510k cleared).

### EXAMPLES

### Example 1: Predicting Altered RNA Expression Levels to Identify PTSD

Blood samples were taken from patients with PTSD and compared to a control group. In this analysis, each subject had approximately 2.5 ml of blood taken. 1003 unique genes were measured per run to determine aberrant expression levels.

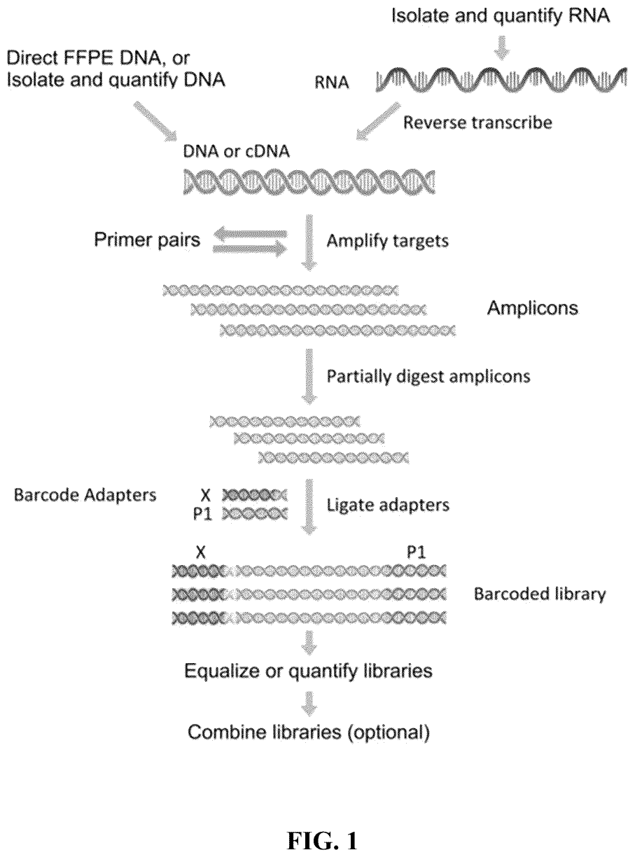

First, RNA from subject blood was subjected to reverse transcription PCR to generate a series of amplicons, or sequences with primer sequences at their 3′ and 5′ ends. Next, the amplicons were partially digested and ligated to adapters in order to quantify. The flow chart is depicted in FIG. 1. The sequences were run on an Illumina sequencing instrument. After the sequencing run, the raw data was collected to generate FASTQ files. The FASTQ files were compared to a bioinformatics system to generate normalized differential gene expression data.

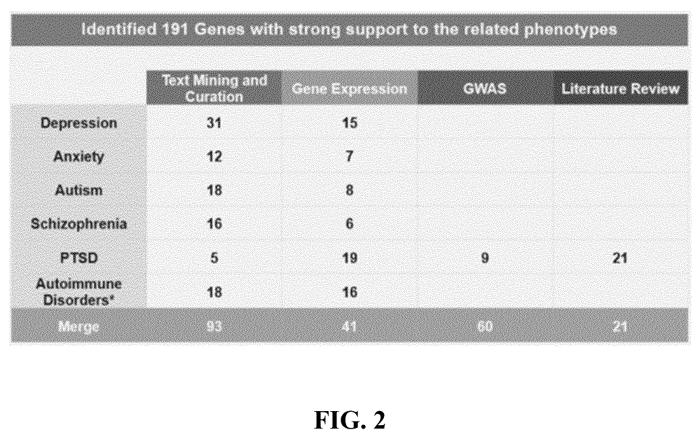

Artificial intelligence and machine learning was used for strategic gene selection as shown in FIG. 2.

A minimum of five million reads per sample were carried out and the percentage of aligned reads showing a minimum of 75% were accurately mapped back to the human genome.

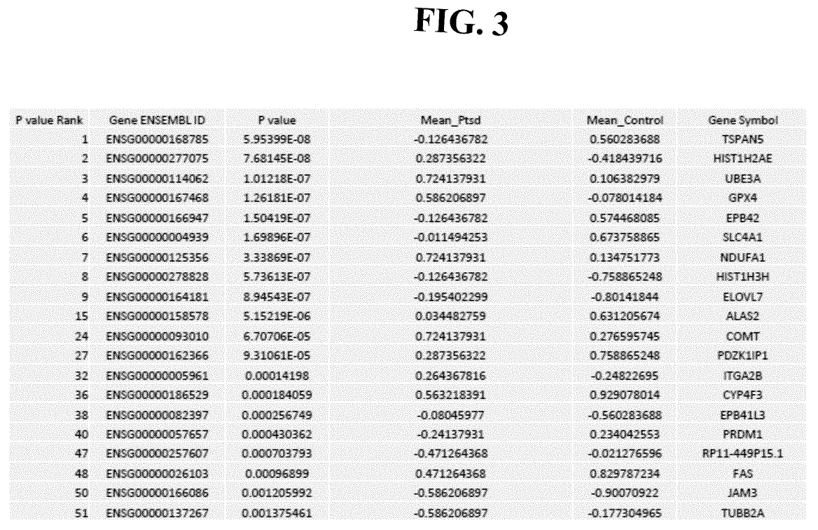

The twenty most predictive genes after expression data that corresponded to subjects having PTSD is depicted in FIG. 3. All genes that were determined to be statistically significant had P values of 2.5×10⁻⁶ or less as shown in FIG. 3.

### Example 2: Generation of an Objective Molecular Detection Assay to Screen for PTSD

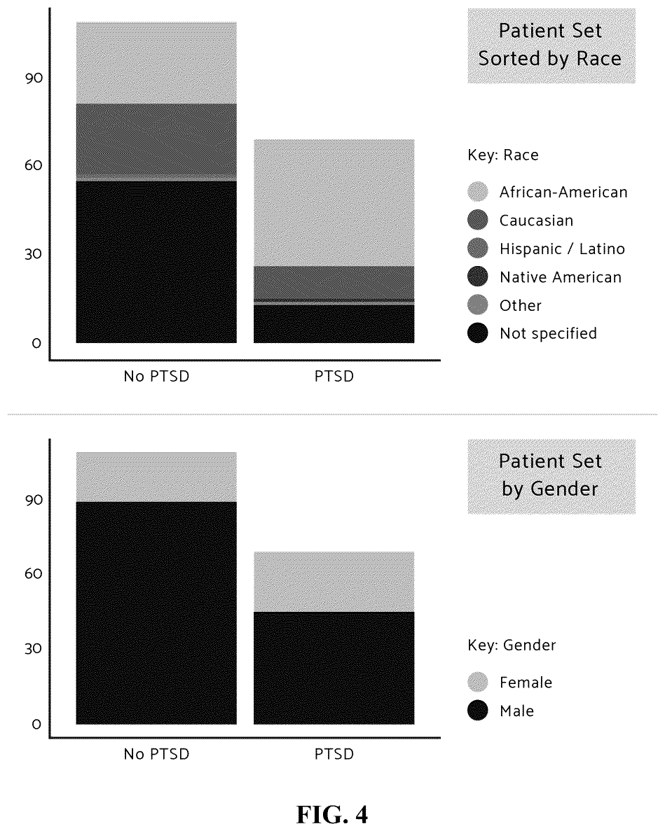

The present example describes the generation of an objective molecular detection assay to screen for PTSD. Blood samples were taken from 87 subjects with PTSD and 141 control subjects (228 subjects total), the demographics of which can be seen in FIG. 4. Each subject had approximately 2-3 ml of blood taken. RNA expression levels and sequencing of 1003 gene targets was carried out using the ADAPT Panel™. The expression data was analyzed using Normal Quantile Transformation (NQT) as described herein (see, the Differential Expression section, referring to Equations 1-7). Specifically, gene expression levels were normalized and the following genes were statistically significantly correlated with PTSD (TABLE 2).

TABLE 2
Exemplary Ensemble IDs for Assayed Genes

| Esemble_ID | Gene Symbol |
|---|---|
| ENSG00000004939 | SLC4A1 |
| ENSG00000005961 | ITGA2B |
| ENSG00000026103 | FAS |
| ENSG00000057657 | PRDM1 |
| ENSG00000082397 | EPB41L3 |
| ENSG00000093010 | COMT |
| ENSG00000114062 | UBE3A |
| ENSG00000125356 | NDUFA1 |
| ENSG00000137267 | TUBB2A |
| ENSG00000158578 | ALAS2 |
| ENSG00000162366 | PDZK1IP1 |
| ENSG00000164181 | ELOVL7 |
| ENSG00000166086 | JAM3 |
| ENSG00000166947 | EPB42 |
| ENSG00000167468 | GPX4 |
| ENSG00000168785 | TSPAN5 |
| ENSG00000186529 | CYP4F3 |
| ENSG00000257607 | RP11-449P15.1 |
| ENSG00000277075 | HIST1H2AE |
| ENSG00000278828 | HIST1H3H |

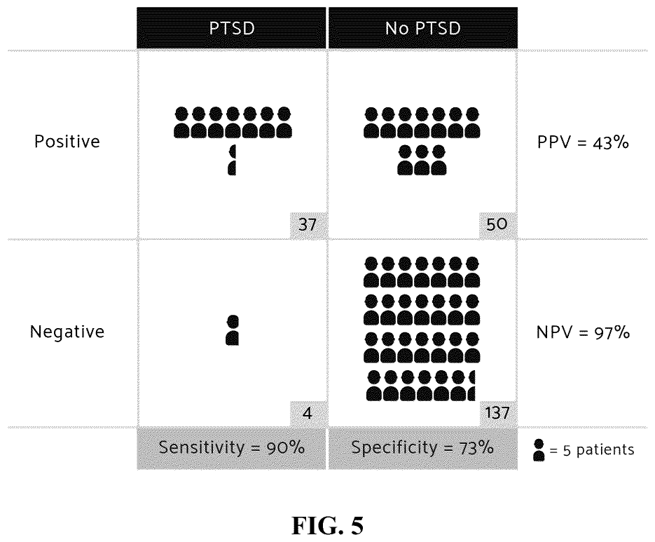

Analysis of the molecular detection assay demonstrate that the sensitivity of the molecular detection assay, or the percentage of subjects correctly identified as having the disorder, is 90% (FIG. 5). Sensitivity of a test is the most important attribute when the test is used for screening purposes and in general, a sensitivity of >80% has been considered adequate by researchers in this field. The surveys currently used, such as those described herein, range in sensitivity between 77 and 91% in research scenarios when the subject is volunteering to participate, however in practice can be biased in either direction by the motivation or fears of the respondent. Analysis also demonstrates that the specificity of the molecular detection assay, or the percentage of subjects correctly identified as not having the disorder, as 73% (FIG. 5). The specificity of the molecular detection assay was found to be better than that of most screening tests currently in use.

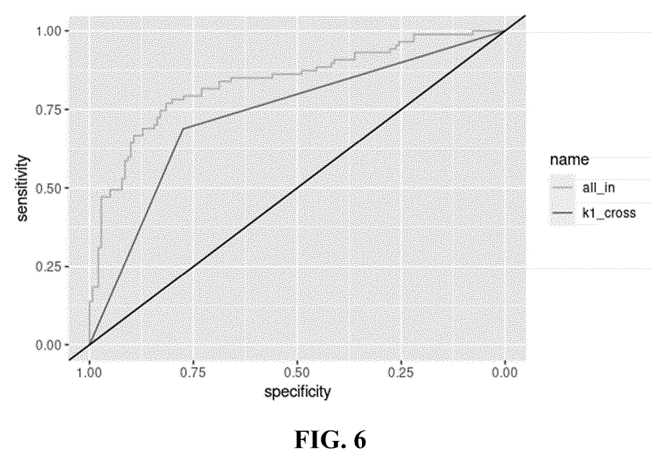

In a previous molecular detection assay with different thresholds using the same genes demonstrated promising results, achieving an Area Under the Receiver Operating Characteristic Curve (AUC) of 85% (FIG. 6).

The results demonstrate that a consistent, reliable and objective molecular detection assay has been generated which indicates whether a subject is predicted to have PTSD without interference from self-reporting reporter bias.

### Example 3: Molecular Detection Assay Validation and Calibration

Validating the molecular detection assay's performance on a larger, independent dataset is needed to provide additional evidence of its usefulness. This will require a larger biorepository with a diverse population to further train and test the molecular detection assay to enhance the molecular detection assay's reliability and generalizability.

### Example 3.1. Validation Relative to Industry Standard Psychological Assessments

The present example describes efforts to be taken to validate the present molecular detection assay relative to clinically accepted PTSD related psychological assessments. The primary assessment is relative to the present example is Clinician-Administered PTSD Scale for DSM-5 (CAPS-5). CAPS-5 is considered the “gold standard” for diagnosis of PTSD, and is a structured interview. The other primary assessment is Posttraumatic Stress Disorder Checklist for DSM-5 (PCL-5). Other psychological assessments include, Patient Health Questionnaire-9 (PHQ-9), Alcohol Use Identifications Test (AUDIT-C), Generalized Anxiety Disorder-7 (GAD-7), and Pittsburgh Sleep Quality Index (PSQI).

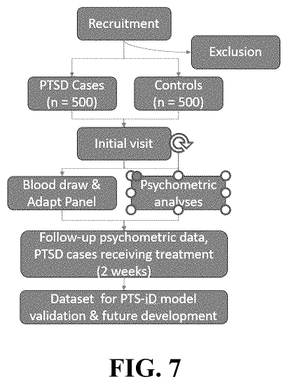

Example 3.1.1. The Study. Five hundred subjects diagnosed with PTSD and five hundred control subjects are recruited. Subjects diagnosed with PTSD are individuals who have a CAPS-5 score that meets the diagnosis criteria for PTSD, and control subjects are individuals who do not have a CAPS-5 score that meets the diagnosis criteria for PTSD. Each subject undergoes PTSD screening by the present RNA screening assay as analyzed by the present molecular detection assay and by psychometric analysis using accepted psychological assessment. For RNA evaluation, ˜2.5 mL of blood is taken, and the RNA expression levels of genes including the genes set forth in TABLE 2 is evaluated. For psychometric analysis, subjects are analyzed using the standard protocols for the corresponding psychological assessment. A schematic diagram of the above described work-flow can be seen in FIG. 7.

Example 3.1.2. Data Reprocessing. The ADAPT Panel is run on NGS platform with an output of a FASQfile. The expression data will be analyzed by using NQT as described herein.

Specifically, the following steps are involved in data preprocessing:
Quality Control,
Data Cleaning,
Normalization,
Feature Selection,
Integration of Genomic and Clinical Data,
Batch Effect Removal,
Dimensionality Reduction,
Data Scaling,
Cross-validation, and
Validation and Reproducibility

As a first step in preprocessing, quality control (QC) checks will be conducted on the collected genomic and clinical data. This will ensure the reliability and integrity of the data by identifying and addressing issues such as missing values, outliers, batch effects, or any other anomalies.

Any data errors, missing values, or outliers identified during the QC process will be addressed through data cleaning procedures. Imputation methods will be used to fill in missing values or, if applicable, exclude data points with severe issues from further analysis.

After data cleaning, data normalization will be performed to remove technical variability while preserving biological significance. In this study, the normal quantile transformation (NQT) is chosen as the normalization method. The NQT is a rank-based procedure that scales data within each quantile separately, known for its reduced bias and robustness compared to other normalization methods for microRNA sequencing data.

Feature selection is essential for reducing dimensionality and selecting the most relevant genomic features (e.g., genes or microRNA species) for the predictive model. We may use various methods, such as statistical tests, correlation analysis, and machine learning algorithms, to identify the most informative features associated with PTSD.

The study will include both genomic and clinical data. Therefore, the two data types will be integrated for comprehensive analysis. This step involves mapping clinical variables to corresponding genomic samples and ensuring data consistency.

Batch effect removal techniques will be applied to account for systematic variations between batches. This will ensure that the observed effects are not confounded by batch-specific variations.

When dealing with a high number of features, dimensionality reduction techniques such as Principal Component Analysis (PCA) or t-distributed Stochastic Neighbor Embedding (t-SNE) may be applied to reduce the data's complexity while preserving important patterns and structures.

For some machine learning algorithms, the data may need to be scaled to ensure that all features have a similar range. Common scaling methods include standardization (mean-centered and scaled by standard deviation) or min-max scaling (scaled to a predefined range).

To evaluate the predictive model's performance accurately, cross-validation techniques (e.g., k-fold cross-validation) can be used to divide the data into subsets for training and testing the model iteratively.

After preprocessing, the model's performance should be validated using an independent dataset to ensure the generalizability and reproducibility of the results._NQT will be used as described in Normal Quantile Transformation (Cer R Z, Herrera-Galeano J E, Anderson J J, Bishop-Lilly K A, Mokashi V P. miRNA Temporal Analyzer (mirnaTA): a bioinformatics tool for identifying differentially expressed microRNAs in temporal studies using normal quantile transformation. Gigascience. 2014 Oct. 13; 3:20. doi: 10.1186/2047-217X-3-20. PMID: 25379175; PMCID: PMC4212236.) Custom pipelines are written in R. In addition, all analyses are written using R Statistical Software (v4.1.2; R Core Team 2021).

Example 3.1.3. The Evaluation. Upon generating the initial molecular detection assay, the model component of the molecular detection assay will be cross-validated as described below, and optionally evaluated and/or adjusted based on one or more of the below tests.

Cross Validation: Cross-validation is performed to assess the molecular detection assay's robustness and generalization to new data. Cross-validation is a widely used technique to assess the performance of a predictive molecular detection assay while mitigating potential issues of overfitting. Here, k-fold cross-validation is employed to validate the algorithm's performance. The dataset was randomly divided into k subsets (folds), where k−1 subsets was used for training the molecular detection assay, and the remaining subset was used for testing. This process is repeated k times, each time using a different fold as the test set. The performance metrics (e.g., accuracy, AUC, precision, recall) is averaged across all folds to obtain an estimate of the molecular detection assay's overall performance.

Hold-Out Validation with Independent Test Sets: In addition to cross-validation, an independent test set will be reserved from the collected data. This test set will be completely separate from the data used for training and cross-validation. Once the molecular detection assay is trained using the cross-validation process, it will be evaluated on the independent test set to assess its generalization performance to new, unseen data. The performance metrics obtained from this evaluation will provide a more realistic indication of the algorithm's ability to predict PTSD in real-world scenarios.

Stratified Sampling: To ensure that both the training and test sets are representative of the overall data distribution, stratified sampling will be employed. Stratified sampling maintains the same class distribution (e.g., controls and PTSD cases) in both the training and test sets, ensuring that the molecular detection assay is not biased towards the majority class.

Performance Metrics: Various performance metrics, such as accuracy, sensitivity (recall), specificity, precision, and Area Under the Receiver Operating Characteristic Curve (AUC), will be used to evaluate the algorithm's performance. These metrics provide a comprehensive assessment of how well the molecular detection assay can classify individuals with and without PTSD.

Overfitting Detection and Model Selection: Cross-validation allows for detecting overfitting, a situation where the model performs well on the training data but poorly on unseen data. If significant overfitting is detected, model hyperparameters can be adjusted or feature selection methods can be refined to improve generalization.

Repeated Cross-Validation (Optional): In situations where the dataset is relatively small, repeated cross-validation can be performed to obtain more robust estimates of performance. This technique involves randomly shuffling the data and running cross-validation multiple times, calculating the average performance metrics.

Example 3.1.4. Model performance metrics. Model performance metrics of the model component of the molecular detection assay are described in TABLE 3 below.

TABLE 3
EXEMPLARY MODEL PERFORMANCE METRICS

| Metric | Definition |
|---|---|
| Accuracy | Accuracy measures the proportion of correct predictions out of all |
| | predictions made by the model. It provides an overall assessment of |
| | the algorithm's ability to classify both PTSD cases and controls |
| | correctly. |
| Sensitivity (recall), | Sensitivity, also known as recall or true positive rate, measures the |
| | proportion of actual PTSD cases correctly identified by the model. It |
| | represents the algorithm's ability to detect individuals who have |
| | PTSD. |
| Specificity | Specificity measures the proportion of actual controls correctly |
| | identified by the model. It represents the algorithm's ability to |
| | correctly classify individuals without PTSD. |
| Precision (Positive | Precision measures the proportion of true PTSD cases among all |
| Predictive Value) | predicted PTSD cases. It indicates the algorithm's accuracy in |
| | identifying true positive cases. |
| Negative Predictive | NPV measures the proportion of true controls among all predicted |
| Value (NPV) | control cases. It represents the algorithm's accuracy in identifying |
| | true negative cases. |
| F1 Score | The F1 score is the harmonic mean of precision and recall. It |
| | provides a balanced measure of both precision and recall, making it |
| | useful when there is an imbalance between the number of PTSD |
| | cases and controls |
| Area Under the | The AUC evaluates the model's ability to distinguish between PTSD |
| Receiver Operating | cases and controls across various classification thresholds. It |
| Characteristic | provides a measure of the algorithm's overall discriminative power. |
| Curve (AUC) | |
| Matthews Correlation | MCC takes into account true positive, true negative, false positive, |
| Coefficient (MCC) | and false negative predictions. It provides a balanced measure of the |
| | model's performance, especially in the case of imbalanced datasets. |

Example 3.1.5. Statistical Analysis Plan. The following describes how the data derived by conducting Example 3.1.1 will be analyzed.

Primary Endpoints. Performance metrics described in TABLE 3 above will be calculated based on the model's predictions and the true labels (PTSD cases and controls) in both the cross-validation and independent test set.

Hypothesis Testing. Each gene will be tested in the panel individually with the null hypothesis that the gene expression on the controls is equal to that on the cases. Contributions with large effects is not expected, testing for it is necessary.

Receiver Operating Characteristic (ROC) Curve Analysis. The ROC curve analysis will be used to visualize the trade-off between sensitivity and specificity at different classification thresholds. The area under the ROC curve (AUC) will be calculated to compare the algorithm's discriminative power with that of existing screening methods. A higher AUC indicates better discrimination ability.

Confidence Intervals. To estimate the uncertainty around the performance metrics, confidence intervals can be calculated. Confidence intervals provide a range of values within which the true performance of the algorithm is likely to lie.

Power Analysis. If the study involves comparing the algorithm's performance with existing methods and a specific effect size is of interest, a power analysis can be conducted to determine the sample size required to achieve adequate statistical power.

Model Calibration. Model calibration refers to the agreement between the predicted probabilities generated by the algorithm and the observed frequencies of positive cases. Calibration plots can be created to assess the model's calibration, helping determine whether the predicted probabilities are accurate.

Subgroup Analysis. If there are specific subgroups of interest (e.g., different age groups, gender, trauma type), subgroup analyses can be performed to evaluate the algorithm's performance in these subgroups. This analysis can provide insights into potential variations in the algorithm's performance based on different participant characteristics.

Comparison of Sensitivity and Specificity. If existing screening methods have different sensitivity and specificity trade-offs, a receiver operating characteristic comparison (ROCC) curve can be used to visualize and compare the two approaches' performances.

Statistical Significance. Appropriate statistical tests will be used to determine the statistical significance of any observed differences in performance metrics between the algorithm and existing screening methods.

Example 3.1.6. Potential Confounders and Bias. The following describes how potential confounders and bias will be treated.

Demographic Factors. Participant demographics, such as age, gender, ethnicity, socioeconomic status, and education level, can influence gene expression patterns and may also be associated with PTSD risk. To mitigate the impact of these factors, subgroup analyses or include demographic variables can be performed as covariates in the predictive model.

Trauma Type. The type of the traumatic events experienced by participants can vary significantly and may affect gene expression profiles differently. Accounting for trauma type as potential confounders can help in isolating the specific influence of gene expression on PTSD risk.

Medication Use. Certain medications, such as antidepressants or anxiolytics, may influence gene expression levels and could potentially affect PTSD outcomes. Participants' medication usage history should be collected and controlled for in the analysis.

Comorbidities. PTSD often co-occurs with other mental health disorders, such as depression or anxiety. These comorbidities could impact gene expression patterns and potentially confound the association with PTSD. Comorbid conditions should be carefully assessed and accounted for in this analysis.

Sample Selection Bias. If the recruitment of participants is not representative of the broader population of individuals with PTSD, the results may not be generalizable. To minimize sample selection bias, diverse and inclusive recruitment strategies should be utilized.

Batch Effects. If the gene expression data were collected in multiple batches, batch effects may introduce systematic variations that are unrelated to PTSD status. Appropriate statistical methods, such as batch effect correction techniques, can be applied to address this issue.

Measurement Errors. Technical errors in RNA gene expression measurements can occur during laboratory processing, leading to measurement noise. Quality control procedures and normalization methods should be applied to minimize measurement errors.

Data Imputation Bias. In cases of missing data, the method of imputation can introduce bias in the results. It is essential to choose an appropriate imputation method and assess the sensitivity of the results to different imputation approaches.

Overfitting. Overfitting occurs when the predictive model performs well on the training data but poorly on new, unseen data. Regularization techniques and proper validation methods, like cross-validation, can help prevent overfitting and ensure the model's generalization ability.

Publication Bias. Publication bias can arise if studies with positive or significant results are more likely to be published, leading to an overestimation of the predictive model's performance. To mitigate publication bias, researchers should consider pre-registering the study and analyzing all relevant outcomes, regardless of their statistical significance.

Example 3.1.7. Results. It is postulated that the present molecular detection assay can achieve a sensitivity above 90%, while maintaining a specificity above 75% with a NPV above 95%, and a PPV>50% across a range of trauma types. These characteristics are ideal for a screening tool because it means that individuals afflicted with PTSD can be correctly identified and immediately referred for treatment. With the current tools, in order to attain this level of sensitivity (>90%), the specificity, or ability to correctly rule out the disease in those who do not have PTSD, drops below 50%. Current tools depend on accurate reporting which subject to reporter bias and can even be impossible (due to repression of painful memories) or self-modified by conscious and/or subconscious fears, concerns and/or stigma. This is likely the reason the precision metrics of the current self-reporting tools vary significantly across trauma types, as some trauma types are more associated with social stigma. The present molecular detection assay is the first and only tool currently available that attains the described precision metrics based on objective measurements.

Without being bound by theory, it is postulated that the present molecular detection assay can achieve a comparable sensitivity and/or specificity relative to one or more psychological assessments, and optionally, achieve an enhanced sensitivity and/or specificity relative to one or more psychological assessments. Accordingly, the present molecular detection assay can be used to detect the incidence of PTSD, select subjects suitable for PTSD treatment, and/or monitor the progress of subjects undergoing PTSD treatment. Moreover, it is postulated that the present molecular detection assay will provide gene targets for the development of novel precision therapeutics related to PTSD treatment.

### Example 3.2. Use of the Molecular Detection Assay to Establish Subject Selection for PTSD Treatment

The present example describes efforts to be taken to establish a molecular detection assay and/or calibrate the present molecular detection assay to determine if a subject is a suitable candidate for PTSD treatment.

The psychological assessment group described in Example 3.1 is treated according to industry standards (e.g. Stellate Ganglion Blockade, Trauma Informed Talk Therapy, Ketamine Infusions alone or in combination with the aforementioned therapies). Data will be collected by comparing the results of a first screening (e.g., a CAPS-5 interview) administered by a trained provider before therapy to that of a second screening (e.g., a PCL-5 or informal interview) administered 30+/−3 days after treatment.

Without being bound by theory, it is postulated that the collected data can train the present molecular detection assay to predict subject responsiveness to a treatment modality, and therefore subject selection for PTSD treatment.

### Example 3.3. Use of the Molecular Detection Assay to Establish PTSD Treatment Responsiveness

The present example describes efforts to be taken to generate a molecular detection assay and/or calibrate the present molecular detection assay to determine if a subject is responsive to PTSD treatment relative to clinically accepted PTSD related psychological assessments.

The groups described in Example 3.1 are treated according to industry standards. Optionally, subjects to be treated are selected based on the molecular detection assay described in Example 3.2.

Periodically (e.g., every 2-3 months, quarterly, bi-annually, and/or after as needed), RNA expression levels are collected and analyzed as described in Example 3.1. Subjects having PTSD with improved RNA expression patterns relative to a baseline level are determined to be responsive to the PTSD treatment. Without being bound by theory, it is postulated that the collected data can train the present molecular detection assay to monitor subject responsiveness to a treatment modality.

### Example 4: PTSD Detection Device or Kit

### Example 4.1. PTSD Detection Device

The present example describes a PTSD detection device. The PTSD detection device includes a molecular genomic test that uses quantitative gene expression to detect clinically relevant changes in RNA expression isolated from human blood collected from individuals 18 years and older with an FDA-cleared collection device, such as an RNA PAXgene Tube, or any other suitable sample collection container such as described in the Device section herein. Molecular genomic tests can include the ADAPT Panel, which uses quantitative next generation sequencing (NGS), or any other molecule genomic tests described herein, and measure the expression level of gene transcripts including the genes set forth in TABLE 2. The device is calibrated to measure the change of transcription pattern relative to a predetermined threshold level using the present molecular detection assay described herein. Upon quantification of the transcription pattern, a result report is generated to indicate whether the subject screens positive or negative for PTSD. Positive results for the detection of PTSD warrant referral to a specialty care provider for further evaluation and diagnosis.

### Example 4.2. PTSD Detection Kit

The present example describes a PTSD detection device. A PTSD detection kit include any component or combination of component described in Example 4.1 and instructions for use to detect PTSD.

### Example 5: Use in Diagnosis, Patient Selection and/or Treatment

### Example 5.1. Diagnosis

The present example describes the use of the present molecular detection assay, device, and/or kit in a method of diagnosis of PTSD. A molecular detection assay as described in Examples 2 or 3, a device as described in Example 4.1, or a kit as described in Example 4.1, is used as a screening tool for PTSD. Positive results for the detection of PTSD warrant referral to a specialty care provider for further evaluation and diagnosis.

### Example 5.2. Patient Selection

The present example describes the use of the present molecular detection assay, device, and/or kit in a method of selecting subjects for PTSD treatment. n molecular detection assay as described in Examples 2 or 3, a device as described in Example 4.1, or a kit as described in Example 4.1, is used as a screening tool to determine if a subject would be responsive to PTSD treatment, and optionally, a specific PTSD treatment.

### Example 5.3. Treatment

The present example describes the use of the present molecular detection assay, device, and/or kit in a method of treating PTSD. Upon diagnosing PTSD in a subject, including by using the method described in Example 5.1, and optionally selecting the subject for a treatment, including by using the method described in Example 5.2, such a subject is treated for PTSD using therapeutically effective treatments or using clinically accepted and/or approved treatments. The molecular detection assay as described in Examples 2 or 3, a device as described in Example 4.1, or a kit as described in Example 4.1, is used to monitor the responsiveness of the subject to the treatment and/or to monitor the progress of the subject undergoing treatment.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the methods presented in the disclosure. It is intended that the following claims define the scope of the disclosure and that methods and structures within the scope of these claims and their equivalents be covered thereby.

1. A method for detecting post traumatic stress disorder (PTSD) in a human subject, the method comprising:
a) obtaining a cell sample obtained from a human subject;
b) measuring with a first computer program, executed on a computer, a test gene expression level of one or more PTSD-associated genes from the cell sample, wherein the PTSD-associated genes are selected from the group consisting of: TSPAN5, HIST1H2AE, UBE3A, GPX4, EPB42, SLC4A1, NDUFA1, HIST1HEH, ELOVL7, ALES, COMPT, PDZK1IP1, ITGA2B, CYP4F3, EPB41L3, PRDM1, RP-11-449P15.1, FAS, TUBB2A, and JAM3;
c) with a second computer program, executed on a computer, comparing the test PTSD-associated expression level(s) to one or more control gene expression level(s);
d) determining the differential expression of the one or more test PTSD-associated gene(s); and
e) determining the positive detection of PTSD based on the differential expression.

2.-3. (canceled)

4. The method of claim 1, wherein the test gene expression level(s) and/or the control gene expression level(s) is of 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or all 20 PTSD-associated genes.

5. The method of claim 1, wherein the PTSD-associated genes are selected from the group consisting of TSPAN5, HIST1H2AE, UBE3A, GXP4, EPB42, SLC4A1, NDUFA1, HIST1H3H, EVOLV7, and JAM3.

6. The method of claim 1, wherein the PTSD-associated genes are selected from the group consisting of TSPAN5, HIST1H2AE, UBE3A, GXP4, and EPB42.

7. The method of claim 1, wherein the test PTSD-associated gene expression level(s) is differentially expressed with a p Value of at less than 2.5×10⁻⁶, at less than 9×10⁻⁷, at less than 1.6×10⁻⁷.

8.-9. (canceled)

10. The method of claim 1, wherein the cell sample is obtained from a blood sample, a saliva sample, a buccal smear sample, a cerebrospinal fluid sample, saliva, skin, cerebrospinal fluid, or any combination thereof.

11. The method of claim 1, wherein the test gene expression is a messenger RNA (mRNA), a small interfering RNA (siRNA), a MicroRNA (miRNA), a small nuclear RNA (snRNA), a U spliceosomal RNA (U-RNA), a Small nucleolar RNA (snoRNA), a Piwi-interacting RNA (piRNA), a repeat associated small interfering RNA (rasiRNA), a small rDNA-derived RNA (srRNA), a transfer RNA derived small RNA (tsRNA), a ribosomal RNA derived small RNA (rsRNA), a large non-coding RNA derived small RNA (lncsRNA), or a messenger RNA derived small RNA (msRNA), a gapmer, a mixmer, a double-stranded RNAs (dsRNA), a single stranded RNAi, (ssRNAi), a DNA-directed RNA interference (ddRNAi), or any combination thereof.

12. The method of claim 1, wherein the expression level is assessed on a transcriptional level, or on a translational level.

13. (canceled)

14. The method of claim 1, wherein the first computer program and the second computer program are the same, or different.

15. (canceled)

16. The method of claim 1, wherein the method is performed more than once.

17. The method of claim 16, wherein the method is performed weekly, monthly, bimonthly, three times per year, four times per year, twice per year, or annually.

18. The method of claim 1, further comprising the step of treating the subject for PTSD.

19. The method of claim 18, wherein treating the subject comprises administering to the subject a pharmaceutical therapy, psychotherapy, or a combination thereof.

20. (canceled)

21. The method of claim 17, wherein the pharmaceutical therapy is an SSRI, a tricyclic antidepressant, an MAOI, an antipsychotic, a beta-blocker, a benzodiazepine, a psychedelic, or a combination thereof.

22.-28. (canceled)

29. A method of screening PTSD in a subject, the method comprising:
a. detecting PTSD in a subject comprising the method of claim 1; and
b. determining said subject should be clinically diagnosed for PTSD based on the positive detection of PTSD in the subject.

30.-39. (canceled)

40. A method of selecting an eligible subject for the treatment of PTSD, the method comprising:
a. detecting PTSD in a subject comprising the method of claim 1; and
b. determining said subject is eligible for PTSD treatment based on the positive detection of PTSD in the subject.

41. (canceled)

42. A method of evaluating treatment efficacy of a subject undergoing PTSD treatment, the method comprising
c. detecting PTSD in a subject comprising the method of claim 1; and
d. determining the efficacy of the PTSD treatment based on the detection of PTSD in the subject, wherein a negative PTSD detection indicates the PTSD treatment is efficacious.

43.-47. (canceled)

48. A method of determining prevalence of PTSD in a population of interest, the method comprising
a. detecting PTSD in each subject in a population of interest comprising the method of claim 1; and
b. determining the prevalence of PTSD in said population of interest based on the positive detection of PTSD in each subject of the population of interest.

49. (canceled)
